(19) United States
(12) Patent Application Publication
(10) Pub. No.: US 2025/0082633 A1
(43) Pub. Date: Mar. 13, 2025

(54) COMPOUNDS AND METHODS FOR MODULATING SPLICING

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen t. Hopper, Lexington, MA (US); Stepan Vyskocil, Benesov (CZ); Benoit Moreau, Newton, MA (US)

(21) Appl. No.: 18/713,561

(22) PCT Filed: Nov. 23, 2022
(86) PCT No.: PCT/US2022/050914

§ 371 Date: May 24, 2024

Related U.S. Application Data
(60) Provisional application No. 63/283,127, filed on Nov. 24, 2021.

(51) Int. Cl. A61K 31/5025; A61K 31/437; A61K 31/438; A61K 31/454; A61K 31/4545; A61K 31/4725; A61K 31/496; A61K 31/4985; A61P 25/28; A61P 35/00; C07D 401/14; C07D 403/04; C07D 403/14; C07D 413/04; C07D 413/14; C07D 417/14; C07D 471/04; C07D 487/04; C07D 519/00

(52) U.S. Cl. CPC A61K 31/5025; A61K 31/437; A61K 31/438; A61K 31/454; A61K 31/4545; A61K 31/4725; A61K 31/496; A61K 31/4985; A61P 25/28; A61P 35/00; C07D 401/14; C07D 403/04; C07D 403/14; C07D 413/04; C07D 413/14; C07D 417/14; C07D 471/04; C07D 487/04; C07D 519/00

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/283,127, filed on Nov. 24, 2021, the contents of which is incorporated herein by reference in its entirety.

### BACKGROUND

Alternative splicing is a major source of protein diversity in higher eukaryotes and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I) (e.g., a compound of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) and pharmaceutically acceptable salts, solvates, hydrates, tautomers, or stereoisomers thereof. The present disclosure additionally provides methods of using the compounds of the invention (e.g., compounds of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formulas (I), ((I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v), and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

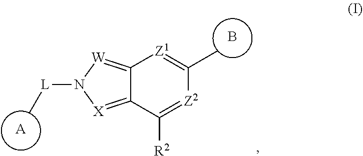

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L is absent, C₁-C₆-alkylene, C₁-C₆-heteroalkylene, C(O), or —C(O)N(R^{B})—, wherein each alkylene and heteroalkylene is optionally substituted with one or more R⁸; W, X, Z¹, and Z² are each independently C(R³) or N, wherein at least one of W and X is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; R² is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ and R⁸ is independently C₁-C₆-alkyl, halo, cyano, oxo, or —OR^{A1}; each R^{A1} is hydrogen or C₁-C₆-alkyl; and x is 0, 1, or 2.

In another aspect, the present invention provides pharmaceutical compositions comprising a compound of Formula (I) (e.g., a compound of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formula (I) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formula (I) alters a target nucleic acid (e.g., DNA or RNA, e.g., a precursor RNA, e.g., a pre-mRNA), target protein, or combination thereof. In some embodiments, the compound of Formula (I) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue). In some embodiments, the presence of a compound of Formula (I) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formula (I), e.g., in a healthy or diseased cell or tissue).

In another aspect, the present disclosure provides methods for preventing and/or treating a disease, disorder, or condition in a subject by administering a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formula (I) (e.g., a compound of Formulas (I), ((I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. In another aspect, the present disclosure provides methods of altering the isoform of a target protein with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure provides compositions for use in preventing and/or treating a disease, disorder, or condition in a subject by administering a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. In another aspect, the present disclosure provides compositions for use in altering the isoform of a target protein with a compound of Formula (I) (e.g., a compound of Formulas (I), (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formula (I) to a biological sample, a cell, or a subject comprises inhibition of cell growth or induction of cell death.

In another aspect, the present disclosure features kits comprising a container with a compound of Formula (I) (e.g., a compound of Formulas (I), ((I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v)) or a pharmaceutically acceptable salt, solvate, hydrate, tautomer, stereoisomer thereof, or a pharmaceutical composition thereof. In certain embodiments, the kits described herein further include instructions for administering the compound of Formula (I) or the pharmaceutically acceptable salt, solvate, hydrate, tautomer, stereoisomer thereof, or the pharmaceutical composition thereof.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀ alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—CI₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized.

The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₈), cubanyl (C₈), bicyclo[1.1.1]pentanyl (C₅), bicyclo[2.2.2]octanyl (C₈), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 8 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5 H₂O)), and polyhydrates (x is a number greater than 1, e.g., dihydrates (R·2 H₂O) and hexahydrates (R·6 H₂O)).

The term “tautomer” refers to compounds that are interchangeable forms of a particular compound structure, and that vary in the displacement of hydrogen atoms and electrons. Thus, two structures may be in equilibrium through the movement of π electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formula (I)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formula (I)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

The present disclosure features a compound of Formula (I):

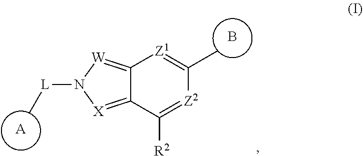

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; L is absent, C₁-C₆-alkylene, C₁-C₆-heteroalkylene, C(O), or —C(O)N(R^{B})—, wherein each alkylene and heteroalkylene is optionally substituted with one or more R⁸; W, X, Z¹, and Z² are each independently C(R³) or N, wherein at least one of W and X is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; R² is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ and R⁸ is independently C₁-C₆-alkyl, halo, cyano, oxo, or —OR^{A1}; each R^{A1} is hydrogen or C₁-C₆-alkyl; and x is 0, 1, or 2

As generally described herein, A and B, are each independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹.

In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

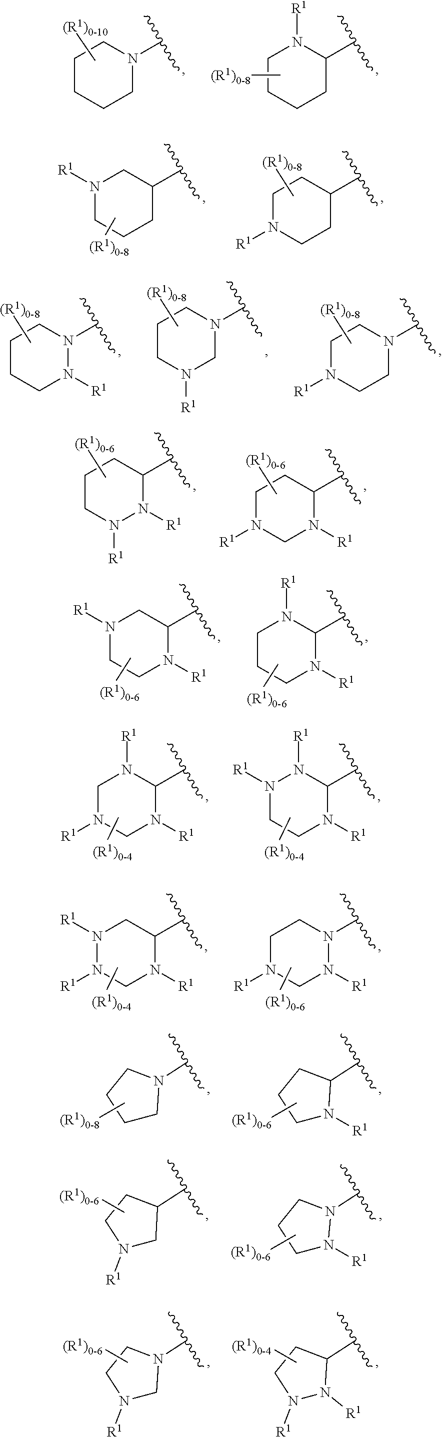

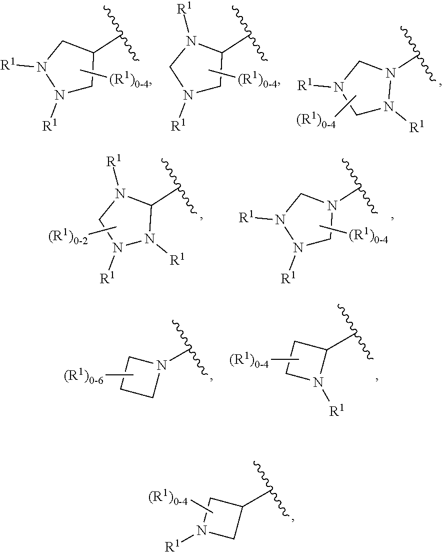

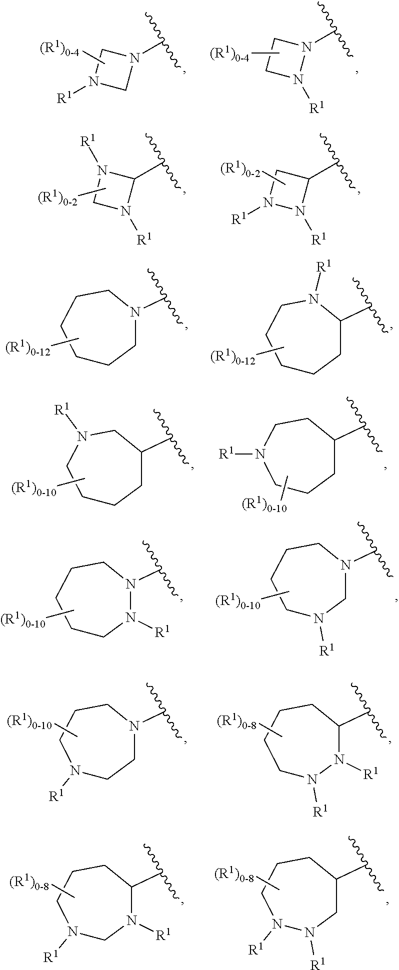

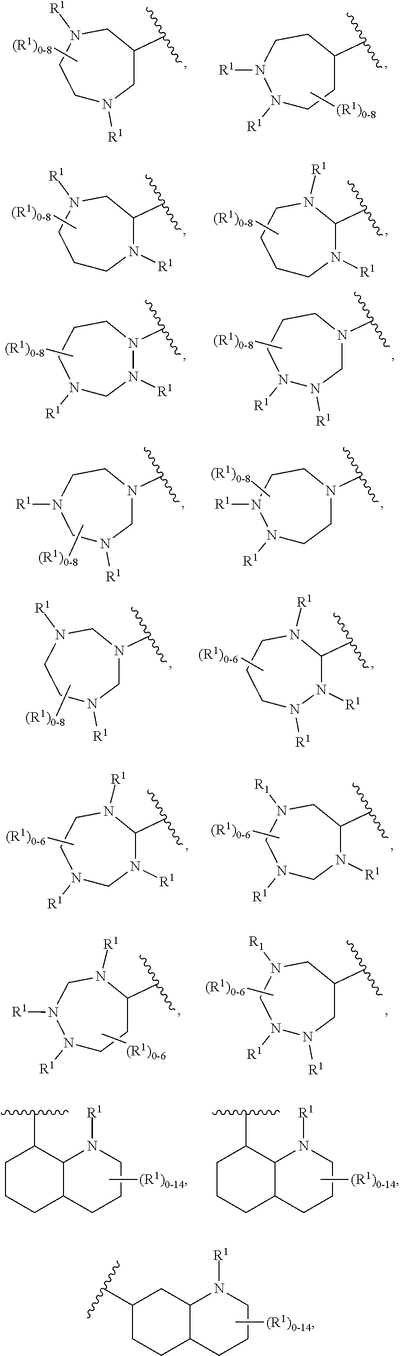

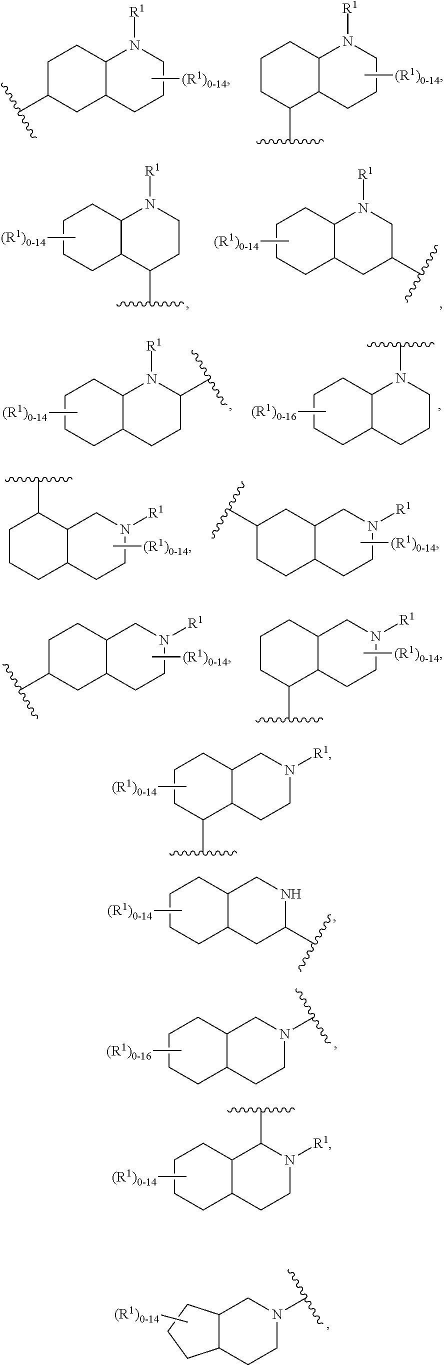

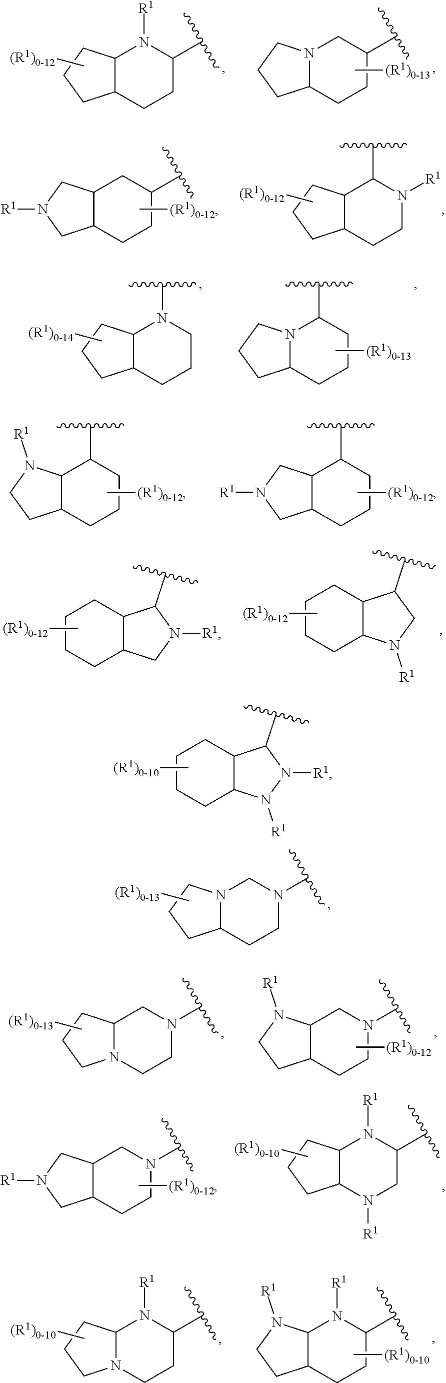

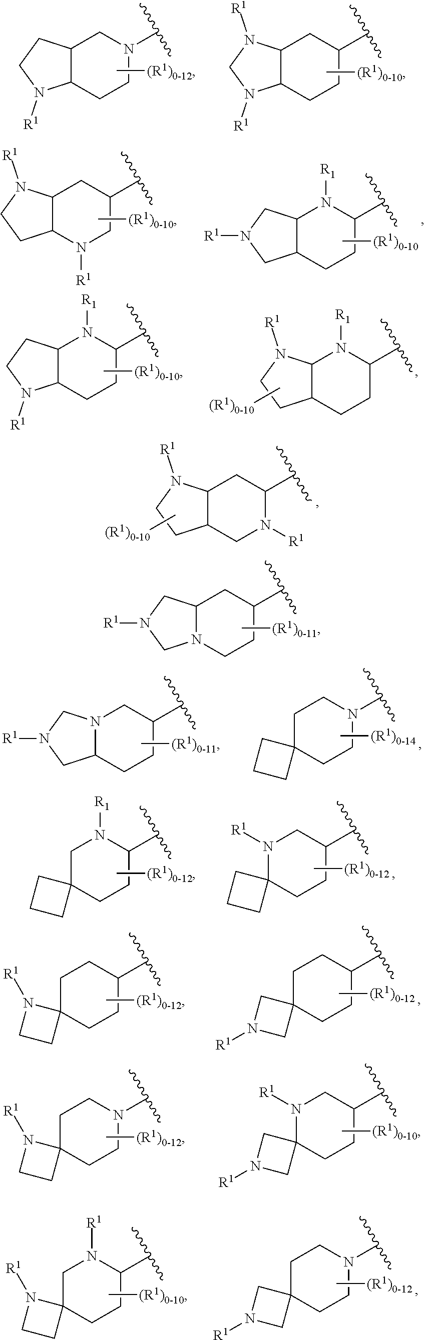

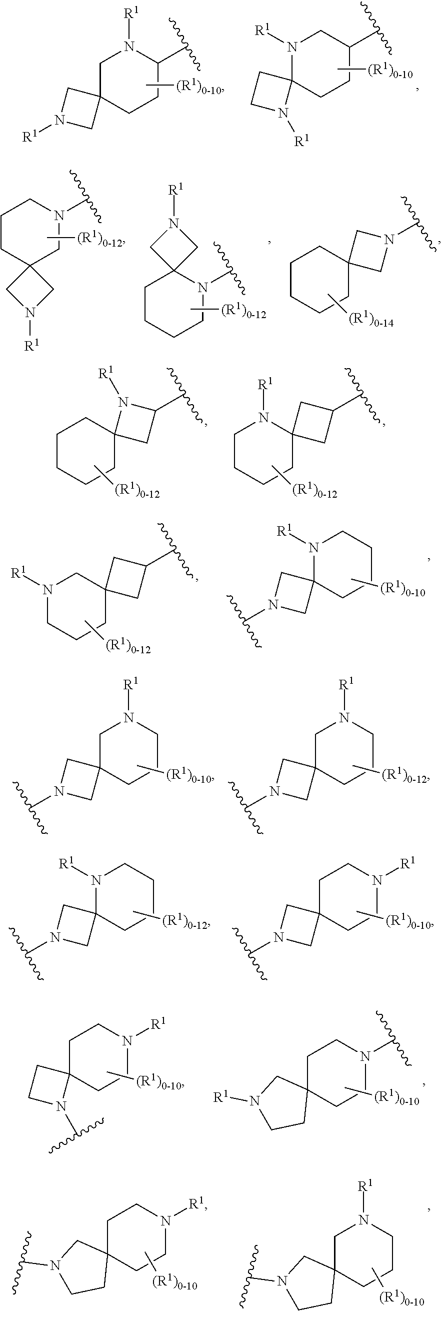

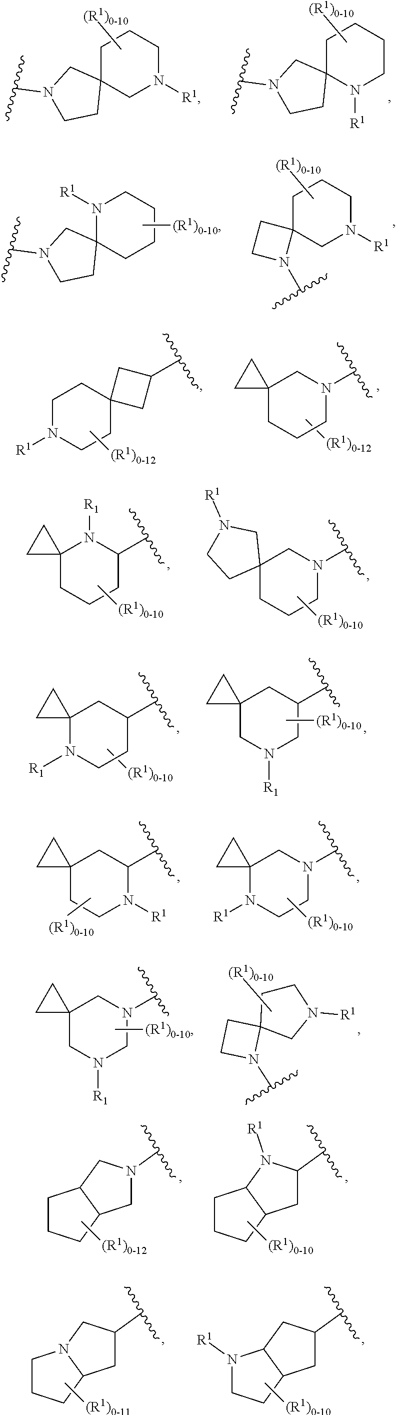

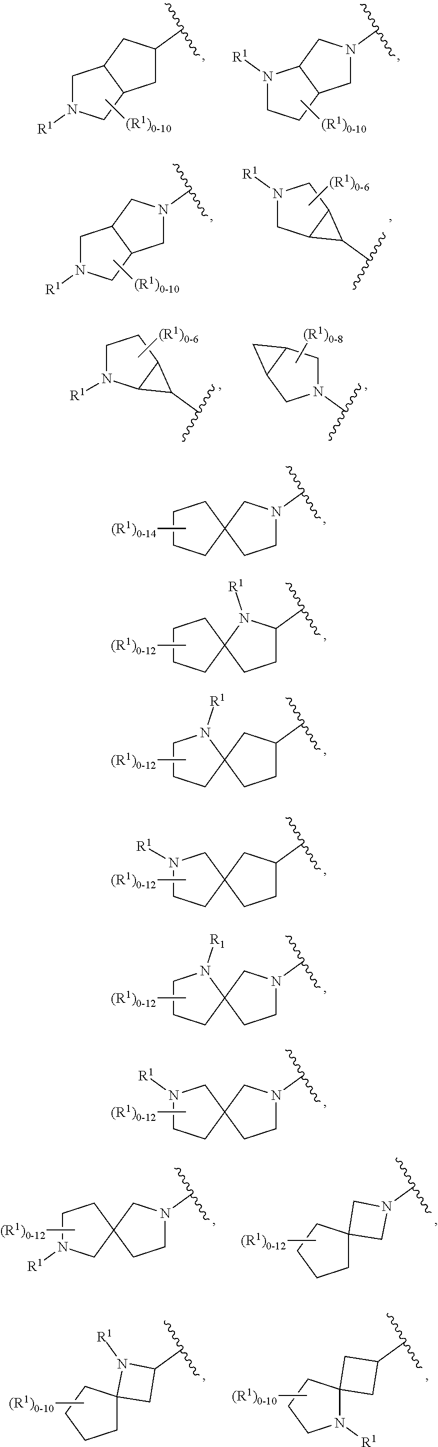

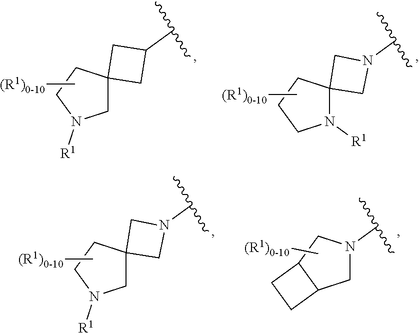

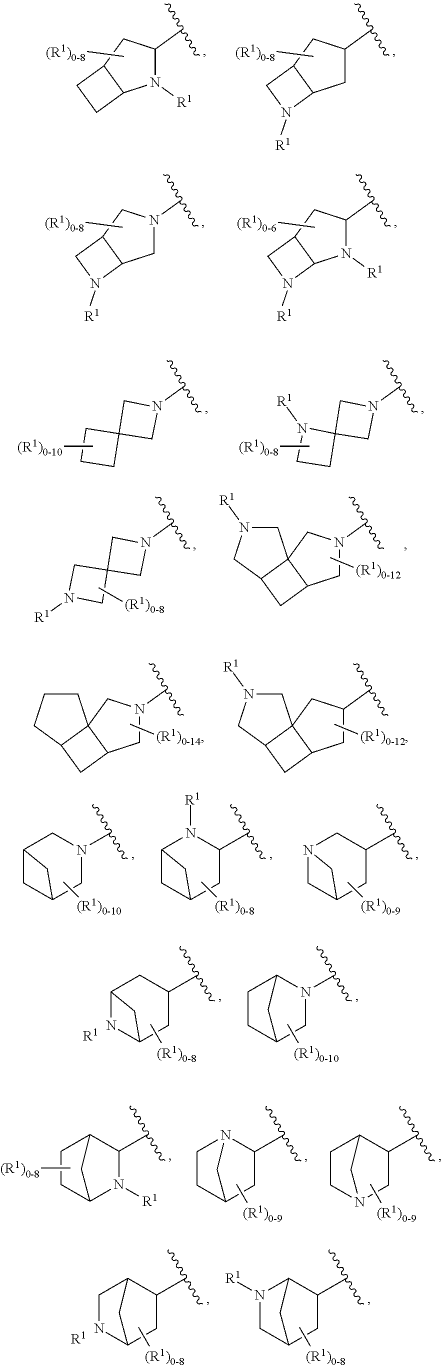

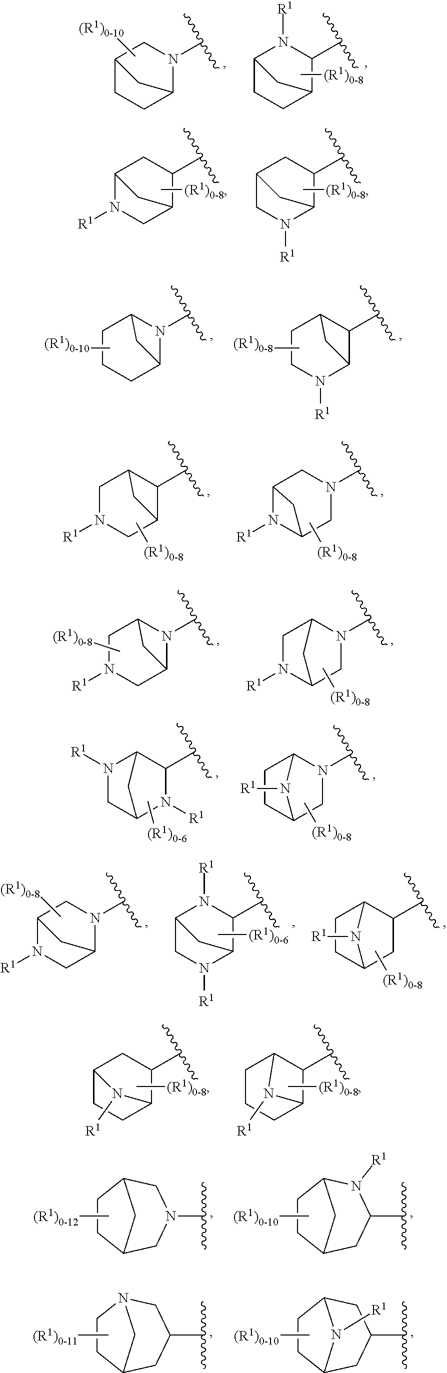

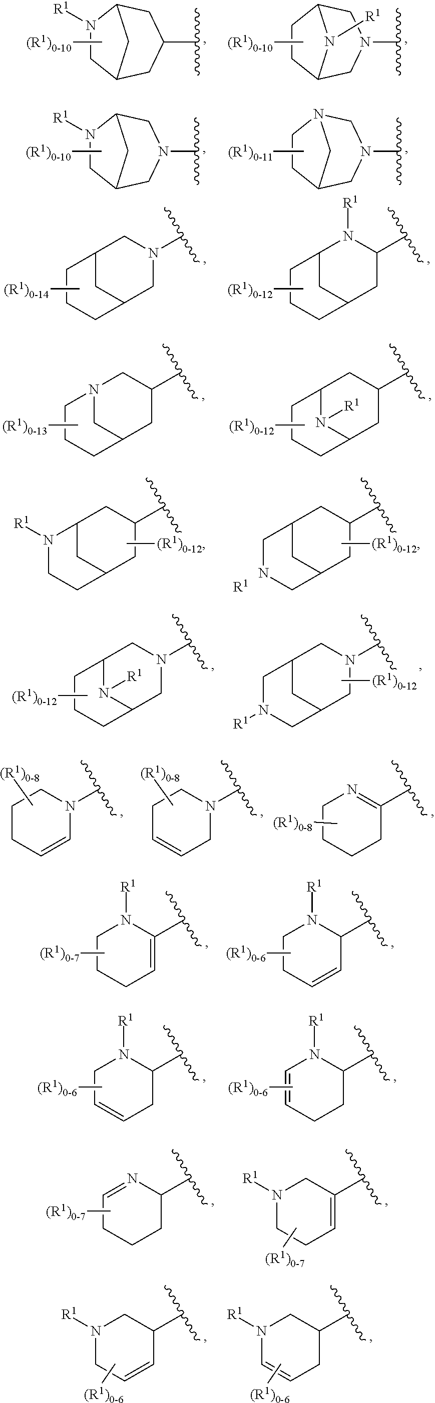

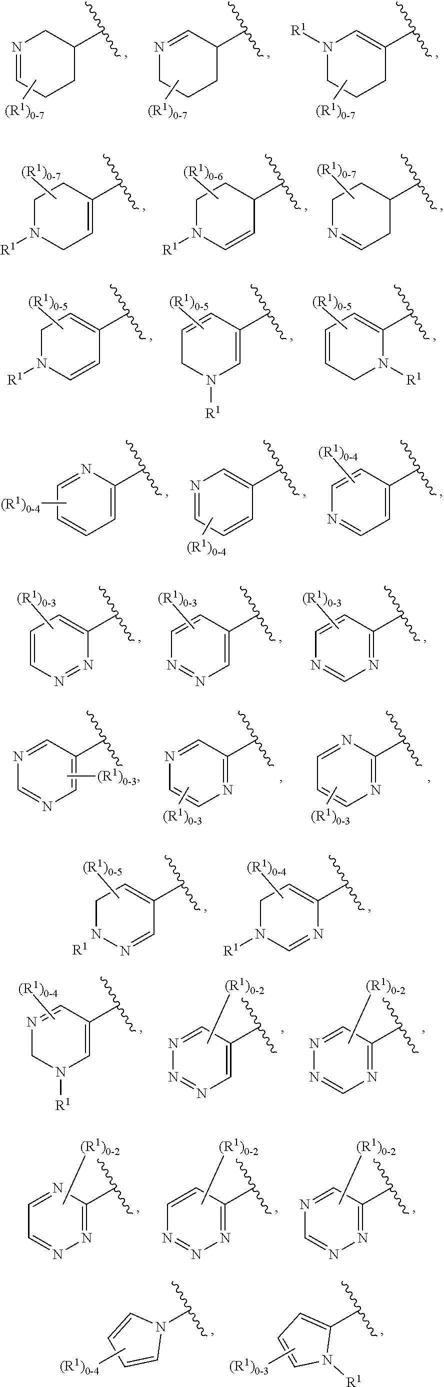

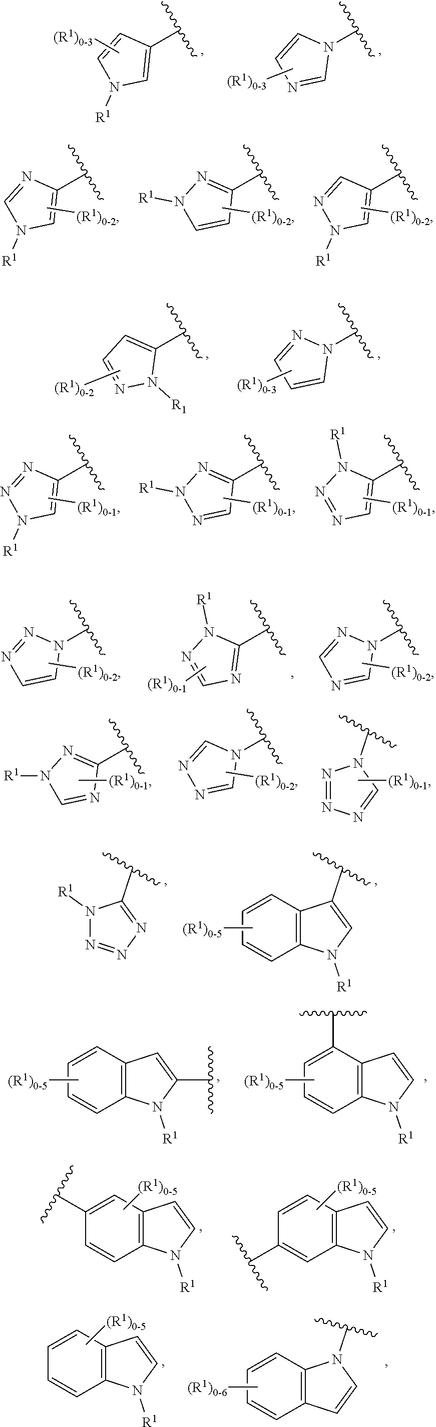

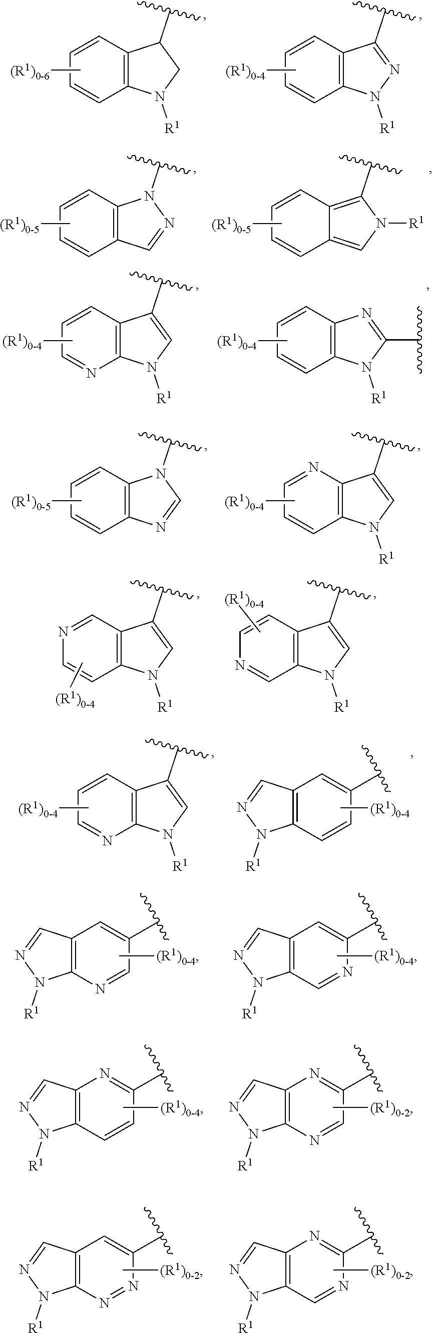

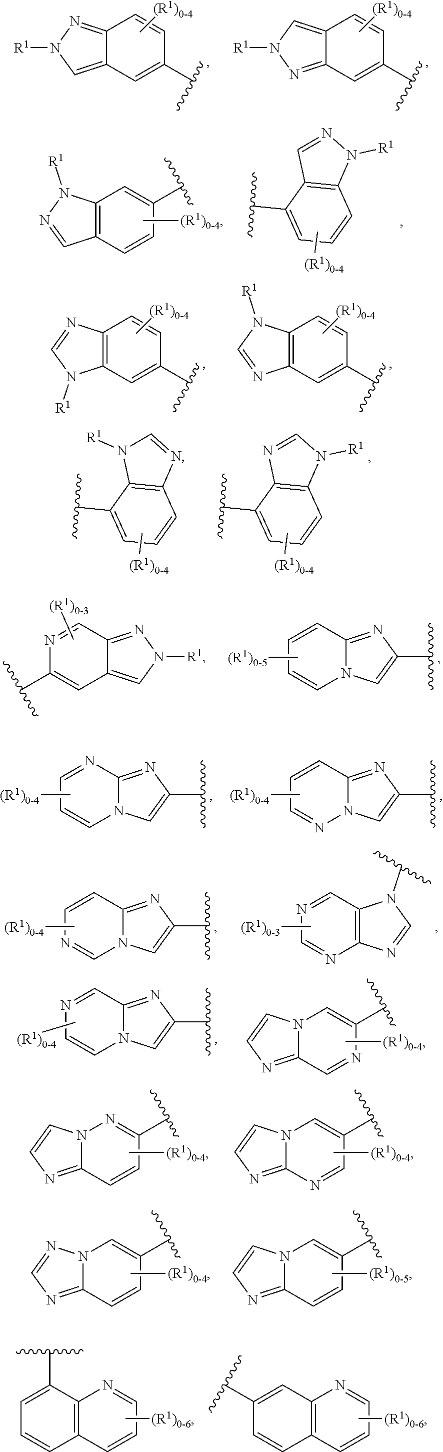

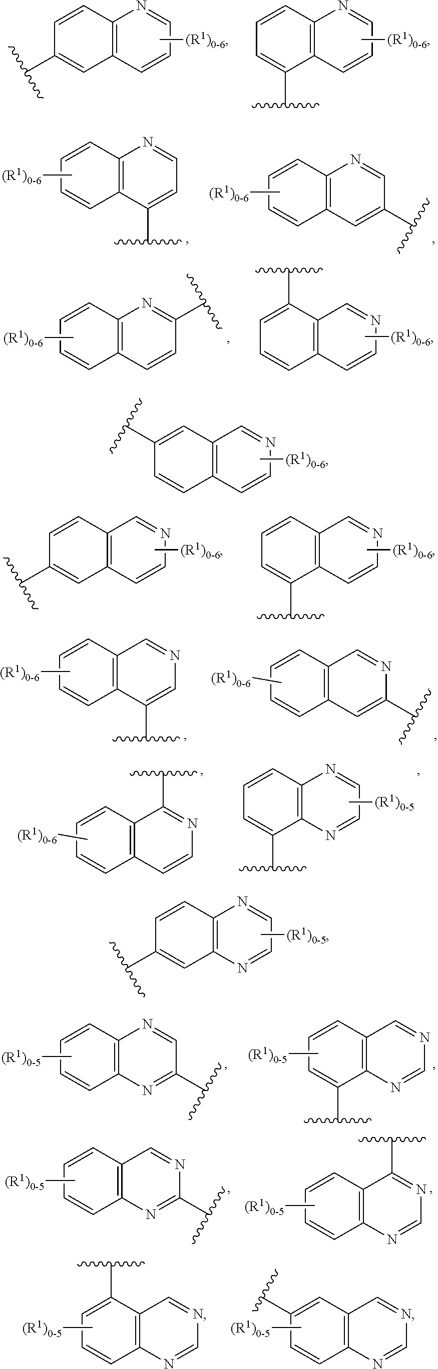

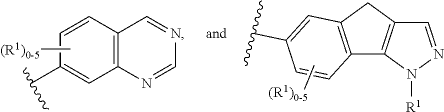

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

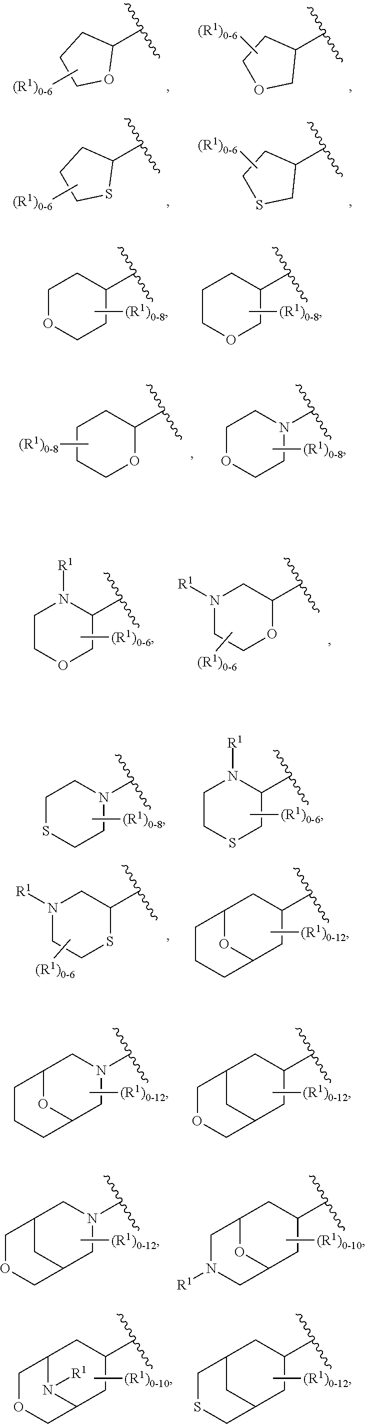

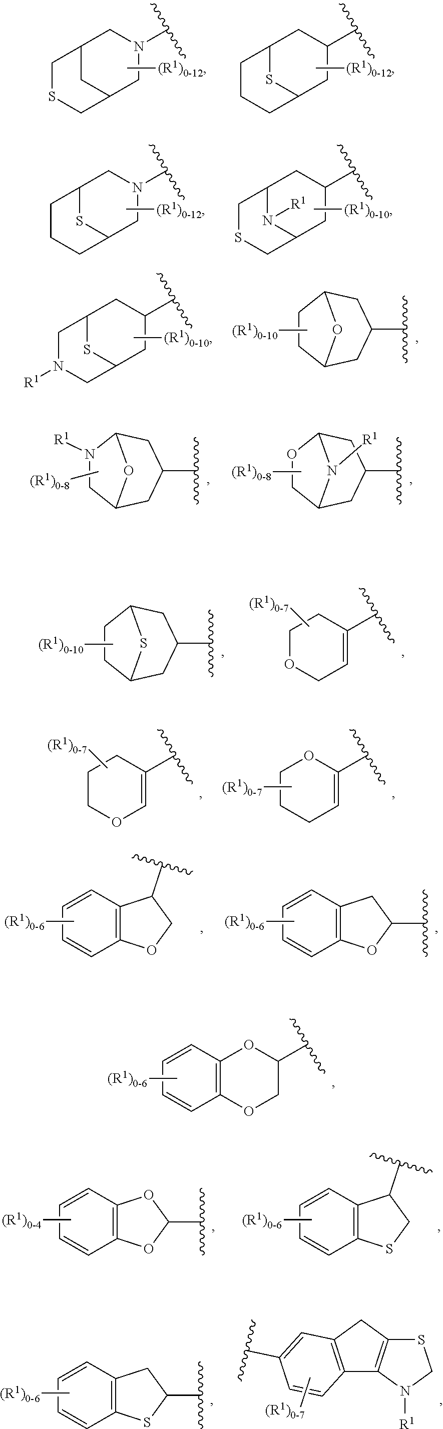

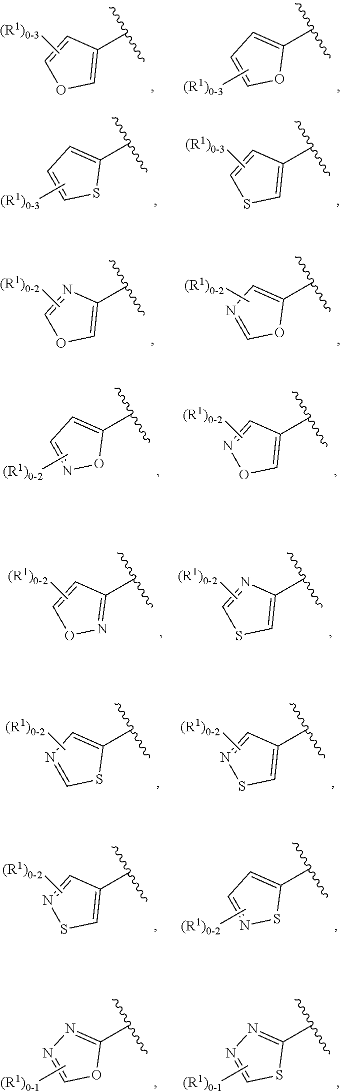

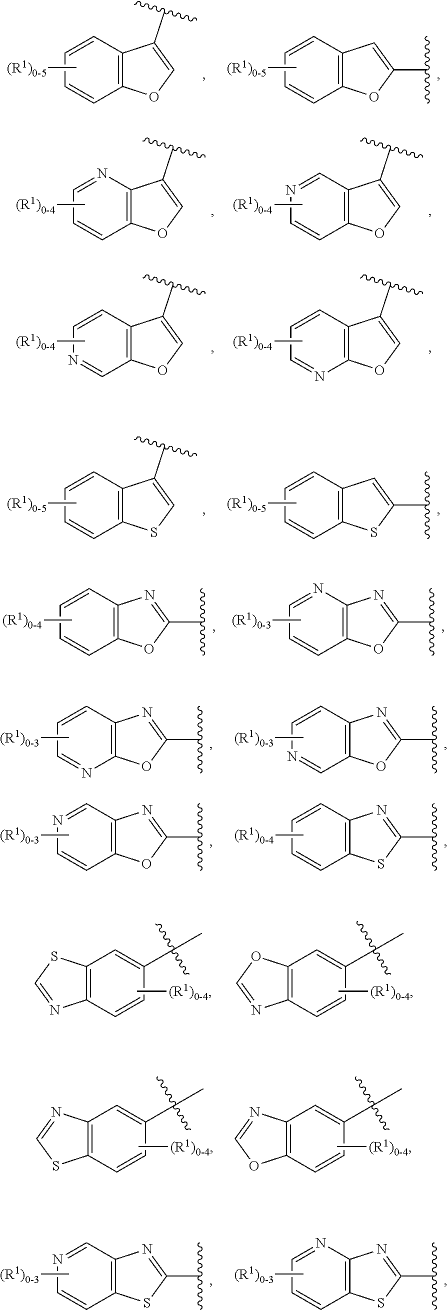

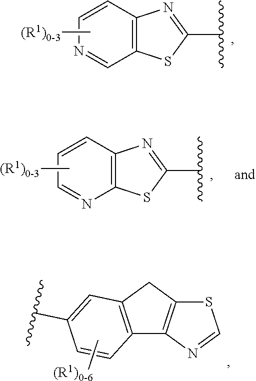

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, one of A and B is independently selected from

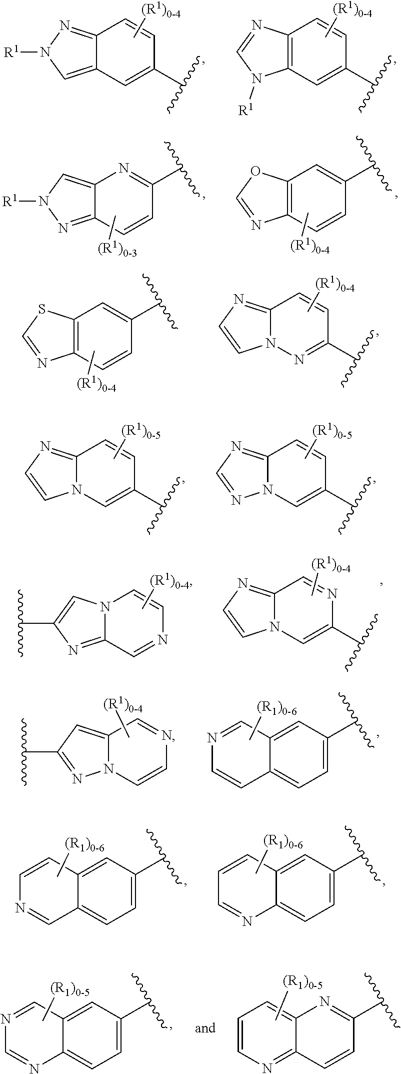

wherein R¹ is as described herein.
In some embodiments, one of A and B is independently selected from

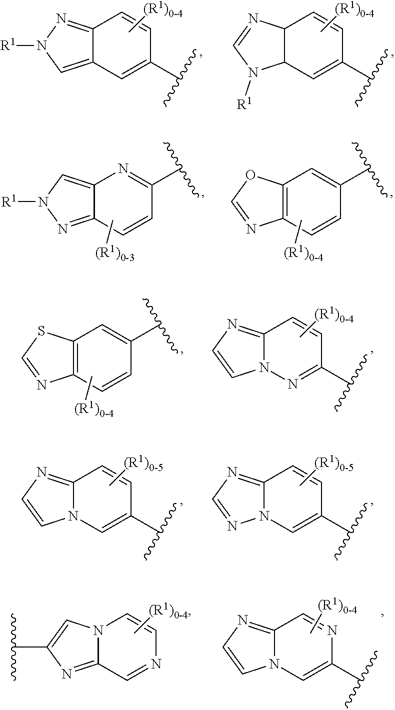

and wherein R¹ is as described herein. In some embodiments, one of A and B is independently selected from

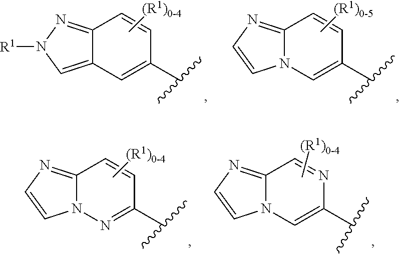

wherein each R¹ is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁴. In some embodiments, one of A and B is independently selected from

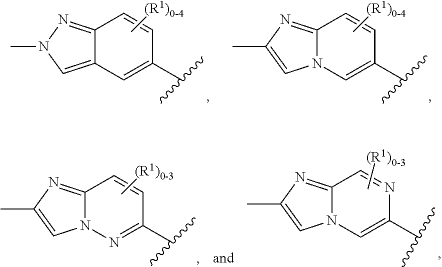

wherein each R¹ is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, one of A and B is independently

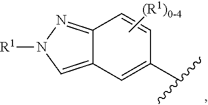

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷. In some embodiments, one of A and B is independently selected from

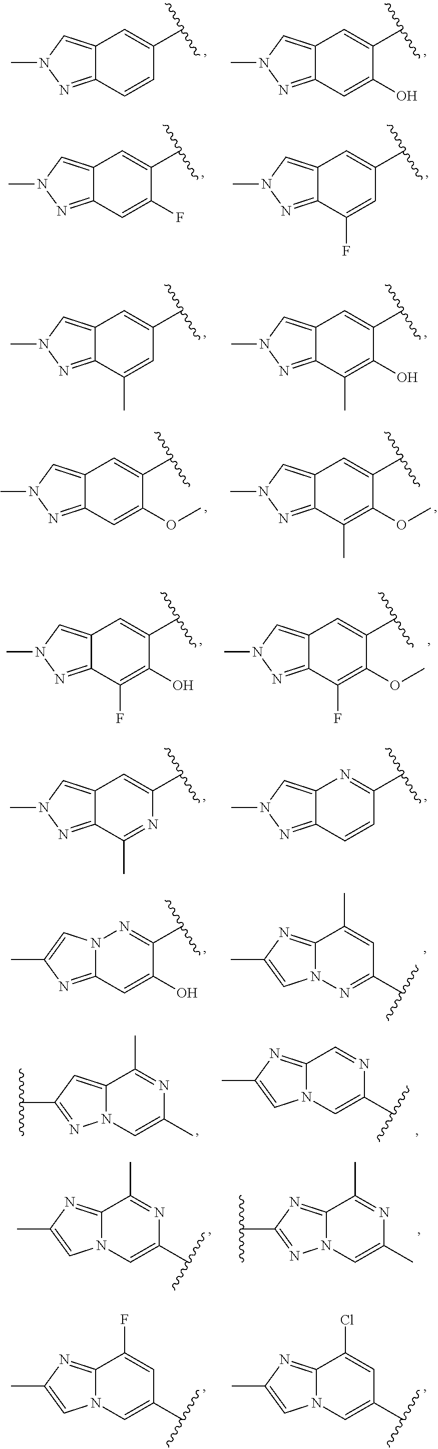

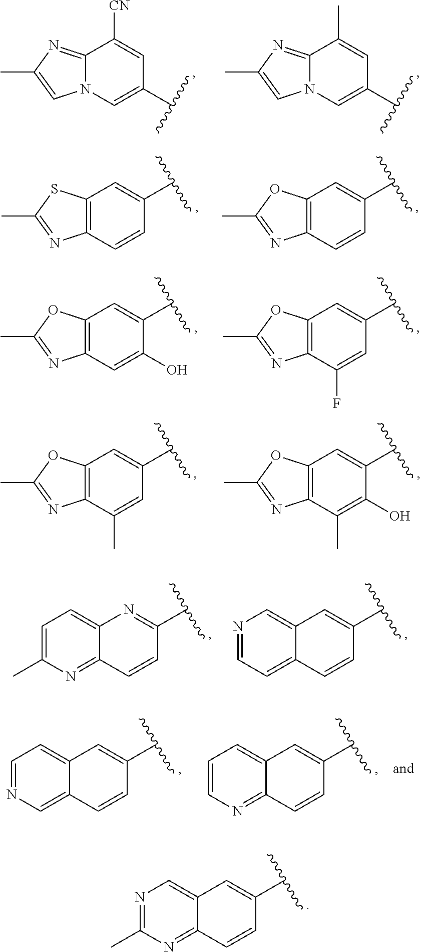

In some embodiments, one of A and B is independently selected from

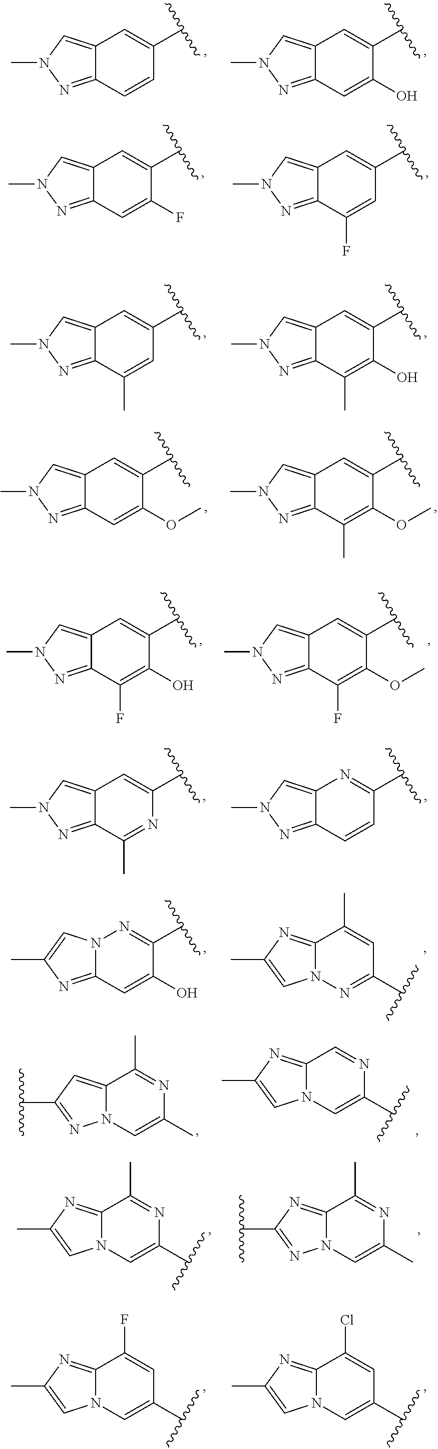

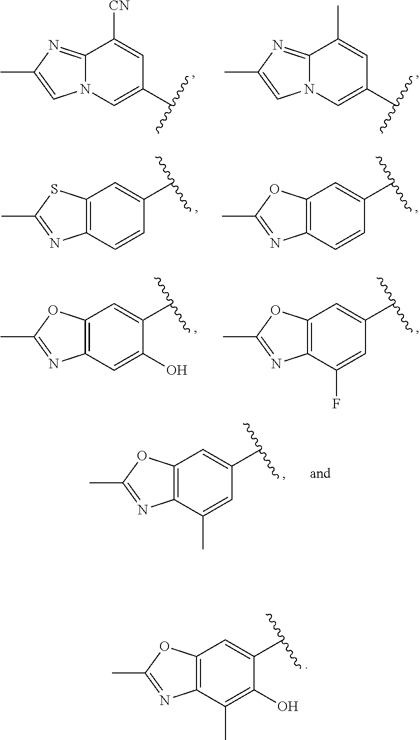

In some embodiments, one of A and B is independently selected from

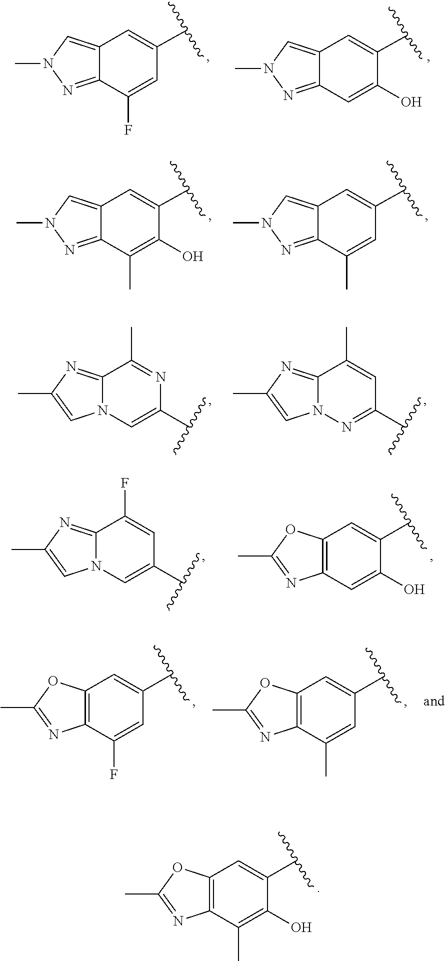

In some embodiments, one of A and B is independently

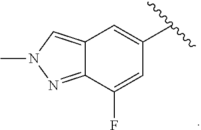 [SMILES: *c1cc(F)c2nn(C)cc2c1]

In some embodiments, one of A and B is independently a monocyclic heterocyclyl or bicyclic heterocyclyl, each of which is optionally substituted with one or more R¹. In some embodiments, one of A and B is independently a nitrogen-containing heterocyclyl optionally substituted with one or more R¹. In some embodiments, one of A and B is independently a 4-8 membered heterocyclyl optionally substituted with one or more R¹. In some embodiments, one of A and B is independently selected from

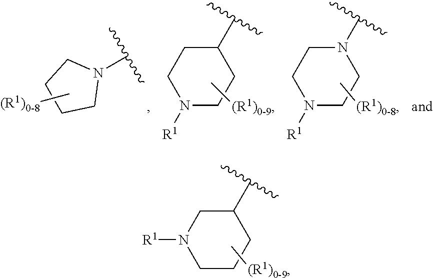

wherein R¹ is as described herein. In some embodiments, one of A and B is independently selected from

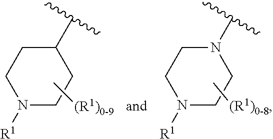

wherein R¹ is as described herein. In some embodiments, one of A and B is

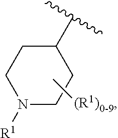

wherein R¹ is as described herein. In some embodiments, A is selected from

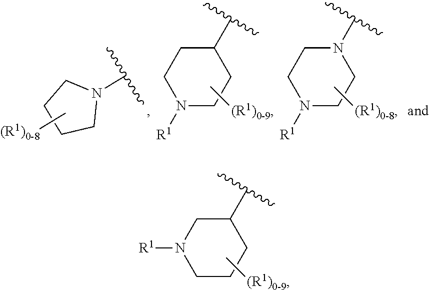

wherein R¹ is as described herein. In some embodiments, B is selected from

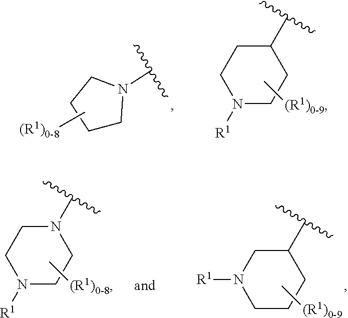

wherein R¹ is as described herein. In some embodiments, B is selected from

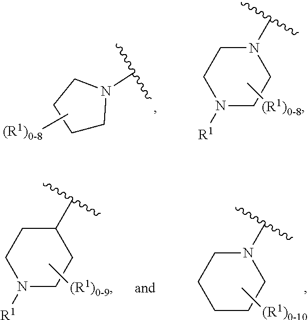

wherein R¹ is as described herein.
In some embodiments, one of A and B is independently selected from

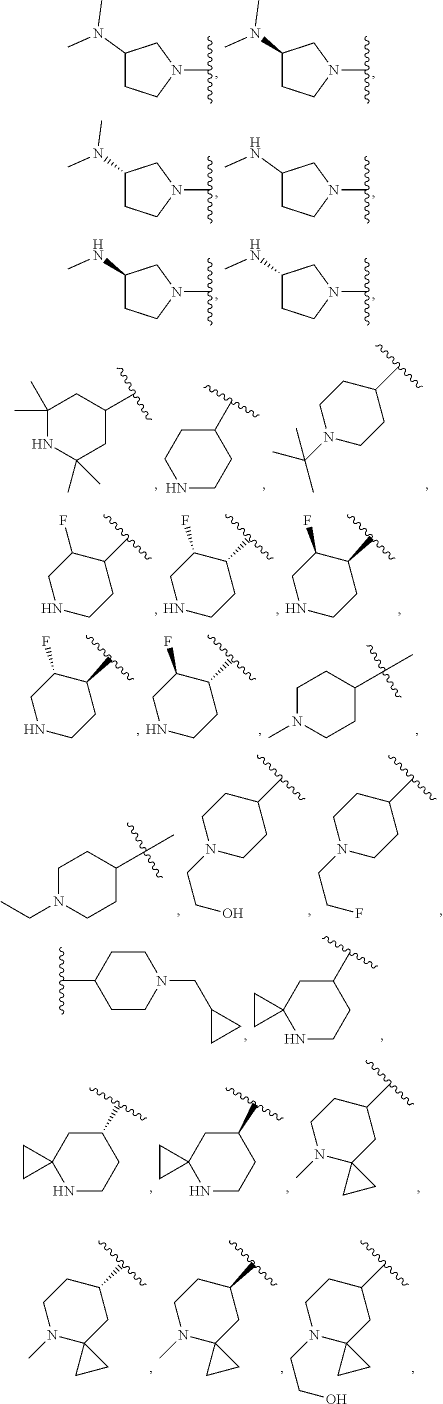

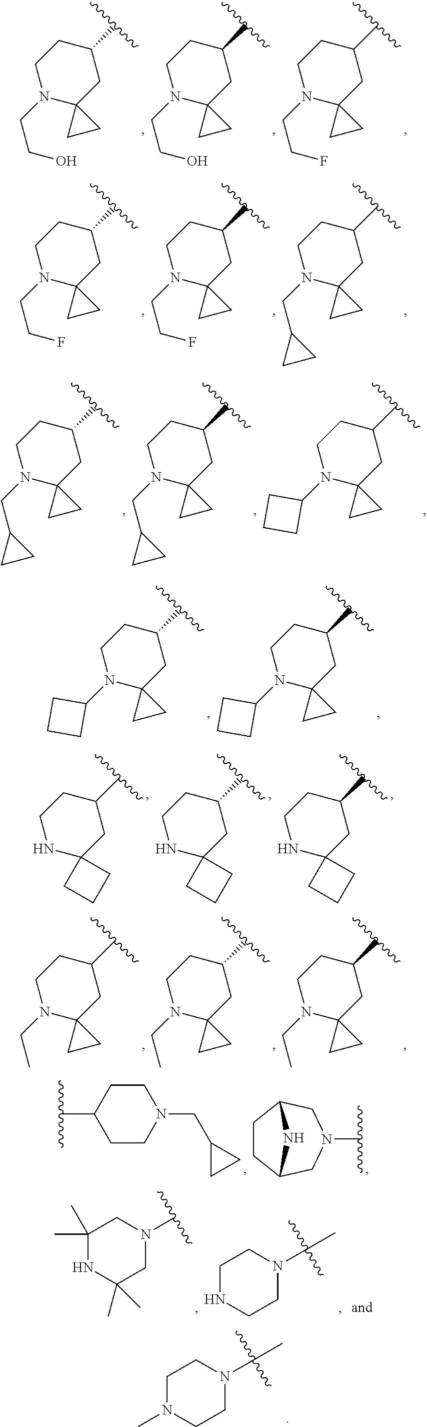

In some embodiments, one of A and B is independently selected from

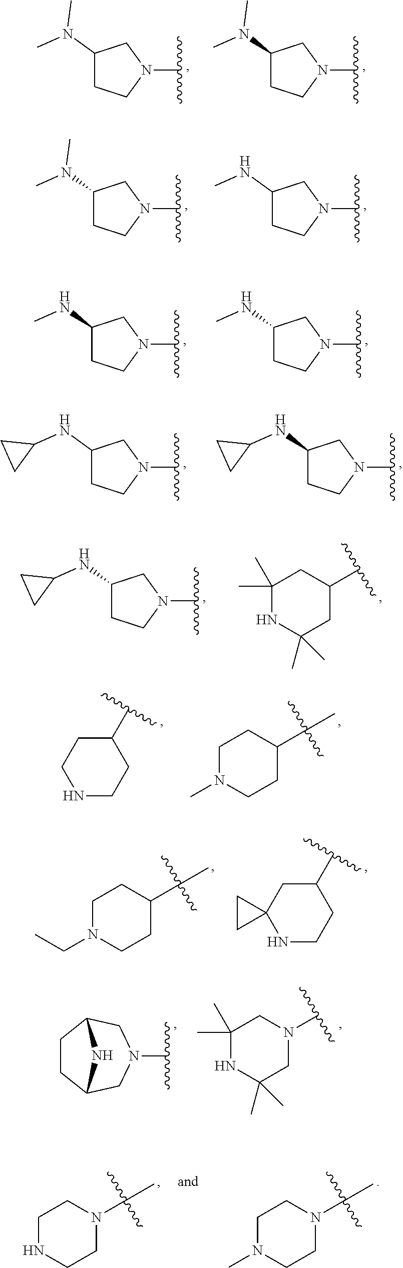

In some embodiments, A is selected one of A and B is independently selected from

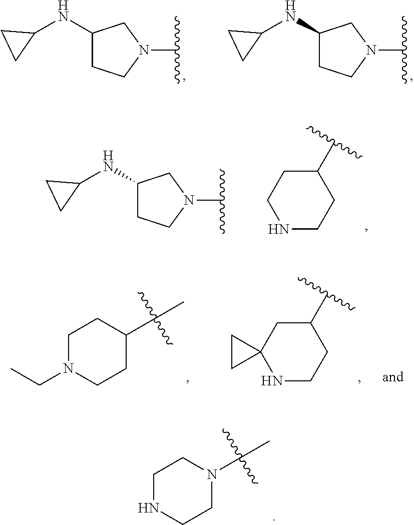

In some embodiments, one of A and B is

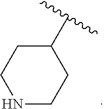 [SMILES: *C1CCNCC1]

In some embodiments, X is N. In some embodiments, W is N. In some embodiments, both of W and X is independently N. In some embodiments, Z¹ is C(R³) (e.g., CH). In some embodiments, Z¹ is N. In some embodiments, X is N and W, Z¹, and Z² are each independently C(R³). In some embodiments, X and Z¹ are each independently N and W and Z² are C(R³). In some embodiments, X and W are each independently N and Z¹ is C(R³). In some embodiments, each of W, X, and Z¹ are each independently N, and Z¹ is C(R³).

In some embodiments, R¹ is hydrogen. In some embodiments, R¹ is C₁-C₆-alkyl. In some embodiments, R¹ is C₂-C₆-alkenyl. In some embodiments, R¹ is C₂-C₆-alkynyl. In some embodiments, R¹ is C₁-C₆-heteroalkyl. In some embodiments, R¹ is C₁-C₆-haloalkyl (e.g., —CF₃). In some embodiments, R¹ is C₁-alkyl (e.g., methyl). In some embodiments, R¹ is unsubstituted C₁-C₆-alkyl, unsubstituted C₂-C₆-alkenyl, unsubstituted C₂-C₆-alkynyl, unsubstituted C₁-C₆-heteroalkyl, or unsubstituted C₁-C₆-haloalkyl. In some embodiments, R¹ is C₁-C₆-alkyl substituted with one or more R⁵. In some embodiments, R¹ is C₂-C₆-alkenyl substituted with one or more R⁵. In some embodiments, R¹ is C₂-C₆-alkynyl substituted with one or more R⁵. In some embodiments, R¹ is C₁-C₆-heteroalkyl substituted with one or more R⁵. In some embodiments, R¹ is C₁-C₆-haloalkyl substituted with one or more R⁵. In some embodiments, R¹ is methyl.

In some embodiments, R¹ is cycloalkyl (e.g., 3-7 membered cycloalkyl). In some embodiments, R¹ is heterocyclyl (e.g., 3-7 membered heterocyclyl). In some embodiments, R¹ is aryl. In some embodiments, R¹ is C₁-C₆ alkylene-aryl (e.g., benzyl). In some embodiments, R¹ is C₁-C₆ alkenylene-aryl. In some embodiments, R¹ is C₁-C₆ alkylene-heteroaryl. In some embodiments, R¹ is heteroaryl. In some embodiments, R¹ is unsubstituted cycloalkyl, unsubstituted heterocyclyl, unsubstituted aryl, unsubstituted C₁-C₆ alkylene-aryl, unsubstituted C₁-C₆ alkenylene-aryl, unsubstituted C₁-C₆ alkylene-heteroaryl, or unsubstituted heteroaryl. In some embodiments, R¹ is cycloalkyl substituted with one or more R⁵. In some embodiments, R¹ is heterocyclyl substituted with one or more R⁵. In some embodiments, R¹ is aryl substituted with one or more R⁵. In some embodiments, R¹ is C₁-C₆ alkylene-aryl substituted with one or more R⁵. In some embodiments, R¹ is C₁-C₆ alkenylene-aryl substituted with one or more R⁵. In some embodiments, R¹ is C₁-C₆ alkylene-heteroaryl substituted with one or more R⁵. In some embodiments, R¹ is heteroaryl substituted with one or more R⁵.

In some embodiments, R¹ is —OR^{A}. In some embodiments, R¹ is —NR^{B}R^{C} (e.g., NH₂ or NMe₂). In some embodiments, R¹ is —NR^{B}C(O)R^{D}. In some embodiments, R¹ is-C(O)NR^{B}R^{C}. In some embodiments, R¹ is —C(O)R^{D}. In some embodiments, R¹ is —C(O)OR^{D}. In some embodiments, R¹ is —SR^{E}. In some embodiments, R¹ is —S(O)ₓR^{D}. In some embodiments, R¹ is halo, e.g., fluoro, chloro, bromo, or iodo. In some embodiments, R¹ is cyano. In some embodiments, R¹ is nitro (—NO₂). In some embodiments, R¹ is oxo.

In some embodiments, two R¹ groups, together with the atoms to which they are attached, form a 3-7-membered cycloalkyl. In some embodiments, two R¹ groups, together with the atoms to which they are attached, form a 3-7-membered heterocyclyl. In some embodiments, two R¹ groups, together with the atoms to which they are attached, form a 5- or 6-membered aryl. In some embodiments, two R¹ groups, together with the atoms to which they are attached, form a 5- or 6-membered heteroaryl. The cycloalkyl, heterocyclyl, aryl, or heteroaryl may be substituted with one or more R⁵.

In some embodiments, R³ is hydrogen. In some embodiments, R³ is C₁-C₆ alkyl. In some embodiments, R³ is C₁-C₆ haloalkyl. In some embodiments, R³ is methyl.

In some embodiments, L is absent. In some embodiments, L is C₁-C₆-alkylene optionally substituted with one or more R⁸. In some embodiments, L is C₁-C₆-heteroalkylene optionally substituted with one or more R⁸. In some embodiments, L is C(O). In some embodiments, L is —C(O)N(R^{B})—.

In some embodiments, R⁵ is C₁-C₆-alkyl. In some embodiments, R⁵ is C₂-C₆-alkenyl. In some embodiments, R⁵ is C₂-C₆-alkynyl. In some embodiments, R⁵ is C₁-C₆-heteroalkyl. In some embodiments, R⁵ is C₁-C₆-haloalkyl. In some embodiments, R⁵ is unsubstituted C₁-C₆-alkyl, unsubstituted C₂-C₆-alkenyl, unsubstituted C₂-C₆-alkynyl, unsubstituted C₁-C₆-haloalkyl, or unsubstituted C₁-C₆-heteroalkyl. In some embodiments, R⁵ is C₁-C₆-alkyl substituted with one or more R⁶. In some embodiments, R⁵ is C₂-C₆-alkenyl substituted with one or more R⁶. In some embodiments, R⁵ is C₂-C₆-alkynyl substituted with one or more R⁶. In some embodiments, R⁵ is C₁-C₆-haloalkyl substituted with one or more R⁶. In some embodiments, R⁵ is C₁-C₆-heteroalkyl substituted with one or more R⁶.

In some embodiments, R⁵ is cycloalkyl. In some embodiments, R⁵ is heterocyclyl. In some embodiments, R⁵ is aryl. In some embodiments, R⁵ is heteroaryl. In some embodiments, R⁵ is unsubstituted cycloalkyl, unsubstituted heterocyclyl, unsubstituted aryl, or unsubstituted heteroaryl. In some embodiments, R⁵ is cycloalkyl substituted with one or more R⁶. In some embodiments, R⁵ is heterocyclyl substituted with one or more R⁶. In some embodiments, R⁵ is aryl substituted with one or more R⁶. In some embodiments, R⁵ is heteroaryl substituted with one or more R⁶.

In some embodiments, R⁵ is halo (e.g., fluoro, chloro, bromo, or iodo). In some embodiments, R⁵ is cyano. In some embodiments, R⁵ is oxo. In some embodiments, R⁵ is —OR^{A}. In some embodiments, R⁵ is —NR^{B}R^{C}. In some embodiments, R⁵ is —NR^{B}C(O)R^{D}. In some embodiments, R⁵ is —NO₂. In some embodiments, R⁵ is —C(O)NR^{B}R^{C}. In some embodiments, R⁵ is —C(O)R^{D}. In some embodiments, R⁵ is —C(O)OR^{D}. In some embodiments, R⁵ is —SR^{E}. In some embodiments, R⁵ is —S(O)ₓR^{D}.

In some embodiments, R⁶ is C₁-C₆-alkyl. In some embodiments, R⁶ is C₁-C₆-heteroalkyl. In some embodiments, R⁶ is C₁-C₆-haloalkyl (e.g., —CF₃ or —CHF₂). In some embodiments, R⁶ is cycloalkyl. In some embodiments, R⁶ is heterocyclyl. In some embodiments, R⁶ is aryl. In some embodiments, R⁶ is heteroaryl. In some embodiments, R⁶ is halo. In some embodiments, R⁶ is cyano. In some embodiments, R⁶ is oxo. In some embodiments, R⁶ is —OR^{A}.

In some embodiments, R^{A} is hydrogen. In some embodiments, R^{A} is C₁-C₆ alkyl (e.g., methyl). In some embodiments, R^{A} is C₁-C₆ haloalkyl. In some embodiments, R^{A} is aryl. In some embodiments, R^{A} is heteroaryl. In some embodiments, R^{A} is C₁-C₆ alkylene-aryl (e.g., benzyl). In some embodiments, R^{A} is C₁-C₆ alkylene-heteroaryl. In some embodiments, R^{A} is C(O)R^{D}. In some embodiments, R^{A} is —S(O)ₓR^{D}.

In some embodiments, R^{B}, R^{C}, or both are independently hydrogen, C₁-C₆-alkyl, C₁-C₆-heteroalkyl, cycloalkyl, heterocyclyl, or —OR^{A}. In some embodiments, each of R^{B} and R^{C} is independently hydrogen. In some embodiments, each of R^{B} and R^{C} is independently C₁-C₆ alkyl. In some embodiments, one of R^{B} and R^{C} is hydrogen, and the other of R^{B} and R^{C} is C₁-C₆ alkyl. In some embodiments, R^{B} and R^{C} together with the atom to which they are attached form a 3-7-membered heterocyclyl ring optionally substituted with one or more of R⁷ (e.g., 1, 2, or 3 R⁷).

In some embodiments, R^{D} is hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl (e.g., benzyl), or C₁-C₆ alkylene-heteroaryl. In some embodiments, R^{D} is hydrogen. In some embodiments, R^{D} is C₁-C₆ alkyl (e.g., methyl). In some embodiments, R^{D} is C₁-C₆ heteroalkyl. In some embodiments, R^{D} is C₁-C₆ haloalkyl. In some embodiments, R^{D} is cycloalkyl. In some embodiments, R^{D} is heterocyclyl. In some embodiments, R^{D} is aryl. In some embodiments, R^{E} is aryl. In some embodiments, R^{D} is heteroaryl. In some embodiments, R^{D} is C₁-C₆ alkylene-aryl (e.g., benzyl). In some embodiments, R^{D} is C₁-C₆ alkylene-heteroaryl.

In some embodiments, x is an integer between 0 and 2 (e.g., 0, 1, or 2). In some embodiments, x is 0. In some embodiments, x is 1. In some embodiments, x is 2.

In some embodiments, the compound of Formula (I) is a compound of Formula (I-a):

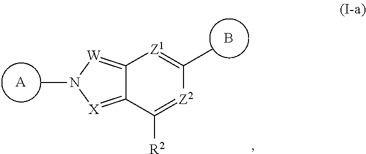

or a pharmaceutically acceptable salt, solvate, hydrate, tautomer, or stereoisomer thereof, A and B are each independently cycloalkyl, heterocyclyl, aryl, or heteroaryl, each of which is optionally substituted with one or more R¹; W, X, Z¹, and Z² are each independently C(R³) or N, wherein at least one of W and X is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; each R² is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-b):

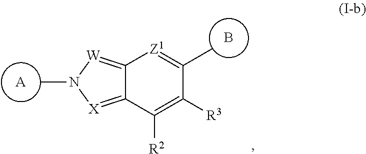

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W, X, Z¹, and Z² are each independently C(R³) or N, wherein at least one of W and X is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; each R² is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-c):

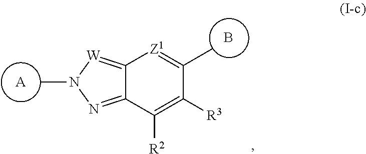

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and Z¹ are each independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; each R² is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-d):

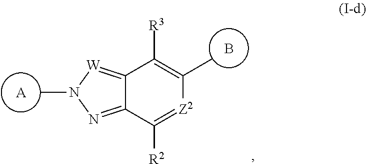

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and Z² are each independently C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; R² is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-e):

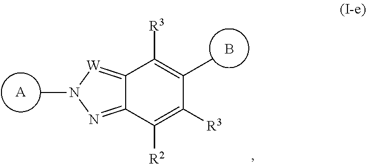

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W is C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; each R² is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-f):

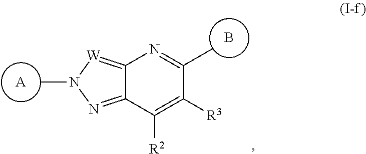

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W is C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; each R² is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-g):

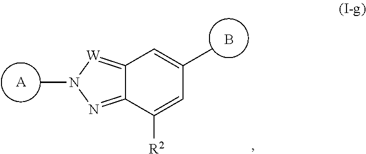

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W is C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; R² is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-h):

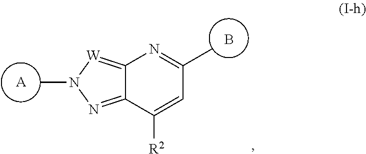

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W is C(R³) or N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴; R² is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, or —OR^{A}; R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵; each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶; each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A}; each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}; each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or R^{B} and R^{C} together with the atom to which they are attached form a 3-7-membered heterocyclyl ring optionally substituted with one or more R⁷; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁷ is C₁-C₆-alkyl, halo, cyano, oxo, or —OR^{A1}; each R^{A1} is hydrogen or C₁-C₆-alkyl; and x is 0, 1, or 2.

In some embodiments, the compound of Formula (I) is a compound of Formula (I-i):

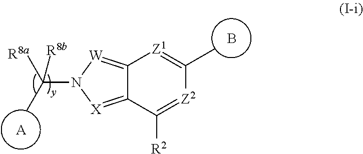

or a pharmaceutically acceptable salt, solvate, hydrate, tautomer, or stereoisomer thereof, wherein A, B, W, X, Z¹, Z², and R² are as defined for Formula (I); each R^{8a} and R^{8b} is independently C₁-C₆-alkyl, halo, cyano, oxo, or —OR^{A1}; and y is 0, 1, 2, 3, or 4.

In some embodiments, the compound of Formula (I) is a compound of Formula (I-j):

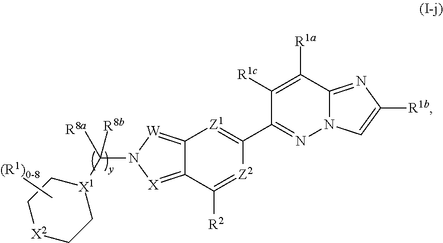

or a pharmaceutically acceptable salt, solvate, hydrate, tautomer, or stereoisomer thereof, wherein each R^{1a}, R^{1b}, R^{1c}, R^{8a} and R^{8b} is independently C₁-C₆-alkyl, halo, cyano, oxo, or —OR^{A1}; X¹ is C(R¹) or N; X² is C(R¹)₂ or N(R¹); y is 0, 1, 2, 3, or 4; and W, X, Z¹, Z², R¹ and R² are as defined for Formula (I).

In some embodiments, the compound of Formula (I) is a compound of Formula (I-k):

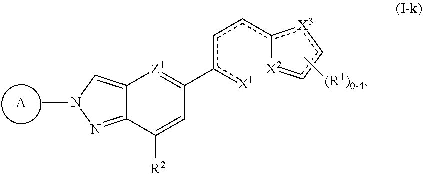

or a pharmaceutically acceptable salt, solvate, hydrate, tautomer, or stereoisomer thereof, wherein each of X¹ and X³ is independently C(R³) or N; X² is C or N; and wherein A, Z¹, R¹, R², and R³ are as defined for Formula (I).

In some embodiments, the compound of Formula (I) is a compound of Formula (I-l):

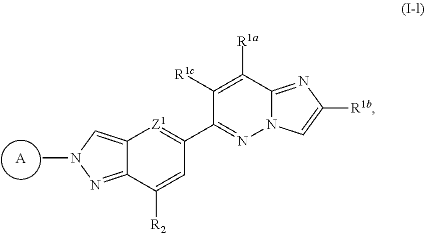

or a pharmaceutically acceptable salt, solvate, hydrate, tautomer, or stereoisomer thereof, wherein each of R^{1a}, R^{1b}, and R^{1c} is independently hydrogen, C₁-C₆-alkyl, halo, or —OR^{A}; and wherein A, R^{A}, Z¹, R¹, R², and R³ are as defined for Formula (I).

In some embodiments, the compound of Formula (I) is a compound of Formula (I-m):

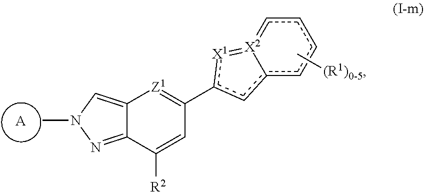

or a pharmaceutically acceptable salt, solvate, hydrate, tautomer, or stereoisomer thereof, wherein each of X¹ and X³ is independently C(R¹) or N; X² is C or N; and wherein A, Z¹, R¹ and R² are as defined for Formula (I).

In some embodiments, the compound of Formula (I) is a compound of Formula (I-n):

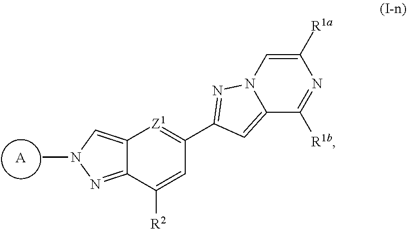

or a pharmaceutically acceptable salt, solvate, hydrate, tautomer, or stereoisomer thereof, wherein each of R^{1a} and R^{1b} is independently hydrogen, C₁-C₆-alkyl, halo, or —OR^{A}; and wherein A, R^{A}, Z¹, R¹, R², and R³ are as defined for Formula (I).

In some embodiments, the compound of Formula (I) is a compound of Formula (I-0):

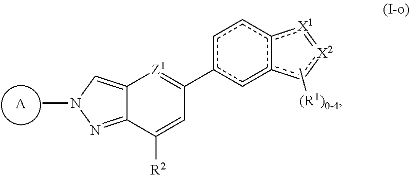

or a pharmaceutically acceptable salt, solvate, hydrate, tautomer, or stereoisomer thereof, wherein each of X¹ and X² is independently C(R¹) or N; and wherein A, Z¹, R¹ and R² are as defined for Formula (I).

In some embodiments, the compound of Formula (I) is a compound of Formula (I-p):

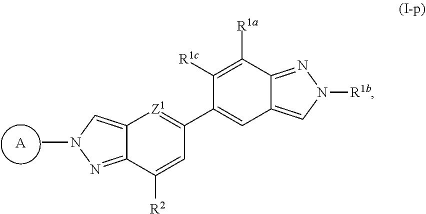

or a pharmaceutically acceptable salt, solvate, hydrate, tautomer, or stereoisomer thereof, wherein each of R^{1a}, R^{1b}, and R^{1c} is independently hydrogen, C₁-C₆-alkyl, halo, or —OR^{A}; and wherein A, R^{A}, Z¹, R¹ and R² are as defined for Formula (I).

In some embodiments, the compound of Formula (I) is a compound of Formula (I-q):

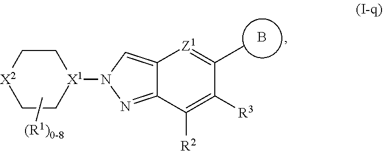

or a pharmaceutically acceptable salt, solvate, hydrate, tautomer, or stereoisomer thereof, wherein X¹ is C(R¹); X² is C(R¹)₂ or N(R¹); and wherein B, Z¹, R¹, R² and R³ are as defined for Formula (I).

In some embodiments, the compound of Formula (I) is a compound of Formula (I-r):

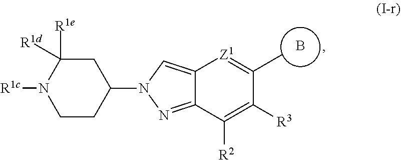

or a pharmaceutically acceptable salt, solvate, hydrate, tautomer, or stereoisomer thereof, wherein each of R^{1c}, R^{1d} and R^{1e} is independently hydrogen, alkyl, halo, or heteroalkyl; or R^{1d} and R^{1e} together with the atoms to which they are attached, form a three or four membered cycloalkyl group; and wherein each of B, Z¹, Z², R², and R³ are as defined as for Formula (I).

In some embodiments, the compound of Formula (I) is a compound of Formula (I-s):

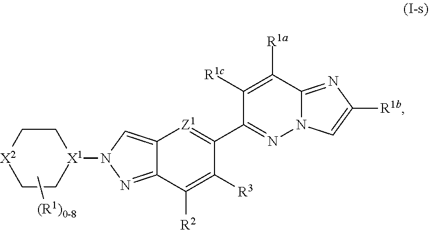

or a pharmaceutically acceptable salt, solvate, hydrate, tautomer, or stereoisomer thereof, wherein X¹ is C(R¹); X² is C(R¹)₂ or N(R¹); each of R^{1a}, R^{1b}, and R^{1c} is independently hydrogen, C₁-C₆-alkyl, halo, or —OR^{A} and wherein R^{A}, Z¹, R¹, R², and R³ are as defined for Formula (I).

In some embodiments, the compound of Formula (I) is a compound of Formula (I-t):

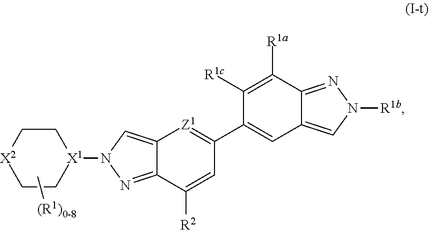

or a pharmaceutically acceptable salt, solvate, hydrate, tautomer, or stereoisomer thereof, wherein X¹ is C(R¹); X² is C(R¹)₂ or N(R¹); each of R^{1a}, R^{1b}, and R^{1c} is independently hydrogen, C₁-C₆-alkyl, halo, or —OR^{A} and wherein R^{A}, Z¹, R¹, and R² are as defined for Formula (I).

In some embodiments, the compound of Formula (I) is a compound of Formula (I-u):

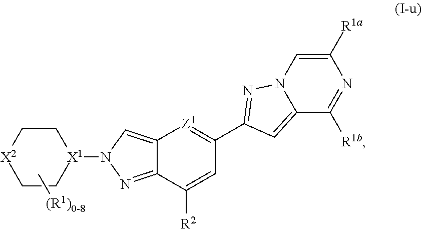

or a pharmaceutically acceptable salt, solvate, hydrate, tautomer, or stereoisomer thereof, wherein X¹ is C(R¹); X² is C(R¹)₂ or N(R¹); each of R^{1a} and R^{1b} is independently hydrogen, C₁-C₆-alkyl, halo, or —OR^{A} and wherein R^{A}, Z¹, R¹, and R² are as defined for Formula (I).

In some embodiments, the compound of Formula (I) is a compound of Formula (I-v):

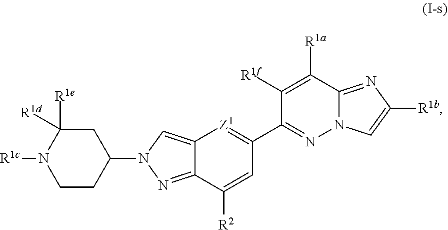

or a pharmaceutically acceptable salt, solvate, hydrate, tautomer, or stereoisomer thereof, wherein each of R^{1a}, R^{1b}, R^{1c}, R^{1d}, R^{1e}, and R^{1f} is independently hydrogen, C₁-C₆-alkyl, halo, or R^{A}; and wherein R^{A}, Z¹, R¹, and R² are as defined in for Formula (I).

In some embodiments of any and all of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), and (I-v) or a pharmaceutically acceptable salt thereof, one of A and B is selected from

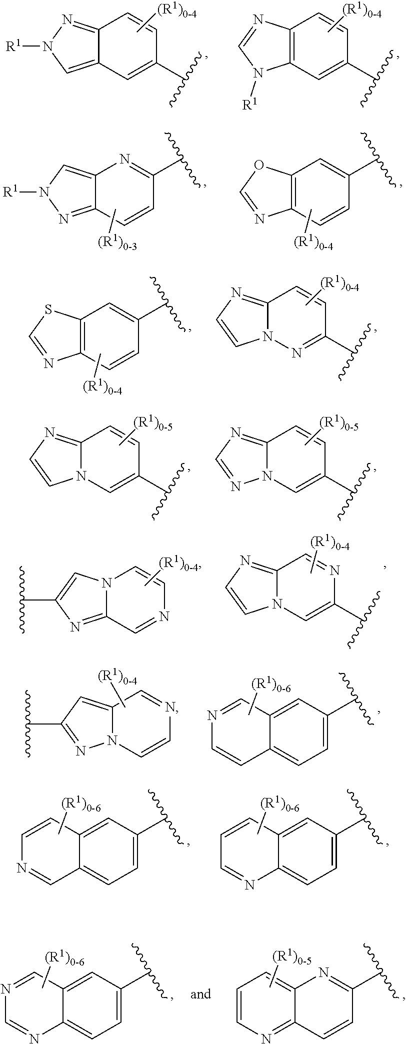

wherein R¹ is as described herein. In some embodiments, one of A and B is independently selected from

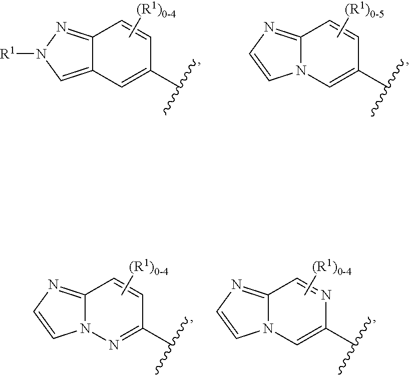

wherein R is as described herein. In some embodiments, one of A and B is independently selected from

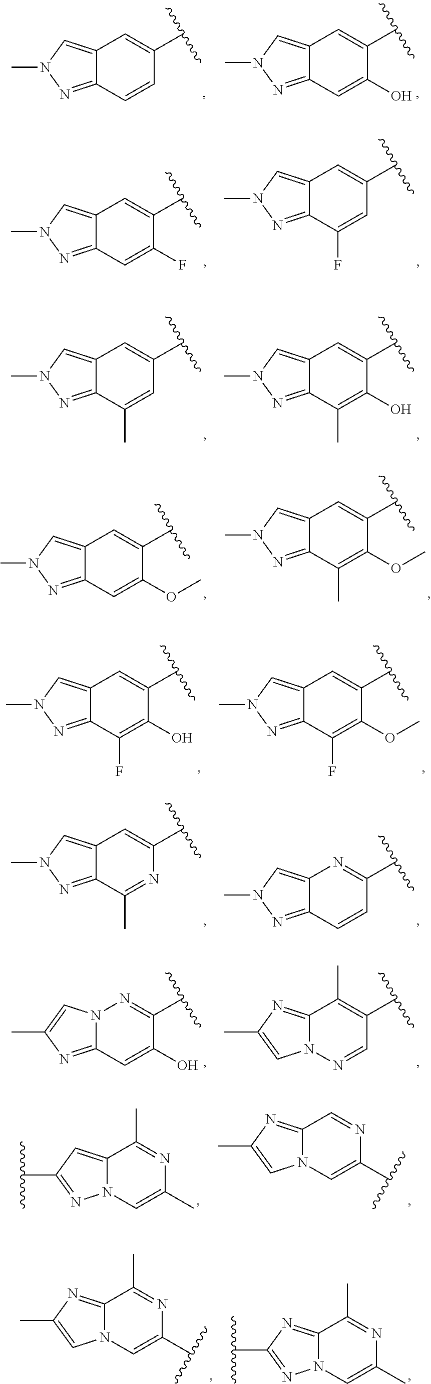

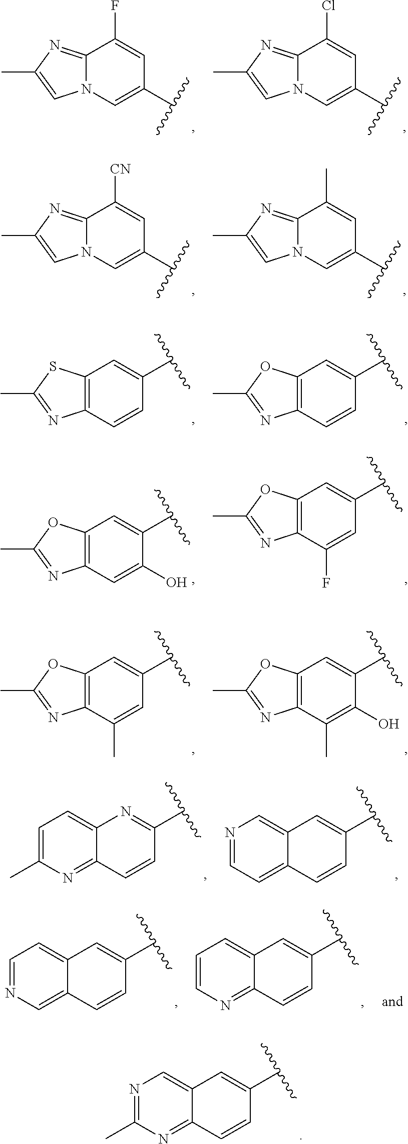

In some embodiments, one of A and B is independently selected from

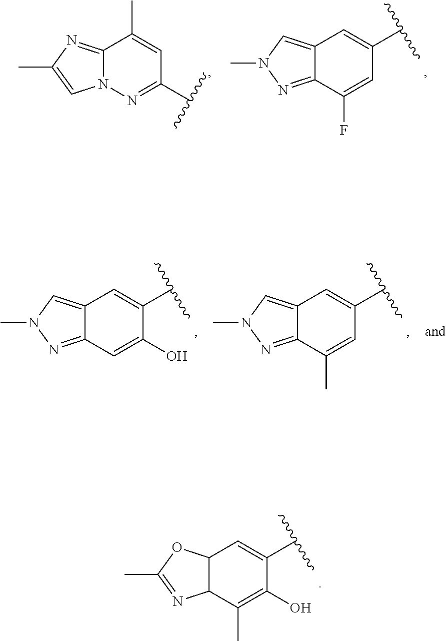 [SMILES: *c1cc(C)c2nc(C)cn2n1 ; *c1cc(F)c2nn(C)cc2c1 ; *c1cc2cn(C)nc2cc1O ; *c1cc(C)c2nn(C)cc2c1 ; *C1=CC2OC(C)=NC2C(C)=C1O]

In some embodiments, one of A and B is independently selected from

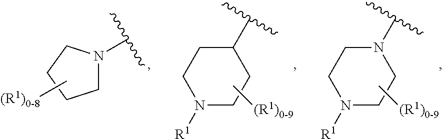

and

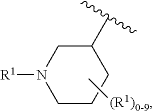

wherein R¹ is as described herein. In some embodiments, one of A and B is independently selected from

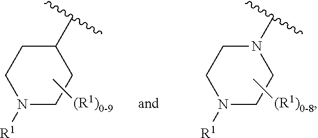

wherein R¹ is as described herein. In some embodiments, one of A and B is independently selected from

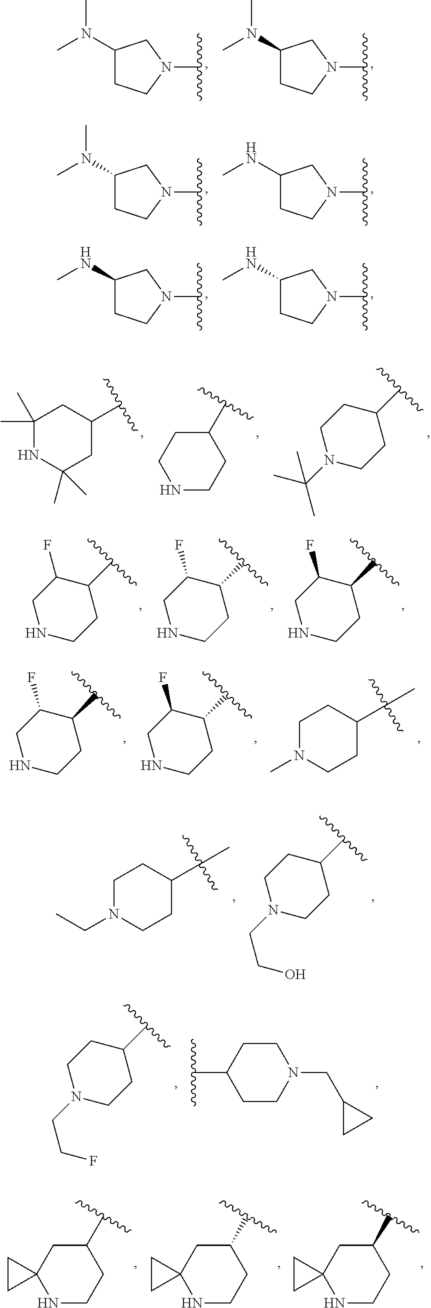

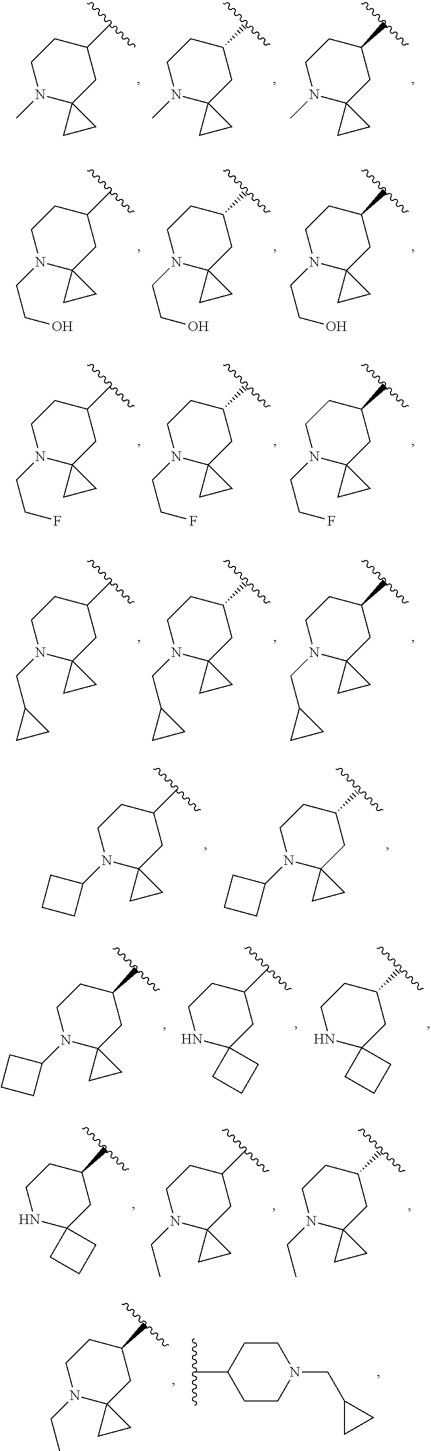

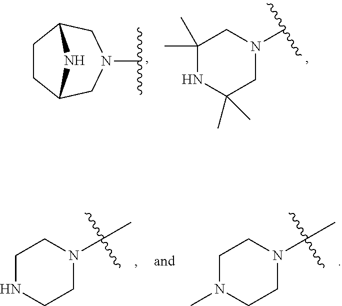

In some embodiments, A is selected from

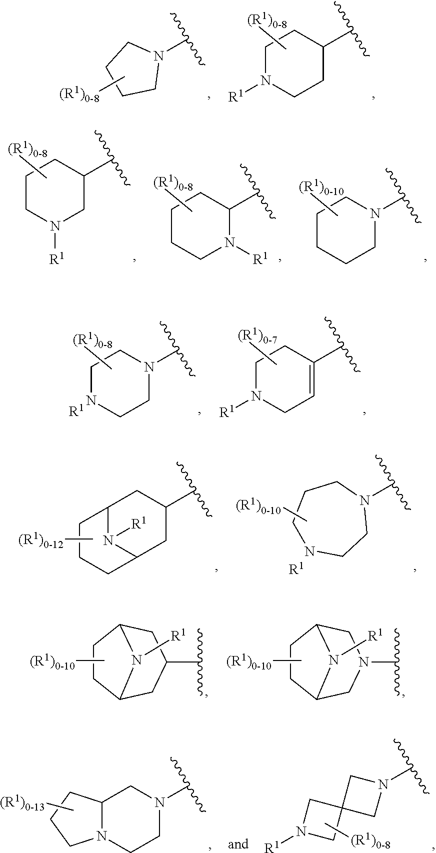

wherein R¹ is as defined herein.

In some embodiments of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v), A is selected from

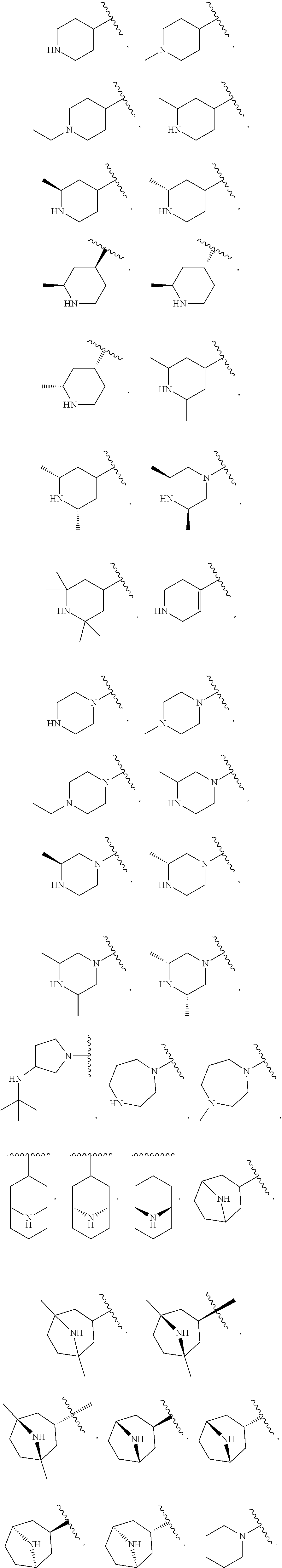

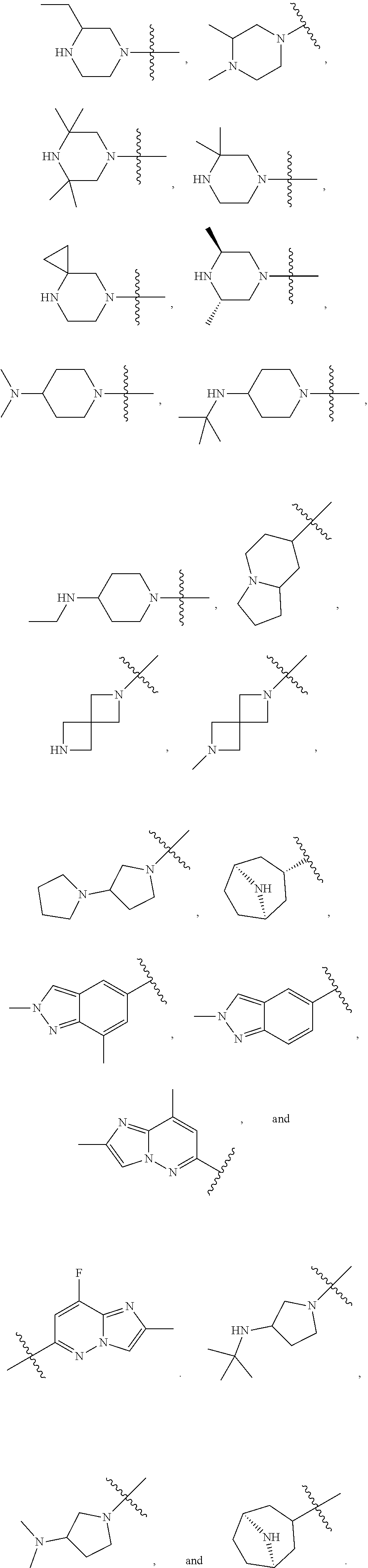

In some embodiments, A is

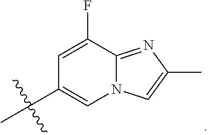 [SMILES: *c1cc(F)c2nc(C)cn2c1]

In some embodiments, A is

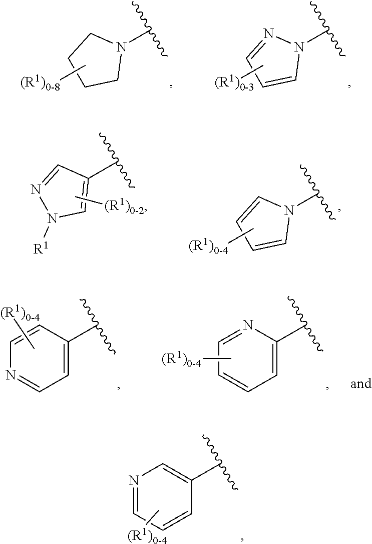

In some embodiments of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v), B is selected from

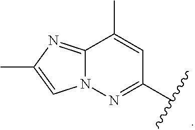

wherein R¹ is as defined herein.

In some embodiments, B is selected from

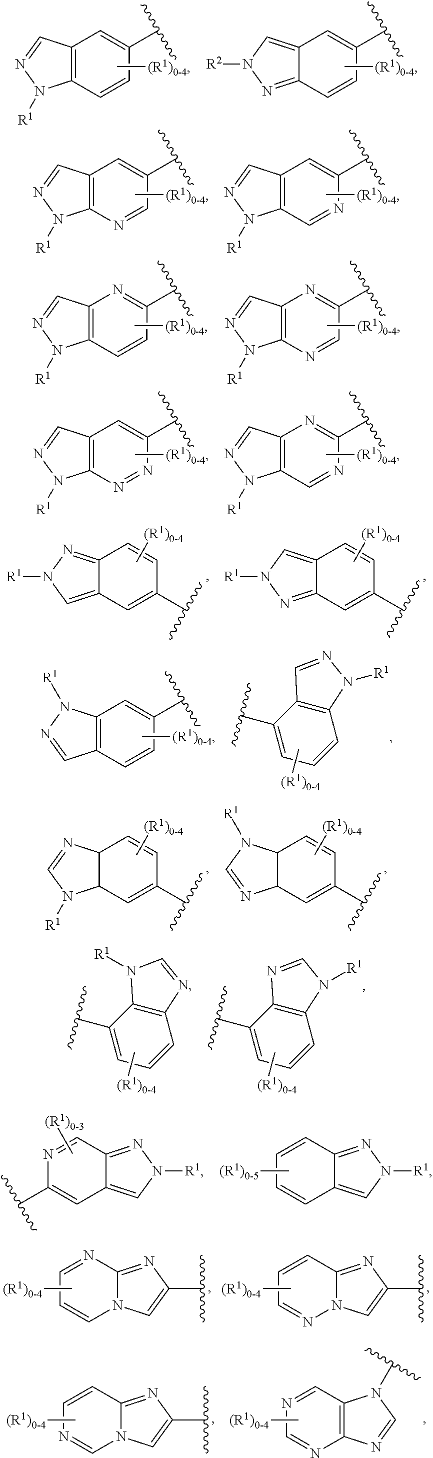

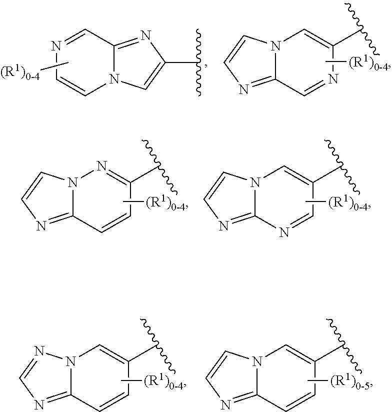

wherein R¹ is as defined herein. In some embodiments, B is selected from

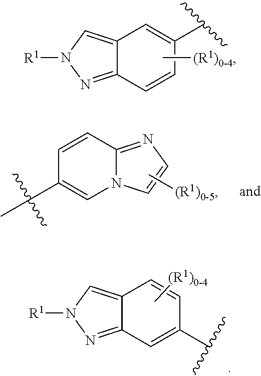

In some embodiments, B is

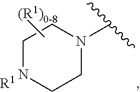

wherein R¹ is as defined herein. In some embodiments, B is selected from

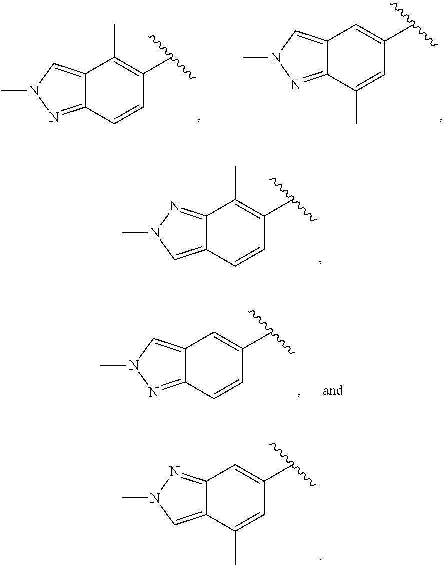 [SMILES: *c1ccc2nn(C)cc2c1C ; *c1cc(C)c2nn(C)cc2c1 ; *c1ccc2cn(C)nc2c1C ; *c1ccc2nn(C)cc2c1 ; *c1cc(C)c2cn(C)nc2c1]

In some embodiments, B is

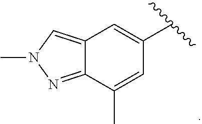 [SMILES: *c1cc(C)c2nn(C)cc2c1]

In some embodiments, B is

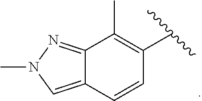 [SMILES: *c1ccc2cn(C)nc2c1C]

In some embodiments, B is

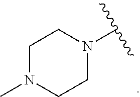 [SMILES: *N1CCN(C)CC1]

In some embodiments, B is

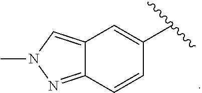 [SMILES: *c1ccc2nn(C)cc2c1]

In some embodiments, B is

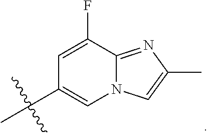 [SMILES: *c1cc(F)c2nc(C)cn2c1]

In some embodiments, B is

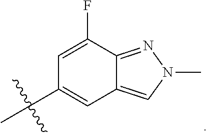 [SMILES: *c1cc(F)c2nn(C)cc2c1]

In some embodiments, B is

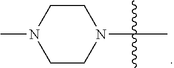 [SMILES: *N1CCN(C)CC1]

In some embodiments, B is

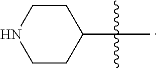 [SMILES: *C1CCNCC1]

In some embodiments, B is

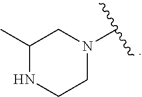 [SMILES: *N1CCNC(C)C1]

In some embodiments, L is absent. In some embodiments, L is C₁-C₆-alkylene (e.g., C₁-alkylene, C₂-alkylene, C₃-alkylene, C₄-alkylene, C₅-alkylene, or C₆-alkylene). In some embodiments, L is unsubstituted C₁-C₆ alkylene. In some embodiments, L is substituted C₁-C₆-alkylene, e.g., C₁-C₆ alkylene substituted with one or more R⁴. In some embodiments, L is C₁-alkylene substituted with one R⁴. In some embodiments, L is —CH₂— (or methylene). In some embodiments, L is —C(O)— (or carbonyl).

In some embodiments, L is absent, C₁-C₆-alkylene, or C₁-C₆-heteroalkylene, wherein each alkylene and heteroalkylene is optionally substituted with one or more R⁴.

In some embodiments of Formulas (I-a), (I-b), (I-c), (I-d), (I-e), (I-f), (I-g), (I-h), (I-i), (I-j), (I-k), (I-l), (I-m), (I-n), (I-o), (I-p), (I-q), (I-r), (I-s), (I-t), (I-u), or (I-v), R² is selected from hydrogen, halo (e.g., fluoro, chloro), C₁-C₆-alkyl (e.g., CH₃), or —OR^{A1} (e.g., —OCH₃). In some embodiments, R² is hydrogen. In some embodiments, R² is halo (e.g., fluoro, chloro). In some embodiments, R² is C₁-C₆-alkyl (e.g., CH₃). In some embodiments, R² is —OR^{A1} (e.g., —OCH₃).

In some embodiments, the compound of Formula (I) is selected from a compound in Table 1, or a pharmaceutically acceptable salt, solvate, hydrate, tautomer, or stereoisomer thereof.

TABLE 1
Exemplary compounds.

| Compound No. | Structure |
|---|---|
| 100 | 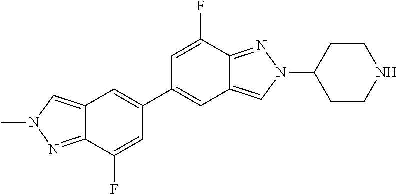 [SMILES: Cn1cc2cc(-c3cc(F)c4nn(C5CCNCC5)cc4c3)cc(F)c2n1] |
| 102 | 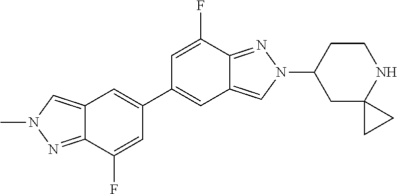 [SMILES: Cn1cc2cc(-c3cc(F)c4nn(C5CCNC6(CC6)C5)cc4c3)cc(F)c2n1] |
| 103 | 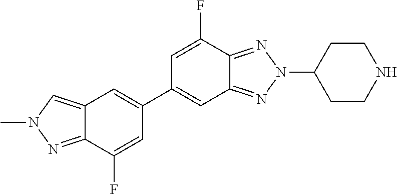 [SMILES: Cn1cc2cc(-c3cc(F)c4nn(C5CCNCC5)nc4c3)cc(F)c2n1] |
| 104 | 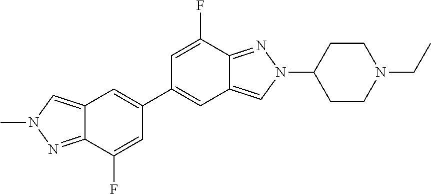 [SMILES: CCN1CCC(n2cc3cc(-c4cc(F)c5nn(C)cc5c4)cc(F)c3n2)CC1] |
| 105 | 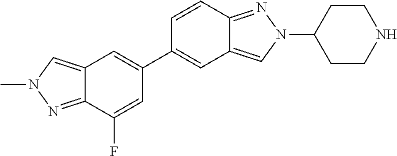 [SMILES: Cn1cc2cc(-c3ccc4nn(C5CCNCC5)cc4c3)cc(F)c2n1] |
| 107 | 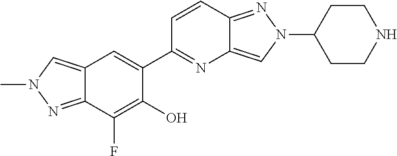 [SMILES: Cn1cc2cc(-c3ccc4nn(C5CCNCC5)cc4n3)c(O)c(F)c2n1] |
| 108 | 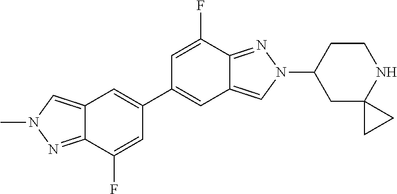 [SMILES: Cn1cc2cc(-c3cc(F)c4nn(C5CCNC6(CC6)C5)cc4c3)cc(F)c2n1] |
| 109 | 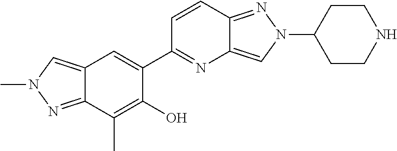 [SMILES: Cc1c(O)c(-c2ccc3nn(C4CCNCC4)cc3n2)cc2cn(C)nc12] |
| 110 | 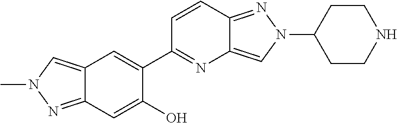 [SMILES: Cn1cc2cc(-c3ccc4nn(C5CCNCC5)cc4n3)c(O)cc2n1] |
| 111 | 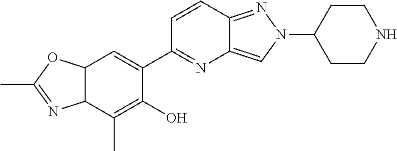 [SMILES: CC1=NC2C(C)=C(O)C(c3ccc4nn(C5CCNCC5)cc4n3)=CC2O1] |
| 112 | 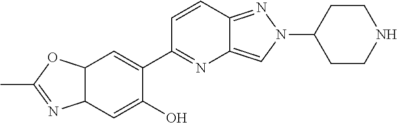 [SMILES: CC1=NC2C=C(O)C(c3ccc4nn(C5CCNCC5)cc4n3)=CC2O1] |
| 113 | 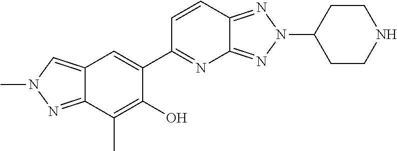 [SMILES: Cc1c(O)c(-c2ccc3nn(C4CCNCC4)nc3n2)cc2cn(C)nc12] |
| 114 | 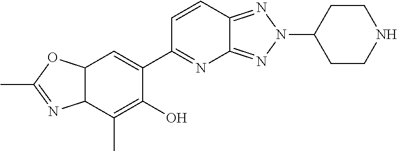 [SMILES: CC1=NC2C(C)=C(O)C(c3ccc4nn(C5CCNCC5)nc4n3)=CC2O1] |
| 115 | 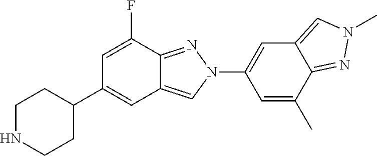 [SMILES: Cc1cc(-n2cc3cc(C4CCNCC4)cc(F)c3n2)cc2cn(C)nc12] |
| 116 | 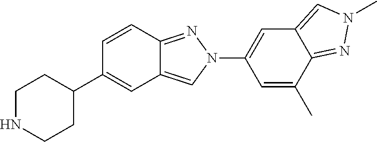 [SMILES: Cc1cc(-n2cc3cc(C4CCNCC4)ccc3n2)cc2cn(C)nc12] |
| 117 | 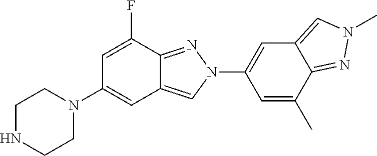 [SMILES: Cc1cc(-n2cc3cc(N4CCNCC4)cc(F)c3n2)cc2cn(C)nc12] |
| 118 | 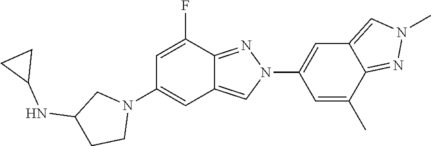 [SMILES: Cc1cc(-n2cc3cc(N4CCC(NC5CC5)C4)cc(F)c3n2)cc2cn(C)nc12] |
| 119 | 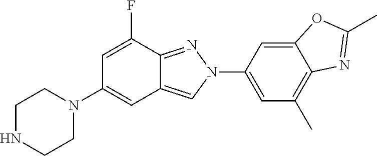 [SMILES: Cc1nc2c(C)cc(-n3cc4cc(N5CCNCC5)cc(F)c4n3)cc2o1] |
| 120 | 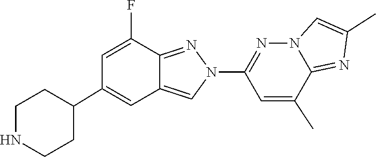 [SMILES: Cc1cn2nc(-n3cc4cc(C5CCNCC5)cc(F)c4n3)cc(C)c2n1] |
| 121 | 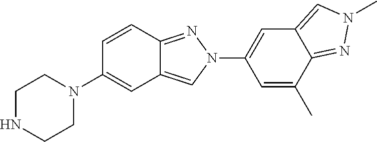 [SMILES: Cc1cc(-n2cc3cc(N4CCNCC4)ccc3n2)cc2cn(C)nc12] |
| 122 | 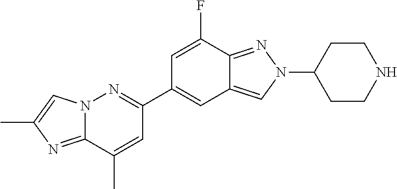 [SMILES: Cc1cn2nc(-c3cc(F)c4nn(C5CCNCC5)cc4c3)cc(C)c2n1] |
| 123 | 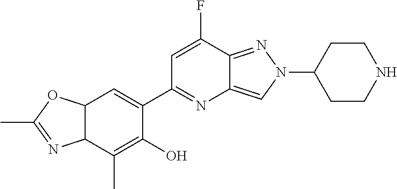 [SMILES: CC1=NC2C(C)=C(O)C(c3cc(F)c4nn(C5CCNCC5)cc4n3)=CC2O1] |
| 124 | 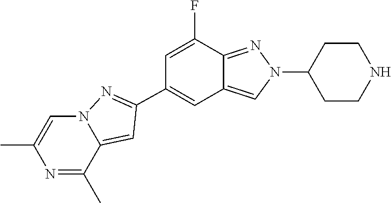 [SMILES: Cc1cn2nc(-c3cc(F)c4nn(C5CCNCC5)cc4c3)cc2c(C)n1] |
| 125 | 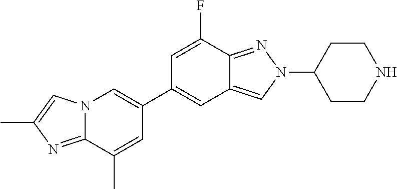 [SMILES: Cc1cn2cc(-c3cc(F)c4nn(C5CCNCC5)cc4c3)cc(C)c2n1] |
| 126 | 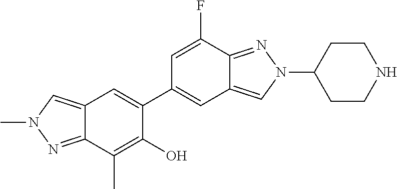 [SMILES: Cc1c(O)c(-c2cc(F)c3nn(C4CCNCC4)cc3c2)cc2cn(C)nc12] |
| 127 | 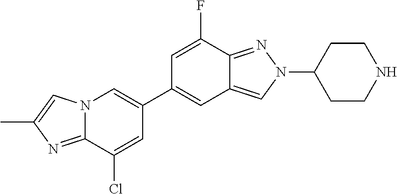 [SMILES: Cc1cn2cc(-c3cc(F)c4nn(C5CCNCC5)cc4c3)cc(Cl)c2n1] |
| 128 | 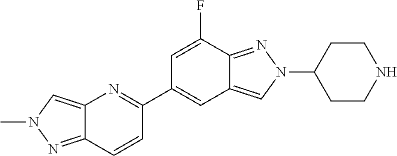 [SMILES: Cn1cc2nc(-c3cc(F)c4nn(C5CCNCC5)cc4c3)ccc2n1] |
| 129 | 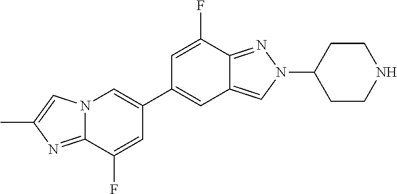 [SMILES: Cc1cn2cc(-c3cc(F)c4nn(C5CCNCC5)cc4c3)cc(F)c2n1] |
| 130 | 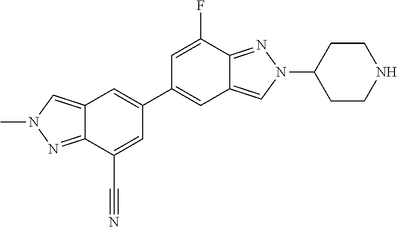 [SMILES: Cn1cc2cc(-c3cc(F)c4nn(C5CCNCC5)cc4c3)cc(C#N)c2n1] |
| 131 | 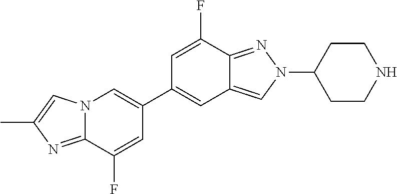 [SMILES: Cc1cn2cc(-c3cc(F)c4nn(C5CCNCC5)cc4c3)cc(F)c2n1] |
| 132 | 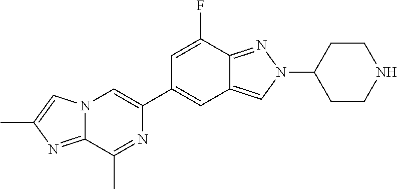 [SMILES: Cc1cn2cc(-c3cc(F)c4nn(C5CCNCC5)cc4c3)nc(C)c2n1] |
| 133 | 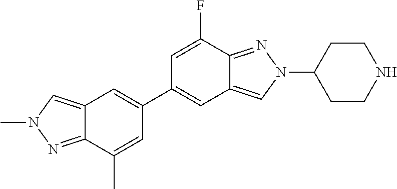 [SMILES: Cc1cc(-c2cc(F)c3nn(C4CCNCC4)cc3c2)cc2cn(C)nc12] |
| 134 | 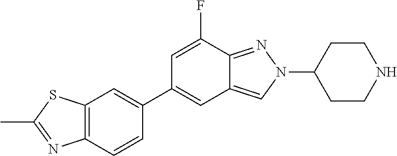 [SMILES: Cc1nc2ccc(-c3cc(F)c4nn(C5CCNCC5)cc4c3)cc2s1] |
| 135 | 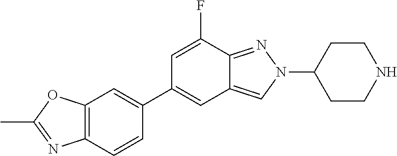 [SMILES: Cc1nc2ccc(-c3cc(F)c4nn(C5CCNCC5)cc4c3)cc2o1] |
| 136 | 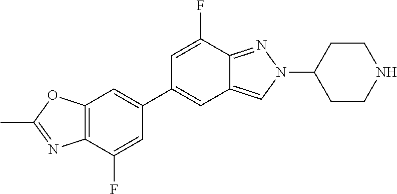 [SMILES: Cc1nc2c(F)cc(-c3cc(F)c4nn(C5CCNCC5)cc4c3)cc2o1] |
| 137 | 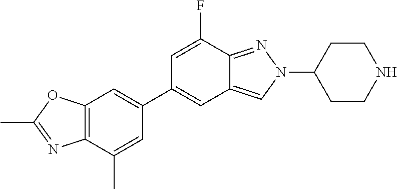 [SMILES: Cc1nc2c(C)cc(-c3cc(F)c4nn(C5CCNCC5)cc4c3)cc2o1] |
| 138 | 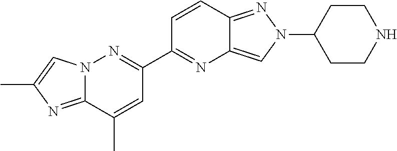 [SMILES: Cc1cn2nc(-c3ccc4nn(C5CCNCC5)cc4n3)cc(C)c2n1] |
| 139 | 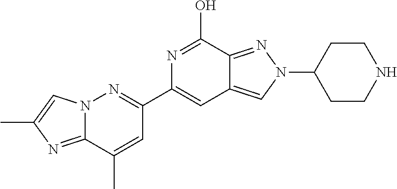 [SMILES: Cc1cn2nc(-c3cc4cn(C5CCNCC5)nc4c(O)n3)cc(C)c2n1] |
| 140 | 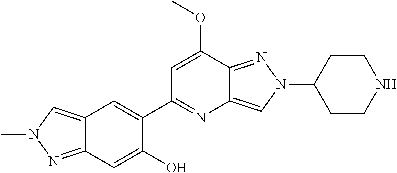 [SMILES: COc1cc(-c2cc3cn(C)nc3cc2O)nc2cn(C3CCNCC3)nc12] |
| 141 | 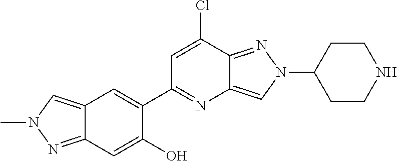 [SMILES: Cn1cc2cc(-c3cc(Cl)c4nn(C5CCNCC5)cc4n3)c(O)cc2n1] |
| 142 | 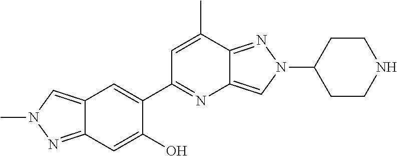 [SMILES: Cc1cc(-c2cc3cn(C)nc3cc2O)nc2cn(C3CCNCC3)nc12] |
| 143 | 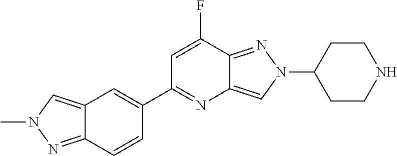 [SMILES: Cn1cc2cc(-c3cc(F)c4nn(C5CCNCC5)cc4n3)ccc2n1] |
| 144 | 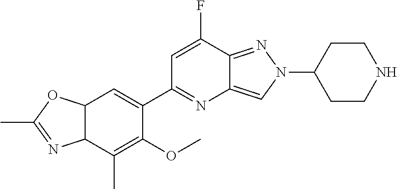 [SMILES: COC1=C(C)C2N=C(C)OC2C=C1c1cc(F)c2nn(C3CCNCC3)cc2n1] |
| 145 | 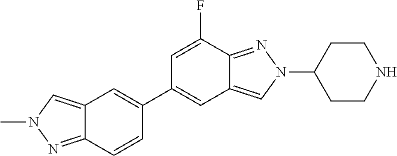 [SMILES: Cn1cc2cc(-c3cc(F)c4nn(C5CCNCC5)cc4c3)ccc2n1] |
| 146 | 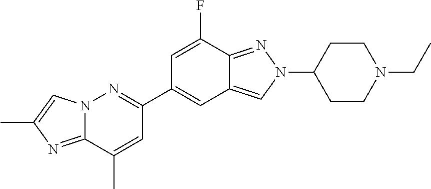 [SMILES: CCN1CCC(n2cc3cc(-c4cc(C)c5nc(C)cn5n4)cc(F)c3n2)CC1] |
| 147 | 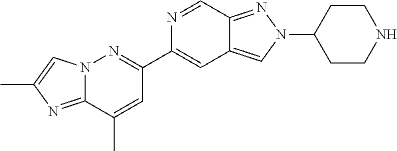 [SMILES: Cc1cn2nc(-c3cc4cn(C5CCNCC5)nc4cn3)cc(C)c2n1] |
| 148 | 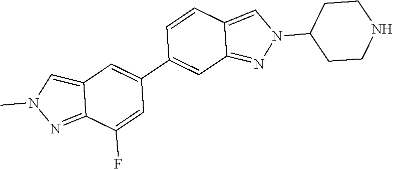 [SMILES: Cn1cc2cc(-c3ccc4cn(C5CCNCC5)nc4c3)cc(F)c2n1] |
| 149 | 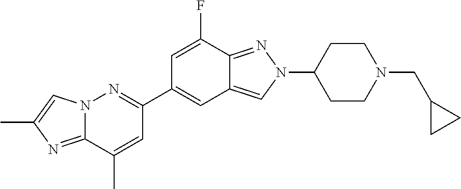 [SMILES: Cc1cn2nc(-c3cc(F)c4nn(C5CCN(CC6CC6)CC5)cc4c3)cc(C)c2n1] |
| 150 | 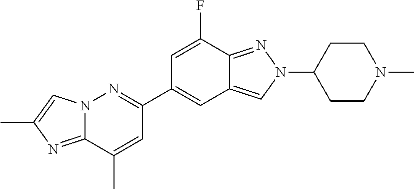 |
| 151 | 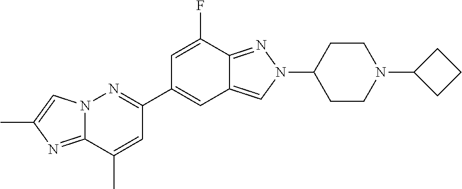 [SMILES: Cc1cn2nc(-c3cc(F)c4nn(C5CCN(C6CCC6)CC5)cc4c3)cc(C)c2n1] |
| 152 | 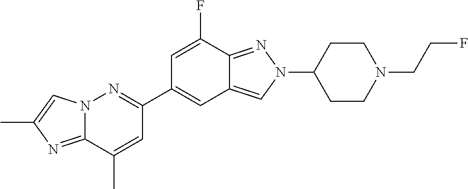 [SMILES: Cc1cn2nc(-c3cc(F)c4nn(C5CCN(CCF)CC5)cc4c3)cc(C)c2n1] |
| 153 | 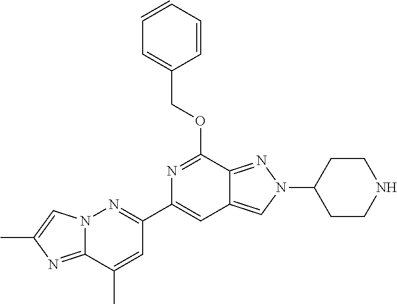 [SMILES: Cc1cn2nc(-c3cc4cn(C5CCNCC5)nc4c(OCc4ccccc4)n3)cc(C)c2n1] |
| 154 | 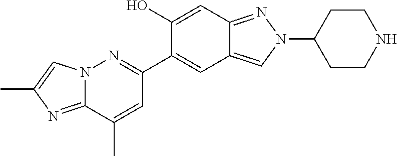 [SMILES: Cc1cn2nc(-c3cc4cn(C5CCNCC5)nc4cc3O)cc(C)c2n1] |
| 155 | 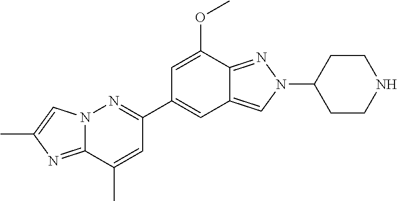 [SMILES: COc1cc(-c2cc(C)c3nc(C)cn3n2)cc2cn(C3CCNCC3)nc12] |
| 156 | 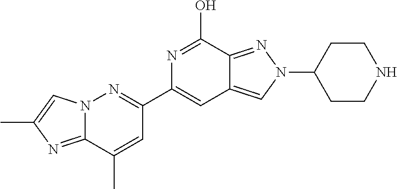 [SMILES: Cc1cn2nc(-c3cc4cn(C5CCNCC5)nc4c(O)n3)cc(C)c2n1] |
| 157 | 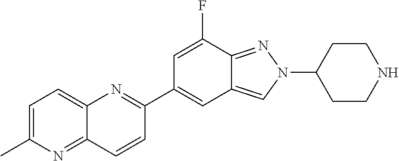 [SMILES: Cc1ccc2nc(-c3cc(F)c4nn(C5CCNCC5)cc4c3)ccc2n1] |
| 158 | 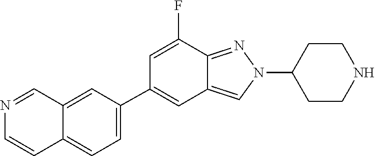 [SMILES: Fc1cc(-c2ccc3ccncc3c2)cc2cn(C3CCNCC3)nc12] |
| 159 | 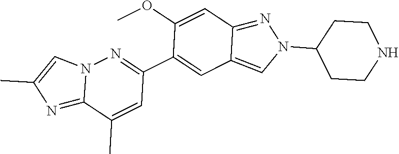 |
| 160 | 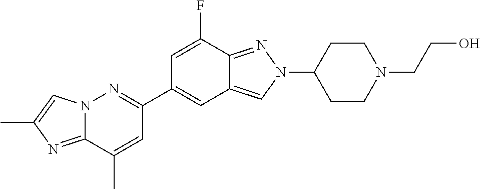 [SMILES: Cc1cn2nc(-c3cc(F)c4nn(C5CCN(CCO)CC5)cc4c3)cc(C)c2n1] |
| 161 | 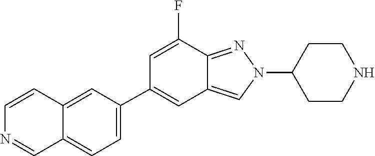 [SMILES: Fc1cc(-c2ccc3cnccc3c2)cc2cn(C3CCNCC3)nc12] |
| 162 | 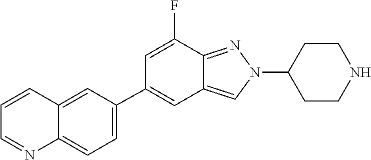 [SMILES: Fc1cc(-c2ccc3ncccc3c2)cc2cn(C3CCNCC3)nc12] |
| 163 | 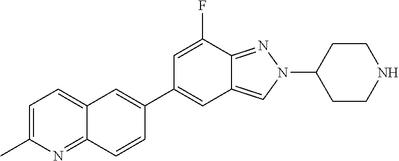 [SMILES: Cc1ccc2cc(-c3cc(F)c4nn(C5CCNCC5)cc4c3)ccc2n1] |
| 165 | 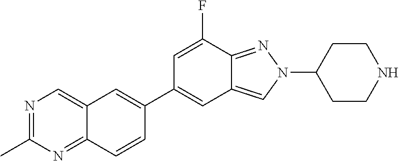 [SMILES: Cc1ncc2cc(-c3cc(F)c4nn(C5CCNCC5)cc4c3)ccc2n1] |
| 166 | 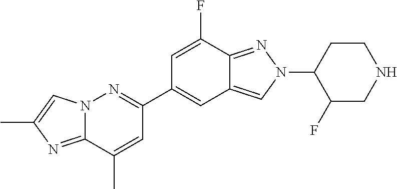 [SMILES: Cc1cn2nc(-c3cc(F)c4nn(C5CCNCC5F)cc4c3)cc(C)c2n1] |
| 167 | 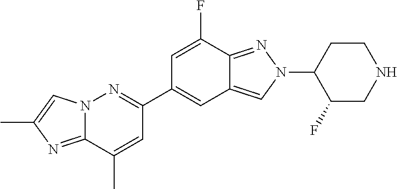 [SMILES: Cc1cn2nc(-c3cc(F)c4nn(C5CCNC[C@@H]5F)cc4c3)cc(C)c2n1] |
| 168 | 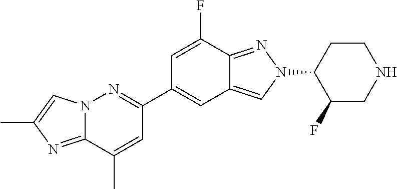 [SMILES: Cc1cn2nc(-c3cc(F)c4nn([C@@H]5CCNC[C@H]5F)cc4c3)cc(C)c2n1] |
| 169 | 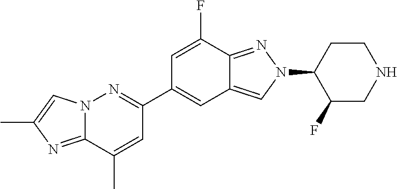 [SMILES: Cc1cn2nc(-c3cc(F)c4nn([C@H]5CCNC[C@H]5F)cc4c3)cc(C)c2n1] |
| 170 | 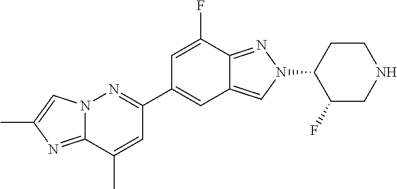 [SMILES: Cc1cn2nc(-c3cc(F)c4nn([C@@H]5CCNC[C@@H]5F)cc4c3)cc(C)c2n1] |
| 171 | 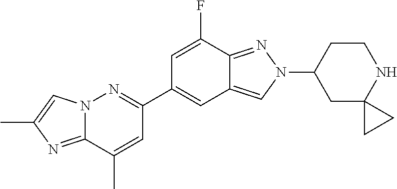 [SMILES: Cc1cn2nc(-c3cc(F)c4nn(C5CCNC6(CC6)C5)cc4c3)cc(C)c2n1] |
| 172 | 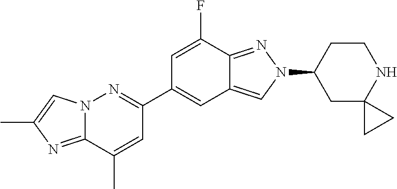 [SMILES: Cc1cn2nc(-c3cc(F)c4nn([C@H]5CCNC6(CC6)C5)cc4c3)cc(C)c2n1] |
| 173 | 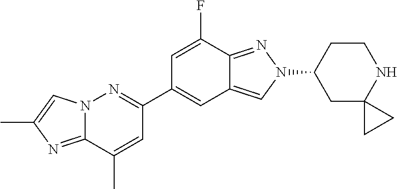 [SMILES: Cc1cn2nc(-c3cc(F)c4nn([C@@H]5CCNC6(CC6)C5)cc4c3)cc(C)c2n1] |
| 174 | 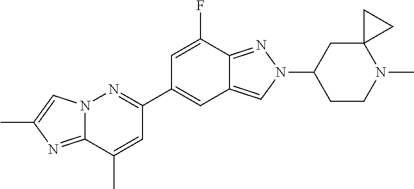 |
| 175 | 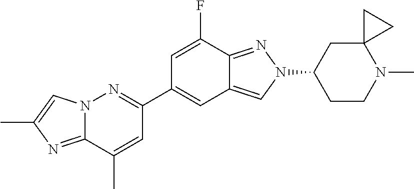 |
| 176 | 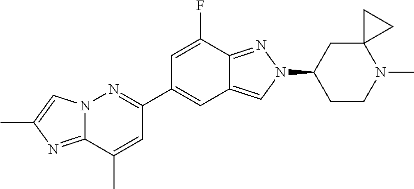 |
| 177 | 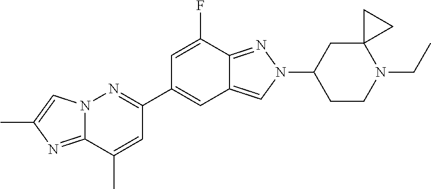 [SMILES: CCN1CCC(n2cc3cc(-c4cc(C)c5nc(C)cn5n4)cc(F)c3n2)CC12CC2] |
| 178 | 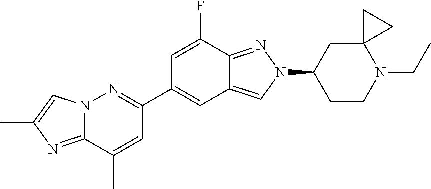 [SMILES: CCN1CC[C@@H](n2cc3cc(-c4cc(C)c5nc(C)cn5n4)cc(F)c3n2)CC12CC2] |
| 179 | 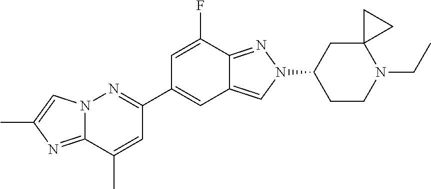 [SMILES: CCN1CC[C@H](n2cc3cc(-c4cc(C)c5nc(C)cn5n4)cc(F)c3n2)CC12CC2] |
| 180 | 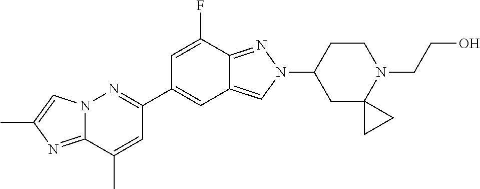 [SMILES: Cc1cn2nc(-c3cc(F)c4nn(C5CCN(CCO)C6(CC6)C5)cc4c3)cc(C)c2n1] |
| 181 | 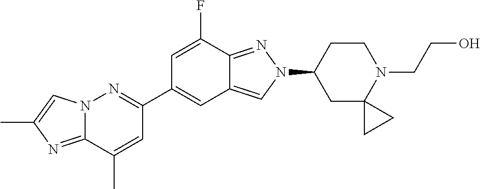 [SMILES: Cc1cn2nc(-c3cc(F)c4nn([C@H]5CCN(CCO)C6(CC6)C5)cc4c3)cc(C)c2n1] |
| 182 | 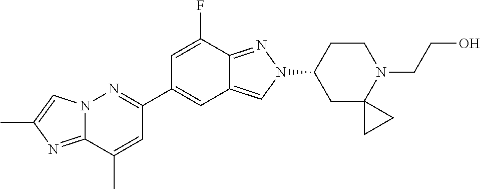 [SMILES: Cc1cn2nc(-c3cc(F)c4nn([C@@H]5CCN(CCO)C6(CC6)C5)cc4c3)cc(C)c2n1] |
| 183 | 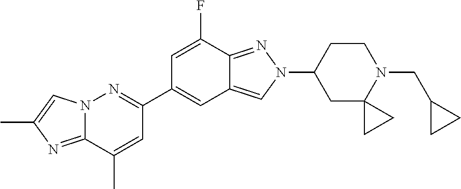 [SMILES: Cc1cn2nc(-c3cc(F)c4nn(C5CCN(CC6CC6)C6(CC6)C5)cc4c3)cc(C)c2n1] |
| 184 | 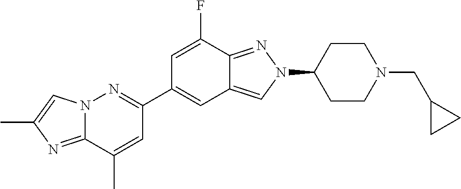 |
| 185 | 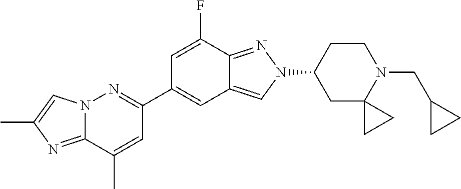 [SMILES: Cc1cn2nc(-c3cc(F)c4nn([C@@H]5CCN(CC6CC6)C6(CC6)C5)cc4c3)cc(C)c2n1] |
| 186 | 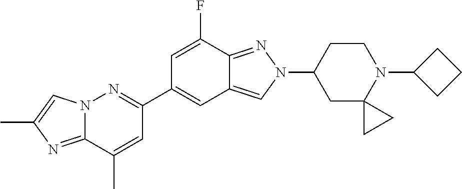 [SMILES: Cc1cn2nc(-c3cc(F)c4nn(C5CCN(C6CCC6)C6(CC6)C5)cc4c3)cc(C)c2n1] |
| 187 | 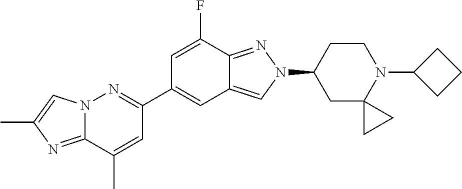 [SMILES: Cc1cn2nc(-c3cc(F)c4nn([C@H]5CCN(C6CCC6)C6(CC6)C5)cc4c3)cc(C)c2n1] |
| 188 | 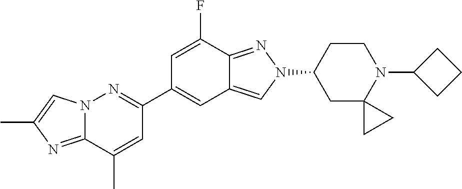 [SMILES: Cc1cn2nc(-c3cc(F)c4nn([C@@H]5CCN(C6CCC6)C6(CC6)C5)cc4c3)cc(C)c2n1] |
| 189 | 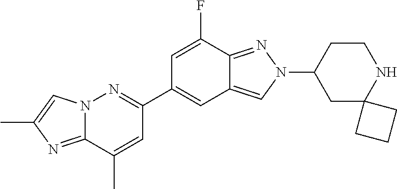 [SMILES: Cc1cn2nc(-c3cc(F)c4nn(C5CCNC6(CCC6)C5)cc4c3)cc(C)c2n1] |
| 190 | 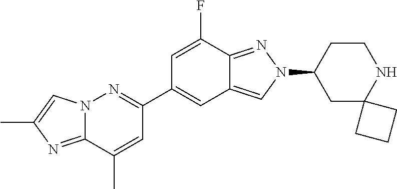 [SMILES: Cc1cn2nc(-c3cc(F)c4nn([C@H]5CCNC6(CCC6)C5)cc4c3)cc(C)c2n1] |
| 191 | 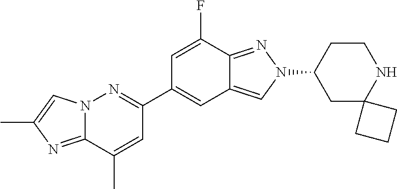 [SMILES: Cc1cn2nc(-c3cc(F)c4nn([C@@H]5CCNC6(CCC6)C5)cc4c3)cc(C)c2n1] |
| 192 | 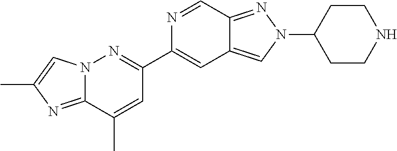 [SMILES: Cc1cn2nc(-c3cc4cn(C5CCNCC5)nc4cn3)cc(C)c2n1] |
| 193 | 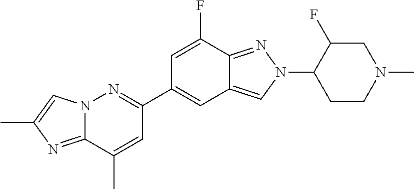 |
| 194 | 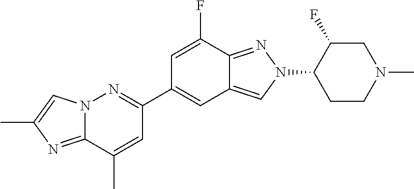 |
| 195 | 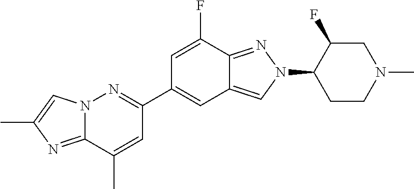 |
| 196 | 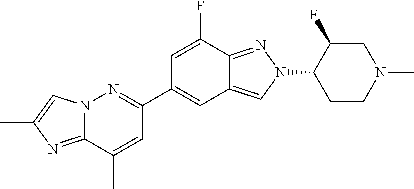 |
| 197 | 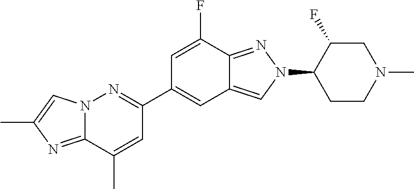 |
| 198 | 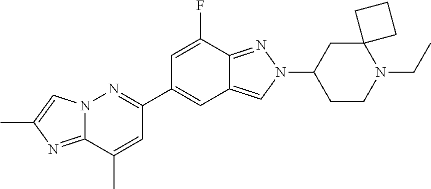 [SMILES: CCN1CCC(n2cc3cc(-c4cc(C)c5nc(C)cn5n4)cc(F)c3n2)CC12CCC2] |
| 199 | 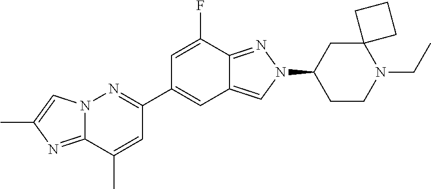 [SMILES: CCN1CC[C@@H](n2cc3cc(-c4cc(C)c5nc(C)cn5n4)cc(F)c3n2)CC12CCC2] |
| 200 | 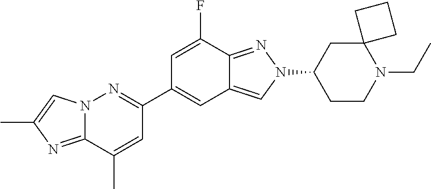 [SMILES: CCN1CC[C@H](n2cc3cc(-c4cc(C)c5nc(C)cn5n4)cc(F)c3n2)CC12CCC2] |
| 201 | 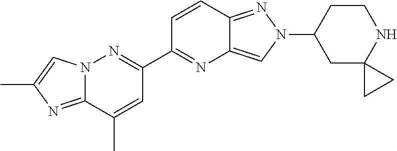 [SMILES: Cc1cn2nc(-c3ccc4nn(C5CCNC6(CC6)C5)cc4n3)cc(C)c2n1] |
| 202 | 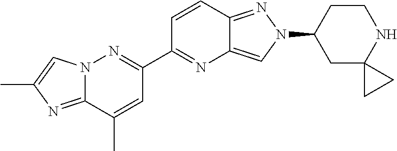 [SMILES: Cc1cn2nc(-c3ccc4nn([C@H]5CCNC6(CC6)C5)cc4n3)cc(C)c2n1] |
| 203 | 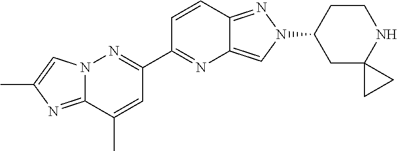 [SMILES: Cc1cn2nc(-c3ccc4nn([C@@H]5CCNC6(CC6)C5)cc4n3)cc(C)c2n1] |
| 204 | 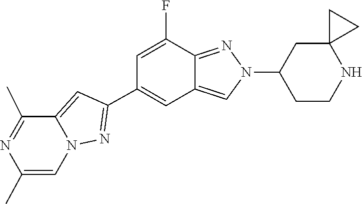 [SMILES: Cc1cn2nc(-c3cc(F)c4nn(C5CCNC6(CC6)C5)cc4c3)cc2c(C)n1] |
| 205 | 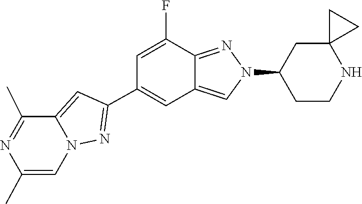 [SMILES: Cc1cn2nc(-c3cc(F)c4nn([C@@H]5CCNC6(CC6)C5)cc4c3)cc2c(C)n1] |
| 206 | 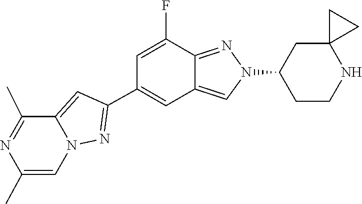 [SMILES: Cc1cn2nc(-c3cc(F)c4nn([C@H]5CCNC6(CC6)C5)cc4c3)cc2c(C)n1] |
| 207 | 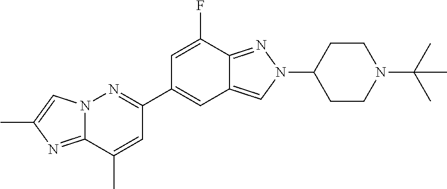 [SMILES: Cc1cn2nc(-c3cc(F)c4nn(C5CCN(C(C)(C)C)CC5)cc4c3)cc(C)c2n1] |
| 208 | 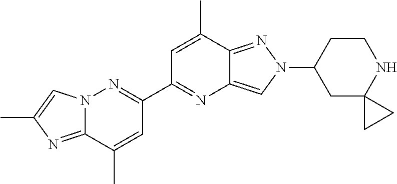 [SMILES: Cc1cn2nc(-c3cc(C)c4nn(C5CCNC6(CC6)C5)cc4n3)cc(C)c2n1] |
| 209 | 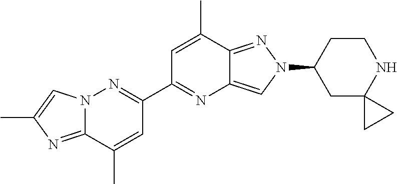 [SMILES: Cc1cn2nc(-c3cc(C)c4nn([C@H]5CCNC6(CC6)C5)cc4n3)cc(C)c2n1] |
| 210 | 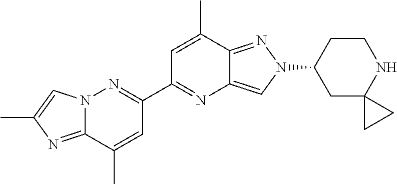 [SMILES: Cc1cn2nc(-c3cc(C)c4nn([C@@H]5CCNC6(CC6)C5)cc4n3)cc(C)c2n1] |
| 211 | 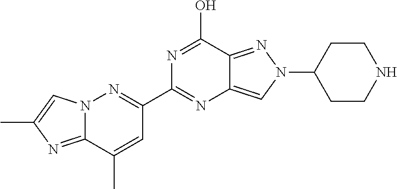 [SMILES: Cc1cn2nc(-c3nc(O)c4nn(C5CCNCC5)cc4n3)cc(C)c2n1] |
| 212 | 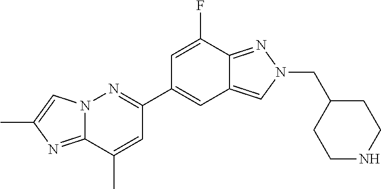 [SMILES: Cc1cn2nc(-c3cc(F)c4nn(CC5CCNCC5)cc4c3)cc(C)c2n1] |
| 213 | 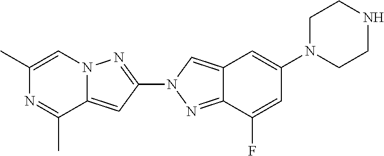 [SMILES: Cc1cn2nc(-n3cc4cc(N5CCNCC5)cc(F)c4n3)cc2c(C)n1] |
| 214 | 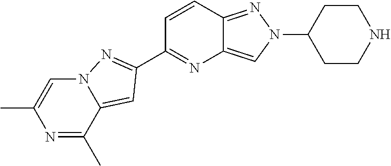 [SMILES: Cc1cn2nc(-c3ccc4nn(C5CCNCC5)cc4n3)cc2c(C)n1] |

### Pharmaceutical Compositions, Kits, and Administration

The present invention provides pharmaceutical compositions comprising a compound of Formula (I), e.g., a compound of Formula (I) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or a pharmaceutically acceptable salt thereof, and optionally a pharmaceutically acceptable excipient. In certain embodiments, the compound of Formula (I) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNVA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASHIL-IT1, ASNSD1, ASPM, ATAD5, ATF1, ATG4A, ATG16L2, ATM ATN1, ATP11C, ATP6V1G3, ATP13A5, ATP7A, ATP7B, ATR, ATXN2, ATXN3, ATXN7, ATXN10, AXIN1, B2M B4GALNT3, BBS4, BCL2, BCL2L1, BCL2-like 11 (BIM), BCL11B, BBOX1, BCS1L, BEMAN, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf11, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDKI1B, CDK13, CEBPZ, CEL, CELSR3, CENPA, CENPI, CENPT, CENTB2, CENTG2, CEP110, CEP170, CEP192, CETP, CFB, CFTR, CFH, CGN, CGNL1, CHAFIA, CHD9, CHIC2, CHL1, CHN1, CHM CLEC16A, CLIC2, CLCN1, CLINT1, CLK1, CLPB, CLPTM1, CMIP, CMYA5, CNGA3, CNOT1, CNOT7, CNTN6, COG3, COL1A1, COL11A2, COL12A1, COL14A1, COL15A1, COL17AM, COL19A1, COL1A1, COL1A2, COL2A, COL3A1, COL4A1, COL4A2, COL4A5, COL4A6, COL5A2, COL6A1, COL7A, COL9A, COL9A2, COL22A1, COL24A1, COL25A1, COL29A1, COLQ, COMTD1, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF, DNAH3, DNAH8, DNAI1, DNAJA4, DNAJC3, DNAJC7, DNMT1, DNTTIP2, DOCK4, DOCK5, DOCK1O, DOCK11, DOTIL, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBX015, FBX018, FBXO38, FCGBP, FECH, FEZ2, FGA, FGD6, FGFR2, FGFR1OP, FGFR1OP2, FGFR2, FGG, FGR, FIX FKBP3, FLI1, FLJ35848, FLJ36070, FLNA, FN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLT1A, GOLT1B, GPATCH1, GPR158, GPR160, GPX4, GRAAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXC10, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, ILIR2, IL5RA, IL7RA, I/MAIT, INPP5D, INSR, INTS3, INTU, IP04, IP08, IQGAP2, IRF2, IRF4, IRF8, IRX3, ISL1, ISL2, ITFG1, ITGA6, ITGAL, ITGB1, ITGB2, 1TGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIF5A, KIE5B, KIF9, KIN, KIR2DL5B, KIR3DL2, KIR3DL3, KIT, KLF3, KLF5, KLF7, KLF10, KLF12, KLF16, KLHL20, KLK12, KLKB1, KMT2A, KMT2B, KPNA5, KRAS, KREMEN1, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF11, MEMO1, MET, MGA, MGAM, MGAT4A, MGAT5, MGC16169, MGC34774, MKKS, MIB1, MIER2, MITF, MKL2, MLANA, MLH1, MLL5, MLX, MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NRIH3, NRIH4, NR4A3, NR5A1, NRXN1, NSMAF, NSMCE2, NT5C, NT5C2, NT5C3, NUBP1, NUBPL, NUDT5, NUMA1, NUP88, NUP98, NUP160, NUPL1, OAT, OAZ1, OBFC2A, OBFC2B, OLIG2, OMA1, OPA1, OPN4, OPTN, OSBPL11, OSBPL8, OSGEPL1, OTC, OTX2, OVOL2, OXT, PA2G4, PADI4, PAH, PAN2, PAOX, PAPOLG, PARD3, PARP1, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PELI2, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPPIR12A, PPP3CB, PPP4C, PPP4RIL, PPP4R2, PRAME, PRC1, PRDM1, PREX1, PREX2, PRIM1, PRIM2, PRKARIA, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RABI1FIP2, RAB23, RAF1, RALBP1, RALGDS, RB1CC1, RBL2, RBM39, RBM45, RBPJ, RBSN, REC8, RELB, RFC4, RFT1, RFTN1, RHOA, RHPN2, RIF1, RIT1, RLN3, RMND5B, RNFI1, RNF32, RAFT1, RNGTT, ROCK1, ROCK2, RORA, RP1, RP6KA3, RP11-265F1, RP13-36C9, RPAP3, RPN1, RPGR, RPL22, RPL22L1, RPS6KA6, RREB1, RRM1, RRPIB, RSK2, RTEL1, RTF, RUFY1, RUNX1, RUNX2, RXRA, RYR3, SAAL1, SAE1, SALL4, SAT1, SATB2, SBCAD, SCN1A, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDB1, SEZ6, SFRS12, SGCE, SGOL2, SGPL1, SH2D1A, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX SMS, SMTN, SNCAIP, SNORD86, SNRK, SNRP70, SNX5, SNX6, SOD1, SOD10, SOS, SOS2, SOX5, SOX6, SOX8, SP1, SP2, SP3, SPH10, SPAG9, SPATA13, SPATA4, SPATS1, SPECC1L, SPDEF, SP11, SPINK5, SPP2, SPTA1, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBP1, SUCLG2, SULF2, SUPT6H, SUPT16H, SV2C, SYCP2, SYT6, SYCP1, SYTL3, SYTL5, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGS1, THAP7, THAP12, THOC2, TIAL1, TIAM2, TIMM50, TLK2, TM4SF20, TM6SF1, TMEM27, TMEM77, TMEM156, TMEM194A, TMF1, TMPRSS6, TNFRSF10A, TNFRSF10B, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPS1, TSC1, TSC2, TSHB, TSPAN7, TTC17, TTF1, TTLL5, TTLL9, TTN, TTPAL, TTR, TUSC3, TXNDC10, UBE3A, UCK1, UGTIA1, UHRFIBP1, UNC45B, UNC5C, USH2A, USF2, USP1, USP6, USP18, USP38, USP39, UTP20, UTP15, UTP18, UTRN, UTX, UTY, UVRAG, UXT, VAPA, VEGFA, VPS29, VPS35, VPS39, VT11A, VTI1B, VWA3B, WDFY2, WDR16, WDR17, WDR26, WDR44, WDR67, WDTC1, WRN, WRNIP1, WT1, WWC3, XBP1, XRN1, XRN2, XX-FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX, ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC0000120.3, KRIT1, AC004076.1, ZNF772, AC004076.9, ZNF772, AC004223.3, RAD51D, AC004381.6, AC006486.1, ERF, AC0007390.5, AC0007780.1, PRKARIA, AC0007998.2, INO80C, AC0009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADH1B, ADIPOR1, ADNP, ADPRH, AGBL5, AGPAT1, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKT1S1, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDH1B1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZL1, AP000311.1, CRYZL1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5F1A, ATP5G2, ATP5J, ATP5MD, ATP5PF, ATP6AP2, ATP6V0B, ATP6V1C1, ATP6V1D, ATP7B, ATXN1, ATXN1L, IST1, ATXN3, ATXN7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOC1S1, RP11-644F5.10, BLOC1S6, AC090527.2, BLOC1S6, RP11-96020.4, BLVRA, BMF, BOLA1, BORCS8-MEF2B, BORCS8, BRCA1, BRD1, BRDT, BRINP3, BROX BTBD10, BTBD3, BTBD9, BTD, BTF3L4, BTNL9, BUB1B-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, C1QB, C1QTNF1, C1S, C1orf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CA5B, CABYR, CALCA, CALCOCO1, CALCOCO2, CALM1, CALM3, CALML4, RP11-315D16.2, CALN1, CALU, CANT1, CANX CAP1, CAPN12, CAPS2, CARD8, CARHSP1, CARNS1, CASC1, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB1IP1, CCNC, CCND3, CCNG1, CCP110, CCR9, CCT7, CCT8, CD151, CD1D, CD200, CD22, CD226, CD276, CD36, CD59, CDC26, CDC42, CDC42SE1, CDC42SE2, CDHR3, CDK10, CDK16, CDK4, CDKAL1, CDKL3, CTD-2410N18.4, CDKN1A, CDKN2A, CDNF, CEBPZOS, CELF1, CEMIP, CENPK, CEP170B, CEP250, CEP57, CEP57L1, CEP63, CERS4, CEL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCP1, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE, CSF2RA, CSGALNACT, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP11A1, CYP2R1, CYP4B1, CYP4F22, DAG1, DAGLB, KDELR2, DARS, DBNL, DCAF11, DCAF8, PEX19, DCLREIC, DCTD, DCTN1, DCTN4, DCUNID2, DDR1, DDX11, DDX19B, AC012184.2, DDX19B, RP11-529K.3, DDX25, DDX39B, ATP6V1G2-DDX39B, SNORD84, DDX42, DDX60L, DEDD, DEDD2, DEFA1, DEFA1B, DEFA1B, DEFA3, DENNDIC, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKNV, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RP11-500M8.7, DZIP1L, E2F6, ECHDC1, ECSIT, ECT2, EDC3, EDEM1, EDEM2, MMP24-AS1, RP4-614O4.1H, EEF1AKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRFI1, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAM111B, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBX018, FBXO22, FBXO31, FBXO41, FBXO44, FBXO45, FBXW9, FCHO1, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLB1L, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBPL, GPER, GPR16, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCYIB3, GUK1, GULP1, GYPC, GYS1, GZF1, HAGH, HA02, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKR1, HLF, HMBOX1, HMGA1, HMGB3, HMGCR, HMGN4, HMOX2, HNRNPC, HNRNPD, HNRNPH1, HNRNPH3, HNRNPR, HOMER3, HOPX, HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, ILI1, IL18BP, IL18RAP, IL1RAP, IL1RL1, IL18R1, ILIRN, IL32, IL4I1, NUP62, AC011452.1, IL4I1, NUP62, CTC-326K19.6, IL6ST, ILVBL, IMAMP1L, IMPDH1, INCA1, ING1, INIP, INPP1, INPP5J, INPP5K, INSIG2, INTS11, INTS12, INTS14, IP6K2, IP6K3, IPOI1, LRRC70, IQCE, IQGAP3, IRAK4, IRF3, IRF5, IRF6, ISG20, IST1, ISYNA1, ITFG2, ITGB1BP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDM1B, KDM4A, AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLK11, KLK2, KLK5, KLK6, KLK7, KNOP1, KRBA2, AC135178.2, KRBA2, RPII-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTL1, LGALS8, LGALS9C, LGMN, LHFPL2, LIG4, LIMCH1, LIMK2, LIMS2, LINC00921, ZNF263, LIPF, LLGL2, LMAN2L, LMCD, LMF1, RP11-161M6.2, LMO1, LM03, LOXHD1, LPAR1, LPAR2, LPAR4, LPAR5, LPAR6, LPHN1, LPIN2, LPIN3, LPP, LRFN5, LRIF1, LRAMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MADIL1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRS1, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGF10, ME11, MEIS2, MELK, MET, METTL13, METTL23, MFF, MFN2, MFSD2A, MGST3, MIB2, MICAL1, MICAL3, MICOSI0, NBL1, MICOSI0-NBL1, MID1, MINA, MINOS1-NBL1, MINOS1, MIOS, MIPOL1, MIS12, MKLN1, MKNK1, MKNK1, MOB3C, MLF2, MLH1, MMP17, MOBP, MOCS1, MOGS, MOK, MORF4L1, MPC1, MPC2, MPG, MP1, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMR10, MTRF1, MTRR, MTUS2, MUTYH, MVK, MX1, MX2, MYH10, MYL12A, MYB, MYD88, MYL5, MYLIP, MYNN, MYO15A, MYO1B, MYOM2, MZF1, N4BP2L2, NAA60, NAB1, NAE1, NAGK, NAP1L1, NAP1L4, NAPG, NARFL, NARG2, NAT1, NAT10, NBPF11, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFV1, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RPII-1212A22.1, NPIPB3, NPIPB4, NPIPB9, NPL, NPM1, NPPA, NQO2, NRIH3, NR2C2, NR2F2, NR4A1, NRDC, NREP, NRF1, NRG4, NRIP1, NSD2, NSDHL, NSG1, NSMCE2, NSRP1, NT5C2, NTF4, NTMT1, NTNG2, NUBP2, NUCB2, NUDT1, NUDT2, NUDT4, NUF2, NUMBL, NUP50, NUP54, NUP85, NVL, NXF1, NXPE1, NXPE3, OARD1, OAT, OAZ2, OCIAD1, OCLN, ODF2, OGDHL, OGFOD2, AC026362.1, OGFOD2, RPII-197N18.2, OLA1, OPRL1, OPTN, OR2H1, ORAI2, ORMDL1, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH1, PARVG, PASK, PAX6, PBRM1, PBXIP1, PCBP3, PCBP4, AC115284.1, PCBP4, RP11-155D18.14, RP11-155D18.12, PCGF3, PCGF5, PCNP, PCSK9, PDCD10, PDCD6, AHRR, PDDC1, PDGFRB, PDIA6, PDIK1L, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEP1, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHA1, PLEKHA2, PLEKHA6, PLEKHG5, PLIN1, PLS1, PLS3, PLSCR1, PLSCR2, PLSCR4, PLXNB1, PLXNB2, PMP22, PMS1, PNISR, PNKP, AKTIS1, PNMT, PNPLA4, PNPLA8, PNPO, PNRC1, POCIB, POFUT1, POLB, POLD1, POLH, POLI, POLL, POLR1B, POM121, POM121C, AC006014.7, POM121C, AC211429.1, POMC, POMT1, POP1, PORCN, POU5F1, PSORS1C3, PPARD, PPARG, PPHLN1, PPIL3, PPIL4, PPM1A, PPM1B, AC013717.1, PPP1CB, PPP1R11, PPPIR13L, PPPIR26, PPPIR9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAG1, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORS1C1, PSPH, PSRCl, PTBP3, PTHLH, PTK2, PTPDC1, PTPRM, PUF60, PUM2, PUS1, PUS10, PXA, PXYLP1, PYCR1, QRICH1, R3HCC1L, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAP1, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBM12B, RBM14, RBM4, RBM14-RBM4, RBM23, RBM4, RBM14-RBM4, RBM47, RBM7, AP002373.1, RBM7, RP11-212D19.4, RBMS2, RBMY1E, RBPJ, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDD1, RHNO1, TULP3, RHOC, AL603832.3, RHOC, RPII-426L16.10, RHOH, RIC8B, RIMKLB, RIN1, RIPK2, RIT1, RLIM, RNASE4, ANG, AL163636.6, RNASEK, RNASEK-C17orf49, RNFI11, RNF123, RNF13, RNF14, RNF185, RNF216, RNF24, RNF32, RNF34, RNF38, RNF4, RNF44, RNH1, RNMT, RNPS1, RO60, ROPN1, ROPN1B, ROR2, RP1-102H19.8, C6orf163, RP1-283E3.8, CDK11A, RPII-120M18.2, PRKARIA, RPII-133K10.2, PAK6, RPII-164J13.1, CAPN3, RP11-21J18.1, ANKRD12, RPII-322E1.6, INO80C, RPII-337C18.10, CHD1L, RPII-432B6.3, TRIM59, RPII-468E2.4, IRF9, RPII-484M3.5, UPK1B, RPII-517H2.6, CCR6, RPII-613M10.9, SLC25A51, RPII-659G9.3, RAB30, RPII-691N7.6, CTNND, RPII-849H4.2, RP11-896J10.3, NKX2-1, RPII-96020.4, SQRDL, RP11-986E7.7, SERPINA3, RP4-769N13.6, GPRASP1, RP4-769N13.6, GPRASP2, RP4-798P15.3, SEC16B, RP5-10211I20.4, ZNF410, RP6-109B7.3, FLJ27365, RPE, RPH3AL, RPL15, RPL17, RPL17-C18orf32, RPL17, RPL23A, RPL36, HSD11B1L, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACM1L, SAMD4B, SAR1A, SARAF, SARNP, RPII-762I7.5, SCAMP5, SCAP, SCAPER, SCFD1, SCGB3A2, SCIN, SCML1, SCNN1D, SCO2, SCOC, SCRN1, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPP1, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLC10A3, SLC12A9, SLC14A1, SLC16A6, SLC1A2, SLC1A6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFN11, SLFN12, SLFNL1, SLMO1, SLTM, SLU7, SMAD2, SMAP2, SMARCA2, SMARCE, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPD1, SNRPD2, SNRPN, SNRPN, SNURF, SNUPN, SNX11, SNX16, SNX17, SOAT1, SOHLH2, CCDC169-SOHLH2, CCDC169, SORBS1, SORBS2, SOX5, SP2, SPART, SPATA20, SPATA21, SPATS2, SPATS2L, SPDYE2, SPECC1, SPECC1L, SPECC1L-ADORA2A, SPECC1L-ADORA2A, ADORA2A, SPEG, SPG20, SPG21, SPIDR, SPIN1, SPOCD1, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPL1, STARD3NL, STAT6, STAU1, STAU2, AC022826.2, STAU2, RP11-463D19.2, STEAP2, STEAP3, STIL, STK25, STK33, STK38L, STK40, STMN, STON, STON-GTF2AIL, STRAP, STRBP, STRC, AC011330.5, STRC, CATSPER2, STRC, CATSPER2, AC011330.5, STRC, STRCP1, STT3A, STX16-NPEPL1, NPEPL1, STX5, STX6, STX8, STXBP6, STYK, SULTIA1, SULTIA2, SUMF2, SUN1, SUN2, SUN2, DNAL4, SUOX, SUPT6H, SUV39H2, SV2B, SYBU, SYNCRIP, SYNJ2, SYT, SYTL4, TAB2, TACC, TADA2B, TAF1C, TAF6, AC073842.2, TAF6, RP11-506M12.1, TAF9, TAGLN, TANK, TAPSAR1, PSMB9, TAPT1, TATDN1, TAZ, TBC1D1, TBC1D12, HELLS, TBC1D15, TBC1D3H, TBC1D3G, TBCID5, TBCID5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCP10L, AP000275.65, TCP11, TCP11L2, TCTN1, TDG, TDP1, TDRD7, TEAD2, TECR, TENC1, TENT4A, TEX264, TEX30, TEX37, TFDP1, TFDP2, TFEB, TFG, TFP1, TF, TFPI, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLR10, TM9SF1, TMBIM1, TMBIM4, TMBIM6, TMC6, TMCC1, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RPII-691N7.6, CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIP1, TNK2, TNNT1, TNRC18, TNS3, TOB2, TOM1L1, TOP1MT, TOP3B, TOX2, TP53, RP11-199F11.2, TP53I1, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMT10B, TRMT2B, TRMT2B-AS1, TRNT1, TRO, TROVE2, TRPS1, TRPT1, TSC2, TSGA10, TSPAN14, TSPAN3, TSPAN4, TSPAN5, TSPAN6, TSPAN9, TSPO, TTC12, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXN11, UBXN7, UGDH, UGGT1, UGP2, UMAD1, AC007161.3, UNC45A, UQCC1, URGCP-MRPS24, URGCP, USMG5, USP16, USP21, USP28, USP3, USP33, USP35, USP54, USP9Y, USPL1, UTP15, VARS2, VASH2, VAV3, VDAC1, VDAC2, VDR, VEZT, VGF, VIL1, VILL, VIPR1, VPS29, VPS37C, VPS8, VPS9D1, VRK2, VWA1, VWA5A, WARS, WASF1, WASHC5, WBP5, WDHD1, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, XRCC3, XRN2, XXYLT1, YIF1A, YIF1B, YIPF1, YIPF5, YPEL5, YWHAB, YWHAZ, YYJAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3H11A, ZBED6, ZC3H13, ZCCHC17, ZCCHC7, ZDHHC11, ZDHHC13, ZEB2, ZFAND5, ZFAND6, ZFP1, ZFP62, ZFX ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYND11, ZNF10, AC026786.1, ZNF133, ZNF146, ZNF16, ZNF177, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXO16, ZNF415, ZNF418, ZNF43, ZNF433-AS1, AC0008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP11-158I13.2, ZNF512B, ZNF512B, SAMD10, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOAM1 gene.

Exemplary genes that may be modulated by the compounds of Formula (I) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugcgug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugegug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugagge (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccucg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054).

Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgegegeg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacacc (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugegge (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccugc (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugagge (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugeg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550).

Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) described herein interacts with (e.g., binds to) a splicing complex component (e.g., a nucleic acid (e.g., an RNA) or a protein). In some embodiments, the splicing complex component is selected from 9G8, A1 hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, U1 snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNP1. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%7, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AIL) (e.g., B-cell AIL, T-cell AIL), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) increases expression of the haploinsufficient gene locus. In an embodiment, a compound of Formula (I) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or a pharmaceutically acceptable salt thereof, e.g., as described herein, has a cell permeability measurement (Papp) of <2×10⁻⁶ cm s⁻¹. In an embodiment, a compound of Formula (I) or a pharmaceutically acceptable salt thereof, e.g., as described herein, has a cell permeability measurement (Papp) of between 2-6×10⁻⁶ cm s⁻¹. In an embodiment, a compound of Formula (I) or a pharmaceutically acceptable salt thereof, e.g., as described herein, has a cell permeability measurement (Papp) of Papp greater than 6×10⁻⁶ cm s⁻¹. In an embodiment, a compound of Formula (I) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or a pharmaceutically acceptable salt thereof, e.g., as described herein, has a cell efflux ratio of less than 1.5. In an embodiment, a compound of Formula (I) or a pharmaceutically acceptable salt thereof, e.g., as described herein, has a cell efflux ratio of between 1.5 and 5. In an embodiment, a compound of Formula (I) or a pharmaceutically acceptable salt thereof, e.g., as described herein, has a cell efflux ratio greater than 5. In an embodiment, a compound of Formula (I) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, modulates the expression of a target protein (e.g., HTT or MYB) in a reference cell or sample. In an embodiment, a compound of Formula (I) or a pharmaceutically acceptable salt thereof, e.g., as described herein, increases the expression of a target protein (e.g., HTT or MYB) in a reference cell or sample. In an embodiment, a compound of Formula (I) or a pharmaceutically acceptable salt thereof, e.g., as described herein, decreases the expression of a target protein (e.g., HTT or MYB) in a reference cell or sample. The effect of an exemplary compound of Formula (I) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or a pharmaceutically acceptable salt thereof, e.g., as described herein, has a protein abundance response less than 100 nM. In an embodiment, a compound of Formula (I) or a pharmaceutically acceptable salt thereof, e.g., as described herein, has a protein abundance response between 100-1000 nM. In an embodiment, a compound of Formula (I) or a pharmaceutically acceptable salt thereof, e.g., as described herein, has a protein abundance response greater than 1000 nM. In an embodiment, a compound of Formula (I) or a pharmaceutically acceptable salt thereof, e.g., as described herein, has a protein abundance response greater than 10 uM. In an embodiment, a compound of Formula (I) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, modulates the viability of a target cell in a subject or sample. In an embodiment, a compound of Formula (I) or a pharmaceutically acceptable salt thereof, e.g., as described herein, increases the viability of a target cell in a subject or sample. In an embodiment, a compound of Formula (I) or a pharmaceutically acceptable salt thereof, e.g., as described herein, decreases the viability of a target cell in a subject or sample. In an embodiment, a compound of Formula (I) or a pharmaceutically acceptable salt thereof, e.g., as described herein, does not impact the viability of a cell (e.g., is non-toxic) in a subject or sample. The effect an exemplary compound of Formula (I) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or a pharmaceutically acceptable salt thereof, e.g., as described herein, is tolerated by a target cell at a concentration of less than 100 nM. In an embodiment, a compound of Formula (I) or a pharmaceutically acceptable salt thereof, e.g., as described herein, is tolerated by a target cell at a concentration of between 100-1000 nM. In an embodiment, a compound of Formula (I) or a pharmaceutically acceptable salt thereof, e.g., as described herein, is tolerated by a target cell at a concentration of greater than 1000 nM. In an embodiment, a compound of Formula (I) or a pharmaceutically acceptable salt thereof, e.g., as described herein, is tolerated by a target cell at a concentration of greater than 10 uM.

In some embodiments, a compound of Formula (I) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,brain}) may be defined as the ratio of unbound brain-free compound concentration to unbound plasma concentration. It is calculated using the following equation:

$${K}_{p , uu , brain} = \frac{{f}_{u , brain} \times {C}_{brain}}{{f}_{u , plasma} \times {C}_{plasma}}$$

C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or a pharmaceutically acceptable salt thereof, e.g., as described herein, has a Kp value of greater than 5. In an embodiment, a compound of Formula (I) or a pharmaceutically acceptable salt thereof, e.g., as described herein, has a Kp value between 1 and 5. In an embodiment, a compound of Formula (I) or a pharmaceutically acceptable salt thereof, e.g., as described herein, has a Kp value between 0.2-1. In an embodiment, a compound of Formula (I) or a pharmaceutically acceptable salt thereof, e.g., as described herein, has a Kp value of less than 0.2. In an embodiment, a compound of Formula (I) or a pharmaceutically acceptable salt thereof, e.g., as described herein, has a Kpuu value of greater than 2.5. In an embodiment, a compound of Formula (I) or a pharmaceutically acceptable salt thereof, e.g., as described herein, has a Kpuu value between 0.5-2.5. In an embodiment, a compound of Formula (I) or a pharmaceutically acceptable salt thereof, e.g., as described herein, has a Kpuu value between 0.1-0.5. In an embodiment, a compound of Formula (I) or a pharmaceutically acceptable salt thereof, e.g., as described herein, has a Kpuu value of less than 0.1. In an embodiment, a compound of Formula (I) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or a pharmaceutically acceptable salt thereof, e.g., as described herein, exhibits selectivity for HTT, e.g., an HTT-related nucleic acid sequence. In an embodiment, a compound of Formula (I) or a pharmaceutically acceptable salt thereof, e.g., as described herein, exhibits selectivity for SMN2, e.g., an SMN2-related nucleic acid sequence. In an embodiment, a compound of Formula (I) or a pharmaceutically acceptable salt thereof, e.g., as described herein, exhibits selectivity for Target C, e.g., a Target C-related nucleic acid sequence. In an embodiment, a compound of Formula (I) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or a pharmaceutically acceptable salt thereof, e.g., as described herein, has a 3-fold greater selectivity for HTT over MYB. In an embodiment, a compound of Formula (I) or a pharmaceutically acceptable salt thereof, e.g., as described herein, has a 3-fold greater selectivity for MYB over HTT. In an embodiment, a compound of Formula (I) or a pharmaceutically acceptable salt thereof, e.g., as described herein, has a 10-fold greater selectivity for HTT over MYB. In an embodiment, a compound of Formula (I) or a pharmaceutically acceptable salt thereof, e.g., as described herein, has a 10-fold greater selectivity for MYB over HTT. In an embodiment, a compound of Formula (I) or a pharmaceutically acceptable salt thereof, e.g., as described herein, has a 3-fold greater selectivity for HTT over SMN2. In an embodiment, a compound of Formula (I) or a pharmaceutically acceptable salt thereof, e.g., as described herein, has a 3-fold greater selectivity for SMN2 over HTT. In an embodiment, a compound of Formula (I) or a pharmaceutically acceptable salt thereof, e.g., as described herein, has a 10-fold greater selectivity for HTT over SMN2. In an embodiment, a compound of Formula (I) or a pharmaceutically acceptable salt thereof, e.g., as described herein, has a 10-fold greater selectivity for SMN2 over HTT. In an embodiment, a compound of Formula (I) or a pharmaceutically acceptable salt thereof, e.g., as described herein, has a 3-fold greater selectivity for MYB over SMN2. In an embodiment, a compound of Formula (I) or a pharmaceutically acceptable salt thereof, e.g., as described herein, has a 3-fold greater selectivity for SMN2 over MYB. In an embodiment, a compound of Formula (I) or a pharmaceutically acceptable salt thereof, e.g., as described herein, has a 10-fold greater selectivity for MYB over SMN2. In an embodiment, a compound of Formula (I) or a pharmaceutically acceptable salt thereof, e.g., as described herein, has a 10-fold greater selectivity for SMN2 over MYB. In an embodiment, a compound of Formula (I) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject.

Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### General Synthetic Scheme

Compounds of the present disclosure may be prepared using a synthetic protocol illustrated in the exemplary scheme shown below.

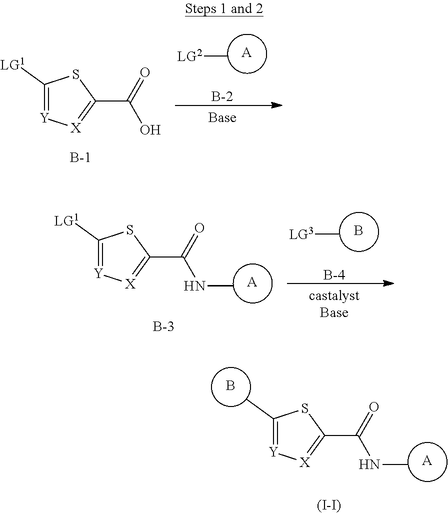

Scheme A. An exemplary method of preparing a compound of Formula (I); wherein A, B, X, and Y are as defined herein; and LG¹, LG², and LG³ are each independently hydrogen or a leaving group (e.g., halo, —B(OR¹²)₂).

An exemplary method of preparing a compound described herein, e.g., a compound of Formula (I-I) is provided in Scheme A. In Step 1, B-3 is prepared by incubating B-1 with B-2 in the presence of a base (e.g., Cs₂CO₃, DIEA, N-methylimidazole, or a suitable alternative). The reaction may be conducted in dimethylformamide or a similar solvent, at a temperature sufficient to provide B-3.

B-3 and B-4 are coupled to provide a compound of Formula (I-I) in Step 2. This coupling reaction may be conducted in the presence of a catalyst, such as a ruthenium, palladium, or copper catalyst (e.g., Pd-PEPPSI-IPentCl 2-methylpyridine, Pd(dppf)Cl₂, Pd₂(dba)₃, XPhos-Pd(II)-G2, XPhos-Pd(II)-G3, ^{t}BuBrettphos-Pd(II)-G3, RuPhos-Pd(II), RuPhos-Pd(II)-G2, RuPhos-Pd(II)-G3, or CuI, or similar reagent), and a base (e.g., Cs₂CO₃, K₂CO₃, BuONa, or a similar reagent). The reaction may be conducted in dimethylformamide or a similar solvent, at 100° C. or a temperature sufficient to provide the compound of Formula (I-I). Each starting material and/or intermediate in Scheme A may be protected and deprotected using standard protecting group methods. In addition, purification and characterization of each intermediate as well as the final compound of Formula (I) may be afforded by any accepted procedure.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC).

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 μm, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH-C18 (C18, Ø4.6×50 mm, 3 μm, 120 Å, 40° C.) operating in ESI(+) ionization mode; flow rate=1.2 mL/min. Mobile phase A: Water/5 mM NH₄HCO₃, Mobile phase B: CH₃CN.)

Analytical chiral HPLC: Analytical chiral HPLC was performed on a Agilent 1260 using column: CHLRALPAK IG-3, CHIRALPAK IC-3 or CHIRALPAK OJ-3, with flow rate=1.2 mL/min. Mobile phase=MTBE(DEA):EtOH=:50:50).

Preparative HPLC purification: prep-HPLC purification was performed using one of the following HPLC conditions:
Condition 1: Column: Xselect CSH OBD Column 30*150 mm 5 um; Mobile Phase A: water (10 mmol/L NH₄HCO₃); Mobile Phase B: acetonitrile; Flow rate: 60 mL/min; Gradient 1: 5 B to 45 B in 6 min; Gradient 2: 5 B to 40 B in 8 min; Gradient 3: 10 B to 50 B in 10 min; Gradient 4: 3 B to 33 B in 8 min.
Condition 2: Column: X-Bridge Prep OBD C18, 30-150 mm 5 um; Mobile phase A: water (10 mmol/L NH₄HCO₃); Mobile phase B: acetonitrile; Gradient 1: 5% Phase B up to 55% in 8 min; Gradient 2: 10% B to 43% B in 8 min; Gradient 3: 10% B to 50% B in 8 min; Gradient 4: 20% B to 52% B in 8 min; Gradient 5: 5% B to 45% B in 8 min; Gradient 6: 10% B to 30% B in 10 min; Gradient 7: 30% B to 70% B in 8 min.
Condition 3: Column: Waters Xbridge BEH C18 250*50 mm*10 um; Mobile Phase A: Water (NH₄HCO₃) Mobile Phase B: Acetonitrile; Gradient 1: 70% B-90% B in 10 min; Gradient 2: 50% B to 70% B in 8 min.
Condition 4: Column: Phenomenex C18 75*30 mm*3 um; Mobile Phase A: Water (NH₄HCO₃) Mobile Phase B: Acetonitrile; Gradient 1: 35% B-65% B in 8 min; Gradient 2: 60% B to 90% B in 8 min; Gradient 3: 1% B to 40% B in 7 min

Preparative chiral HPLC: purification by chiral HPLC was performed on a Gilson-GX 281 using column: CHIRALPAK IG-3, CHIRALPAK IC-3 or CHIRALPAK OJ-3.
Condition 1: Column: IG 2 cm×25 cm; Mobile Phase A: methyl tert-butyl ether; Mobile Phase B: ethanol; Gradient 1:1; Flow rate: 18 mL/min.
Condition 2: Gilson 281 semi-preparative: Column: Phenomenex Luna 80×30 mm×3 μm; Mobile Phase A: Water (4.8 mmol/L HCl), Mobile Phase B: Acetonitrile; Gradient 1:1% B to 30% B in 8 min.
Condition 3: Column, Kinetex EVO C18 Column, 30×150, 5 um; Mobile Phase A: Water (10 mmol/L NH₄HCO₃), Mobile Phase B: Acetonitrile; Gradient 1: 20% B to 55% B in 8 min.

### Example 1: Synthesis of Compound 100

### Synthesis of Intermediate B3

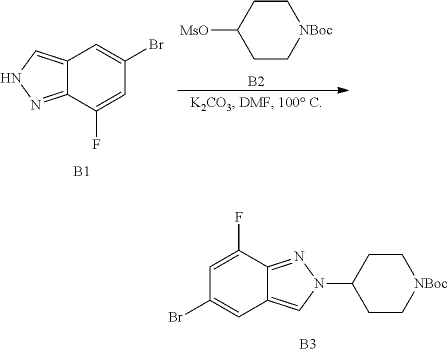

To a stirred solution of 5-bromo-7-fluoro-2H-indazole (B1, 500 mg, 2.32 mmol, 1 equiv) and tert-butyl 4-(methanesulfonyloxy) piperidine-1-carboxylate (B2, 974.36 mg, 3.48 mmol, 1.5 equiv) in DMF (10 mL) was added K₂CO₃ (964.11 mg, 6.97 mmol, 3 equiv) dropwise at room temperature under nitrogen atmosphere. The resulting mixture was stirred overnight at 100° C., and the reaction was quenched with water at room temperature. The resulting mixture was extracted with EtOAc (2×30 mL), then the combined organic layers were washed with brine (2×50 mL), dried over anhydrous Na₂SO₄, filtered, and concentrated under reduced pressure to afford tert-butyl 4-(5-bromo-7-fluoroindazol-2-yl) piperidine-1-carboxylate (B3, 800 mg, 86%) as a solid.

### Synthesis of Intermediate B5

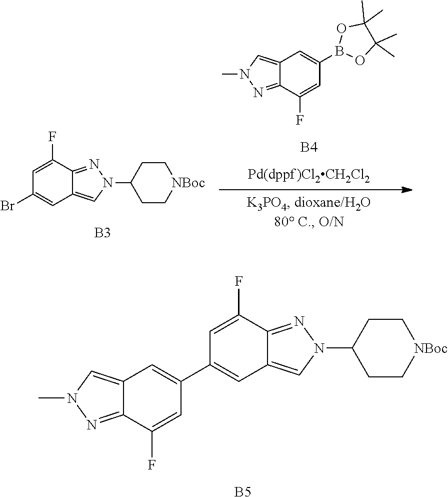

To a stirred solution of tert-butyl 4-(5-bromo-7-fluoroindazol-2-yl) piperidine-1-carboxylate (B3, 700 mg, 1.75 mmol, 1 equiv) and 7-fluoro-2-methyl-5-(4,4,5,5-tetramethyl-1,3,2-dioxaborolan-2-yl) indazole (B4, 727.95 mg, 2.63 mmol, 1.5 equiv) in dioxane (7 mL) were added Pd(dppf)Cl₂CH₂Cl₂ (143.18 mg, 0.17 mmol, 0.1 equiv) and K₃PO₄ (1119.22 mg, 5.27 mmol, 3 equiv) and H₂O (2 mL) dropwise at room temperature under nitrogen atmosphere. The resulting mixture was stirred overnight at 80° C. After filtration, the filtrate was concentrated under reduced pressure and the residue was purified by silica gel column chromatography, eluted with CH₂Cl₂/MeOH (10:1) to afford tert-butyl 4-{7,7 [symbol] difluoro-2 [symbol] methyl-[5,5 [symbol] biindazol]-2-yl}piperidine-1-carboxylate (B5, 500 mg, 60%) as a solid.

### Synthesis of Compound 100

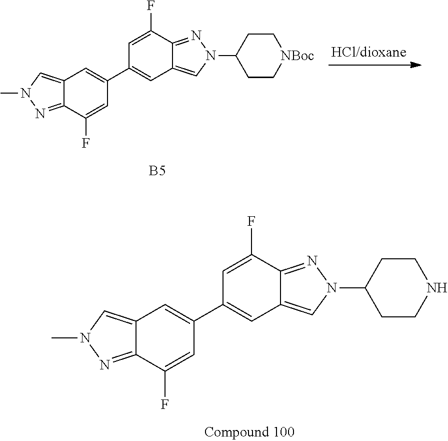

To a stirred solution of tert-butyl 4-{7,7 [symbol] difluoro-2 [symbol] methyl-[5,5 [symbol] biindazol]-2-yl}piperidine-1-carboxylate (B5, 500 mg, 1.06 mmol, 1 equiv) in MeOH (5 mL) was added HCl (gas) in 1,4-dioxane (5 mL) dropwise at room temperature under nitrogen atmosphere. The resulting mixture was stirred for 1 h at room temperature, and the resulting mixture was concentrated under reduced pressure. The crude product was purified by Chiral-Prep-HPLC (Condition 1, Gradient 1) to afford 7 [symbol] difluoro-2 [symbol] methyl-1-(piperidin-4-yl)-5,5 [symbol] biindazole (Compound 100, 19.6 mg, 4%) as a solid. LCMS: (ES, m/z): 368 [M+H]⁺. ¹H NMR (400 MHz, Chloroform-d) δ 8.07 (d, J=2.7 Hz, 1H), 7.99 (d, J=2.6 Hz, 1H), 7.61 (dd, J=8.1, 1.4 Hz, 2H), 7.29 (m, 1H), 7.21 (m, 1H), 4.61 (tt, J=11.7, 4.0 Hz, 1H), 4.27 (s, 3H), 3.38-3.29 (m, 2H), 2.87 (td, J=12.6, 2.5 Hz, 2H), 2.38-2.29 (m, 2H), 2.15-2.04 (m, 2H).

### Example 2: Synthesis of Compound 103

### Synthesis of Intermediate B7

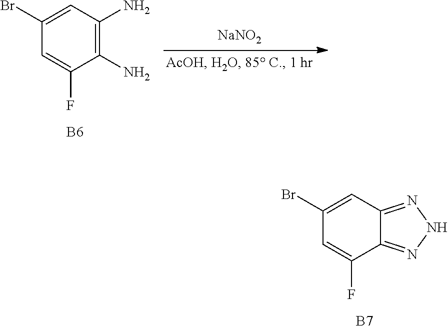

To a solution of 5-bromo-3-fluoro-benzene-1,2-diamine (B6, 5 g, 24.39 mmol, 1 equiv) in H₂O (50 mL) was added a solution of NaNO₂ (1.85 g, 26.83 mmol, 1.1 equiv) in H₂O (50 mL) at 15° C. The mixture was stirred at 15° C. for 15 min. Then AcOH (18.90 g, 314.73 mmol, 18 mL, 12.91 equiv) was added dropwise at 15° C. The resulting mixture was stirred at 85° C. for 1 hr. The reaction mixture was diluted with water (50 mL) and extracted with ethyl acetate (3×50 mL). The combined organic layers were washed with brine (3×50 mL), dried over Na₂SO₄, filtered and concentrated under reduced pressure to afford 6-bromo-4-fluoro-2H-benzotriazole (B7, 5 g, 94%) as a solid. LCMS: (ES, m/z): 215.9 [M+H]⁺. ¹H NMR (400 MHz, DMSO-d₆) δ ppm 8.01 (d, J=1.19 Hz, 1H) 7.52 (dd, J=10.01, 1.19 Hz, 1H).

### Synthesis of Intermediate B8

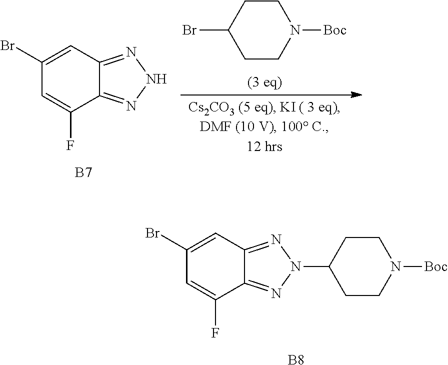

A mixture of 6-bromo-4-fluoro-2H-benzotriazole (B7, 200 mg, 925.88 umol, 1 equiv), tert-butyl 4-bromopiperidine-1-carboxylate (733.74 mg, 2.78 mmol, 3 equiv), Cs₂CO₃ (1.51 g, 4.63 mmol, 5 equiv) and KI (461.09 mg, 2.78 mmol, 3 equiv) in DMF (2 mL) was stirred at 100° C. for 12 hrs under nitrogen atmosphere. The reaction mixture was diluted with ethyl acetate (3 mL) and water (3 mL), extracted with ethyl acetate (3×3 mL). The combined organic layers were washed with brine (2×5 mL), dried over anhydrous Na₂SO₄, filtered, and concentrated under reduced pressure to give a residue. The residue was purified by silica gel column chromatography, eluted with petroleum ether/ethyl acetate (10: 1) to afford tert-butyl 4-(6-bromo-4-fluoro-benzotriazol-2-yl)piperidine-1-carboxylate (B8, 170 mg, 44%) as an oil. LCMS: (ES, m/z): 343 [M+H−56]⁺. ¹H NMR (400 MHz, Chloroform-d) δ ppm 7.85 (d, J=0.6 Hz, 1H), 7.18 (dd, J=9.4, 0.6 Hz, 1H), 4.93 (quin, J=7.6 Hz, 1H), 4.24 (d, J=8.8 Hz, 2H), 3.06 (d, J=7.0 Hz, 2H), 2.25-2.33 (m, 4H), 1.49 (s, 9H). ¹⁹F NMR (400 MHz, Chloroform-d) δ ppm −121.64.

### Synthesis of Intermediate B10

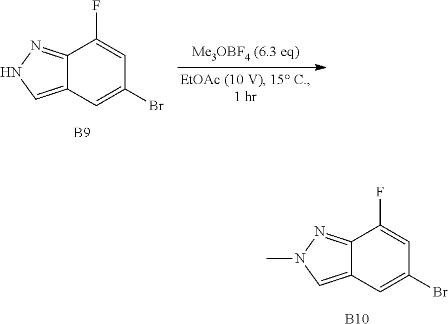

To a solution of 5-bromo-7-fluoro-2H-indazole (B9, 500 mg, 2.33 mmol, 1 equiv) in ethyl acetate (30 mL) was added Me₃OBF₄ (2.17 g, 14.65 mmol, 6.3 equiv). The mixture was stirred at 15° C. for 1 hr. The reaction mixture was diluted with water (20 mL) and extracted with ethyl acetate (3×50 mL). The combined organic layers were washed with brine (3×50 mL), dried over Na₂SO₄, filtered and concentrated under reduced pressure to give a residue. The residue was purified by column chromatography (SiO₂, Petroleum ether/Ethyl acetate=5/1 to 0/1) to obtain 5-bromo-7-fluoro-2-methyl-indazole (B10, 290 mg, 54%) as a solid. LCMS: (ES, m/z): 229[M+H]⁺. ¹H NMR (400 MHz, DMSO-d₆) δ ppm 8.47 (d, J=2.74 Hz, 1H) 7.83 (d, J=1.07 Hz, 1H) 7.25 (dd, J=10.97, 1.07 Hz, 1H) 4.20 (s, 3H)

### Synthesis of Intermediate B11

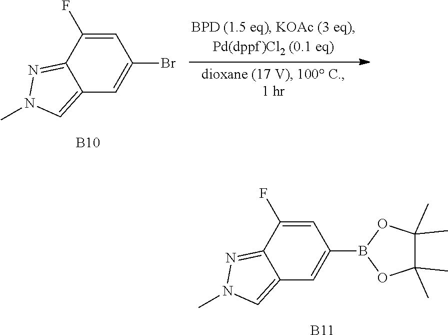

A mixture of 5-bromo-7-fluoro-2-methyl-indazole (B10, 30 mg, 130.98 umol, 1 equiv), 4,4,5,5-tetramethyl-2-(4,4,5,5-tetramethyl-1,3,2-dioxaborolan-2-yl)-1,3,2-dioxaborolane (49.89 mg, 196.46 umol, 1.5 equiv), KOAc (38.56 mg, 392.93 umol, 3 equiv), Pd(dppf)Cl₂ (9.58 mg, 13.10 umol, 0.1 equiv) in dioxane (0.5 mL) was stirred at 100° C. for 1 hr under nitrogen atmosphere. The reaction mixture was filtered through a pad of Celite and the filter cake was washed with dioxane (3×5 mL). The combined filtrates were concentrated to dryness to afford 7-fluoro-2-methyl-5-(4,4,5,5-tetramethyl-1,3,2-dioxaborolan-2-yl)indazole (B11, 60 mg, 91%) as a solid. LCMS: (ES, m/z): 277 [M+H]⁺.

### Synthesis of Intermediate B12

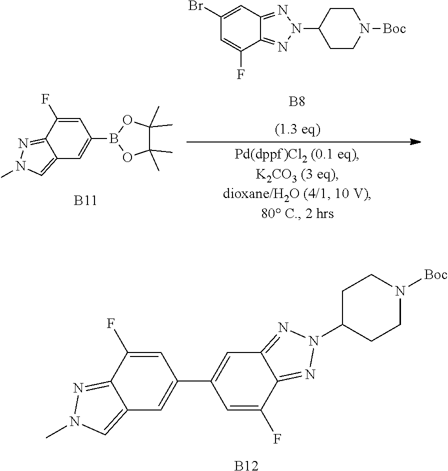

A mixture of 7-fluoro-2-methyl-5-(4,4,5,5-tetramethyl-1,3,2-dioxaborolan-2-yl)indazole (B11, 60 mg, 119.52 μmol), tert-butyl 4-(6-bromo-4-fluoro-benzotriazol-2-yl)piperidine-1-carboxylate (B8, 62.03 mg, 155.37 umol, 1.3 equiv), Pd(dppf)Cl₂ (8.75 mg, 11.95 umol, 0.1 equiv), K₂CO₃ (49.55 mg, 358.55 umol, 3 equiv) in dioxane (0.48 mL) and H₂O (0.12 mL) was stirred at 80° C. for 2 hr under nitrogen atmosphere. The reaction mixture was concentrated to give the crude product. The crude product was purified by silica gel column chromatography, eluted with petroleum ether/ethyl acetate (2:3) to afford tert-butyl 4-[4-fluoro-6-(7-fluoro-2-methyl-indazol-5-yl)benzotriazol-2-yl]piperidine-1-carboxylate (B12, 18 mg, 28%) as a solid. LCMS: (ES, m/z): 469 [M+H]⁺. ¹H NMR (400 MHz, Methanol-d₄) δ ppm 8.38 (d, J=2.6 Hz, 1H), 7.93 (d, J=1.1 Hz, 1H), 7.85 (d, J=1.1 Hz, 1H), 7.50 (dd, J=11.9, 1.04 Hz, 1H), 7.41 (dd, J=12.8, 1.2 Hz, 1H), 5.03-5.17 (m, 1H), 4.27 (s, 3H), 4.21 (d, J=13.8 Hz, 2H), 3.08-3.23 (m, 2H), 2.30-2.38 (m, 2H), 2.17-2.28 (m, 2H), 1.51 (s, 9H). ¹⁹F NMR (400 MHz, Methanol-d₄) δ ppm −130.90, 126.40.

### Synthesis of Compound 103

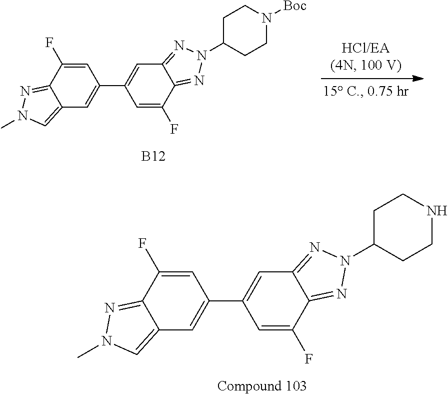

To a solution of tert-butyl 4-[4-fluoro-6-(7-fluoro-2-methyl-indazol-5-yl)benzotriazol-2-yl]piperidine-1-carboxylate (B12, 18 mg, 38.42 umol, 1 equiv) in ethyl acetate (1 mL) was added HCl (g)/ethyl acetate (4 M, 1 mL) at 15° C. The mixture was stirred at 15° C. for 0.75 hr. The reaction mixture was filtered and the filter cake was washed with ethyl acetate (3×3 mL). The filter cake was dried in vacuum to give 4-fluoro-6-(7-fluoro-2-methyl-indazol-5-yl)-2-(4-piperidyl) benzotriazole (Compound 103, 12.28 mg, 69%) as a solid. LCMS: (ES, m/z): 369 [M+H]⁺. ¹H NMR (400 MHz, Methanol-d₄) δ ppm 8.41 (d, J=2.7 Hz, 1H), 7.95 (d, J=1.0 Hz, 1H), 7.87 (d, J=1.0 Hz, 1H), 7.54 (dd, J=12.0, 0.8 Hz, 1H), 7.43 (dd, J=12.7, 1.1 Hz, 1H), 5.29 (tt, J=9.2, 4.7 Hz, 1H), 4.28 (s, 3H), 3.62 (dt, J=13.2, 4.2 Hz, 2H), 3.33-3.40 (m, 2H), 2.56-2.67 (m, 4H). ¹⁹F NMR (400 MHz, Methanol-d₄) δ ppm −130.78, 126.25.

### Example 3: Synthesis of Compound 109

### Synthesis of Intermediate B14

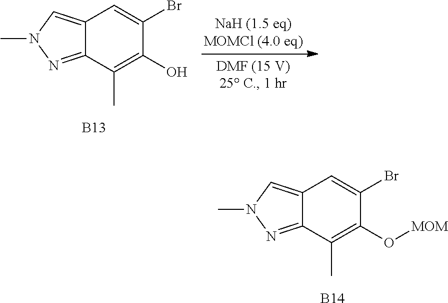

To a solution of 5-bromo-2,7-dimethyl-indazol-6-ol (B13, 1 g, 4.15 mmol, 1 equiv) in DMF (10 mL) was added NaH (497.70 mg, 6.22 mmol, 60%, 1.5 equiv) at 0° C. under N₂ atmosphere. The reaction mixture was stirred at 0° C. for 30 mins. To the reaction mixture was added MOMCl (1.34 g, 16.59 mmol, 1.26 mL, 4 equiv) at 0° C. under N₂ atmosphere. The reaction mixture was stirred at 25° C. for 1 hr. The mixture was quenched with saturated NaHCO₃ solution (30 mL), and extracted with ethyl acetate (3×30 mL). The combined organic phases were washed with brine (30 mL), dried over Na₂SO₄, filtered and the filtrate was concentrated to give crude product, which was purified by column chromatography on silica gel, eluted with petroleum ether/ethyl acetate (6:1) to give 5-bromo-6-(methoxymethoxy)-2,7-dimethyl-indazole (B14, 900 mg, 68%) as a solid. LCMS: (ES, m/z): 285 [M+H]⁺, ¹HNMR (400 MHz, DMSO-d₆) δ ppm 8.25 (s, 1H) 7.85 (s, 1H) 4.99 (s, 2H) 4.11 (s, 3H) 3.54 (s, 3H) 2.44 (s, 3H).

### Synthesis of Intermediate B15

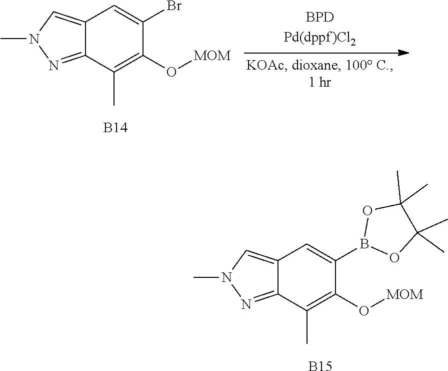

To a stirred solution of 5-bromo-6-(methoxymethoxy)-2,7-dimethyl-indazole (B14, 100 mg, 350.71 umol, 1 equiv) and 4,4,5,5-tetramethyl-2-(4,4,5,5-tetramethyl-1,3,2-dioxaborolan-2-yl)-1,3,2-dioxaborolane (133.59 mg, 526.06 umol, 1.5 equiv) in dioxane (1.5 mL) were added Pd(dppf)Cl₂ (25.66 mg, 35.07 umol, 0.1 equiv) and KOAc (103.26 mg, 1.05 mmol, 3 equiv) at 15° C. under nitrogen atmosphere. The resulting mixture was stirred at 100° C. for 2 hr. After filtration, the filtrate was concentrated under reduced pressure to afford 6-(methoxymethoxy)-2,7-dimethyl-5-(4,4,5,5-tetramethyl-1,3,2-dioxaborolan-2-yl)indazole (B15, 100 mg, 50%) as an oil, The product was used in the next step without further purification. LCMS: (ES, m/z): 333 [M+H]⁺.

### Synthesis of Intermediate B17

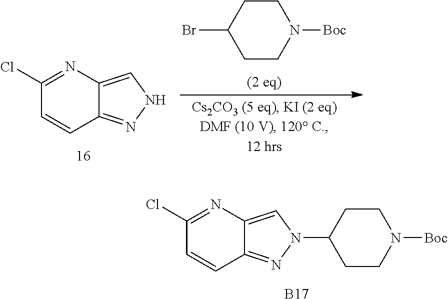

A mixture of tert-butyl 4-bromopiperidine-1-carboxylate (10.32 g, 39.07 mmol, 2 equiv), Cs₂CO₃ (31.82 g, 97.68 mmol, 5 equiv), KI (6.49 g, 39.07 mmol, 2 equiv) and 5-chloro-2H-pyrazolo[4,3-b]pyridine (B16, 3 g, 19.54 mmol, 1 equiv) in DMF (30 mL) was stirred at 120° C. for 12 hr. The mixture was quenched with H₂O (150 mL) and extracted with dichloromethane (3×100 mL). The combined organic phases were washed with brine (150 mL), dried over Na₂SO₄, filtered and the filtrate was concentrated to give crude product, which was purified by column chromatography on silica gel, eluted with petroleum ether/ethyl acetate (1:10 to 1:2) to give tert-butyl 4-(5-chloropyrazolo[4,3-b]pyridin-2-yl)piperidine-1-carboxylate (B17, 0.4 g, 5%) as a solid. LCMS: (ES, m/z): 337 [M+H]⁺. ¹H NMR (400 MHz, Methanol-d₄) δ 8.48 (s, 1H), 8.09 (dd, 1H, J=0.8, 9.0 Hz), 7.30 (d, 1H, J=9.0 Hz), 4.75 (tt, 1H, J=4.3, 11.4 Hz), 4.27 (d, 2H, J=13.4 Hz), 3.03 (s, 2H), 2.0-2.3 (m, 4H), 1.49 (s, 9H).

### Synthesis of Intermediate B18

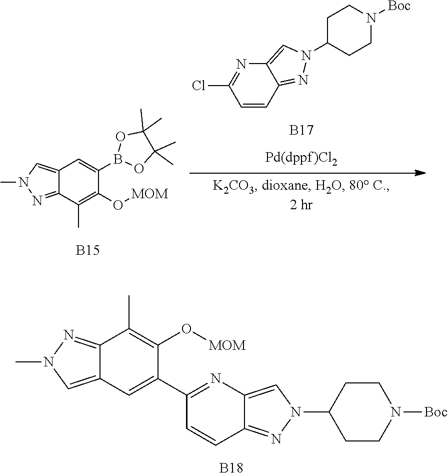

To a stirred solution of 6-(methoxymethoxy)-2,7-dimethyl-5-(4,4,5,5-tetramethyl-1,3,2-dioxaborolan-2-yl)indazole (B15, 100 mg, 301.02 umol, 1 equiv) and tert-butyl 4-(5-chloropyrazolo[4,3-b]pyridin-2-yl)piperidine-1-carboxylate (B17, 101.39 mg, 301.02 umol, 1 equiv) in dioxane (1.6 mL) and H₂O (0.4 mL) were added Pd(dppf)Cl₂ (22.03 mg, 30.10 umol, 0.1 equiv) and K₂CO₃ (124.81 mg, 903.07 umol, 3 equiv) at 25° C. under nitrogen atmosphere. The resulting mixture was stirred at 80° C. for 2 hrs. After filtration, the filtrate was concentrated under reduced pressure to get a residue. The residue was purified by silica gel column chromatography, eluted with ethyl acetate/MeOH (5:1) to afford tert-butyl 4-[5-[5-(methoxymethoxy)-2,7-dimethyl-indazol-6-yl]pyrazolo[4,3-b]pyridin-2-yl]piperidine-1-carboxylate (B18, 25 mg, 80%) as a solid. LCMS: (ES, m/z): 507 [M+H]⁺.

### Synthesis of Compound 109

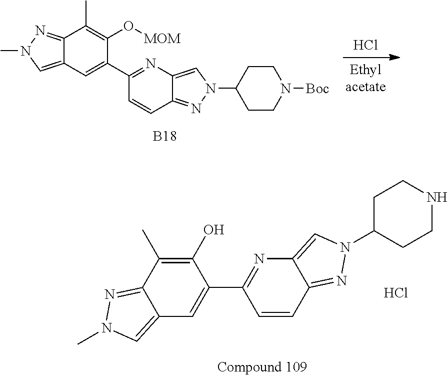

To a stirred solution of tert-butyl 4-[5-[5-(methoxymethoxy)-2,7-dimethyl-indazol-6-yl]pyrazolo[4,3-b]pyridin-2-yl]piperidine-1-carboxylate (B18, 25 mg, 49.35 umol, 1 equiv) in ethyl acetate (0.1 mL) was added HCl/ethyl acetate (4 M, 0.5 mL) dropwise at 25° C. under nitrogen atmosphere. The resulting mixture was stirred at 25° C. for 20 min. After filtration, the filter cake was collected and dried in vacuum to afford 2,7-dimethyl-5-[2-(4-piperidyl)pyrazolo[4,3-b]pyridin-5-yl]indazol-6-ol (Compound 109, 19.53 mg, 98%) as a solid. LCMS: (ES, m/z): 363 [M+H]⁺. ¹H NMR (400 MHz, DMSO-d₆) δ 9.03-9.19 (m, 1H), 8.84-8.99 (m, 1H), 8.81 (s, 1H), 8.46 (s, 1H), 8.40 (s, 1H), 8.34 (d, J=9.4 Hz, 1H), 8.22 (d, J=9.6 Hz, 1H), 4.93 (t, J=7.3 Hz, 1H), 4.15 (s, 3H), 3.47 (d, J=12.28 Hz, 2H), 3.07-3.23 (m, 2H), 2.36 (d, J=3.70 Hz, 7H).

### Example 4: Synthesis of Compound 122

### Synthesis of Intermediate B19

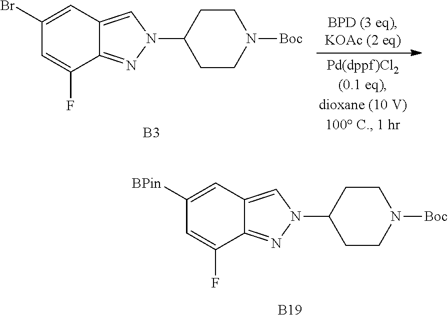

To a mixture of tert-butyl 4-(5-bromo-7-fluoro-2H-indazol-2-yl)-piperidine-1-carboxylate (B3, 2.5 g, 6.28 mmol, 1 equiv) and Bis(pinacolato)diboron (4.78 g, 18.83 mmol, 3 equiv) in dioxane (25 mL) was added potassium acetate (1.23 g, 12.55 mmol, 2 equiv) and [1,1′-Bis(diphenylphosphino)ferrocene]-dichloropalladium (II) (493.89 mg, 627.72 umol, 0.1 equiv) successively. The reaction mixture was bubbled with argon for 5 mins and stirred at 100° C. for 1 hr. The reaction mixture was filtered and the filtrate was concentrated in vacuum to get a brown oil. The brown oil was purified by column chromatography on silica gel eluted with petroleum ether/ethyl acetate (1/0 to 5/1) to give tert-butyl-4-(7-fluoro-5-(4,4,5,5-tetramethyl-1,3,2-dioxaborolan-2-yl)-2H-indazol-2-yl)piperidine-1-carboxylate (B19, 3 g, 91%) as an oil. LCMS: (ESI, m/z): 319 [M+H]⁺. ¹H NMR (400 MHz, DMSO) δ ppm 8.66 (d, J=2.81 Hz, 1H) 7.95 (s, 1H) 7.07 (d, J=12.23 Hz, 1H) 4.76 (s, 1H) 4.10 (br d, J=12.10 Hz, 2H) 2.97 (br s, 2H) 2.10-2.20 (m, 2H) 1.89-1.98 (m, 2H) 1.43 (s, 9H) 1.30 (s, 12H)

### Synthesis of Intermediate B21

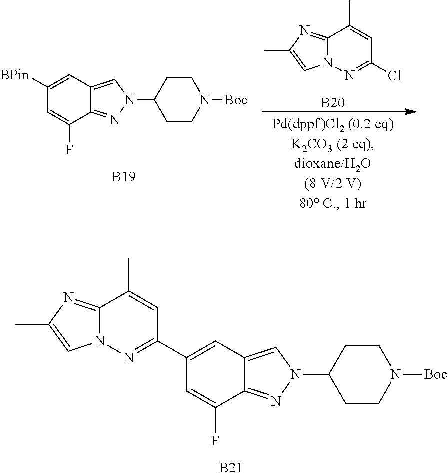

To a mixture of tert-butyl 4-(7-fluoro-5-(4,4,5,5-tetramethyl-1,3,2-dioxaborolan-2-yl)-2H-indazol-2-yl)piperidine-1-carboxylate (B19, 300 mg, 673.65 umol, 2 equiv) and 6-chloro-2,8-dimethylimidazo[1,2-b]pyridazine (B20, 61.17 mg, 336.83 umol, 1 equiv) in dioxane (2.4 mL) and H₂O (0.6 mL) was added K₂CO₃ (93.10 mg, 673.65 umol, 2 equiv) and [1,1′-Bis(diphenylphosphino)ferrocene]-dichloropalladium (II) (49.29 mg, 67.37 umol, 0.2 equiv) successively. The reaction mixture was stirred at 80° C. for 1 hr. The reaction mixture was quenched with water (10 mL), and extracted with ethyl acetate (3×10 mL). The organic layer was dried over Na₂SO₄, filtered and concentrated under reduced pressure to give an oil. The oil was purified by column chromatography on silica gel eluted with petroleum ether/ethyl acetate (10/1 to 1/0) to give tert-butyl 4-(5-(2,8-dimethylimidazo [1,2-b]pyridazin-6-yl)-7-fluoro-2H-indazol-2-yl) piperidine-1-carboxylate (B21, 150 mg, 70%) as an oil. LCMS: (ESI, m/z): 465 [M+H]⁺. ¹H NMR (400 MHz, DMSO) δ ppm 8.74 (d, J=2.81 Hz, 1H) 8.29 (s, 1H) 8.03 (s, 1H) 7.66-7.77 (m, 2H) 3.77 Hz, 1H) 4.80 (tt, J=11.39, 4.13 (br d, J=11.74 Hz, 2H) 3.72-3.86 (m, 1H) 2.60 (s, 3H) 2.40 (s, 3H) 2.11-2.21 (m, 2H) 2.01 (br d, J=4.28 Hz, 1H) 1.91-1.98 (m, 1H) 1.63-1.73 (m, 1H) 1.44 (s, 9H).

### Synthesis of Compound 122

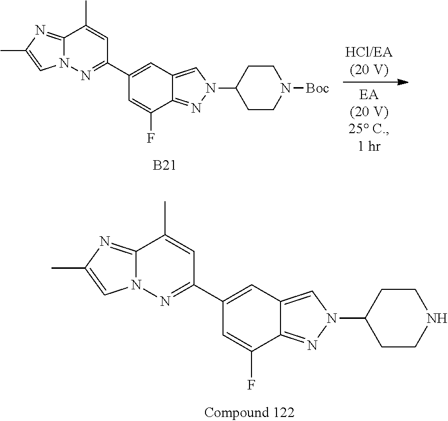

To a solution of tert-butyl 4-(5-(2,8-dimethylimidazo[1,2-b]pyridazin-6-yl)-7-fluoro-2H-indazol-2-yl)piperidine-1-carboxylate (B21, 100 mg, 215.27 umol, 1 equiv) in ethyl acetate (2 mL) was added HCl/ethyl acetate (4 M, 2.02 mL, 37.5 equiv). The reaction mixture was stirred at 25° C. for 1 hr. The reaction mixture was filtered and the filter cake was dried in vacuum to get a yellow residue. The crude product was purified by prep-HPLC (Condition 2, Gradient 1) to afford 6-(7-fluoro-2-(piperidin-4-yl)-2H-indazol-5-yl)-2,8-dimethylimidazo [1,2-b]pyridazine hydrochloride salt (Compound 122, 24.02 mg, 19%) as a solid. LCMS: (ESI, m/z): 365 [M+H]⁺. ¹H NMR (400 MHz, methanol-d₄) δ ppm 8.66 (d, J=2.57 Hz, 1H) 8.43 (d, J=1.22 Hz, 1H) 8.30 (dd, J=4.71, 1.04 Hz, 2H) 7.85 (dd, J=12.84, 1.34 Hz, 1H) 5.00 (tt, J=10.03, 5.07 Hz, 1H) 3.62-3.73 (m, 2H) 3.32-3.37 (m, 2H) 2.82 (d, J=0.98 Hz, 1H) 2.78 (d, J=0.98 Hz, 3H) 2.65 (d, J=0.86 Hz, 3H) 2.44-2.58 (m, 4H). ¹⁹F NMR (400 MHz, methanol-d₄) δ ppm −129.67 (s, 1F).

### Example 5: Synthesis of EVAL-0122-0003

### Synthesis of Intermediate B23

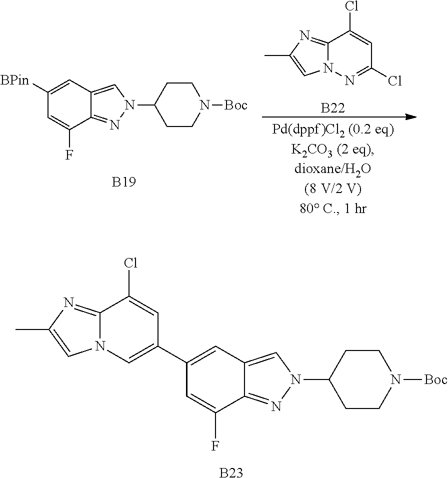

To a mixture of tert-butyl 4-(7-fluoro-5-(4,4,5,5-tetramethyl-1,3,2-dioxaborolan-2-yl)-2H-indazol-2-yl)piperidine-1-carboxylate (B19, 250 mg, 561.38 umol, 1.5 equiv) and 6-bromo-8-chloro-2-methylimidazo[1,2-a]pyridine (B22, 91.88 mg, 374.25 umol, 1 equiv) in dioxane (2 mL) and H₂O (0.5 mL) was added K₂CO₃ (103.45 mg, 748.50 umol, 2 equiv) and [1,1′-Bis(diphenylphosphino)ferrocene]-dichloropalladium (II) (54.77 mg, 74.85 umol, 0.2 equiv) successively. The reaction mixture was stirred at 80° C. for 1 hr under N₂ protection. The reaction mixture was diluted with water (10 mL) and extracted with ethyl acetate (3×10 mL). The organic layer was dried over Na₂SO₄, filtered and concentrated under reduced pressure to give an oil. The oil was purified by column chromatography on silica gel eluted with petroleum ether/ethyl acetate (5/1 to 0/1) to give tert-butyl-4-(5-(8-chloro-2-methylimidazo [1,2-a]pyridin-6-yl)-7-fluoro-2H-indazol-2-yl) piperidine-1-carboxylate (B23, 160 mg, 66%) as an oil. LCMS: (ESI, m/z): 484 [M+H]⁺. ¹H NMR (400 MHz, methanol-d4) δ ppm 8.64 (d, J=1.50 Hz, 1H) 8.48 (d, J=2.63 Hz, 1H) 7.71-7.86 (m, 3H) 7.34 (dd, J=12.63, 1.25 Hz, 1H) 4.74 (tt, J=11.43, 4.14 Hz, 1H) 4.29 (br d, J=13.76 Hz, 2H) 3.04 (br s, 2H) 2.45 (s, 3H) 2.07-2.26 (m, 4H) 1.50 (s, 9H). ¹⁹F NMR (400 MHz, methanol-d4) δ ppm −130.23 (s, 1F).

### Synthesis of Compound 127

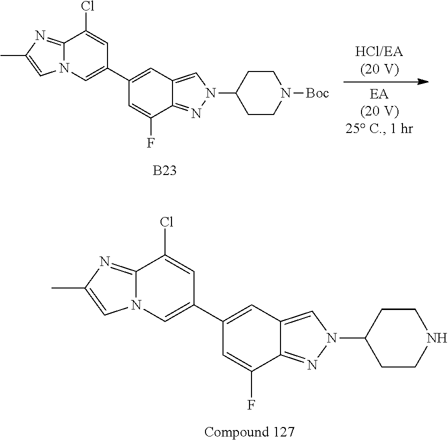

To a solution of tert-butyl 4-(5-(8-chloro-2-methylimidazo[1,2-a]pyridin-6-yl)-7-fluoro-2H-indazol-2-yl)piperidine-1-carboxylate (B23, 150 mg, 309.94 umol, 1 equiv) in ethyl acetate (3 mL) was added HCl/ethyl acetate (4 M, 3.01 mL, 38.9 equiv). The reaction was stirred at 25° C. for 1 hr. The reaction mixture was filtered and the filter cake was concentrated in vacuum to give a residue. The residue was triturated with methyl tert-butyl ether (3 mL) to afford 5-(8-chloro-2-methylimidazo[1,2-a]pyridin-6-yl)-7-fluoro-2-(piperidin-4-yl)-2H-indazole hydrochloride salt (Compound 127, 101.70 mg, 72%) as a solid. LCMS: (ESI, m/z): 384 [M+H]⁺. ¹H NMR (400 MHz, methanol-d₄) δ ppm 9.06 (d, J=1.31 Hz, 1H) 8.61 (d, J=2.62 Hz, 1H) 8.46 (d, J=1.31 Hz, 1H) 8.10 (d, J=0.95 Hz, 1H) 7.99 (d, J=1.31 Hz, 1H) 7.45 (dd, J=12.46, 1.25 Hz, 1H) 5.00 (dt, J=10.10, 5.02 Hz, 1H) 3.59-3.72 (m, 2H) 3.32-3.37 (m, 2H) 2.62 (d, J=0.72 Hz, 3H) 2.44-2.56 (m, 4H).

### Example 6: Synthesis of Compound 129

### Synthesis of Intermediate B25

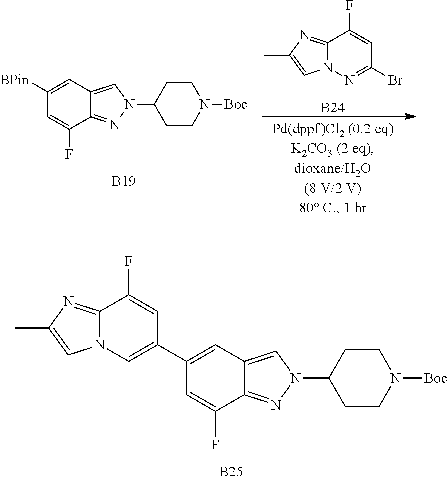

To a mixture of tert-butyl 4-(7-fluoro-5-(4,4,5,5-tetramethyl-1,3,2-dioxaborolan-2-yl)-2H-indazol-2-yl)piperidine-1-carboxylate (B19, 250 mg, 561.38 umol, 1.5 equiv) and 6-bromo-8-fluoro-2-methylimidazo[1,2-a]pyridine (B24, 85.72 mg, 374.25 umol, 1 equiv) in dioxane (2 mL) and H₂O (0.5 mL) was added K₂CO₃ (103.45 mg, 748.50 umol, 2 equiv) and [1,1′-Bis(diphenylphosphino)ferrocene]-dichloropalladium (II) (54.77 mg, 74.85 umol, 0.2 equiv) successively. The reaction mixture was stirred at 80° C. for 1 hr. The reaction mixture was diluted with water (10 mL) and extracted with ethyl acetate (3×10 mL). The organic layer was dried over Na₂SO₄, filtered and concentrated under reduced pressure to give an oil. The oil was purified by column chromatography on silica gel eluted with petroleum ether/ethyl acetate (5/1 to 0/1) to give tert-butyl 4-(7-fluoro-5-(8-fluoro-2-methylimidazo[1,2-a]pyridin-6-yl)-2H-indazol-2-yl)piperidine-1-carboxylate (B25, 110 mg, 49%) as an oil. LCMS: (ESI, m/z): 468 [M+H]⁺. 1H NMR (400 MHz, methanol-d4) δ ppm 8.54 (d, J=1.13 Hz, 1H) 8.48 (d, J=2.63 Hz, 1H) 7.80 (s, 1H) 7.74 (d, J=2.75 Hz, 1H) 7.44 (dd, J=12.01, 1.13 Hz, 1H) 7.35 (dd, J=12.63, 1.13 Hz, 1H) 4.74 (s, 1H) 4.29 (br d, J=13.51 Hz, 2H) 3.04 (br s, 2H) 2.44 (s, 3H) 2.05-2.32 (m, 4H) 1.50 (s, 9H). ¹⁹F NMR (400 MHz, methanol-d4) δ ppm −134.04 (s, 1F) −130.31 (s, 1F)

### Synthesis of Compound 129

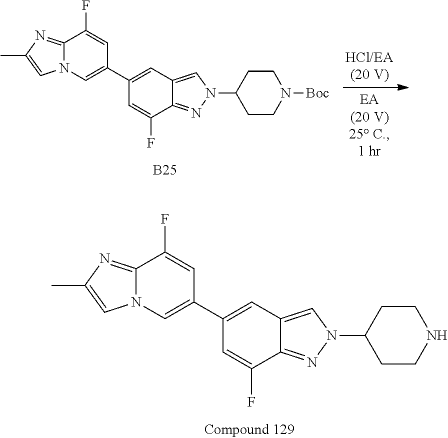

To a solution of tert-butyl 4-(7-fluoro-5-(8-fluoro-2-methylimidazo[1,2-a]pyridin-6-yl)-2H-indazol-2-yl)piperidine-1-carboxylate (B25, 100 mg, 213.9 umol, 1 equiv) in ethyl acetate (2 mL) was added HCl/ethyl acetate (4 M, 2.01 mL, 37.5 equiv). The reaction was stirred at 25° C. for 1 hr. The reaction mixture was filtered and the filter cake was dried in vacuum to get a residue. The residue was triturated with methyl tert-butyl ether (3 mL) to afford 7-fluoro-5-(8-fluoro-2-methylimidazo[1,2-a]pyridin-6-yl)-2-(piperidin-4-yl)-2H-indazole (Compound 129, 58.46 mg, 60%) as a solid. LCMS: (ESI, m/z): 368 [M+H]⁺. ¹H NMR (400 MHz methanol-d4) δ ppm 8.97 (s, 1H) 8.61 (d, J=2.45 Hz, 1H) 8.23 (d, J=11.13 Hz, 1H) 8.11 (s, 1H) 7.99 (s, 1H) 7.45 (d, J=12.47 Hz, 1H) 4.95-5.05 (m, 1H) 3.61-3.72 (m, 2H) 3.32-3.37 (m, 2H) 2.61 (s, 3H) 2.43-2.56 (m, 4H). ¹⁹F NMR (400 MHz methanol-d4) δ ppm −134.40 (s, 1F) −129.50 (s, 1F).

### Example 7: Synthesis of Compound 128

### Synthesis of Intermediate B27

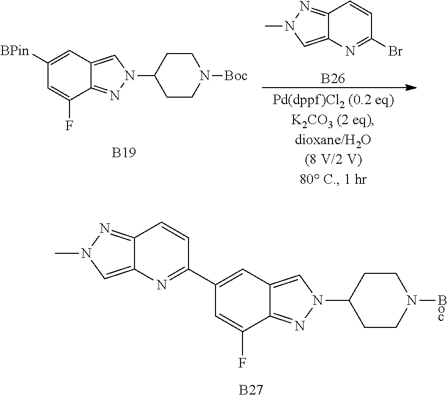

To a mixture of tert-butyl 4-(7-fluoro-5-(4,4,5,5-tetramethyl-1,3,2-dioxaborolan-2-yl)-2H-indazol-2-yl)piperidine-1-carboxylate (B19, 300 mg, 673.65 umol, 1.5 equiv) and 5-chloro-2-methyl-2H-pyrazolo[4,3-b]pyridine (B26, 75.27 mg, 449.10 umol, 1 equiv) in dioxane (2.4 mL) and H₂O (0.6 mL) was added K₂CO₃ (124.14 mg, 898.2 umol, 2 equiv) and [1,1′-Bis(diphenylphosphino)ferrocene]-dichloropalladium (II) (65.72 mg, 89.82 umol, 0.2 equiv) successively. The reaction mixture was stirred at 80° C. for 1 hr. The reaction mixture was diluted with water (10 mL) and extracted with ethyl acetate (3×10 mL). The organic layer was dried over Na₂SO₄, filtered and concentrated under reduced pressure to give an oil. The oil was purified by column chromatography on silica gel eluted with petroleum ether/ethyl acetate (5/1 to 0/1) to give tert-butyl 4-(7-fluoro-5-(2-methyl-2H-pyrazolo [4,3-b]pyridin-5-yl)-2H-indazol-2-yl) piperidine-1-carboxylate (B27, 140 mg, 64%) as an oil. LCMS: (ESI, m/z): 451 [M+H]⁺. ¹H NMR (400 MHz, methanol-d₄) δ ppm 8.52 (d, J=2.75 Hz, 1H) 8.44 (s, 1H) 8.11-8.19 (m, 2H) 7.91 (d, J=9.13 Hz, 1H) 7.79 (dd, J=13.20 Hz, 1.19 Hz, 1H) 4.61-4.81 (m, 1H) 4.24-4.34 (m, 5H) 3.04 (br s, 2H) 2.07-2.30 (m, 4H) 1.51 (s, 9H).

### Synthesis of Compound 128

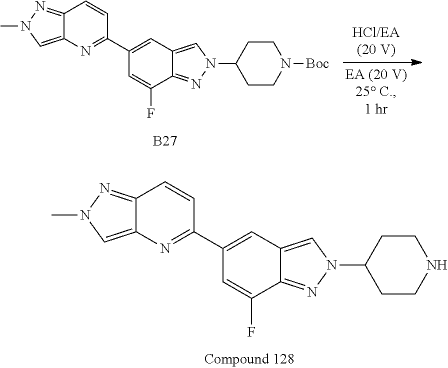

To a solution of tert-butyl 4-(7-fluoro-5-(2-methyl-2H-pyrazolo[4,3-b]pyridin-5-yl)-2H-indazol-2-yl)piperidine-1-carboxylate (B27, 140 mg, 310.76 umol, 1 equiv) in ethyl acetate (2.3 mL) was added HCl/ethyl acetate (4 M, 2.84 mL, 36.5 equiv). The reaction was stirred at 25° C. for 1 hr. The reaction mixture was filtered and the filter cake was dried in vacuum to get residue. The residue was triturated with methyl tert-butyl ether (3 mL) to afford 5-(7-fluoro-2-(piperidin-4-yl)-2H-indazol-5-yl)-2-methyl-2H-pyrazolo[4,3-b]pyridine hydrochloride salt (Compound 128, 123.43 mg, 93%) as a solid. LCMS: (ESI, m/z): 351 [M+H]⁺. ¹HNMR (400 MHz, methanol-d₄) δ ppm 8.89 (dd, J=9.06, 0.60 Hz, 1H) 8.77-8.82 (m, 2H) 8.44 (d, J=1.43 Hz, 1H) 8.24 (d, J=9.06 Hz, 1H) 7.72 (dd, J=12.16, 1.43 Hz, 1H) 5.06 (dt, J=10.04, 5.05 Hz, 1H) 4.44 (s, 3H) 3.61-3.73 (m, 2H) 3.32-3.39 (m, 2H) 2.44-2.62 (m, 4H). ¹⁹F NMR (400 MHz, methanol-d₄) δ ppm −128.14 (s, 1F).

### Example 8: Synthesis of Compound 171

### Synthesis of Intermediate B29

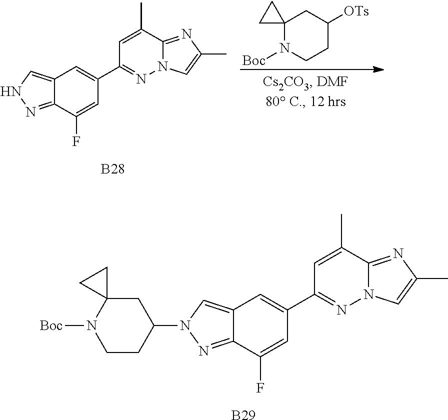

To a mixture of 6-(7-fluoro-2H-indazol-5-yl)-2,8-dimethyl-imidazo[1,2-b]pyridazine (400 mg, 1.42 mmol, 1.0 eq), tert-butyl 7-(p-tolylsulfonyloxy)-4-azaspiro[2.5]octane-4-carboxylate (596 mg, 1.56 mmol, 1.10 eq) in dimethyl formamide (10.0 mL) was added cesium carbonate (1.39 g, 4.27 mmol, 3.0 eq). The reaction was stirred at 80° C. for 12 hrs under nitrogen. The reaction mixture was diluted with water (50.0 mL), extracted with ethyl acetate (3×50.0 mL), washed with brine (100 mL), dried over Na₂SO₄, filtered and the filtrate was concentrated under reduced pressure to give a residue. The residue was purified by prep-HPLC (Condition 3, Gradient 1) to afford tert-butyl7-[5-(2,8-dimethylimidazo[1,2-b]pyridazin-6-yl)-7-fluoro-indazol-2-yl]-4-azaspiro [2.5]octane-4-carboxylate (0.150 g, 22%) as a solid. LCMS: (ESI, m/z): 491.4 [M+H]⁺. ¹H NMR (400 MHz, DMSO-d6) δ ppm 8.74 (d, J=2.7 Hz, 1H), 8.29 (s, 1H), 8.03 (s, 1H), 7.76-7.70 (m, 2H), 5.03-4.93 (m, 1H), 4.11-4.02 (m, 1H), 3.09-3.00 (m, 1H), 2.60 (s, 3H), 2.40 (s, 3H), 2.22-2.14 (m, 1H), 2.06-1.97 (m, 1H), 1.64-1.51 (m, 2H), 1.45 (s, 9H), 1.24-1.16 (m, 1H), 0.94-0.90 (m, 1H), 0.73-0.68 (m, 1H), 0.62 (br d, J=1.6 Hz, 1H)

### Synthesis of Compound 171

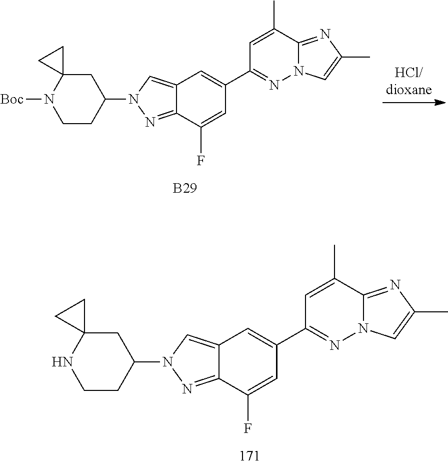

To a solution of tert-butyl 7-[5-(2,8-dimethylimidazo[1,2-b]pyridazin-6-yl)-7-fluoro-indazol-2-yl]-4-azaspiro[2.5]octane-4-carboxylate (100 mg, 203 umol, 1.0 eq) in dioxane (0.400 mL) was added HCl/dioxane (4.0 M, 0.800 mL). The reaction was stirred at 25° C. for 1 hr. The reaction mixture was filtered and the filter cake was dried over under reduced pressure to give 6-[2-(4-azaspiro[2.5]octan-7-yl)-7-fluoro-indazol-5-yl]-2,8-dimethyl-imidazo[1,2-b]pyridazine (34.8 mg, 42%) as a solid. LCMS: (ESI, m/z): 391.1 [M+H]⁺. ¹H NMR (400 MHz, methanol-d4) δ ppm 8.67 (d, J=2.3 Hz, 1H), 8.43 (s, 1H), 8.31 (d, J=7.3 Hz, 2H), 7.85 (d, J=12.9 Hz, 1H), 5.17-5.06 (m, 1H), 3.72-3.67 (m, 1H), 3.66-3.58 (m, 1H), 3.48-3.39 (m, 1H), 2.99-2.88 (m, 1H), 2.78 (s, 3H), 2.65 (s, 3H), 2.61-2.51 (m, 2H), 1.96 (dd, J=3.7, 13.9 Hz, 1H), 1.16 (s, 1H), 1.11-1.05 (m, 1H), 1.02-0.95 (m, 1H)

### Example 9: Synthesis of Compound 174

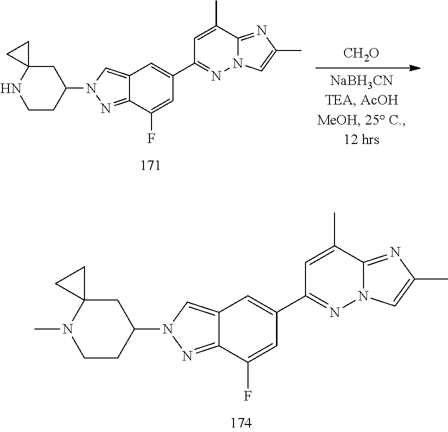

To a solution of 6-[2-(4-azaspiro[2.5]octan-7-yl)-7-fluoro-indazol-5-yl]-2,8-dimethyl-imidazo [1,2-b]pyridazine (50.0 mg, 128 umol, 1.0 eq) in methanol (1.00 mL) was added trimethylamine (35.6 uL, 256 umol, 2.0 eq), the mixture was stirred at 30° C. for 30 minutes. Then, acetic acid (7.32 uL, 128 umol, 1.0 eq), sodium cyanoborohydride (8.05 mg, 128 umol, 1.0 eq) and formaldehyde (7.69 mg, 256 umol, 2.0 eq) was added to the above mixture. The reaction mixture was stirred at 25° C. for 12 hrs. The reaction mixture was quenched with water (3.00 mL), extracted with ethyl acetate (3×3.00 mL), washed with brine (3.00 mL), dried over Na₂SO₄, filtered and the filtrate was concentrated under reduced pressure to give a residue. The residue was purified by prep-HPLC (Condition 2, Gradient 7) to afford 6-[7-fluoro-2-(4-methyl-4-azaspiro[2.5]octan-7-yl)indazol-5-yl]-2,8-dimethyl-imidazo[1,2-b]pyridazine (8.53 mg, 16%) as a solid. LCMS: (ESI, m/z): 405.3 [M+H]⁺. ¹H NMR (400 MHz, methanol-d4) δ ppm 8.56 (d, J=2.7 Hz, 1H), 8.21 (d, J=1.2 Hz, 1H), 7.90 (d, J=0.6 Hz, 1H), 7.77 (dd, J=1.2, 13.1 Hz, 1H), 7.63 (d, J=1.0 Hz, 1H), 4.85-4.81 (m, 1H), 3.27-3.21 (m, 1H), 3.19-3.10 (m, 1H), 2.87 (t, J=12.5 Hz, 1H), 2.66 (d, J=1.0 Hz, 3H), 2.65 (s, 3H), 2.58 (dd, J=4.2, 12.4 Hz, 1H), 2.48 (s, 3H), 2.09-2.01 (m, 1H), 1.46-1.39 (m, 1H), 0.86-0.79 (m, 2H), 0.77-0.71 (m, 1H), 0.67-0.58 (m, 1H)

### Example 10: Synthesis of Compound 177

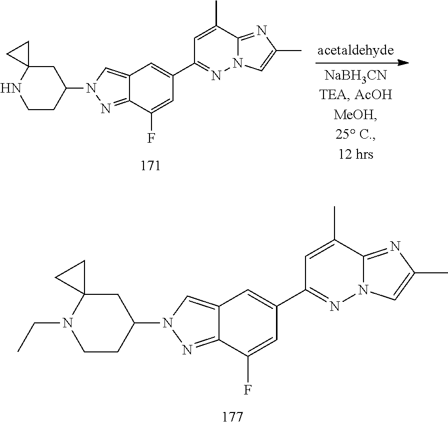

To a solution of 6-[2-(4-azaspiro[2.5]octan-7-yl)-7-fluoro-indazol-5-yl]-2,8-dimethyl-imidazo [1,2-b]pyridazine (50.0 mg, 128 umol, 1.0 eq) in methanol (1.0 mL) was added trimethylamine (35.6 uL, 256 umol, 2.0 eq), the mixture was stirred at 25° C. for 30 minutes. Then, acetic acid (7.32 uL, 128 umol, 1.0 eq), sodium cyanoborohydride (8.05 mg, 128 umol, 1.0 eq) and acetaldehyde (11.2 mg, 256 umol, 2.0 eq) was added to the above mixture. The reaction mixture was stirred at 25° C. for 11.5 hrs. The reaction mixture was quenched with water (3.00 mL), extracted with ethyl acetate (3×3.00 mL), washed with brine (3.00 mL), dried over Na₂SO₄, filtered and the filtrate was concentrated under reduced pressure to give a residue. The residue was purified by prep-HPLC (Condition 2, Gradient 7) to afford 6-[2-(4-ethyl-4-azaspiro[2.5]octan-7-yl)-7-fluoro-indazol-5-yl]-2,8-dimethyl-imidazo[1,2-b]pyridazine (11.6 mg, 22%) as a solid. LCMS: (ESI, m/z): 419.3 [M+H]⁺. ¹H NMR (400 MHz, methanol-d4) δ ppm 8.56 (d, J=2.7 Hz, 1H), 8.21 (d, J=1.2 Hz, 1H), 7.90 (d, J=0.7 Hz, 1H), 7.77 (dd, J=1.2, 13.1 Hz, 1H), 7.63 (d, J=1.0 Hz, 1H), 4.85-4.80 (m, 1H), 3.25 (br s, 1H), 3.17-3.08 (m, 2H), 2.94-2.76 (m, 2H), 2.66 (d, J=1.0 Hz, 3H), 2.48 (d, J=0.6 Hz, 4H), 1.99-1.90 (m, 1H), 1.35 (br dd, J=2.6, 13.1 Hz, 1H), 1.15 (t, J=7.3 Hz, 3H), 0.86-0.73 (m, 2H), 0.70-0.58 (m, 2H)

### Example 11: Synthesis of Compound 180

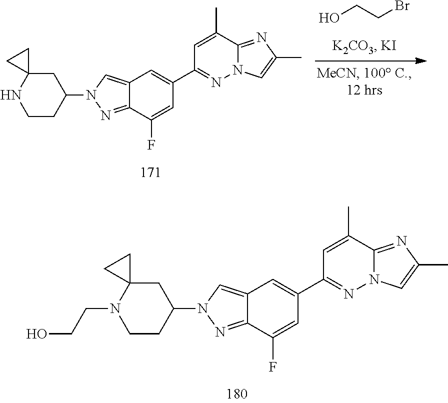

A mixture of 2-bromoethanol (90.9 uL, 1.28 mmol, 5.0 eq), 6-[2-(4-azaspiro[2.5]octan-7-yl)-7-fluoro-indazol-5-yl]-2,8-dimethyl-imidazo[1,2-b]pyridazine (100 mg, 256 umol, 1.0 eq), potassium carbonate (176 mg, 1.28 mmol, 5.0 eq), potassium iodide (212 mg, 1.28 mmol, 5.0 eq) in dimethyl formamide (2.00 mL) was stirred at 100° C. for 12 hrs. The reaction mixture was diluted with water (3.00 mL), extracted with ethyl acetate (3×3.00 mL), washed with brine (3.00 mL), dried over Na₂SO₄, filtered and the filtrate was concentrated under reduced pressure to give residue. The residue was purified by prep-HPLC (Condition 4, Gradient 1) to afford 2-[7-[5-(2,8-dimethylimidazo[1,2-b]pyridazin-6-yl)-7-fluoro-indazol-2-yl]-4-azaspiro[2.5]octan-4-yl]ethanol (18.0 mg, 16%) as a solid. LCMS: (ESI, m/z): 435.3 [M+H]⁺. ¹H NMR (400 MHz, methanol-d4) δ ppm 8.55 (d, J=2.8 Hz, 1H), 8.20 (d, J=1.0 Hz, 1H), 7.89 (s, 1H), 7.75 (dd, J=1.1, 13.1 Hz, 1H), 7.62 (d, J=0.9 Hz, 1H), 4.82-4.79 (m, 1H), 3.66-3.56 (m, 2H), 3.24-3.07 (m, 3H), 2.90 (td, J=6.8, 13.4 Hz, 1H), 2.72 (t, J=12.4 Hz, 1H), 2.66 (s, 3H), 2.52-2.46 (m, 4H), 1.93 (br dd, J=1.9, 12.7 Hz, 1H), 1.40-1.33 (m, 1H), 0.84-0.72 (m, 2H), 0.66-0.52 (m, 2H)

### Example 12: Synthesis of Compound 183

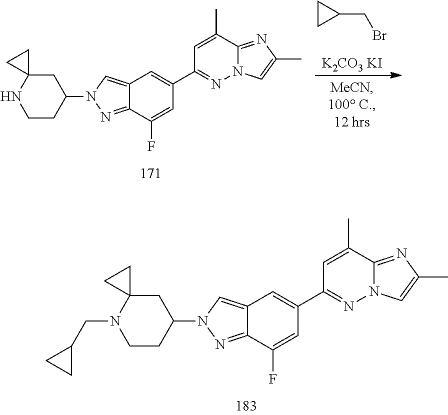

A mixture of bromomethylcyclopropane (172 mg, 1.28 mmol, 5.0 eq), 6-[2-(4-azaspiro [2.5]octan-7-yl)-7-fluoro-indazol-5-yl]-2,8-dimethyl-imidazo[1,2-b]pyridazine (100 mg, 256 umol, 1.0 eq), potassium carbonate (176 mg, 1.28 mmol, 5.0 eq), potassium iodide (212 mg, 1.28 mmol, 5.0 eq) in dimethyl formamide (2.00 mL) was stirred at 100° C. for 12 hrs. The reaction mixture was diluted with water (3.00 mL), extracted with ethyl acetate (3×3.00 mL), washed with brine (3.00 mL), dried over Na₂SO₄, filtered and the filtrate was concentrated under reduced pressure to give a residue. The residue was purified by prep-HPLC (Condition 4, Gradient 2) to afford 6-[2-[4-(cyclopropylmethyl)-4-azaspiro[2.5]octan-7-yl]-7-fluoro-indazol-5-yl]-2,8-dimethyl-imidazo[1,2-b]pyridazine (5.32 mg, 5%) as a solid. LCMS: (ESI, m/z): 445.3 [M+H]⁺. ¹H NMR (400 MHz, methanol-d4) δ ppm 8.55 (d, J=2.8 Hz, 1H), 8.20 (d, J=1.1 Hz, 1H), 7.90 (d, J=0.6 Hz, 1H), 7.76 (dd, J=1.2, 13.1 Hz, 1H), 7.62 (d, J=0.9 Hz, 1H), 4.90-4.85 (m, 1H), 3.52-3.43 (m, 1H), 3.19-3.10 (m, 1H), 2.84-2.70 (m, 3H), 2.66 (d, J=0.8 Hz, 3H), 2.53-2.42 (m, 4H), 1.97-1.87 (m, 1H), 1.34-1.26 (m, 1H), 0.94-0.75 (m, 3H), 0.66-0.51 (m, 4H), 0.34-0.17 (m, 2H)

### Example 13: Synthesis of Compound 186

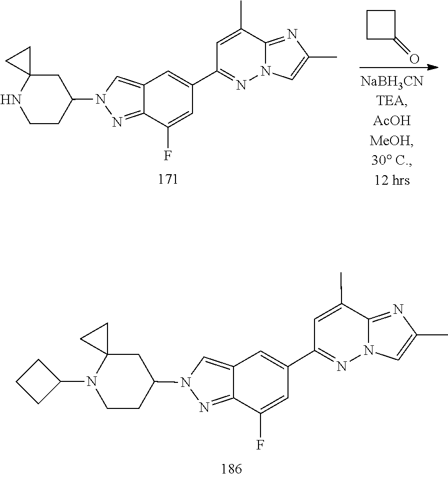

To a solution of 6-[2-(4-azaspiro[2.5]octan-7-yl)-7-fluoro-indazol-5-yl]-2,8-dimethyl-imidazo [1,2-b]pyridazine (50.0 mg, 128 umol, 1.0 eq) in methanol (1.0 mL) was added trimethylamine (35.6 uL, 256 umol, 2.0 eq), the mixture was stirred at 30° C. for 30 mins. Then, acetic acid (7.32 uL, 128 umol, 1.0 eq), sodium cyanoborohydride (8.05 mg, 128 umol, 1.0 eq) and cyclobutanone (38.2 uL, 512 umol, 2.0 eq) was added to the above mixture. The reaction mixture was stirred at 30° C. for 11.5 hrs. LCMS showed the reaction was completed. The reaction mixture was quenched with water (3.00 mL), extracted with ethyl acetate (3×3.00 mL), washed with brine (3.00 mL), dried over Na₂SO₄, filtered and the filtrate was concentrated under reduced pressure to give residue. The residue was purified by prep-HPLC (Condition 4, Gradient 3) to give a residue. The residue was purified by prep-HPLC (Condition 4, Gradient 2) to give (6-[2-(4-cyclo butyl-4-azaspiro[2.5]octan-7-yl)-7-fluoro-indazol-5-yl]-2,8-dimethyl-imidazo[1,2-b]pyridazine (11.2 mg, 10%) as a solid. LCMS: (ESI, m/z): 445.3 [M+H]⁺. ¹H NMR (400 MHz, methanol-d4) δ ppm 8.56 (d, J=2.6 Hz, 1H), 8.21 (d, J=1.0 Hz, 1H), 7.91 (s, 1H), 7.77 (dd, J=1.1, 13.1 Hz, 1H), 7.64 (d, J=0.8 Hz, 1H), 4.03-3.92 (m, 1H), 3.17-3.11 (m, 1H), 3.01 (dt, J=2.6, 13.4 Hz, 1H), 2.90-2.81 (m, 1H), 2.67 (d, J=0.8 Hz, 3H), 2.49 (s, 3H), 2.45-2.32 (m, 1H), 2.25-2.11 (m, 2H), 1.97-1.81 (m, 3H), 1.79-1.68 (m, 2H), 1.32-1.24 (m, 1H), 0.97-0.91 (m, 1H), 0.66-0.58 (m, 1H), 0.56-0.49 (m, 2H)

### Example 14: Synthesis of Compound 192 Synthesis of Intermediate B31

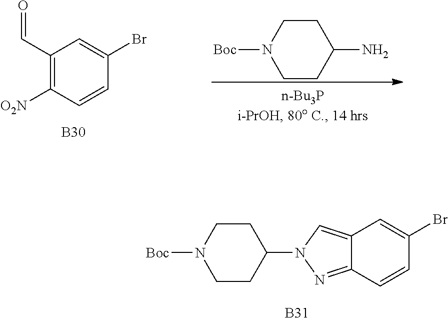 [SMILES: "B30" ; O=Cc1cc(Br)ccc1[N+](=O)[O-] ; "B31" ; CC(C)(C)OC(=O)N1CCC(n2cc3cc(Br)ccc3n2)CC1 ; CC(C)(C)OC(=O)N1CCC(N)CC1]

To a mixture of 5-bromo-2-nitro-benzaldehyde (1.0 g, 4.35 mmol, 1.0 eq) and tert-butyl 4-aminopiperidine-1-carboxylate (41-2, 957 mg, 4.78 mmol, 1.10 eq) in propan-2-ol (15.0 mL) was stirred at 80° C. for 2 hrs. The reaction was cooled to 25° C., and added tributylphosphane (2.64 g, 13.0 mmol, 3.22 mL, 3.0 eq). The reaction mixture was then stirred at 80° C. for 12 hrs. TLC (petroleum ether/ethyl acetate=5/1, Rf=0.4) showed the reaction was completed. The reaction mixture was quenched with water (30.0 mL), extracted with ethyl acetate (3×30.0 mL), washed with brine (30.0 mL), dried over Na₂SO₄, filtered and the filtrate was concentrated under reduced pressure to give residue. The residue was purified by flash column chromatography (petroleum ether/ethyl acetate=5/1) to give tert-butyl 4-(5-bromoindazol-2-yl) piperidine-1-carboxylate (1.20 g, 73% yield) as an oil. LCMS: (ESI, m/z): 381.2 [M+1]⁺. ¹H NMR (400 MHz, chloroform-d) δ ppm 7.91 (s, 1H), 7.82 (d, J=0.9 Hz, 1H), 7.59 (d, J=9.1 Hz, 1H), 7.34 (dd, J=1.6, 9.1 Hz, 1H), 4.55 (tt, J=4.0, 11.5 Hz, 1H), 2.95 (br t, J=11.6 Hz, 2H), 2.29-2.20 (m, 2H), 2.14-2.06 (m, 2H), 1.76-1.56 (m, 2H), 1.49 (s, 9H)

### Synthesis of Intermediate B32

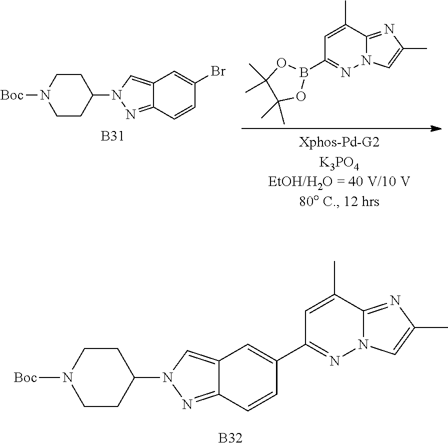

To a mixture of 2,8-dimethyl-6-(4,4,5,5-tetramethyl-1,3,2-dioxaborolan-2-yl)imidazo[1,2-b]pyridazine (143 mg, 525 umol, 1.0 eq), tert-butyl 4-(5-bromoindazol-2-yl)piperidine-1-carboxylate (200 mg, 525 umol, 1.0 eq) and potassium phosphate (446 mg, 2.10 mmol, 4.0 eq) in ethanol (20.0 mL) and H₂O (5.00 mL) was added Chloro(2-Dicyclohexylphosphino-2 [symbol] [symbol] [symbol] Triisopropyl-1,1 [symbol] phenyl)[2-(2 Amino-1,1 [symbol] Biphenyl)]Palladium(Ii) (41.3 mg, 52.5 umol, 0.100 eq). The reaction mixture was then stirred at 80° C. for 12 hrs. The reaction mixture was diluted with water (50.0 mL), extracted with ethyl acetate (3×50.0 mL), washed with brine (30.0 mL), dried over Na₂SO₄, filtered and the filtrate was concentrated under reduced pressure to give residue. The residue was purified by flash column chromatography (petroleum ether/ethyl acetate=5/1) to give tert-butyl 4-[5-(2,8-dimethylimidazo[1,2-b]pyridazin-6-yl)indazol-2-yl]piperidine-1-carboxylate (200 mg, 43%) as a solid. LCMS: (ESI, m/z): 447.3 [M+H]⁺. ¹H NMR (400 MHz, chloroform-d) δ ppm 8.21 (s, 1H), 8.07 (s, 1H), 7.94 (dd, J=1.6, 9.1 Hz, 1H), 7.82 (d, J=9.1 Hz, 1H), 7.77 (s, 1H), 7.31 (s, 1H), 4.67-4.55 (m, 1H), 3.03-2.92 (m, 2H), 2.72 (s, 3H), 2.54 (s, 3H), 2.33-2.25 (m, 3H), 2.20-2.10 (m, 2H), 2.07 (s, 1H), 1.50 (s, 9H)

### Synthesis of Compound 192

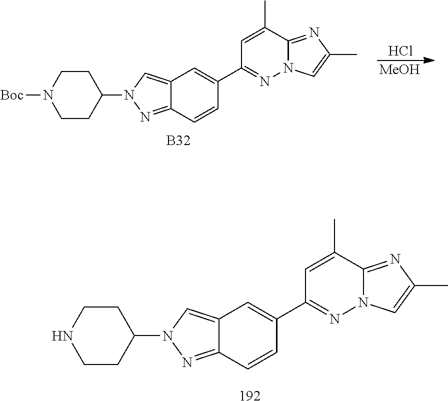

A solution of tert-butyl 4-[5-(2,8-dimethylimidazo[1,2-b]pyridazin-6-yl)indazol-2-yl]piperidine-1-carboxylate (41-4, 200 mg, 447 umol, 1.0 eq) in HCl/methanol (4.0 M, 0.100 mL) was stirred at 25° C. for 1 hr. LCMS showed the reaction was completed. The reaction mixture was filtered and the filter cake was dried over reduced pressure to give residue. The residue was triturated with methyl tert-butyl ether (5.00 mL) and filtered. The filter cake was dried over in vacuum to give 2,8-dimethyl-6-[2-(4-piperidyl)indazol-5-yl]imidazo[1,2-b]pyridazine (69.9 mg, 43%) as a solid. LCMS: (ESI, m/z): 347.1 [M+H]⁺. ¹H NMR (400 MHz, methanol-d4) δ ppm 8.61-8.54 (m, 2H), 8.30 (dd, J=0.9, 6.1 Hz, 2H), 8.13 (dd, J=1.7, 9.2 Hz, 1H), 7.81 (d, J=9.3 Hz, 1H), 5.02-4.93 (m, 1H), 3.71-3.62 (m, 2H), 3.37-3.33 (m, 2H), 2.79 (d, J=1.0 Hz, 3H), 2.65 (d, J=0.8 Hz, 3H), 2.54-2.44 (m, 4H)

### Example 15: Synthesis of Compounds 190 and 191

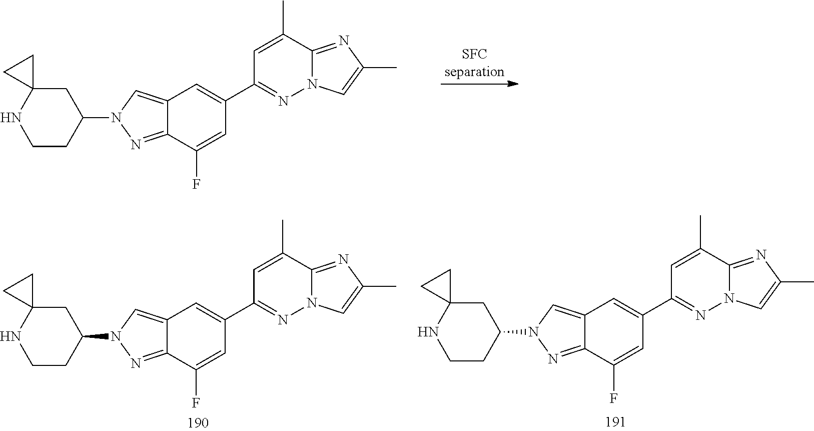

6-[2-(4-azaspiro[2.5]octan-7-yl)-7-fluoro-indazol-5-yl]-2,8-dimethyl-imidazo[1,2-b]pyridazine (EVAL-0167-0028, 500 mg) was separated by SFC (column: DAICEL CHIRALCEL OJ (250 mm*30 mm, 10 um); mobile phase: [0.1% NH₃H₂O in ethanol]; B %: 20%-40%, 13 min) to give 6-[2-[(7S)-4-azaspiro[2.5]octan-7-yl]-7-fluoro-indazol-5-yl]-2,8-dimethylimidazo[1,2-b]pyridazine (35.5 mg, 7.11% yield) as white solid and 6-[2-[(7R)-4-azaspiro[2.5]octan-7-yl]-7-fluoro-indazol-5-yl]-2,8-dimethyl-imidazo[1,2-b]pyridazine (33.7 mg, 7% yield) as solid. Compound 190: LCMS (ESI, m/z): 391.1[M+H]⁺. ¹H NMR (400 MHz, methanol-d4) δ ppm 8.53 (d, J=2.8 Hz, 1H), 8.20 (d, J=1.0 Hz, 1H), 7.89 (d, J=0.6 Hz, 1H), 7.75 (dd, J=1.2, 13.1 Hz, 1H), 7.62 (d, J=0.9 Hz, 1H), 4.84-4.80 (m, 1H), 3.26-3.19 (m, 1H), 3.02-2.94 (m, 1H), 2.66 (d, J=0.9 Hz, 3H), 2.61-2.53 (m, 1H), 2.48 (s, 3H), 2.33-2.27 (m, 1H), 2.24-2.15 (m, 1H), 1.71-1.64 (m, 1H), 0.79-0.74 (m, 1H), 0.73-0.69 (m, 1H), 0.67-0.62 (m, 2H) Compound 191: LCMS (ESI, m/z): 391.1[M+H]⁺. ¹H NMR (400 MHz, methanol-d4) δ ppm 8.53 (d, J=2.8 Hz, 1H), 8.21 (d, J=1.1 Hz, 1H), 7.90 (d, J=0.6 Hz, 1H), 7.76 (dd, J=1.2, 13.1 Hz, 1H), 7.63 (d, J=1.0 Hz, 1H), 4.83-4.78 (m, 1H), 3.22-3.16 (m, 1H), 2.99-2.89 (m, 1H), 2.66 (d, J=0.9 Hz, 3H), 2.59-2.50 (m, 1H), 2.48 (s, 3H), 2.31-2.25 (m, 1H), 2.21-2.13 (m, 1H), 1.68-1.61 (m, 1H), 0.75-0.71 (m, 1H), 0.68-0.63 (m, 1H), 0.63-0.59 (m, 2H)

### Example 16: Synthesis of Compound 201

### Synthesis of Intermediate B34

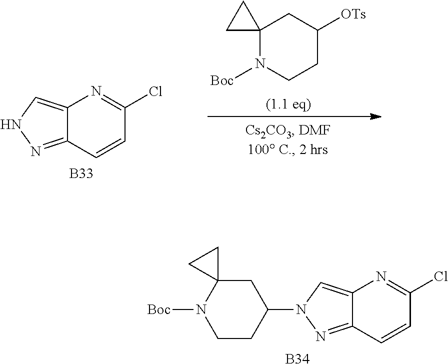

To a suspension of 5-chloro-2H-pyrazolo[4,3-b]pyridine (300 mg, 1.95 mmol, 1.00 eq), tert-butyl 7-(p-tolylsulfonyloxy)-4-azaspiro[2.5]octane-4-carboxylate (819 mg, 2.15 mmol, 1.10 eq) in dimethyl formamide (7.50 mL) was added cesium carbonate (1.91 g, 5.86 mmol, 3.00 eq). The reaction was stirred at 100° C. for 2 hrs. The reaction mixture was diluted with water (20.0 mL), extracted with ethyl acetate (3×20.0 mL), washed with brine (20.0 mL), dried over Na₂SO₄, filtered and concentrated under reduced pressure to give residue. The residue was purified by prep-HPLC (Condition 3, Gradient 2) to afford tert-butyl 7-(5-chloropyrazolo[4,3-b]pyridin-2-yl)-4-azaspiro[2.5]octane-4-carboxylate (180 mg, 25%) as a solid. LCMS: (ESI, m/z): 363.6 [M+H]⁺. ¹H NMR (400 MHz, chloroform-d) δ ppm 8.10 (s, 1H), 7.99 (d, J=9.0 Hz, 1H), 7.20 (d, J=9.0 Hz, 1H), 4.86-4.74 (m, 1H), 4.27 (br d, J=13.5 Hz, 1H), 3.13-3.02 (m, 1H), 2.61-2.49 (m, 1H), 2.26 (br dd, J=2.2, 12.8 Hz, 1H), 2.17-2.04 (m, 1H), 1.60 (br s, 1H), 1.50 (s, 9H), 1.44-1.35 (m, 1H), 1.02-0.93 (m, 1H), 0.72-0.58 (m, 2H)

### Synthesis of Intermediate B35

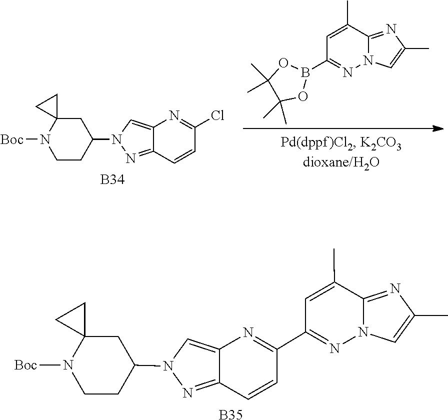

To a mixture of tert-butyl 7-(5-chloropyrazolo[4,3-b]pyridin-2-yl)-4-azaspiro[2.5]octane-4-carboxylate (40-4, 90.0 mg, 248 umol, 1.00 eq), 2,8-dimethyl-6-(4,4,5,5-tetramethyl-1,3,2-dioxaborolan-2-yl)imidazo[1,2-b]pyridazine (A2, 241 mg, 248 umol, 1.00 eq), potassium carbonate (137 mg, 992 umol, 4.00 eq) in dioxane (1.20 mL) and H₂O (0.300 mL) was added 1,1 [symbol] bis(diphenylphosphino)ferrocenedichloro palladium(II) (18.1 mg, 24.8 umol, 0.100 eq). The reaction was stirred at 80° C. for 2 hrs. The reaction mixture was quenched with water (10.0 mL), extracted with ethyl acetate (3×10.0 mL), washed with brine (10.0 mL), dried over Na₂SO₄, filtered and the filtrate was concentrated under reduced pressure to give residue. The residue was purified by flash column chromatography (petroleum ether/ethyl acetate=5/1) to give tert-butyl 7-[5-(2,8-dimethylimidazo[1,2-b]pyridazin-6-yl)pyrazolo[4,3-b]pyridin-2-yl]-4-azaspiro[2.5]octane-4-carboxylate (40-3, 200 mg, 85%) as a solid. LCMS: (ESI, m/z): 474.6 [M+1]⁺. ¹H NMR (400 MHz, chloroform-d) δ ppm 8.36-8.28 (m, 2H), 8.15 (d, J=9.2 Hz, 1H), 8.07 (br s, 1H), 7.78 (s, 1H), 4.87 (tt, J=4.3, 11.7 Hz, 1H), 4.29 (br d, J=12.2 Hz, 1H), 3.11 (br t, J=12.0 Hz, 1H), 2.76 (s, 3H), 2.57 (s, 3H), 2.37-2.27 (m, 1H), 2.20-2.08 (m, 1H), 1.52 (s, 9H), 1.45-1.34 (m, 2H), 1.30-1.27 (m, 1H), 1.04-0.94 (m, 1H), 0.74-0.61 (m, 2H)

### Synthesis of Compound 201

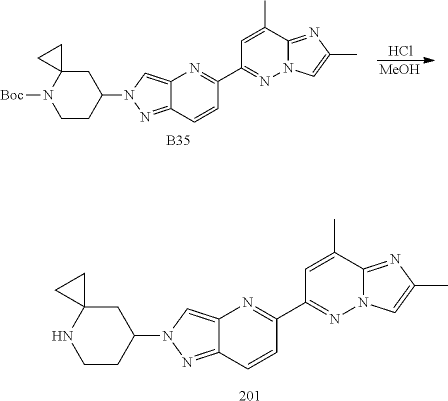

A solution of tert-butyl 7-[5-(2,8-dimethylimidazo[1,2-b]pyridazin-6-yl)pyrazolo[4,3-b]pyridin-2-yl]-4-azaspiro[2.5]octane-4-carboxylate (40-3, 150 mg, 316 umol, 1.00 eq) in HCl/MeOH (4.0 M, 1.50 mL) was stirred at 25° C. for 1 hr. The reaction mixture was concentrated under reduced pressure to give residue. The residue was purified by prep-HPLC (Condition 4, Gradient 3) to afford 6-[2-(4-azaspiro[2.5]octan-7-yl)pyrazolo[4,3-b]pyridine-5-yl]-2,8-dimethyl-imidazo[1,2-b]pyridazine (70.0 mg, 59.1%) as a solid. LCMS: (ESI, m/z): 374.2 [M+H]⁺. ¹H NMR (400 MHz, methanol-d4) δ ppm 8.77 (s, 1H), 8.73 (s, 1H), 8.41 (d, J=9.2 Hz, 1H), 8.34 (s, 1H), 8.28 (d, J=9.2 Hz, 1H), 5.15 (ddd, J=4.3, 11.0, 15.4 Hz, 1H), 3.69 (td, J=3.6, 12.8 Hz, 1H), 3.47-3.40 (m, 1H), 2.98-2.88 (m, 1H), 2.80 (d, J=0.9 Hz, 3H), 2.68-2.62 (m, 3H), 2.62-2.53 (m, 2H), 1.97 (dd, J=3.8, 13.7 Hz, 1H), 1.16 (t, J=2.8 Hz, 2H), 1.13-1.06 (m, 1H), 1.04-0.96 (m, 1H)

### Example 17: Synthesis of Compound 207

### Synthesis of Intermediate B36

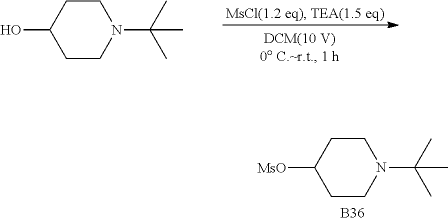

Into a 50 mL bottles were added 1-tert-butylpiperidin-4-ol (900 mg, 5.723 mmol, 1 equiv) and TEA (868.71 mg, 8.585 mmol, 1.5 equiv) in DCM (9 mL) at room temperature. To the above mixture was added MsCl (786.63 mg, 6.868 mmol, 1.2 equiv) dropwise over 1 min at 0° C. The resulting mixture was stirred for additional 1 h at room temperature. The reaction was quenched with water at room temperature. The reaction was quenched with NaHCO₃ at room temperature. The resulting mixture was extracted with DCM (3×18 mL). The combined organic layers were washed with NaCl solution (2×20 mL), dried over anhydrous Na₂SO₄. After filtration. The resulting mixture was concentrated under reduced pressure. This resulted in 1-tert-butylpiperidin-4-yl methanesulfonate (800 mg, 59%) as a solid. LCMS: (ESI, m/z): 235[M+H]⁺

### Synthesis of Intermediate B37

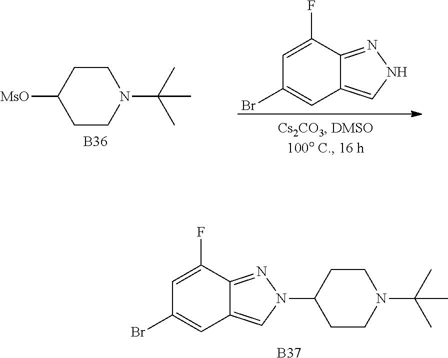 [SMILES: "B36" ; CC(C)(C)N1CCC(OS(C)(=O)=O)CC1 ; "B37" ; CC(C)(C)N1CCC(n2cc3cc(Br)cc(F)c3n2)CC1 ; Fc1cc(Br)cc2c[nH]nc12]

Into a 50 mL bottles were added 1-tert-butylpiperidin-4-yl methanesulfonate (840 mg, 3.569 mmol, 1.5 equiv) and 5-bromo-7-fluoro-2H-indazole (511.66 mg, 2.379 mmol, 1 equiv) and Cs₂CO₃ (2325.89 mg, 7.138 mmol, 3 equiv) in DMSO (9 mL) at room temperature. The resulting mixture was stirred for additional 16 h at 100° C. The mixture was allowed to cool down to room temperature. The reaction was quenched with H₂O at room temperature. The resulting mixture was extracted with EA (3×20 mL). The combined organic layers were washed with NaCl solution (2×20 mL), dried over anhydrous Na₂SO₄. After filtration, the filtrate was concentrated under reduced pressure. The residue was purified by silica gel column chromatography, eluted with PE/EA (1:1) to afford 5-bromo-2-(1-tert-butylpiperidin-4-yl)-7-fluoroindazole (110 mg) as a solid. LCMS: (ESI, m/z): 353[M+H]⁺

### Synthesis of Compound 207

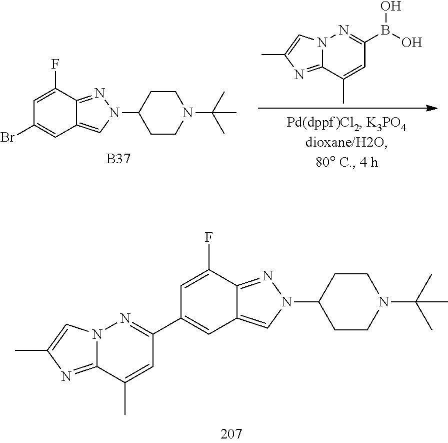 [SMILES: "B37" ; CC(C)(C)N1CCC(n2cc3cc(Br)cc(F)c3n2)CC1 ; Cc1cn2nc(-c3cc(F)c4nn(C5CCN(C(C)(C)C)CC5)cc4c3)cc(C)c2n1 ; Cc1cn2nc(B(O)O)cc(C)c2n1]

A solution of 5-bromo-2-(1-tert-butylpiperidin-4-yl)-7-fluoroindazole (90 mg, 0.254 mmol, 1 equiv), Pd(dppf)Cl₂ (20.7 mg, 0.025 mmol, 0.10 equiv), 2,8-dimethylimidazo[1,2-b]pyridazin-6-ylboronic acid (72.78 mg, 0.381 mmol, 1.50 equiv) and K₃PO₄ (134.81 mg, 0.635 mmol, 2.50 equiv) in dioxane (5 mL) and H₂O (1 mL) was stirred for 4 h at 80° C. under nitrogen atmosphere. The mixture was allowed to cool down to room temperature. The resulting mixture was dried over anhydrous Na₂SO₄. After filtration, the filtrate was concentrated under reduced pressure. The residue was purified by silica gel column chromatography, eluted with CH₂Cl₂/MeOH (10:1). The crude product was purified by Chiral-Prep-HPLC (Condition 3, Gradient 1) to afford 2-(1-tert-butylpiperidin-4-yl)-5-{2,8-dimethylimidazo[1,2-b]pyridazin-6-yl}-7-fluoroindazole (26.0 mg, 24%) as a solid. LCMS: (ESI, m/z): 420[M+H]^{+ 1}H NMR: (400 MHz, DMSO-d₆) δ 8.73 (d, J=2.8 Hz, 1H), 8.28 (d, J=1.3 Hz, 1H), 8.04 (d, J=1.0 Hz, 1H), 7.77-7.69 (m, 2H), 4.52 (s, 1H), 3.16 (d, J=11.0 Hz, 2H), 2.60 (d, J=1.1 Hz, 3H), 2.40 (d, J=0.8 Hz, 3H), 2.25 (t, J=11.3 Hz, 2H), 2.16-2.04 (m, 4H), 1.08 (s, 9H).

### Example 18: Synthesis of Compound 104

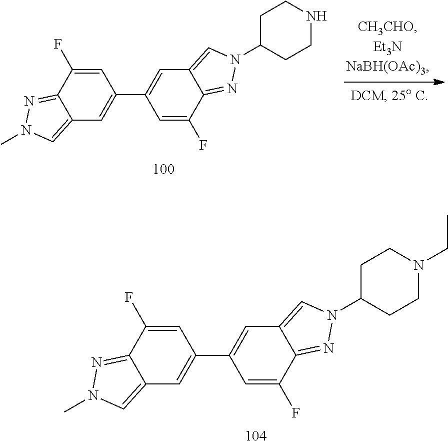 [SMILES: Cn1cc2cc(-c3cc(F)c4nn(C5CCNCC5)cc4c3)cc(F)c2n1 ; CCN1CCC(n2cc3cc(-c4cc(F)c5nn(C)cc5c4)cc(F)c3n2)CC1]

To a solution of acetaldehyde (3.90 mg, 35.4 umol, 4.97 uL, 40% purity, 1.10 eq) in DCM (1.30 mL) was added dropwise TEA (3.26 mg, 32.1 umol, 4.48 uL, 1 eq) at 15° C. over 5 min. The mixture was stirred at this temperature for 5 min, and then 7-fluoro-5-(7-fluoro-2-methyl-indazol-5-yl)-2-(4-piperidyl)indazole (EVAL-0123-36, 13.0 mg, 32.1 umol, 1 eq, HCl) was added at 15° C. After addition, the mixture was stirred at this temperature for 20 min, and then NaBH(OAc)₃ (13.6 mg, 64.3 umol, 2 eq) was added dropwise at 15° C. The resulting mixture was stirred at 15° C. for 3 hrs. LCMS showed desired MS was detected. The reaction mixture was quenched by addition H₂O (0.5 mL) at 0° C., then diluted with dichloromethane (500 uL) and extracted with dichloromethane (3×500 uL). The combined organic layers were washed with brine (1.00 mL), dried over Na₂SO₄, filtered and concentrated under reduced pressure to give a residue. The residue was purified by prep-HPLC (column: Phenomenex Luna 80*30 mm*3 um; mobile phase: [water(HCl)—CH₃CN]; B %: 1%-30%, 8 min) to give 2-(1-ethyl-4-piperidyl)-7-fluoro-5-(7-fluoro-2-methyl-indazol-5-yl)indazole (EVAL-0123-0004, 4.30 mg, 34%) as a solid. LCMS: (ESI, m/z): 396.3 [M+H]⁺. ¹H NMR (400 MHz, Pyr) δ ppm 8.54 (d, J=2.13 Hz, 1H) 8.35 (d, J=2.38 Hz, 1H) 7.86 (br d, J=7.25 Hz, 2H) 7.64 (br d, J=5.13 Hz, 2H) 4.88 (br s, 1H) 4.16 (s, 3H) 3.38-3.53 (m, 2H) 2.86 (br d, J=4.75 Hz, 6H) 2.56 (br s, 2H) 1.30 (br t, J=7.00 Hz, 3H). ¹⁹F NMR (376 MHz, Pyr) δ ppm −128.583, −128.610.

### Example 19: Synthesis of Compound 105

### Synthesis of Intermediate C3

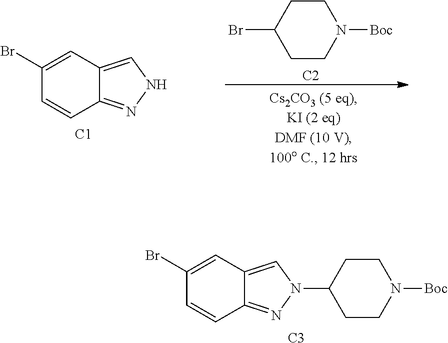

A mixture of 5-bromo-2H-indazole (1.00 g, 5.08 mmol, 1 eq), tert-butyl 4-bromopiperidine-1-carboxylate (4.02 g, 15.2 mmol, 2 eq), Cs₂CO₃ (8.27 g, 25.3 mmol, 5 eq), KI (1.69 g, 10.1 mmol, 2 eq) in DMF (10.0 mL) was degassed and purged with N₂ for 3 times, and then the mixture was stirred at 100° C. for 12 hrs under N₂ atmosphere. TLC (petroleum ether/ethyl acetate=3/1, Rf=0.27) showed the starting material was consumed and a new major spot was generated. The mixture was filtered and the filtrate was concentrated under reduced pressure. The residue was purified by prep-HPLC (column: Phenomenex C18 75*30 mm*3 um; mobile phase: [water(NH₄HCO₃)—CH₃CN]; B %: 45%-65%, 8 min) to give tert-butyl 4-(5-bromoindazol-2-yl)piperidine-1-carboxylate (100 mg) as a solid LCMS: (ESI, m/z): 324.0, 326.0 [M+H−t-Bu]^{+ 1}H NMR (CDCl₃, 400 MHz) δ ppm 7.90 (s, 1H) 7.82 (d, 1H, J=1.3 Hz) 7.59 (d, 1H, J=9.0 Hz) 7.34 (dd, 1H, J=1.7, 9.2 Hz) 4.55 (tt, 1H, J=4.0, 11.6 Hz) 4.2-4.5 (m, 2H) 2.95 (br t, 2H, J=11.3 Hz) 2.2-2.3 (m, 2H) 2.09 (dq, 2H, J=4.5, 12.2 Hz) 1.49 (s, 9H)

### Synthesis of Intermediate C5

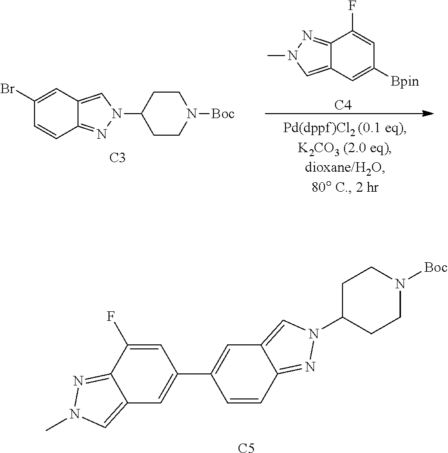

To a solution of tert-butyl 4-(5-bromoindazol-2-yl)piperidine-1-carboxylate (EVAL-0123-24, 100 mg, 262 umol, 1 eq) and 7-fluoro-2-methyl-5-(4,4,5,5-tetramethyl-1,3,2-dioxaborolan-2-yl)indazole (72.6 mg, 262 umol, 1 eq) in dioxane (1.00 mL) was added Pd(dppf)Cl₂ (19.2 mg, 26.3 umol, 0.1 eq) and K₂CO₃ (72.6 mg, 525 umol, 2 eq) under N₂ atmosphere. The mixture was stirred at 80° C. for 2 hrs. TLC (ethyl acetate, Rf=0.44) showed the starting material was consumed and a new major spot was generated. The reaction mixture was cooled to 25° C. and quenched by addition water (10.0 mL) at 25° C., and then extracted with ethyl acetate (3×30.0 mL). The combined organic layers were dried over MgSO₄, filtered and concentrated under reduced pressure to give a residue, which was purified by silica gel column chromatography (petroleum ether/ethyl acetate=1:1) to give tert-butyl 4-[5-(7-fluoro-2-methyl-indazol-5-yl)indazol-2-yl]piperidine-1-carboxylate (20.0 mg, 16.9%) as a solid. LCMS: (ESI, m/z): 450.3 [M+H]^{+ 1}H NMR (CDCl₃, 400 MHz) δ ppm 8.0-8.0 (m, 2H), 7.84 (s, 1H), 7.79 (d, 1H, J=9.2 Hz), 7.6-7.7 (m, 2H), 7.30 (s, 1H), 4.5-4.6 (m, 1H), 4.2-4.4 (m, 5H), 2.9-3.1 (m, 2H), 2.2-2.3 (m, 2H), 2.14 (dq, 2H, J=4.4, 11.9 Hz), 1.50 (s, 9H)

### Synthesis of Compound 105

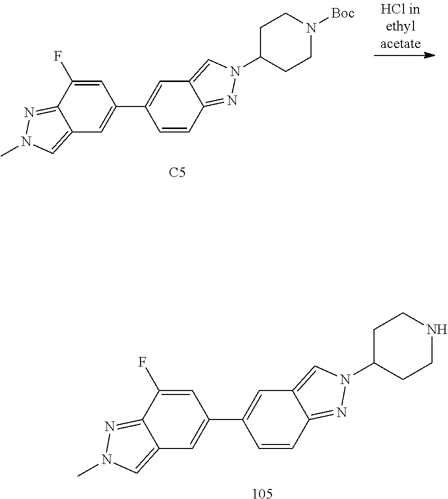 [SMILES: Cn1cc2cc(-c3ccc4nn(C5CCN(C(=O)OC(C)(C)C)CC5)cc4c3)cc(F)c2n1 ; "C5" ; Cn1cc2cc(-c3ccc4nn(C5CCNCC5)cc4c3)cc(F)c2n1 ; "105"]

To a solution of tert-butyl 4-[5-(7-fluoro-2-methyl-indazol-5-yl) indazol-2-yl]piperidine-1-carboxylate (20.0 mg, 44.4 umol, 1 eq) in ethyl acetate (1.00 mL) was added HCl/ethyl acetate (4 M, 1.00 mL) under N₂ atmosphere. The mixture was stirred at 25° C. for 6 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 7-fluoro-2-methyl-5-[2-(4-piperidyl) indazol-5-yl]indazole (EVAL-0123-0003, 17.2 mg, 91.6%) as solid. LCMS: (ESI, m/z): 350.1 [M+H]^{+×1}H NMR (DMSO-d₆, 400 MHz) δ ppm 8.48 (d, 1H, J=2.7 Hz) 8.46 (s, 1H) 8.00 (s, 1H) 7.8-7.8 (m, 1H) 7.7-7.7 (m, 1H) 7.6-7.7 (m, 1H) 7.41 (d, 1H, J=13.3 Hz) 4.8-4.9 (m, 1H) 4.20 (s, 3H) 3.47 (br d, 2H, J=12.8 Hz) 3.1-3.2 (m, 2H) 2.32 (br d, 4H, J=3.4 Hz)

### Example 20: Synthesis of Compound 107

### Synthesis of Intermediate C7

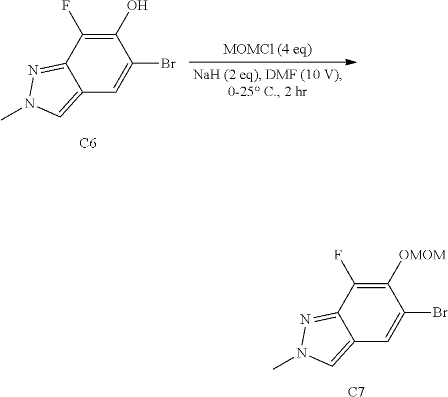

To a solution of 5-bromo-7-fluoro-2-methyl-indazol-6-ol (1.50 g, 6.12 mmol, 1 eq) in DMF (15.0 mL) was added NaH (489 mg, 12.2 mmol, 60% purity, 2 eq) at 0° C. under N₂ protection. The mixture was stirred at 0° C. for 1 hr. Then MOMCl (1.97 g, 24.4 mmol, 1.86 mL, 4 eq) was added to the above mixture at 0° C. under N₂ protection. The mixture was allowed warm to 25° C. and stirred for 1 hr. The reaction was quenched by water (50.0 mL) and the resulting mixture was extracted with ethyl acetate (3×50.0 mL). The combined organic phases were washed with brine (50.0 mL), dried over Na₂SO₄, filtered and filtrate was concentrated under reduced pressure to give 5-bromo-7-fluoro-6-(methoxymethoxy)-2-methyl-indazole (1.60 g, 90%) as a solid. LCMS: (ESI, m/z): 289, 231 [M+H]⁺. ¹H NMR (400 MHz, CDCl₃) δ ppm 7.86 (s, 1H), 7.68 (s, 1H), 5.24 (s, 2H), 4.21 (s, 3H), 3.68 (s, 3H). ¹⁹F NMR (376 MHz, CDCl₃) δ ppm −143.538 (s, 1F)

### Synthesis of Intermediate C8

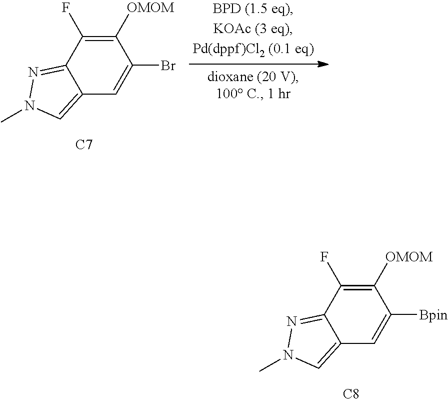

A mixture of 5-bromo-7-fluoro-6-(methoxymethoxy)-2-methyl-indazole (750 mg, 2.59 mmol, 1 eq) and 4,4,5,5-tetramethyl-2-(4,4,5,5-tetramethyl-1,3,2-dioxaborolan-2-yl)-1,3,2-dioxaborolane (988 mg, 3.89 mmol, 1.5 eq) in dioxane (7.50 mL) was added potassium;acetate (763 mg, 7.78 mmol, 3 eq) and Pd(dppf)Cl₂ (189.82 mg, 259.43 umol, 0.1 eq) under N₂ protection. The mixture was stirred at 100° C. for 1 hr. The reaction mixture was filtered and concentrated under reduced pressure to give 7-fluoro-6-(methoxymethoxy)-2-methyl-5-(4,4,5,5-tetramethyl-1,3,2-dioxaborolan-2-yl)indazole (850 mg, 82.8%) as an oil. The crude product was used directly without any purification. LCMS: (ESI, m/z): 337 [M+H]⁺.

### Synthesis of Intermediate C10

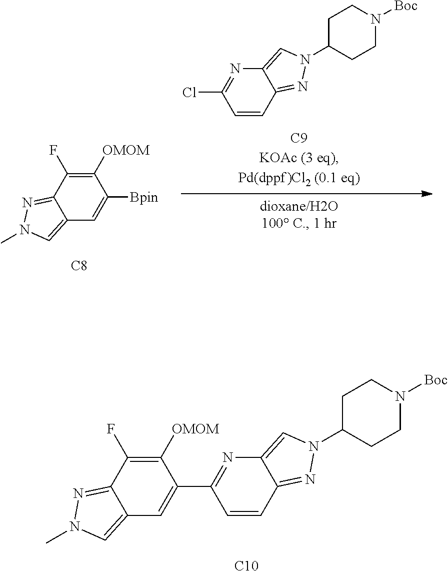

A mixture of 7-fluoro-6-(methoxymethoxy)-2-methyl-5-(4,4,5,5-tetramethyl-1,3,2-dioxaborolan-2-yl)indazole (100 mg, 297 umol, 1 eq) and tert-butyl 4-(5-chloropyrazolo[4,3-b]pyridin-2-yl)piperidine-1-carboxylate (50.1 mg, 148 umol, 0.5 eq) in dioxane (1.50 mL) was added a solution of dipotassium;carbonate (123 mg, 892 umol, 3 eq) in H₂O (300 uL). After purging with N₂ for 3 times, and then cyclopentyl(diphenyl)phosphane;dichloropalladium;iron (21.7 mg, 29.7 umol, 0.1 eq) was added to the above mixture and the mixture was stirred at 100° C. for 1 hr under N₂ atmosphere. The reaction mixture was filtered and concentrated under reduced pressure to give a residue. The residue was purified by column chromatography (SiO₂, petroleum ether/ethyl acetate=10/1 to 0/1) to give tert-butyl 4-[5-[7-fluoro-6-(methoxymethoxy)-2-methyl-indazol-5-yl]pyrazolo [4, 3-b]pyridin-2-yl]piperidine-1-carboxylate (20.0 mg, 13%) as an oil. LCMS: (ESI, m/z): 511 [M+H]⁺.

### Synthesis of Compound 107

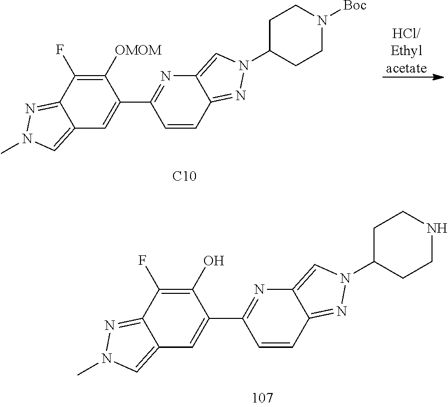

A mixture of tert-butyl 4-[5-[7-fluoro-6-(methoxymethoxy)-2-methyl-indazol-5-yl]pyrazolo[4,3-b]pyridin-2-yl]piperidine-1-carboxylate (EVAL-0123-3, 20.0 mg, 39.1 umol, 1 eq) in HCl/EtOAc (1.00 mL, 4M) was stirred at 20° C. for 1 hr. LCMS showed the starting material was consumed completely. The reaction mixture was filtered and the filter cake was concentrated under reduced pressure to give 7-fluoro-2-methyl-5-[2-(4-piperidyl) pyrazolo [4,3-b]pyridin-5-yl]indazol-6-ol (EVAL-0123-0005, 13.0 mg, 82.3%) as a yellow solid. LCMS: (ESI, m/z): 367 [M+H]⁺. ¹H NMR (400 MHz, DMSO-d₆) δ ppm 8.97-9.18 (m, 1H), 8.81-8.94 (m, 2H), 8.51 (d, J=2.5 Hz, 1H), 8.44 (s, 1H), 8.37 (d, J=9.3 Hz, 1H), 8.23 (d, J=9.5 Hz, 1H), 4.83-5.03 (m, 1H), 4.16 (s, 3H), 3.43-3.50 (m, 2H), 3.09-3.22 (m, 2H), 2.34-2.40 (m, 4H). ¹⁹F NMR (400 MHz, DMSO-d₆) δ ppm −157.953 (m, 1F),

### Example 21: Synthesis of Compound 108

### Synthesis of Intermediate C12

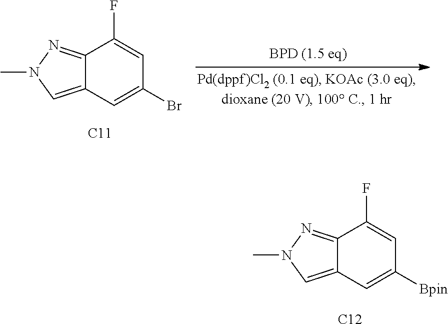

A mixture of 5-bromo-7-fluoro-2-methyl-indazole (800 mg, 3.49 mmol, 1 eq), BPD (887 mg, 4.63 mmol, 1.5 eq), KOAc (1.03 g, 10.5 mmol, 3 eq), Pd(dppf)Cl₂ (142 mg, 349 umol, 0.1 eq) in dioxane (6.00 mL) was degassed and purged with N₂ for 3 times, and then the mixture was stirred at 100° C. for 1 hr under N₂ atmosphere. The mixture was filtered and the filtrate was concentrated under reduced pressure to give a residue. The residue was purified by column chromatography (SiO₂, Petroleum ether/Ethyl acetate=10/1 to 1/1) to give 7-fluoro-2-methyl-5-(4,4,5,5-tetramethyl-1,3,2-dioxaborolan-2-yl)indazole (890 mg, 92%) as a solid. LCMS: (ESI, m/z): 277.1 [M+H]⁺. ¹H NMR (400 MHz, CDCl₃) δ ppm 7.89-8.07 (m, 2H) 7.25-7.28 (m, 1H) 4.26 (s, 3H) 1.38 (s, 12H). ¹⁹F NMR (376 MHz, CDCl₃) δ ppm −130.794.

### Synthesis of Intermediate C14

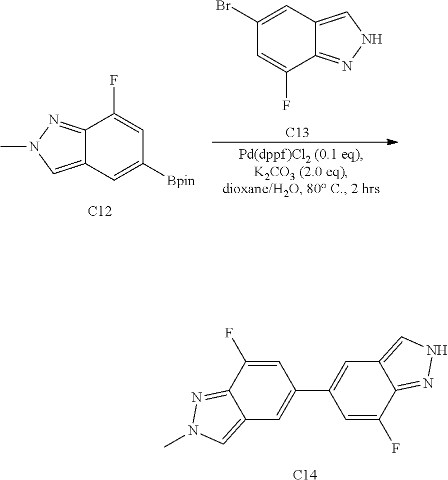

A mixture of 7-fluoro-2-methyl-5-(4,4,5,5-tetramethyl-1,3,2-dioxaborolan-2-yl)indazole (700 mg, 2.54 mmol, 1.1 eq), 5-bromo-7-fluoro-2H-indazole (495 mg, 2.30 mmol, 1 eq), K₂CO₃ (637 mg, 4.61 mmol, 2 eq) and Pd(dppf)Cl₂ (168 mg, 230 umol, 0.1 eq) in dioxane (7.00 mL) and H₂O (700 uL) was degassed and purged with N₂ for 3 times, and then the mixture was stirred at 80° C. for 2 hrs. LCMS showed desired MS was detected. The reaction mixture was filtered and the filtrate was concentrated under reduced pressure to give a residue. The residue was purified by column chromatography (SiO₂, Petroleum ether/Ethyl acetate=1/0 to 1/1) to give 7-fluoro-5-(7-fluoro-2H-indazol-5-yl)-2-methyl-indazole (EVAL-0123-26, 210 mg, 32.0%) as a solid. LCMS: (ESI, m/z): 285.1 [M+H]⁺. ¹H NMR (400 MHz, CDCl₃) δ ppm 10.03-10.53 (m, 1H) 8.17 (d, J=3.30 Hz, 1H) 8.01 (d, J=2.57 Hz, 1H) 7.74 (d, J=1.10 Hz, 1H) 7.62 (d, J=1.22 Hz, 1H) 7.39 (dd, J=11.92, 1.16 Hz, 1H) 6.65-6.75 (m, 1H) 4.29 (s, 3H). ¹⁹F NMR (376 MHz, CDCl₃) δ ppm −126.56, −128.35, −131.73

### Synthesis of Intermediate C16

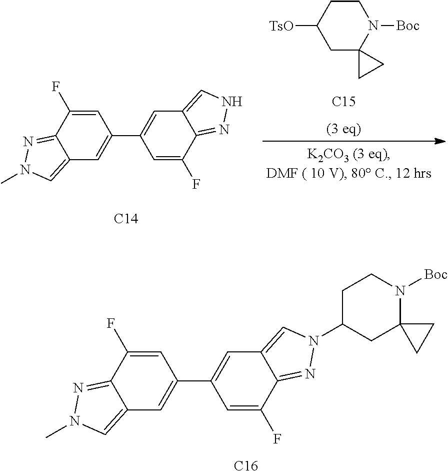

A mixture of 7-fluoro-5-(7-fluoro-2H-indazol-5-yl)-2-methyl-indazole (50.0 mg, 175 umol, 1 eq), tert-butyl 7-(p-tolylsulfonyloxy)-4-azaspiro[2.5]octane-4-carboxylate (201 mg, 527 umol, 3 eq), K₂CO₃ (72.9 mg, 527 umol, 3 eq) in DMF (500 uL) was degassed and purged with N₂ for 3 times, and then the mixture was stirred at 80° C. for 12 hrs under N₂ atmosphere The reaction mixture was quenched by addition H₂O (20.0 mL) at 0° C., and then diluted with ethyl acetate (3.00 mL) and extracted with ethyl acetate (3×4.00 mL). The combined organic layers were washed with brine (3×4.00 mL), dried over Na₂SO₄, filtered and concentrated under reduced pressure to give a residue. The residue was purified by prep-HPLC (column: Waters Xbridge Prep OBD C18 150*40 mm*10 um; mobile phase: [water(NH₄HCO₃)—CH₃CN]; B %: 50%-70%, 8 min) to give tert-butyl 7-[7-fluoro-5-(7-fluoro-2-methyl-indazol-5-yl)indazol-2-yl]-4-azaspiro[2.5]octane-4-carboxylate (17.0 mg, 13.9%) as a solid. LCMS: (ESI, m/z): 494.2 [M+H]⁺. ¹H NMR (400 MHz, CDCl₃) δ ppm 8.04 (d, J=2.03 Hz, 1H), 8.00 (d, J=2.62 Hz, 1H), 7.69 (d, J=1.19 Hz, 1H), 7.61 (d, J=1.19 Hz, 1H), 7.34 (dd, J=13.47, 1.19 Hz, 1H), 7.24 (dd, J=12.40, 1.19 Hz, 1H), 5.03-5.18 (m, 1H), 4.21-4.36 (m, 4H), 3.10 (br t, J=12.99 Hz, 1H), 2.69 (br t, J=12.40 Hz, 1H), 2.23-2.41 (m, 1H), 2.12 (br dd, J=12.93, 1.73 Hz, 1H), 1.52 (s, 9H), 1.33-1.45 (m, 2H), 0.85-0.99 (m, 1H), 0.57-0.73 (m, 2H). ¹⁹F NMR (400 MHz, CDCl₃) δ ppm −128.430.

### Synthesis of Compound 108

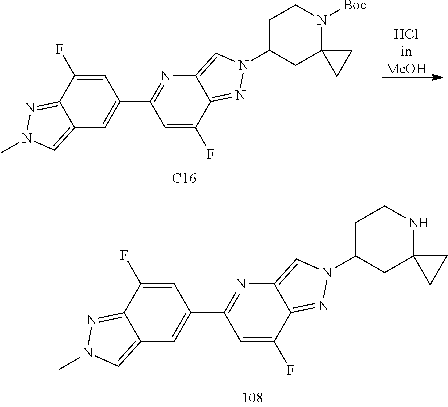 [SMILES: Cn1cc2cc(-c3cc(F)c4nn(C5CCN(C(=O)OC(C)(C)C)C6(CC6)C5)cc4n3)cc(F)c2n1 ; "HClinMeOH" ; "C16" ; Cn1cc2cc(-c3cc(F)c4nn(C5CCNC6(CC6)C5)cc4n3)cc(F)c2n1]

A mixture of tert-butyl 7-[7-fluoro-5-(7-fluoro-2-methyl-indazol-5-yl)indazol-2-yl]-4-azaspiro[2.5]octane-4-carboxylate (15.0 mg, 30.3 umol, 1 eq) in HCl/MeOH (4 M, 1.52 mL, 2. eq) was degassed and purged with N₂ for 3 times, and then the mixture was stirred at 15° C. for 4 hrs under N₂ atmosphere. The reaction mixture was concentrated under reduced pressure to remove solvent. The residue was purified by prep-HPLC (column: Phenomenex Luna 80*30 mm*3 um; mobile phase: [water(HCl)—CH₃CN]; B %: 1%-50%, 8 min) to give 2-(4-azaspiro[2.5]octan-7-yl)-7-fluoro-5-(7-fluoro-2-methyl-indazol-5-yl)indazole (EVAL-0123-28, 7.92 mg, 61%) as a solid. LCMS: (ESI, m/z): 394.0 [M+H]⁺. ¹H NMR (400 MHz, MeOD-d₄) δ ppm 8.46 (d, J=2.63 Hz, 1H) 8.34 (d, J=2.63 Hz, 1H) 7.77 (dd, J=3.25, 1.25 Hz, 2H) 7.38 (ddd, J=12.95, 8.69, 1.38 Hz, 2H) 5.02-5.09 (m, 1H) 4.26 (s, 3H) 3.61-3.70 (m, 1H) 3.42 (br dd, J=7.38, 2.75 Hz, 1H) 2.84-2.97 (m, 1H) 2.46-2.63 (m, 2H) 1.95 (dd, J=14.07, 3.69 Hz, 1H) 1.13-1.22 (m, 2H) 0.96-1.12 (m, 2H). ¹⁹F NMR (376 MHz, MeOD-d₄) δ ppm −131.198, −131.239.

### Example 22: Synthesis of Compound 110

### Synthesis of Intermediate C18

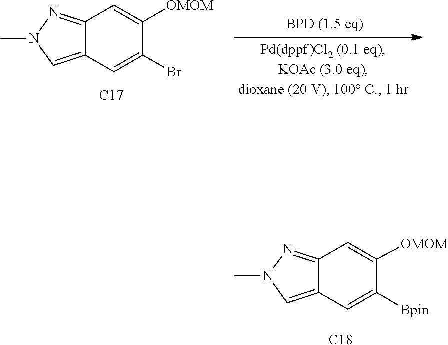

To a mixture of 4,4,5,5-tetramethyl-2-(4,4,5,5-tetramethyl-1,3,2-dioxaborolan-2-yl)-1,3,2-dioxaborolane (210 mg, 829 umol, 1.5 eq), 5-bromo-6-(methoxymethoxy)-2-methyl-indazole (150 mg, 553 umol, 1 eq) and cyclopentyl(diphenyl)phosphane;dichloromethane;dichloro palladium;iron (45.1 mg, 55.3 umol, 0.1 eq) in 1,4-dioxane (2.00 mL) was added potassium acetate (162 mg, 1.66 mmol, 3.0 eq) under nitrogen atmosphere. The resulting mixture was stirred at 100° C. for 1 hr. The reaction mixture was diluted with ethyl acetate (30.0 mL) and filtered, the filtrate was concentrated under reduced pressure to give 6-(methoxymethoxy)-2-methyl-5-(4,4,5,5-tetramethyl-1,3,2-dioxaborolan-2-yl)indazole (300 mg) as a solid. The crude product was used directly in the next step without further purification. LCMS: (ESI, m/z): 319.1 [M+H]⁺.

### Synthesis of Intermediate C20

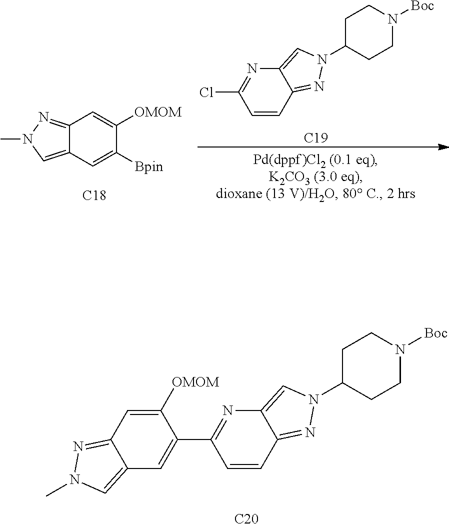

To a mixture of tert-butyl 4-(5-chloropyrazolo[4,3-b]pyridin-2-yl)piperidine-1-carboxylate (185 mg, 550 umol, 1.00 eq), 6-(methoxymethoxy)-2-methyl-5-(4,4,5,5-tetramethyl-1,3,2-dioxaborolan-2-yl)indazole (175 mg, 0.550 mmol, 1 eq) and K₂CO₃ (228 mg, 1.65 mmol, 3.0 eq) in 1,4-dioxane (2.00 mL) and H₂O (500 uL) was added ditert-butyl(cyclopentyl)phosphane;dichloropalladium;iron (35.8 mg, 55.0 umol, 0.1 eq) under nitrogen atmosphere. The resulting mixture was stirred for 2 hrs at 80° C. The reaction mixture was cooled to 20° C. and quenched with water (30.0 mL), the mixture was extracted with ethyl acetate (5×10.0 mL). The organic layer was dried over Na₂SO₄, filtered and the filtrate was concentrated under reduced pressure. The reaction mixture was purified by prep-TLC (SiO₂, ethyl acetate:methanol=10:1) to give tert-butyl 4-(5-(6-(methoxymethoxy)-2-methyl-2H-indazol-5-yl)-2H-pyrazolo[4,3-b]pyridin-2-yl)piperidine-1-carboxylate (40.0 mg, 14.7% yield) as a solid. LCMS: (ESI, m/z): 493.2 [M+H]⁺. ¹H NMR (400 MHz, CDCl₃) δ ppm 8.25 (s, 1H) 8.03 (d, J=9.2 Hz, 1H) 7.92 (s, 1H) 7.88 (s, 1H) 7.66 (d, J=9.0 Hz, 1H) 7.38 (s, 1H), 5.23 (s, 2H) 4.61 (tt, J=4.0, 11.5 Hz, 1H) 4.45-4.29 (m, 2H) 4.20 (s, 3H) 3.44 (s, 3H) 3.07-2.90 (m, 2H) 2.33-2.22 (m, 2H) 2.21-2.11 (m, 2H) 1.50 (s, 9H)

### Synthesis of Compound 110

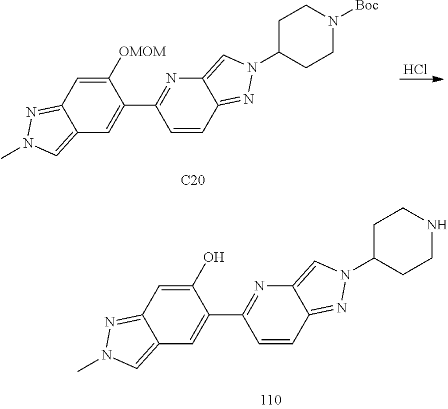

To a solution of tert-butyl 4-[5-[6-(methoxymethoxy)-2-methyl-indazol-5-yl]pyrazolo [4,3-b]pyridin-2-yl]piperidine-1-carboxylate (008-3, 40.0 mg, 81.2 umol, 1 eq) in DCM (2.00 mL) was added the solution of HCl/dioxane (2 M, 2.00 mL) at 25° C. for 2 hrs. The reaction mixture was filtered. The filter cake was triturated with ethyl acetate (10.0 mL) at 25° C. for 60 min to give 2-methyl-5-[2-(4-piperidyl)pyrazolo[4,3-b]pyridin-5-yl]indazol-6-ol (22.1 mg, 70.9%) as a solid. LCMS: (ESI, m/z): 349.1 [M+H]⁺. ¹H NMR (400 MHz, MeOD-d₄) δ ppm 8.99 (s, 1H) 8.93 (d, J=9.17 Hz, 1H) 8.80 (s, 1H) 8.52 (s, 1H) 8.24 (d, J=9.17 Hz, 1H) 7.19 (s, 1H) 5.14-5.33 (m, 1H) 4.35 (s, 3H) 3.62-3.79 (m, 2H) 3.34-3.47 (m, 2H) 2.50-2.70 (m, 4H)

### Example 23: Synthesis of Compound 114

### Synthesis of Intermediate C22

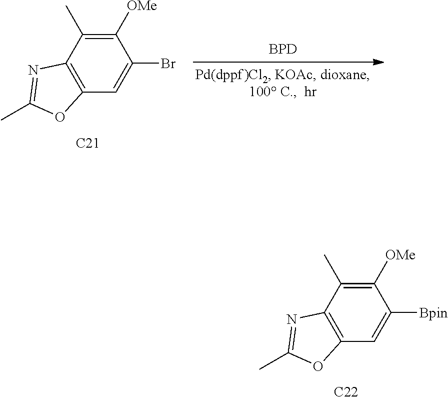

To a solution of 6-bromo-5-methoxy-2,4-dimethyl-1,3-benzoxazole (300 mg, 1.17 mmol, 1 eq) in dioxane (6.00 mL) were added 4,4,5,5-tetramethyl-2-(4,4,5,5-tetramethyl-1,3,2-dioxaborolan-2-yl)-1,3,2-dioxaborolane (446 mg, 1.76 mmol, 1.5 eq), KOAc (344 mg, 3.51 mmol, 3 eq) and Pd(dppf)Cl₂ (85.7 mg, 117 umol, 0.1 eq). The reaction was stirred at 100° C. for 1 hr. The reaction mixture was filtered and concentrated under reduced pressure to give a residue. The residue was purified column chromatographed on silica gel eluted with petroleum ether/ethyl acetate (10/1 to 3/1) to give 5-methoxy-2,4-dimethyl-6-(4,4,5,5-tetramethyl-1,3,2-dioxaborolan-2-yl)-1,3-benzoxazole (150 mg, 42.2%) as a solid. ¹H NMR (400 MHz, CDCl₃) δ ppm 7.65 (s, 1H) 3.80 (s, 3H) 2.64 (s, 3H) 2.50 (s, 3H) 1.39 (s, 12H)

### Synthesis of Intermediate C24

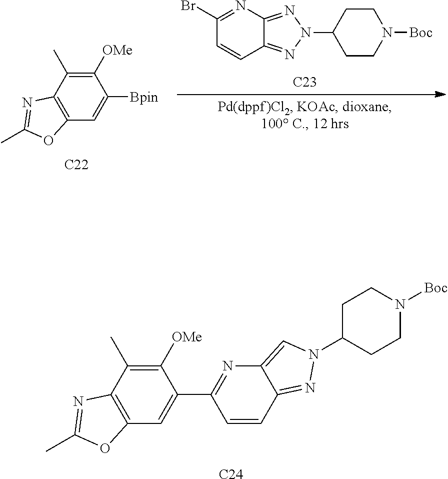

To a solution of tert-butyl 4-(5-bromotriazolo[4,5-b]pyridin-2-yl)piperidine-1-carboxylate (253 mg, 663 umol, 1.5 eq) in dioxane (2.80 mL) were added 5-methoxy-2,4-dimethyl-6-(4,4,5,5-tetramethyl-1,3,2-dioxaborolan-2-yl)-1,3-benzoxazole (134 mg, 442 umol, 1 eq) and KOAc (130 mg, 1.33 mmol, 3 eq) and ditert butyl(cyclopentyl)phosphane;dichloropalladium;iron (28.8 mg, 44.2 umol, 0.1 eq). The reaction mixture was stirred at 120° C. for 12 hrs. The reaction mixture was filtered and concentrated under reduced pressure. The residue was purified column chromatographed on silica gel eluted with petroleum ether/ethyl acetate (10/1 to 3/1) to give tert-butyl 4-[5-(5-methoxy-2,4-dimethyl-1,3-benzoxazol-6-yl)triazolo[4,5-b]pyridin-2-yl]piperidine-1-carboxylate (90.0 mg, 42.5%) as a solid. LCMS: (ESI, m/z): 479.3 [M+H]⁺.

### Synthesis of Compound 114

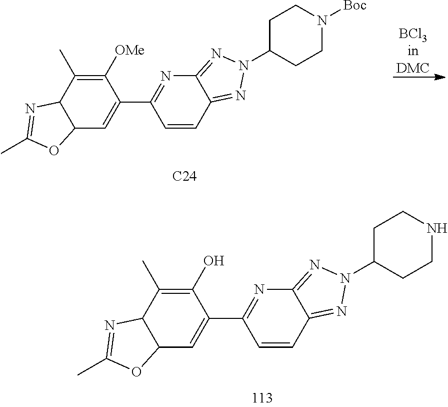 [SMILES: COC1=C(C)C2N=C(C)OC2C=C1c1ccc2nn(C3CCN(C(=O)OC(C)(C)C)CC3)nc2n1 ; "BCl3inDMC" ; "C24" ; CC1=NC2C(C)=C(O)C(c3ccc4nn(C5CCNCC5)nc4n3)=CC2O1]

To a solution of tert-butyl 4-[5-(5-methoxy-2, 4-dimethyl-1, 3-benzoxazol-6-yl) triazolo [4, 5-b]pyridin-2-yl]piperidine-1-carboxylate (80.0 mg, 167 umol, 1 eq) in DCM (8.00 mL) were added BCl₃ (1 M in DCM, 501 uL, 3 eq). The reaction was stirred at 40° C. for 5 hrs. The reaction mixture was filtered and the filter cake was purified by Prep-HPLC (column: Phenomenex C18 75*30 mm*3 um; mobile phase: [water(HCl)—CH₃CN]; B %: 5%-55%, 8 min) to give 5-methoxy-2,4-dimethyl-6-[2-(4-piperidyl)triazolo[4,5-b]pyridin-5-yl]-1,3-benzoxazole (1.95 mg, 3%) as a solid. LCMS: (ESI, m/z): 365.0 [M+H]⁺. ¹H NMR (400 MHz, MeOD-d₄) δ ppm 8.48 (d, J=9.1 Hz, 1H) 8.27 (d, J=9.4 Hz, 1H) 8.17 (s, 1H) 5.13-5.00 (m, 1H) 3.30 (br s, 2H) 3.02-2.89 (m, 2H) 2.65 (s, 3H) 2.49 (s, 3H) 2.45-2.28 (m, 4H).

### Example 24: Synthesis of Compound 113

### Synthesis of Intermediate C26

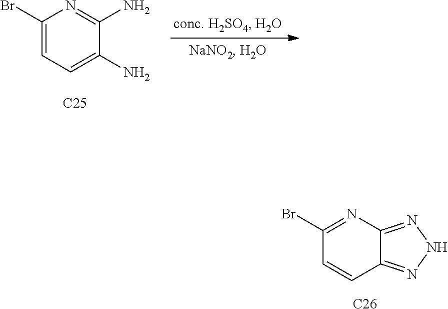 [SMILES: Nc1ccc(Br)nc1N ; "C25" ; Brc1ccc2n[nH]nc2n1 ; "C26"]

To a solution of H₂SO₄ (64.0 mL) in H₂O (214 mL) were added 6-bromopyridine-2, 3-diamine (5.00 g, 26.6 mmol, 1 eq) at 5° C. The reaction was stirred for 0.5 hr at this temperature. Then, a solution of NaNO₂ (2.16 g, 31.38 mmol, 1.18 eq) in H₂O (64.0 mL) was added and the reaction mixture was stirred at 25° C. for 16 hrs. TLC (petroleum ether/ethyl acetate=1/1, Rf=0.5) showed all of the starting material was consumed and a new major spot was generated. The reaction mixture was filtered and the filter cake was washed with water and then dry under vacuum to give 5-bromo-2H-triazolo [4, 5-b]pyridine (3.00 g, 57%) as a solid. LCMS: (ESI, m/z): 198.9, 209.9 [M+H]⁺. ¹H NMR (400 MHz, MeOD-d₄) δ ppm 8.27 (d, J=8.6 Hz, 1H) 7.66 (d, J=8.6 Hz, 1H).

### Synthesis of Intermediate C28

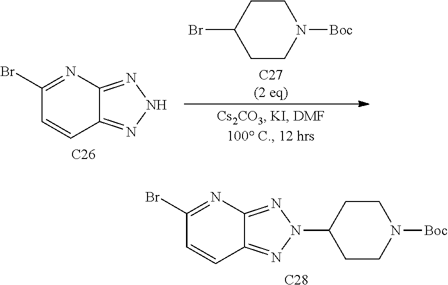

To a solution of 5-bromo-2H-triazolo [4, 5-b]pyridine (3.00 g, 15.0 mmol, 1 eq) in DMF (40.0 mL) were added tert-butyl 4-bromopiperidine-1-carboxylate (7.97 g, 30.1 mmol, 2 eq), KI (5.00 g, 30.1 mmol, 2 eq) and Cs₂CO₃ (24.5 g, 75.3 mmol, 5 eq). The reaction was stirred at 100° C. for 12 hrs. TLC (petroleum ether/ethyl acetate=1/1, Rf=0.8) showed all of the starting material was consumed and a new major spot was generated. The reaction solution was filtered and the filtrate and concentrated. The residue was purified by Prep-HPLC (column: Phenomenex luna C18 80*40 mm*3 um; mobile phase: [water(HCl)—CH₃CN]; B %: 40%-80%, 7 min) to give tert-butyl 4-(5-bromotriazolo[4,5-b]pyridin-2-yl)piperidine-1-carboxylate (2.40 g, 41.6%) as white solid. LCMS: (ESI, m/z): 326.0, 328.0 [M+H−tBu]⁺. ¹H NMR (400 MHz, CDCl₃) δ ppm 8.09 (d, J=8.7 Hz, 1H) 7.56-7.45 (m, 1H) 5.02-4.86 (m, 1H) 4.35-4.13 (m, 2H) 3.15-2.98 (m, 2H) 2.40-2.18 (m, 4H) 1.50 (s, 9H).

### Synthesis of Intermediate C30

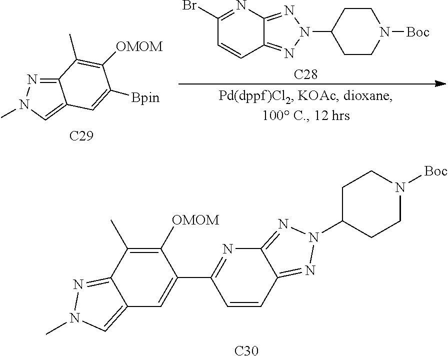

To a solution of 6-(methoxymethoxy)-2,7-dimethyl-5-(4,4,5,5-tetramethyl-1,3,2-dioxaborolan-2-yl)indazole (50.0 mg, 150 umol, 1 eq) in dioxane (2.00 mL) were added tert-butyl 4-(5-bromotriazolo[4,5-b]pyridin-2-yl)piperidine-1-carboxylate (86.3 mg, 225 umol, 1.5 eq), KOAc (44.2 mg, 451 umol, 3 eq) and ditert-butyl(cyclopentyl)phosphane;dichloropalladium;iron (9.81 mg, 15.0 umol, 0.1 eq). The reaction was stirred at 100° C. for 12 hrs. The reaction mixture was filtered and the filtrate was concentrated to give tert-butyl 4-[5-(6-hydroxy-2,7-dimethyl-indazol-5-yl)triazolo[4,5-b]pyridin-2-yl]piperidine-1-carboxylate (40.0 mg, 13.0%) as an oil. The crude product was used directly in next step without any purification. LCMS: (ESI, m/z): 508.3 [M+H]⁺

### Synthesis of Compound 113

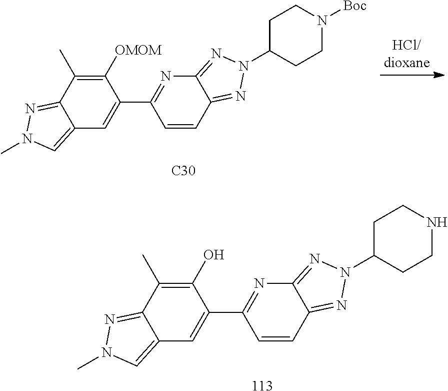

To the crude tert-butyl 4-[5-(6-hydroxy-2, 7-dimethyl-indazol-5-yl) triazolo[4, 5-b] pyridin-2-yl]piperidine-1-carboxylate (40.0 mg, 80.0 umol, 1 eq) was added HCl/dioxane (2.00 mL) and the mixture was stirred at 25° C. for 1 hr. The reaction mixture was filtered and concentrated under reduced pressure. The residue was purified by Prep-HPLC (column: Phenomenex luna C18 80*40 mm*3 um; mobile phase: [water(HCl)—CH₃CN]; B %: 30%-80%, 7 min) to give 2,7-dimethyl-5-[2-(4-piperidyl)triazolo[4,5-b]pyridin-5-yl]indazol-6-ol (11.2 mg) as a solid. LCMS: (ESI, m/z): 364.1 [M+H]⁺. ¹H NMR (400 MHz, MeOD-d₄) δ ppm 8.85 (s, 1H) 8.76 (s, 1H) 8.61 (d, J=9.4 Hz, 1H) 8.42 (d, J=9.4 Hz, 1H) 5.32-5.37 (m, 1H) 4.35 (s, 3H) 3.64 (td, J=4.1, 13.2 Hz, 2H) 3.47-3.35 (m, 2H) 2.78-2.55 (m, 4H) 2.49 (s, 3H).

### Example 25: Synthesis of Compound 115

### Synthesis of Intermediate C32

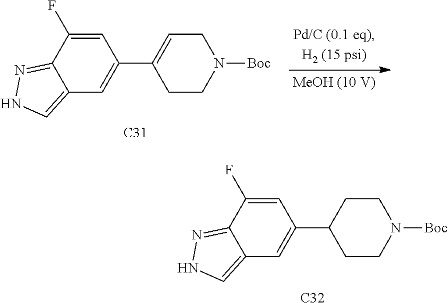 [SMILES: CC(C)(C)OC(=O)N1CC=C(c2cc(F)c3n[nH]cc3c2)CC1 ; "C31" ; CC(C)(C)OC(=O)N1CCC(c2cc(F)c3n[nH]cc3c2)CC1 ; "C32"]

To a solution of tert-butyl 4-(7-fluoro-2H-indazol-5-yl)-3,6-dihydro-2H-pyridine-1-carboxylate (40.0 mg, 126 umol, 1 eq) in MeOH (4.00 mL) was added Pd/C (29.7 mg, 12.6 umol, 5% purity, 0.1 eq) under N₂ atmosphere. The suspension was degassed and purged with H₂ for 3 times. The mixture was stirred under H₂ (15 Psi) at 25° C. for 12 hrs. TLC (petroleum ether/ethyl acetate=3/1, Rf=0.38) showed the starting material was consumed and a new major spot was generated. After filtration via filter Celite pad, the organic layer was concentrated under reduced pressure to afford tert-butyl 4-(7-fluoro-2H-indazol-5-yl)piperidine-1-carboxylate (600 mg, 59.6%) as an oil. LCMS: (ESI, m/z): 264.0 [M+H−tBu]^{+ 1}H NMR (METHANOL-d₄, 400 MHz) δ ppm 7.99-8.12 (1H, m), 7.39-7.62 (1H, m), 7.06 (1H, d, J=12.3 Hz), 4.23 (2H, br d, J=13.4 Hz), 2.76-2.97 (3H, m), 1.89 (2H, br d, J=12.5 Hz), 1.63 (2H, qd, J=12.6 Hz, J=4.3 Hz), 1.49 (s, 9H)

### Synthesis of Intermediate C34

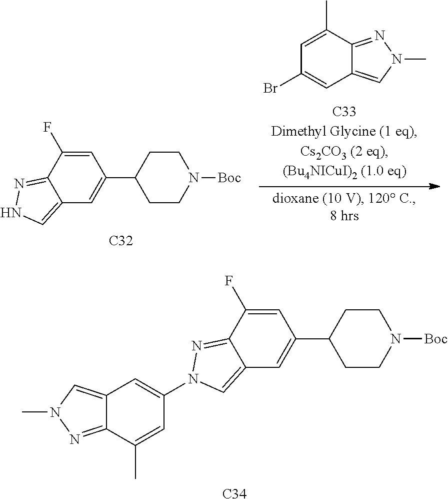 [SMILES: Cc1cc(Br)cc2cn(C)nc12 ; CC(C)(C)OC(=O)N1CCC(c2cc(F)c3n[nH]cc3c2)CC1 ; "C32" ; Cc1cc(-n2cc3cc(C4CCN(C(=O)OC(C)(C)C)CC4)cc(F)c3n2)cc2cn(C)nc12 ; "C34"]

A mixture of tert-butyl 4-(7-fluoro-2H-indazol-5-yl)piperidine-1-carboxylate (50.0 mg, 156 umol, 1 eq), 5-bromo-2,7-dimethyl-indazole (35.2 mg, 156 umol, 1 eq), dimethyl glycine (16.1 mg, 156 umol, 1 eq), iodocopper;tetrabutylammonium;diiodide (175 mg, 156 umol, 1 eq) in dioxane (2.00 mL) was degassed and purged with N₂ for 3 times, and then the mixture was stirred at 120° C. for 8 hrs under N₂ atmosphere. TLC (petroleum ether/ethyl acetate=3/1, Rf=0.38) showed the starting material was consumed and a major new spot was generated. After filtration via filter celite pad, the organic layer was concentrated under reduced pressure evaporated to dryness to afford a residue. The residue was purified by prep-HPLC (column: Phenomenex C18 75*30 mm*3 um; mobile phase: [water(NH₄HCO₃)—CH₃CN]; B %: 40%-70%, 8 min) to give tert-butyl 4-[2-(2,7-dimethylindazol-5-yl)-7-fluoro-indazol-5-yl]piperidine-1-carboxylate (16.0 mg, 19.8%) as a solid. LCMS: (ESI, m/z): 464.2 [M+H]^{+ 1}H NMR (CHLOROFORM-d, 400 MHz) δ ppm 8.40 (1H, d, J=2.4 Hz), 8.00 (2H, d, J=8.4 Hz), 7.64 (1H, s), 7.37 (1H, br s), 6.87 (1H, d, J=12.6 Hz), 4.29 (5H, s), 2.78-2.92 (2H, m), 2.66-2.77 (4H, m), 1.91 (2H, br d, J=12.5 Hz), 1.60-1.74 (2H, m), 1.49-1.52 (9H, m)

### Synthesis of Compound 115

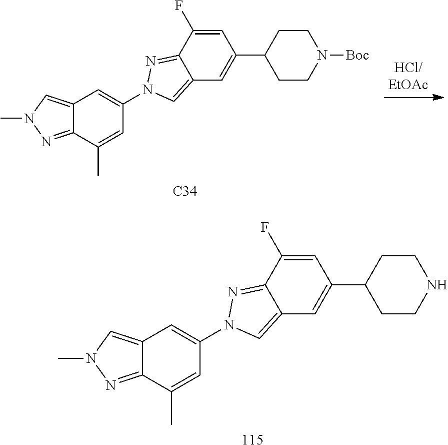 [SMILES: Cc1cc(-n2cc3cc(C4CCN(C(=O)OC(C)(C)C)CC4)cc(F)c3n2)cc2cn(C)nc12 ; "C34" ; Cc1cc(-n2cc3cc(C4CCNCC4)cc(F)c3n2)cc2cn(C)nc12 ; "115"]

To a solution of tert-butyl 4-[2-(2, 7-dimethylindazol-5-yl)-7-fluoro-indazol-5-yl] piperidine-1-carboxylate (16.0 mg, 34.5 umol, 1 eq) in EtOAc (500 uL) was added HCl/EtOAc (4 M, 1.23 mL) under N₂ atmosphere. The mixture was stirred at 25° C. for 12 hrs. TLC (petroleum ether/ethyl acetate=1/1, Rf=0.01) showed the starting material was consumed and a new major spot was generated. The reaction mixture was concentrated under reduced pressure to remove ethyl acetate. The crude product was purified by re-crystallization from MeOH (10.0 mL) at 25° C. to give 2-(2,7-dimethylindazol-5-yl)-7-fluoro-5-(4-piperidyl)indazole (13.4 mg, 95.9%, HCl) as a solid. LCMS: (ESI, m/z): 364.1 [M+H]^{+ 1}H NMR (MeOD-d₄, 400 MHz) δ ppm 8.88 (d, 1H, J=2.7 Hz), 8.59 (s, 1H) 8.22 (d, 1H, J=1.5 Hz) 7.92 (d, 1H, J=0.7 Hz) 7.50 (s, 1H) 7.05 (d, 1H, J=12.6 Hz) 4.36 (s, 3H) 3.56 (br d, 2H, J=12.7 Hz) 3.1-3.3 (m, 2H) 3.0-3.1 (m, 1H) 2.7-2.8 (m, 3H) 2.20 (br d, 2H, J=13.6 Hz) 1.9-2.1 (m, 2H) ¹⁹F NMR (MeOD-d₄, 376 MHz) δ ppm −130.491 (s, 1F)

### Example 26: Synthesis of Compound 117

### Synthesis of Intermediate C36

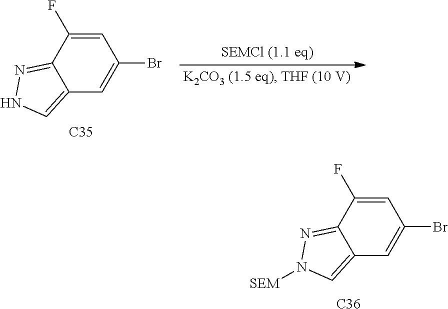 [SMILES: Fc1cc(Br)cc2c[nH]nc12 ; "C35" ; C[Si](C)(C)CCOCn1cc2cc(Br)cc(F)c2n1 ; "C36"]

A mixture of 5-bromo-7-fluoro-2H-indazole (1.00 g, 4.65 mmol, 1 eq), SEM-C₁ (852 mg, 5.12 mmol, 905 μL, 1.1 eq), K₂CO₃ (964 mg, 6.98 mmol, 1.5 eq) in THE (10.0 mL) was degassed and purged with N₂ for 3 times, and then the mixture was stirred at 25° C. for 12 hrs under N₂ atmosphere. TLC (petroleum ether/ethyl acetate=4/1, Rf=0.60) showed the starting material was consumed and a new major spot was generated. The reaction mixture was quenched by addition H₂O at 25° C., and then diluted with EtOAc (10.0 mL) and extracted with ethyl acetate (3×30.0 mL). The combined organic layers were dried over Na₂SO₄, filtered and concentrated under reduced pressure. The residue was triturated with methyl tert-butyl ether (5.00 mL). to afford 2-[(5-bromo-7-fluoro-indazol-2-yl)methoxy]ethyl-trimethyl-silane (900 mg, 50.4%) as yellow solid. LCMS: (ESI, m/z): 345.0, 347.0 [M+H]^{+ 1}H NMR (CDCl₃, 400 MHz) δ ppm 7.91-8.25 (m, 1H) 7.62-7.78 (m, 1H) 7.11-7.36 (m, 1H) 4.63-5.89 (m, 2H) 3.52-3.77 (m, 2H) 0.80-1.08 (m, 2H) 0.02-0.04 (m, 9H)

### Synthesis of Intermediate C37

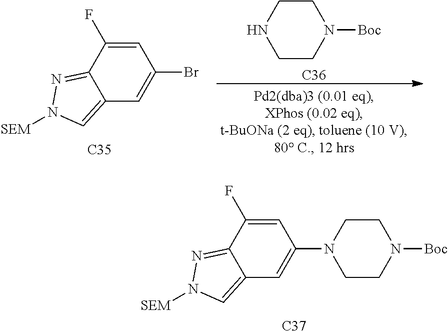 [SMILES: "C36" ; C[Si](C)(C)CCOCn1cc2cc(Br)cc(F)c2n1 ; CC(C)(C)OC(=O)N1CCNCC1 ; "C35" ; CC(C)(C)OC(=O)N1CCN(c2cc(F)c3nn(COCC[Si](C)(C)C)cc3c2)CC1 ; "C37"]

A mixture of 2-[(5-bromo-7-fluoro-indazol-2-yl)methoxy]ethyl-trimethyl-silane (900 mg, 2.61 mmol, 1 eq), tert-butyl piperazine-1-carboxylate (631 mg, 3.39 mmol, 1.3 eq), t-BuONa (500 mg, 5.21 mmol, 2 eq), dicyclohexyl-[2-(2,4,6-triisopropylphenyl)phenyl]phosphane (248 mg, 521 umol, 0.2 eq) and tris(dibenzylideneacetone)dipalladium (238 mg, 260 umol, 0.1 eq) in toluene (10.0 mL) was degassed and purged with N₂ for 3 times, and then the mixture was stirred at 80° C. for 12 hrs under N₂ atmosphere. TLC (petroleum ether/ethyl acetate=3/1, Rf=0.73) showed the starting material was consumed and a new major spot was generated. The reaction mixture was quenched by addition H₂O (30.0 mL) at 25° C., and then diluted with EtOAc (10.0 mL) and extracted with ethyl acetate (3×30.0 mL). The combined organic layers were dried over Na₂SO₄, filtered and concentrated under reduced pressure to give a residue. After filtration via filter celite pad, the organic layer was concentrated under reduced pressure to afford tert-butyl 4-[7-fluoro-2-(2-trimethylsilylethoxymethyl) indazol-5-yl]piperazine-1-carboxylate (900 mg, 77%) as a solid. LCMS: (ESI, m/z): 451.2 [M+H]^{+ 1}H NMR (CHLOROFORM-d, 400 MHz) δ ppm 7.91-8.04 (1H, m), 6.69-6.95 (2H, m), 5.66-5.81 (2H, m), 3.61 (4H, br t, J=5.0 Hz), 3.05-3.13 (4H, m), 1.50 (9H, s), 1.26-1.29 (2H, m), 0.95 (2H, dd, J=7.5 Hz, J=3.6 Hz), −0.08-0.02 (9H, m)

### Synthesis of Intermediate C38

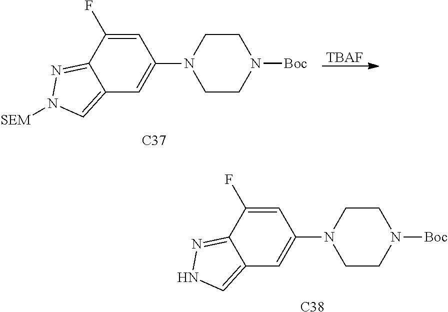

A mixture of tert-butyl 4-[7-fluoro-2-(2-trimethylsilylethoxymethyl)indazol-5-yl]piperazine-1-carboxylate (50.0 mg, 110 umol, 1 eq) and TBAF (1 M, 554 μL, 5 eq) in THE (500 uL) was degassed and purged with N₂ for 3 times, and then the mixture was stirred at 80° C. for 12 hrs under N₂ atmosphere. TLC (petroleum ether/ethyl acetate=3/1, Rf=0.30) showed the starting material was consumed and a new major spot was generated. The reaction mixture was quenched by addition H₂O (20.0 mL) at 25° C. and extracted with ethyl acetate (3×100 mL). The combined organic layers were dried over Na₂SO₄, filtered and concentrated under reduced pressure to give a residue. The residue was purified by column chromatography (SiO₂, Petroleum ether/Ethyl acetate=5/1 to 2/1) to give tert-butyl 4-(7-fluoro-2H-indazol-5-yl) piperazine-1-carboxylate (300 mg, 42.2%) as a solid. LCMS: (ESI, m/z): 321.1 [M+H]^{+ 1}H NMR (CDCl₃, 400 MHz) δ ppm 9.8-10.7 (m, 1H) 8.00 (d, 1H, J=3.4 Hz) 6.8-7.0 (m, 2H) 3.5-3.7 (m, 4H) 3.0-3.2 (m, 4H) 1.50 (s, 9H)

### Synthesis of Intermediate C40

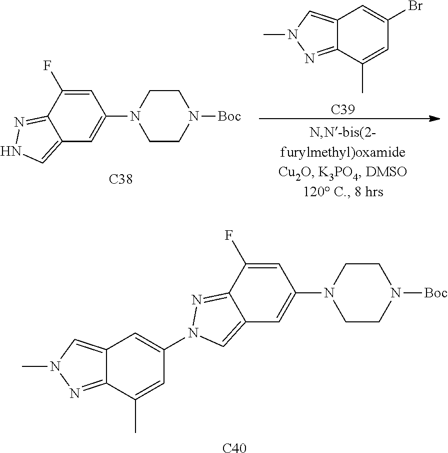 [SMILES: "C39" ; CC(C)(C)OC(=O)N1CCN(c2cc(F)c3n[nH]cc3c2)CC1 ; Cc1cc(Br)cc2cn(C)nc12 ; "C38" ; Cc1cc(-n2cc3cc(N4CCN(C(=O)OC(C)(C)C)CC4)cc(F)c3n2)cc2cn(C)nc12 ; "C40"]

A mixture of tert-butyl 4-(7-fluoro-2H-indazol-5-yl)piperazine-1-carboxylate (100 mg, 312 umol, 1.5 eq), 5-bromo-2,7-dimethyl-indazole (46.8 mg, 208 umol, 1 eq), Cu₂O (2.98 mg, 20.8 umol, 2.13 uL, 0.1 eq), N,N [symbol] bis(2-furylmethyl)oxamide (5.17 mg, 20.8 umol, 0.1 eq), K₃PO₄ (88.3 mg, 416 umol, 2 eq) in DMSO (2.00 mL) was degassed and purged with N₂ for 3 times, and then the mixture was stirred at 120° C. for 8 hrs under N₂ atmosphere. The reaction mixture was quenched by addition water (10.0 mL) at 25° C., and then extracted with ethyl acetate (3×15.0 mL). The combined organic layers were dried over Na₂SO₄, filtered and concentrated under reduced pressure to give a residue. The residue was purified by prep-HPLC (column: Phenomenex C18 75*30 mm*3 um; mobile phase: [water(NH₄HCO₃)—CH₃CN]; B %: 40%-70%, 8 min) to give tert-butyl 4-[2-(2,7-dimethylindazol-5-yl)-7-fluoro-indazol-5-yl]piperazine-1-carboxylate (10.0 mg, 9.83%) as a solid. LCMS: (ESI, m/z): 465.2 [M+H]^{+ 1}H NMR (CDCl₃, 400 MHz) δ ppm 8.31 (d, 1H, J=2.7 Hz) 8.00 (s, 1H) 7.95 (d, 1H, J=1.3 Hz) 7.64 (d, 1H, J=0.9 Hz) 6.85 (dd, 1H, J=1.8, 13.3 Hz) 6.75 (d, 1H, J=1.7 Hz) 4.2-4.4 (m, 3H) 3.6-3.7 (m, 4H) 3.0-3.2 (m, 4H) 2.72 (s, 3H) 1.51 (s, 9H) ¹⁹F NMR (CDCl₃, 376 MHz) δ ppm −127.29 (s, 1F)

### Synthesis of Compound 117

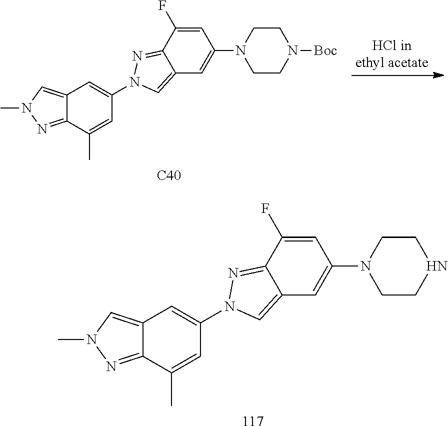 [SMILES: Cc1cc(-n2cc3cc(N4CCN(C(=O)OC(C)(C)C)CC4)cc(F)c3n2)cc2cn(C)nc12 ; "C40" ; Cc1cc(-n2cc3cc(N4CCNCC4)cc(F)c3n2)cc2cn(C)nc12 ; "117"]

To a solution of tert-butyl 4-[2-(2,7-dimethylindazol-5-yl)-7-fluoro-indazol-5-yl]piperazine-1-carboxylate (10.0 mg, 21.5 umol, 1 eq) in ethyl acetate (500 uL) was added HCl/EtOAc (4 M, 1.00 mL) under N₂ atmosphere. The mixture was stirred at 25° C. for 12 hrs. TLC (petroleum ether/ethyl acetate=1/1, Rf=0.01) showed the starting material was consumed and a new major spot was generated. The reaction mixture was concentrated under reduced pressure to remove ethyl acetate. The crude product was purified by re-crystallization from MeOH (5.00 mL) at 25° C. to give 2-(2,7-dimethylindazol-5-yl)-7-fluoro-5-piperazin-1-yl-indazole (6.55 mg, 73.9%, HCl) as a solid. LCMS: (ESI, m/z): 365.1 [M+H]^{+ 1}H NMR (DMSO-d₆, 400 MHz) δ ppm 8.90 (d, 1H, J=2.8 Hz) 8.44 (s, 1H) 8.11 (s, 1H) 7.72 (s, 1H) 7.12 (d, 1H, J=12.4 Hz) 6.88 (s, 1H) 4.19 (s, 3H) 3.30-3.32 (m, 4H) 3.24-3.28 (m, 4H) 2.59 (s, 3H) ¹⁹F NMR (MeOD-d₄, 376 MHz) δ ppm −129.37 (s, 1F).

### Example 27: Synthesis of Compound 119

### Synthesis of Intermediate C43

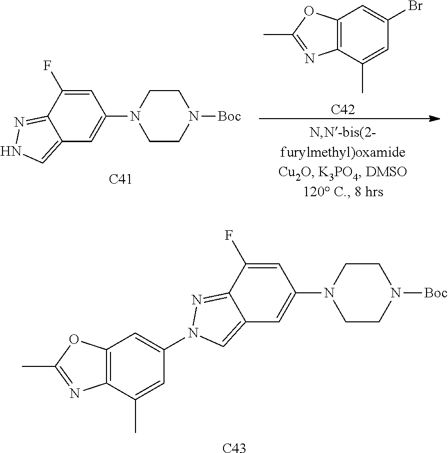 [SMILES: "C42" ; CC(C)(C)OC(=O)N1CCN(c2cc(F)c3n[nH]cc3c2)CC1 ; Cc1nc2c(C)cc(Br)cc2o1 ; "C41" ; Cc1nc2c(C)cc(-n3cc4cc(N5CCN(C(=O)OC(C)(C)C)CC5)cc(F)c4n3)cc2o1 ; "C43"]

A mixture of tert-butyl 4-(7-fluoro-2H-indazol-5-yl)piperazine-1-carboxylate (100 mg, 312 umol, 1.5 eq), 6-bromo-2,4-dimethyl-1,3-benzoxazole (47.0 mg, 208 umol, 1 eq), N,N [symbol] bis(2-furylmethyl)oxamide (5.17 mg, 20.8 umol, 0.1 eq), Cu₂O (2.98 mg, 20.8 umol, 2.13 uL, 0.1 eq), K₃PO₄ (88.3 mg, 416 umol, 2 eq) in DMSO (2.00 mL) was degassed and purged with N₂ for 3 times, and then the mixture was stirred at 120° C. for 8 hrs under N₂ atmosphere. LC-MS showed the starting material was consumed. Several new peaks were shown on LC-MS and 73% of desired compound was detected. The reaction mixture was quenched by addition water (10.0 mL) at 25° C., and then diluted with ethyl acetate (10.0 mL) and extracted with ethyl acetate (3×5.00 mL). The combined organic layers were washed with brine (5.00 mL), dried over Na₂SO₄, filtered and concentrated under reduced pressure to give a residue. The residue was purified by prep-HPLC (column: Phenomenex C18 75*30 mm*3 um; mobile phase: [water(NH₄HCO₃)—CH₃CN]; B %: 40%-70%, 8 min) to give tert-butyl 4-[2-(2,4-dimethyl-1,3-benzoxazol-6-yl)-7-fluoro-indazol-5-yl]piperazine-1-carboxylate (10.0 mg, 9.29%) as a solid. LCMS: (ESI, m/z): 466.2 [M+H]^{+ 1}H NMR (CDCl₃, 400 MHz) δ ppm 8.31 (d, 1H, J=2.6 Hz) 7.86 (d, 1H, J=1.8 Hz) 7.68 (s, 1H) 6.86 (dd, 1H, J=1.7, 13.3 Hz) 6.73 (d, 1H, J=1.6 Hz) 3.6-3.7 (m, 4H) 3.1-3.1 (m, 4H) 2.69 (d, 6H, J=6.4 Hz) 1.51 (s, 9H).

### Synthesis of Compound 119

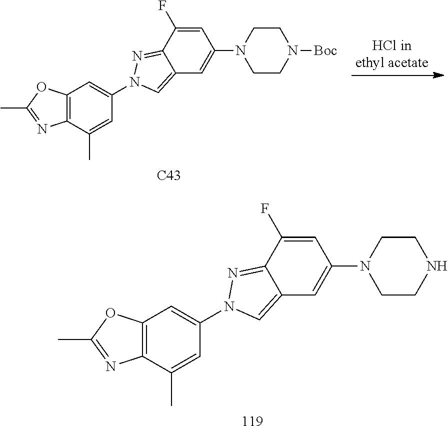 [SMILES: Cc1nc2c(C)cc(-n3cc4cc(N5CCN(C(=O)OC(C)(C)C)CC5)cc(F)c4n3)cc2o1 ; "C43" ; Cc1nc2c(C)cc(-n3cc4cc(N5CCNCC5)cc(F)c4n3)cc2o1 ; "119"]

To a solution of tert-butyl 4-[2-(2,4-dimethyl-1,3-benzoxazol-6-yl)-7-fluoro-indazol-5-yl]piperazine-1-carboxylate (10.0 mg, 21.5 umol, 1 eq) in ethyl acetate (500 uL) was added HCl/ethyl acetate (4 M, 1.00 mL) under N₂ atmosphere. The mixture was stirred at 25° C. for 12 hrs.

TLC (petroleum ether/ethyl acetate=1/1, Rf=0.01) showed the starting material was consumed and a new major spot was generated. The reaction mixture was concentrated under reduced pressure. The crude product was purified by re-crystallization from MeOH (10.0 mL) at 25° C. to give 6-(7-fluoro-5-piperazin-1-yl-indazol-2-yl)-2,4-dimethyl-1,3-benzoxazole (7.55 mg, 96%) as a solid. LCMS: (ESI, m/z): 366.1 [M+H]^{+ 1}H NMR (MeOD-d₄, 400 MHz) δ ppm 8.76 (d, 1H, J=2.6 Hz) 8.02 (d, 1H, J=1.6 Hz) 7.82 (d, 1H, J=1.0 Hz) 7.05 (d, 1H, J=13.6 Hz) 6.97 (d, 1H, J=1.8 Hz) 3.42 (s, 8H) 2.69 (s, 3H) 2.66 (s, 3H) ¹⁹F NMR (MeOD-d₄, 376 MHz) δ ppm −129.308 (s, 1F)

### Example 28: Synthesis of Compound 120

### Synthesis of Intermediate C46

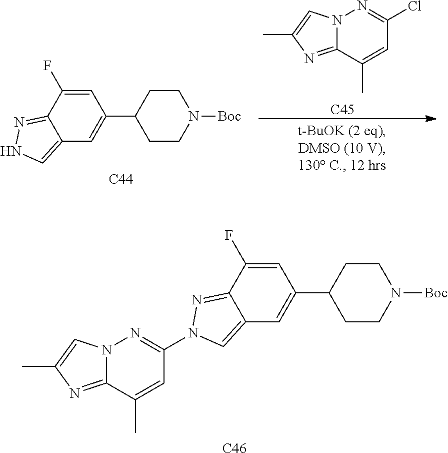 [SMILES: "C45" ; CC(C)(C)OC(=O)N1CCC(c2cc(F)c3n[nH]cc3c2)CC1 ; Cc1cn2nc(Cl)cc(C)c2n1 ; "C44" ; Cc1cn2nc(-n3cc4cc(C5CCN(C(=O)OC(C)(C)C)CC5)cc(F)c4n3)cc(C)c2n1 ; "C46"]

To a solution of tert-butyl 4-(7-fluoro-2H-indazol-5-yl)piperidine-1-carboxylate (E100 mg, 313 umol, 1 eq) in DMSO (2.00 mL) was added t-BuOK (70.2 mg, 626 umol, 2 eq) and 6-chloro-2,8-dimethyl-imidazo[1,2-b]pyridazine (56.8 mg, 313 umol, 1 eq). The reaction mixture was quenched by addition water (10.0 mL) at 25° C., and then diluted with ethyl acetate (10.0 mL) and extracted with ethyl acetate (3×15.0 mL). The combined organic layers were dried over Na₂SO₄, filtered and concentrated under reduced pressure to give a residue. The residue was purified by prep-HPLC (column: Phenomenex C18 75*30 mm*3 um; mobile phase: [water(NH₄HCO₃)—CH₃CN]; B %: 45%-75%, 8 min) to give tert-butyl 4-[2-(2,8-dimethylimidazo[1,2-b]pyridazin-6-yl)-7-fluoro-indazol-5-yl]piperidine-1-carboxylate (15.0 mg, 10%) as a solid. LCMS: (ESI, m/z): 465.3 [M+H]^{+ 1}H NMR (CDCl₃, 400 MHz) δ ppm 8.94 (d, 1H, J=2.7 Hz) 8.02 (d, 1H, J=0.9 Hz) 7.74 (s, 1H) 7.30 (br s, 1H) 6.90 (d, 1H, J=12.3 Hz) 4.1-4.5 (m, 2H) 2.7-2.9 (m, 6H) 2.5-2.6 (m, 3H) 1.8-2.0 (m, 2H) 1.6-1.7 (m, 2H) 1.51 (s, 9H) ¹⁹F NMR (CDCl₃, 376 MHz) δ ppm −128.21 (s, 1F).

### Synthesis of Compound 120

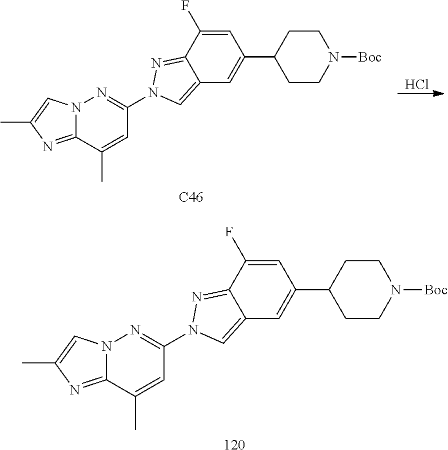 [SMILES: Cc1cn2nc(-n3cc4cc(C5CCN(C(=O)OC(C)(C)C)CC5)cc(F)c4n3)cc(C)c2n1 ; "HCl" ; "C46" ; Cc1cn2nc(-n3cc4cc(C5CCN(C(=O)OC(C)(C)C)CC5)cc(F)c4n3)cc(C)c2n1 ; "120"]

To a solution of tert-butyl 4-[2-(2,8-dimethylimidazo[1,2-b]pyridazin-6-yl)-7-fluoro-indazol-5-yl]piperidine-1-carboxylate (15.0 mg, 32.2 umol, 1 eq) in EtOAc (500 uL) was added HCl/EtOAc (4 M, 1.00 mL) under N₂ atmosphere. The mixture was stirred at 25° C. for 12 hrs. TLC (petroleum ether/ethyl acetate=1/1, Rf=0.01) showed the starting material was consumed and a new major spot was generated. The reaction mixture was concentrated under reduced pressure. The crude product was purified by re-crystallization from MeOH (10.0 mL) at 25° C. to give 6-[7-fluoro-5-(4-piperidyl)indazol-2-yl]-2,8-dimethyl-imidazo[1,2-b]pyridazine (12.4 mg, 92.4%) as a solid. LCMS: (ESI, m/z): 365.1 [M+H]^{+ 1}H NMR (MeOD-d₄, 400 MHz) δ ppm 9.27 (d, 1H, J=2.6 Hz), 8.67 (d, 1H, J=1.1 Hz), 8.36 (d, 1H, J=0.7 Hz), 7.51 (s, 1H), 7.1-7.2 (m, 1H), 3.55 (br d, 2H, J=12.8 Hz), 3.1-3.2 (m, 2H), 3.0-3.1 (m, 1H), 2.85 (d, 3H, J=0.9 Hz), 2.68 (d, 3H, J=0.6 Hz), 2.18 (br d, 2H, J=14.2 Hz), 1.9-2.0 (m, 2H) ¹⁹F NMR (MeOD-d₄, 376 MHz) δ ppm −129.69 (s, 1F)

### Example 29: Synthesis of Compound 121

### Synthesis of Intermediate C49

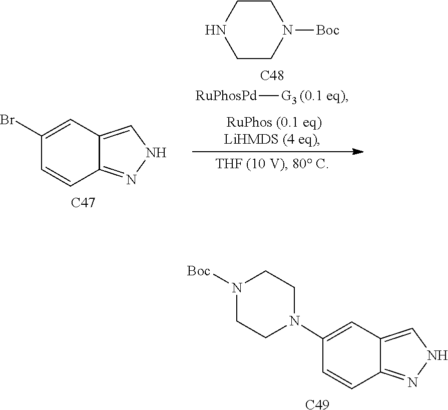

A mixture of tert-butyl piperazine-1-carboxylate (1.13 g, 6.09 mmol, 1.2 eq), LiHMDS (1 M, 20.1 mL, 4 eq), 5-bromo-2H-indazole (1.00 g, 5.08 mmol, 1 eq) [2-(2-aminophenyl)phenyl]-methylsulfonyloxy-palladium;dicyclohexyl-[2-(2,6-diisopropoxyphenyl)phenyl]phosphane (424 mg, 507 umol, 0.1 eq), dicyclohexyl-[2-(2,6-diisopropoxyphenyl)phenyl]phosphane (236 mg, 507 umol, 0.1 eq) in THE (10.0 mL) was degassed and purged with N₂ for 3 times, and then the mixture was stirred at 80° C. for 12 hrs under N₂ atmosphere. The reaction mixture was quenched by addition water (10.0 mL) at 25° C., and then extracted with ethyl acetate (3×20.0 mL). The combined organic layers were dried over Na₂SO₄, filtered and concentrated under reduced pressure to give a residue. The residue was purified by column chromatography (SiO₂, petroleum ether/ethyl acetate=10/1 to 3/1) to afford tert-butyl 4-(2H-indazol-5-yl) piperazine-1-carboxylate (500 mg, 29%) as a solid. LCMS: (ESI, m/z): 303.1 [M+H]^{+ 1}H NMR (DMSO-d₆, 400 MHz) δ ppm 12.85 (br s, 1H) 7.90 (s, 1H) 7.42 (br d, 1H, J=9.0 Hz) 7.2-7.2 (m, 1H) 7.12 (s, 1H) 3.48 (br s, 4H) 3.01 (br d, 4H, J=4.3 Hz) 1.42 (s, 9H)

### Synthesis of Intermediate C50

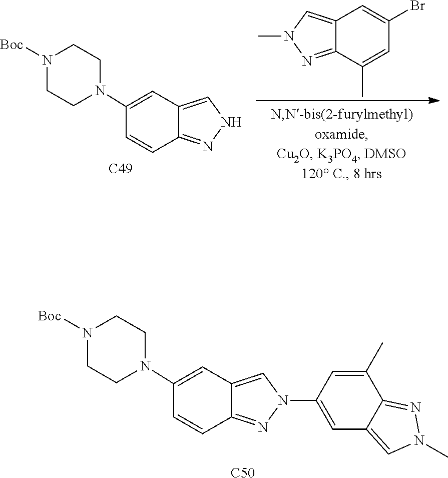 [SMILES: CC(C)(C)OC(=O)N1CCN(c2ccc3n[nH]cc3c2)CC1 ; Cc1cc(Br)cc2cn(C)nc12 ; "C49" ; Cc1cc(-n2cc3cc(N4CCN(C(=O)OC(C)(C)C)CC4)ccc3n2)cc2cn(C)nc12 ; "C50"]

A mixture of tert-butyl 4-(2H-indazol-5-yl)piperazine-1-carboxylate (150 mg, 496 umol, 1.5 eq), 5-bromo-2,7-dimethyl-indazole (74.4 mg, 330 umol, 1 eq), N,N [symbol] bis(2-furylmethyl)oxamide (8.21 mg, 33.0 umol, 0.1 eq), K₃PO₄ (140.4 mg, 661 umol, 2 eq) and Cu₂O (4.73 mg, 33.0 umol, 3.38 uL, 0.1 eq) in DMSO (2.00 mL) was degassed and purged with N₂ for 3 times, and then the mixture was stirred at 120° C. for 8 hrs under N₂ atmosphere. LC-MS showed the starting material was consumed and 36% of desired compound was detected. The reaction mixture was quenched by addition water (10.0 mL) at 25° C., and then diluted with ethyl acetate (10.0 mL) and extracted with ethyl acetate (3×5.00 mL). The combined organic layers were washed with brine (5.00 mL), dried over Na₂SO₄, filtered and concentrated under reduced pressure to give a residue. The residue was purified by prep-HPLC (column: Phenomenex C18 75*30 mm*3 um; mobile phase: [water(NH₄HCO₃)-ACN]; B %: 45%-65%, 8 min) to give tert-butyl 4-[2-(2,7-dimethylindazol-5-yl)indazol-5-yl]piperazine-1-carboxylate (12.0 mg, 7.31%) as a solid. LCMS: (ESI, m/z): 447.2 [M+H]^{+ 1}H NMR (CDCl₃, 400 MHz) δ ppm 8.28 (s, 1H), 7.99 (s, 1H), 7.90 (d, 1H, J=1.5 Hz), 7.72 (d, 1H, J=9.3 Hz), 7.63 (d, 1H, J=0.6 Hz), 7.18 (dd, 1H, J=2.1, 9.5 Hz), 6.97 (d, 1H, J=1.7 Hz), 4.28 (s, 3H), 3.6-3.7 (m, 4H), 3.1-3.1 (m, 4H), 2.73 (s, 3H), 1.51 (s, 9H)

### Synthesis of Compound 121

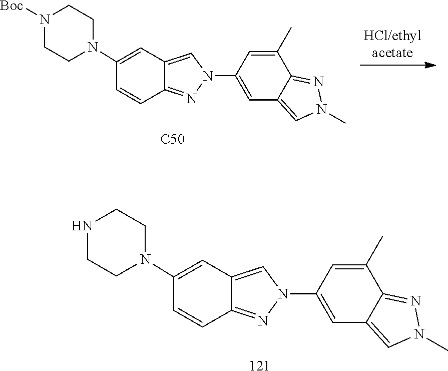 [SMILES: Cc1cc(-n2cc3cc(N4CCN(C(=O)OC(C)(C)C)CC4)ccc3n2)cc2cn(C)nc12 ; "C50" ; Cc1cc(-n2cc3cc(N4CCNCC4)ccc3n2)cc2cn(C)nc12 ; "121"]

To a solution of tert-butyl 4-[2-(2,7-dimethylindazol-5-yl)indazol-5-yl]piperazine-1-carboxylate (12.0 mg, 26.8 umol, 1 eq) in ethyl acetate (500 uL) was added HCl/ethyl acetate (4 M, 958 uL) under N₂ atmosphere. The mixture was stirred at 25° C. for 12 hrs. TLC (petroleum ether/ethyl acetate=1/1, Rf=0.01) showed the starting material was consumed and a new major spot was generated. The reaction mixture was concentrated under reduced pressure. The crude product was purified by re-crystallization from MeOH (10.0 mL) at 25° C. to give 2,7-dimethyl-5-(5-piperazin-1-ylindazol-2-yl)indazole (8.95 mg, 93.2%) as a solid. LCMS: (ESI, m/z): 347.1 [M+H]^{+ 1}H NMR (DMSO-d₆, 400 MHz) δ ppm 9.21 (br s, 2H), 8.84 (s, 1H), 8.45 (s, 1H), 8.11 (d, 1H, J=1.4 Hz), 7.77 (s, 1H), 7.64 (d, 1H, J=9.3 Hz), 7.24 (dd, 1H, J=2.1, 9.5 Hz), 7.04 (d, 1H, J=1.5 Hz), 4.21 (s, 3H), 3.35 (br d, 4H, J=5.2 Hz), 3.27 (br s, 4H), 2.60 (s, 3H)

### Example 30: Synthesis of Compound 123

### Synthesis of Intermediate C53

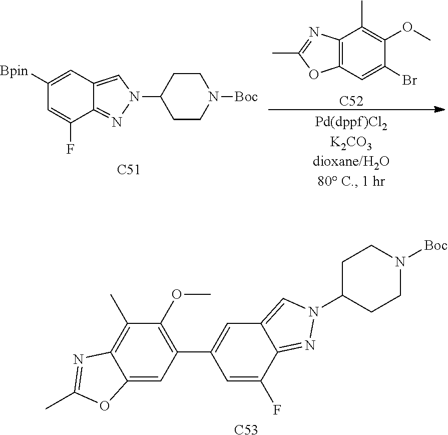

To a mixture of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1.50 eq) and 6-bromo-5-methoxy-2,4-dimethyl-1,3-benzoxazole (76.7 mg, 299 umol, 1.0 eq) in dioxane (1.60 mL) was added a solution of K₂CO₃ (82.7 mg, 599 umol, 2.0 eq) in H₂O (0.400 mL). Then, 1,1-bis(diphenylphosphino)ferrocene]dichloropalladium(II) (43.8 mg, 59.9 umol, 0.20 eq) was added to the above mixture under N₂ protection. The reaction mixture was stirred at 80° C. for 1 hr. The reaction mixture was diluted with H₂O (10.0 mL), extracted with ethyl acetate (3×10.0 mL), dried over Na₂SO₄, filtered and concentrated under reduced pressure to give an oil. The oil was purified by column chromatography on silica gel eluted with petroleum ether/ethyl acetate (1/0 to 5/1) to give tert-butyl 4-[7-fluoro-5-(5-methoxy-2,4-dimethyl-1,3-benzoxazol-6-yl)indazol-2-yl]piperidine-1-carboxylate (130 mg, 82.5%) as an oil. LCMS: (ESI, m/z): 495.3 [M+H]^{+ 1}H NMR (400 MHz, METHANOL-d₄) δ ppm 8.45 (d, J=2.62 Hz, 1H) 7.68 (d, J=1.07 Hz, 1H) 7.41 (s, 1H) 7.27 (dd, J=12.87, 1.19 Hz, 1H) 4.74 (s, 1H) 4.30 (br d, J=13.95 Hz, 2H) 3.39 (s, 3H) 3.05 (br s, 2H) 2.65 (s, 3H) 2.53 (s, 3H) 2.07-2.29 (m, 4H) 1.51 (s, 9H).

### Synthesis of Compound 123

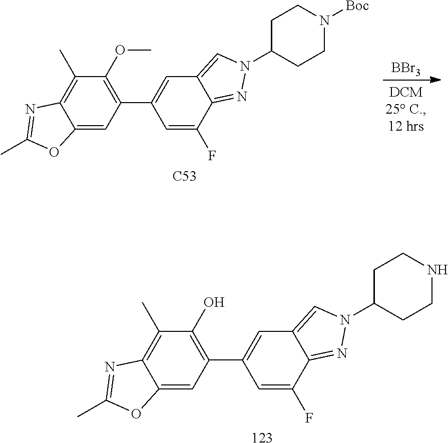

To a solution of tert-butyl 4-[7-fluoro-5-(5-methoxy-2,4-dimethyl-1,3-benzoxazol-6-yl)indazol-2-yl]piperidine-1-carboxylate (60.0 mg, 121 umol, 1.00 eq) in dichloromethane (6.00 mL) was added BBr₃ (152 mg, 607 umol, 5.00 eq) at 0° C. The reaction mixture was stirred at 25° C. for 12 hrs. The reaction mixture was filtered, the filter cake was purified by prep-HPLC (column: waters Xbridge Prep OBD C18 150*40 mm*10 um; mobile phase: [water (NH₄HCO₃)-acetonitrile]; B %: 35%-55%, 8 min) to give 6-[7-fluoro-2-(4-piperidyl)indazol-5-yl]-2,4-dimethyl-1,3-benzoxazol-5-ol (EVAL-0122-0016, 25.3 mg, 55%) as a solid. LCMS: (ESI, m/z): 381.2 [M+H]^{+ 1}HNMR (400 MHz, METHANOL-d₄) δ ppm 8.41 (d, J=2.75 Hz, 1H) 7.61 (d, J=1.00 Hz, 1H) 7.30 (s, 1H) 7.21 (dd, J=12.76, 1.13 Hz, 1H) 4.62-4.73 (m, 1H) 3.27 (br s, 2H) 2.88 (td, J=12.54, 2.31 Hz, 2H) 2.62 (s, 3H) 2.48 (s, 3H) 2.26 (br s, 2H) 2.10-2.22 (m, 2H).

### Example 31: Synthesis of Compound 125

### Synthesis of Intermediate C56

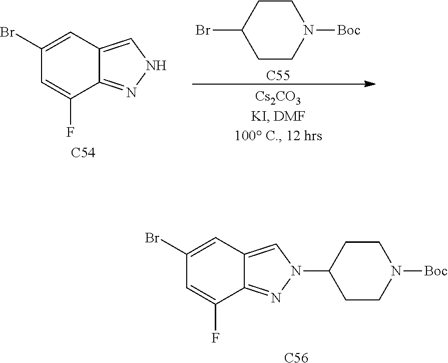 [SMILES: "C55" ; Fc1cc(Br)cc2c[nH]nc12 ; CC(C)(C)OC(=O)N1CCC(Br)CC1 ; "C54" ; CC(C)(C)OC(=O)N1CCC(n2cc3cc(Br)cc(F)c3n2)CC1 ; "C56"]

A solution of 5-bromo-7-fluoro-2H-indazole (5.00 g, 23.2 mmol, 1.0 eq) in N,N-dimethylformamide (50.0 mL) was added tert-butyl 4-bromopiperidine-1-carboxylate (18.4 g, 69.7 mmol, 3.0 eq), KI (11.5 g, 69.7 mmol, 3.0 eq) and Cs₂CO₃ (37.8 g, 116 mmol, 5.0 eq). The reaction mixture was stirred at 100° C. for 12 hrs. The reaction mixture was diluted with H₂O (100 mL), extracted with ethyl acetate (3×100 mL), washed with brine (2×100 mL), dried over Na₂SO₄ filtered and concentrated under reduced pressure to give an oil. The oil was purified by column chromatography on silica gel eluted with petroleum ether/ethyl acetate (50/1 to 5/1) to give yellow oil. The yellow oil was purified by prep-HPLC (column: Xtimate C18 10u 250 mm*80 mm; mobile phase: [water (NH₄HCO₃)-acetonitrile]; B %: 60%-95%, 25 min) to afford tert-butyl 4-(5-bromo-7-fluoro-indazol-2-yl) piperidine-1-carboxylate (3.00 g, 29%) as a solid. LCMS: (ESI, m/z): 342.0 [M+H]⁺. ¹H NMR (400 MHz, DMSO-d₆) δ ppm 8.58 (d, J=2.86 Hz, 1H) 7.81 (d, J=1.55 Hz, 1H) 7.26 (dd, J=11.03, 1.49 Hz, 1H) 4.69-4.84 (m, 1H) 4.09 (br d, J=11.68 Hz, 2H) 2.95 (br s, 2H) 2.12 (br dd, J=12.10, 2.09 Hz, 2H) 1.88-1.97 (m, 2H) 1.43 (s, 9H)

### Synthesis of Intermediate C57

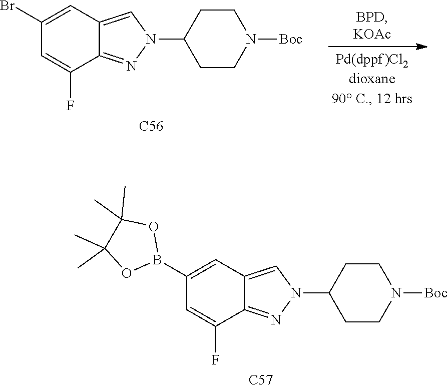 [SMILES: CC(C)(C)OC(=O)N1CCC(n2cc3cc(Br)cc(F)c3n2)CC1 ; "C56" ; CC(C)(C)OC(=O)N1CCC(n2cc3cc(B4OC(C)(C)C(C)(C)O4)cc(F)c3n2)CC1 ; "C57"]

A mixture of tert-butyl 4-(5-bromo-7-fluoro-indazol-2-yl)piperidine-1-carboxylate (2.50 g, 6.28 mmol, 1.0 eq) and bis(pinacolato)diboron (4.78 g, 18.8 mmol, 3.0 eq) in dioxane (25.0 mL) was added potassium acetate (1.23 g, 12.5 mmol, 2.0 eq). The mixture was bubbled with argon for 5 min. Then chloro(2-dicyclohexylphosphino-2′,4′,6′-triisopropyl-1,1′-biphenyl)[2-(2′-amino-1,1′-biphenyl)]palladium(II) (493 mg, 627 umol, 0.10 eq) was added in one portion and the mixture was bubbled with argon for additional 5 min. The resulting mixture was stirred 100° C. for 1 hr. LCMS showed the reaction was completed. The reaction mixture was filtered and the filtrate was concentrated in vacuum to afford an oil. The oil was purified by column chromatography on silica gel eluted with petroleum ether/ethyl acetate (1/0 to 5/1) to give tert-butyl 4-[7-fluoro-5-(4,4,5,5-tetramethyl-1,3,2-dioxaborolan-2-yl)indazol-2-yl]piperidine-1-carboxylate (3.00 g, 91.2%) as an oil. LCMS: (ESI, m/z): 446.2 [M+H]^{+ 1}H NMR (400 MHz, DMSO-d6) δ ppm 8.66 (d, J=2.81 Hz, 1H) 7.95 (s, 1H) 7.07 (d, J=12.23 Hz, 1H) 4.76 (s, 1H) 4.10 (br d, J=12.10 Hz, 2H) 2.97 (br s, 2H) 2.10-2.20 (m, 2H) 1.89-1.98 (m, 2H) 1.43 (s, 9H) 1.30 (s, 12H)

### Synthesis of Intermediate C59

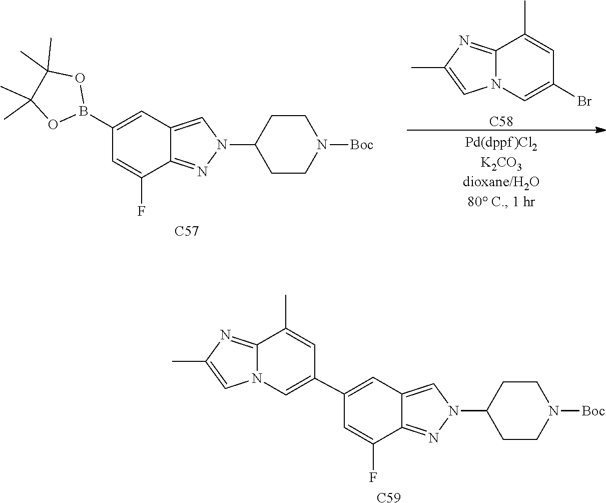 [SMILES: "C58" ; CC(C)(C)OC(=O)N1CCC(n2cc3cc(B4OC(C)(C)C(C)(C)O4)cc(F)c3n2)CC1 ; Cc1cn2cc(Br)cc(C)c2n1 ; "C57" ; Cc1cn2cc(-c3cc(F)c4nn(C5CCN(C(=O)OC(C)(C)C)CC5)cc4c3)cc(C)c2n1 ; "C59"]

To a mixture of tert-butyl 4-[7-fluoro-5-(4,4,5,5-tetramethyl-1,3,2-dioxaborolan-2-yl)indazol-2-yl]piperidine-1-carboxylate (0.20 g, 449 umol, 1.50 eq) and 6-bromo-2,8-dimethyl-imidazo[1,2-a]pyridine (67.4 mg, 299 umol, 1.0 eq) in dioxane (1.60 mL) was added a solution of K₂CO₃ (82.8 mg, 599 umol, 2.0 eq) in H₂O (0.400 mL). Then, 1,1-bis(diphenylphosphino)ferrocene]dichloropalladium(II) (43.8 mg, 59.9 umol, 0.20 eq) was added to the above mixture under N₂ protection. The reaction mixture was stirred at 80° C. for 1 hr. The reaction mixture was diluted with H₂O (10.0 mL), extracted with ethyl acetate (3×10.0 mL), dried over Na₂SO₄ filtered and concentrated under reduced pressure to give an oil that was purified by column chromatography on silica gel eluted with petroleum ether/ethyl acetate (1/0 to 5/1) to give tert-butyl4-[5-(2,8-dimethylimidazo[1,2-a]pyridin-6-yl)-7-fluoro-indazol-2-yl]piperidine-1-carboxylate (0.15 g, 73.4%) as an oil. LCMS: (ESI, m/z): 464.2 [M+H]⁺. ¹H NMR (400 MHz, DMSO-d₆) δ ppm 8.72 (s, 1H) 8.64 (d, J=2.41 Hz, 1H) 7.84 (s, 1H) 7.65 (s, 1H) 7.38-7.47 (m, 2H) 4.77 (brt, J=11.29 Hz, 1H) 4.07-4.18 (m, 2H) 3.33 (s, 3H) 2.90-3.07 (m, 2H) 2.35 (s, 3H) 2.11-2.17 (m, 2H) 1.95-2.04 (m, 2H) 1.44 (s, 9H)

### Synthesis of Compound 125

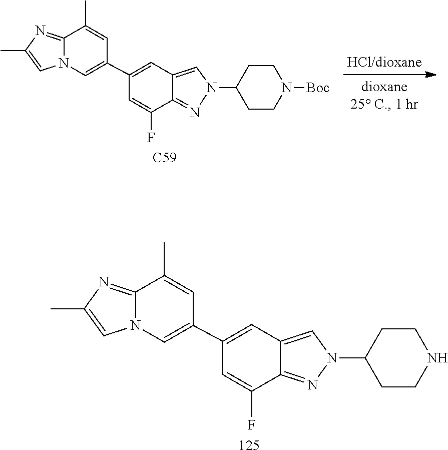

To a solution of tert-butyl 4-[5-(2,8-dimethylimidazo[1,2-a]pyridin-6-yl)-7-fluoro-indazol-2-yl]piperidine-1-carboxylate (65.0 mg, 140 umol, 1.0 eq) in dioxane (2.00 mL) was added HCl/dioxane (4.0 M, 1.30 mL, 37.1 eq) dropwise under N₂ protection. The reaction mixture was stirred at 25° C. for 1 hr. LCMS showed the reaction was completed. The reaction mixture was filtered and the filter cake was dried in vacuum to afford a solid. The solid was purified by prep-HPLC (column: Phenomenex Luna 80*30 mm*3 um; mobile phase: [water (HCl)-acetonitrile]; B %: 1%-35%, 8 min) to get 5-(2,8-dimethylimidazo[1,2-a]pyridin-6-yl)-7-fluoro-2-(4-piperidyl)indazole (35.4 mg, 62.6%) as a solid. LCMS: (ESI, m/z): 364.1 [M+H]⁺. ¹H NMR (400 MHz, METHANOL-d₄) δ ppm 8.90 (s, 1H) 8.59 (d, J=2.63 Hz, 1H) 8.11 (s, 1H) 7.97 (dd, J=12.38, 1.13 Hz, 2H) 7.43 (dd, J=12.51, 1.38 Hz, 1H) 5.00 (dt, J=10.04, 5.05 Hz, 1H) 3.61-3.73 (m, 2H) 3.32 (br s, 2H) 2.71 (s, 3H) 2.60 (d, J=0.88 Hz, 3H) 2.43-2.55 (m, 4H). ¹⁹F NMR (377 MHz, METHANOL-d4) δ ppm −129.79 (s, 1F)

### Example 32: Synthesis of Compound 126

### Synthesis of Intermediate C62

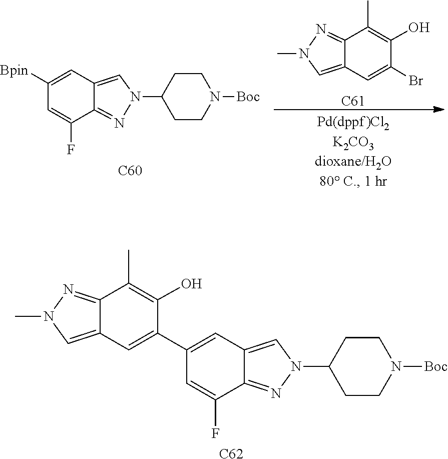

To a mixture of tert-butyl 4-[7-fluoro-5-(4,4,5,5-tetramethyl-1,3,2-dioxaborolan-2-yl)indazol-2-yl]piperidine-1-carboxylate (BC, 300 mg, 674 umol, 1.50 eq) and 5-bromo-2,7-dimethyl-indazol-6-ol (108 mg, 449 umol, 1.00 eq) in dioxane (2.40 mL) was added a solution of K₂CO₃ (124 mg, 898 umol, 2.00 eq) in H₂O (0.600 mL) and 1,1-bis(diphenylphosphino) ferrocene] dichloropalladium(II) (65.7 mg, 89.8 umol, 0.200 eq) successively under N₂ protection. The reaction mixture was stirred at 80° C. for 1 hr. The reaction mixture was diluted with H₂O (10.0 mL), extracted with ethyl acetate (3×10.0 mL), dried over Na₂SO₄, filtered and concentrated under reduced pressure to give brown solid. The oil was triturated with dichloromethane (5.00 mL) and filtered. The filter cake was dried over in vacuum to give tert-butyl 4-[7-fluoro-5-(6-hydroxy-2,7-dimethyl-indazol-5-yl)indazol-2-yl]piperidine-1-carboxylate (65.0 mg, 27.7%) as a solid. LCMS: (ESI, m/z): 480.1 [M+H]⁺. ¹H NMR (400 MHz, DMSO-d₆) δ ppm 8.58 (d, J=2.74 Hz, 1H) 8.20 (d, J=15.74 Hz, 2H) 7.54 (s, 1H) 7.40 (s, 1H) 7.11-7.19 (m, 1H) 4.76 (br t, J=3.81 Hz, 1H) 4.11 (s, 5H) 2.98 (br d, J=1.31 Hz, 2H) 2.39 (s, 3H) 2.14 (br d, J=10.01 Hz, 2H) 1.98 (qd, J=12.16, 4.29 Hz, 2H) 1.44 (s, 9H).

### Synthesis of Compound 126

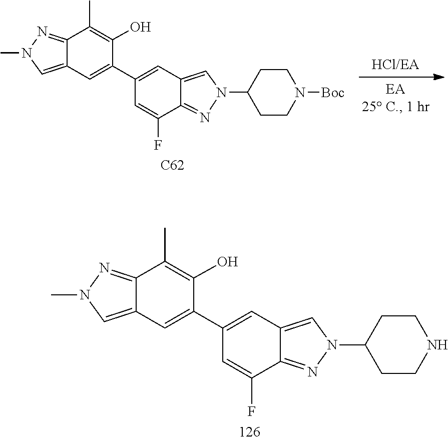

To a solution of tert-butyl 4-[7-fluoro-5-(6-hydroxy-2,7-dimethyl-indazol-5-yl)indazol-2-yl]piperidine-1-carboxylate (50.0 mg, 104 umol, 1.00 eq) in ethyl acetate (1.00 mL) was added HCl/ethyl acetate (4.00 M, 1.00 mL, 38.5 eq). The reaction mixture was stirred at 25° C. for 1 hr. The reaction mixture was filtered and the filter cake was concentrated in vacuum to afford a solid. The solid was purified by prep-HPLC (column: Phenomenex Luna 80*30 mm*3 um; mobile phase: [water (HCl)-acetonitrile]; B %: 5%-30%, 8 min) to give 5-[7-fluoro-2-(4-piperidyl)indazol-5-yl]-2,7-dimethyl-indazol-6-ol (30.9 mg, 60.1%) as a solid. LCMS: (ESI, m/z): 380.1 [M+H]⁺. ¹HNMR (400 MHz, METHANOL-d₄) δ ppm 8.73 (s, 1H) 8.49 (d, J=2.62 Hz, 1H) 7.74 (s, 1H) 7.65 (d, J=1.07 Hz, 1H) 7.18 (dd, J=12.64, 1.19 Hz, 1H) 4.96 (dt, J=9.95, 4.92 Hz, 1H) 4.36 (s, 3H) 3.66 (br d, J=13.35 Hz, 2H) 3.32-3.36 (m, 2H) 2.41-2.57 (m, 7H).

### Example 33: Synthesis of Compound 133

### Synthesis of Intermediate C64

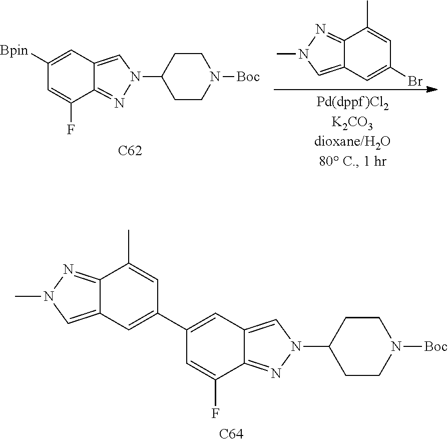

To a solution of 6-bromo-2,8-dimethyl-imidazo[1,2-a]pyrazine (60.9 mg, 269 umol, 1.00 eq) and tert-butyl 4-[7-fluoro-5-(4,4,5,5-tetramethyl-1,3,2-dioxaborolan-2-yl)indazol-2-yl]piperidine-1-carboxylate (BC, 180 mg, 404 umol, 1.50 eq) in dioxane (1.50 mL) was added a solution of K₂CO₃ (74.4 mg, 538 umol, 2.00 eq) in H₂O (0.400 mL) and 1,1-bis (diphenylphosphino)ferrocene]dichloropalladium(II) (39.4 mg, 53.9 umol, 0.200 eq) successively under N₂ protection. The reaction mixture was stirred at 80° C. for 1 hr. The reaction mixture was diluted with H₂O (10.0 mL), extracted with ethyl acetate (3×10.0 mL), dried over Na₂SO₄ filtered and concentrated under reduced pressure to give an oil. The oil was purified by column chromatography on silica gel eluted with petroleum ether/ethyl acetate (1/0 to 5/1) to give tert-butyl 4-[5-(2,8-dimethylimidazo[1,2-a]pyrazin-6-yl)-7-fluoro-indazol-2-yl]piperidine-1-carboxylate (80.0 mg, 55%) as an oil. LCMS: (ESI, m/z): 464.3 [M+H]⁺. 1H NMR (400 MHz, METHANOL-d₄) δ ppm 8.41 (d, J=2.69 Hz, 1H) 8.19 (s, 1H) 7.73 (dd, J=3.12, 1.04 Hz, 2H) 7.27-7.45 (m, 2H) 4.71 (tt, J=11.48, 4.17 Hz, 1H) 4.20-4.33 (m, 5H) 3.03 (br s, 2H) 2.63 (s, 3H) 2.07-2.28 (m, 4H) 1.50 (s, 9H).

### Synthesis of Compound 133

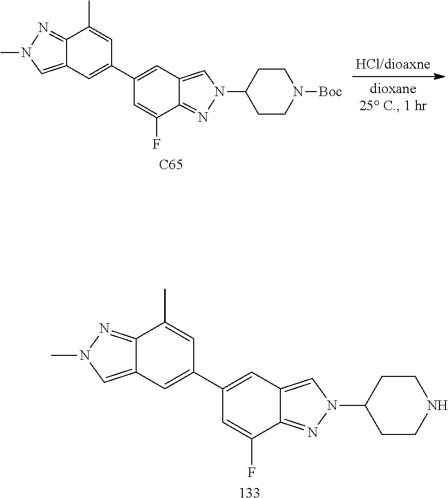

To a solution of tert-butyl 4-[5-(2,7-dimethylindazol-5-yl)-7-fluoro-indazol-2-yl]piperidine-1-carboxylate (80.0 mg, 173 umol, 1.00 eq) in dioxane (1.60 mL) was added HCl/dioxane (4.00 M, 1.64 mL, 38.0 eq). The reaction mixture was stirred at 25° C. for 1 hr. The reaction mixture was filtered and filter cake was dried in vacuum to get 5-(2,7-dimethylindazol-5-yl)-7-fluoro-2-(4-piperidyl)indazole (36.7 mg, 58%) as a solid. LCMS: (ESI, m/z): 364.1 [M+H]⁺. ¹H NMR (400 MHz, Deuterium oxide) δ ppm 8.15 (d, J=2.62 Hz, 1H) 7.92 (s, 1H) 7.30 (d, J=19.67 Hz, 2H) 7.09 (s, 1H) 6.93 (d, J=13.35 Hz, 1H) 4.70 (s, 1H) 4.03 (s, 3H) 3.61 (br d, J=13.47 Hz, 2H) 3.20 (td, J=12.64, 3.58 Hz, 2H) 2.21-2.41 (m, 7H).

### Example 34: Synthesis of Compound 132

### Synthesis of Intermediate C67

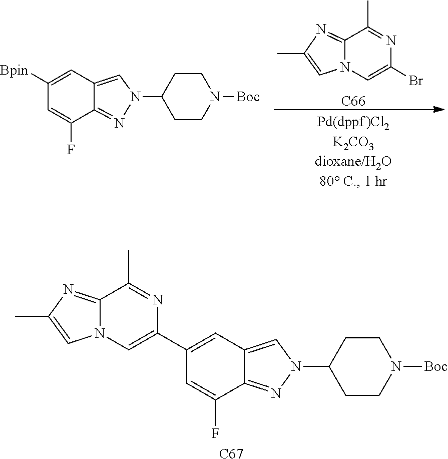

To a mixture of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1.50 eq) and 6-bromo-2,8-dimethyl-imidazo[1,2-a]pyrazine (67.7 mg, 299 umol, 1.0 eq) in dioxane (1.60 mL) was added a solution of K₂CO₃ (82.8 mg, 599 umol, 2.0 eq) in H₂O (0.400 mL). Then, 1,1-bis(diphenylphosphino)ferrocene]dichloropalladium(II) (43.8 mg, 59.9 umol, 0.20 eq) was added to the above mixture under N₂ protection. The reaction mixture was stirred at 80° C. for 1 hr. The reaction mixture was diluted with H₂O (10.0 mL), extracted with ethyl acetate (3×10.0 mL), dried over Na₂SO₄ filtered and concentrated under reduced pressure to give an oil. The oil was purified by column chromatography on silica gel eluted with petroleum ether/ethyl acetate (1/0 to 5/1) to give tert-butyl 4-[5-(2,8-dimethylimidazo[1,2-a]pyrazin-6-yl)-7-fluoro-indazol-2-yl]piperidine-1-carboxylate (85.0 mg, 55%) as a solid. LCMS: (ES, m/z): 465.2 [M+H]⁺. ¹H NMR (400 MHz, METHANOL-d₄) δ ppm 8.71 (s, 1H) 8.48 (d, J=2.63 Hz, 1H) 8.16 (d, J=0.88 Hz, 1H) 7.76 (s, 1H) 7.61 (dd, J=13.13, 1.00 Hz, 1H) 4.67-4.79 (m, 1H) 4.29 (br d, J=13.38 Hz, 2H) 3.04 (br s, 2H) 2.85 (s, 3H) 2.49 (s, 3H) 2.07-2.28 (m, 4H) 1.50 (s, 9H).

### Synthesis of Compound 132

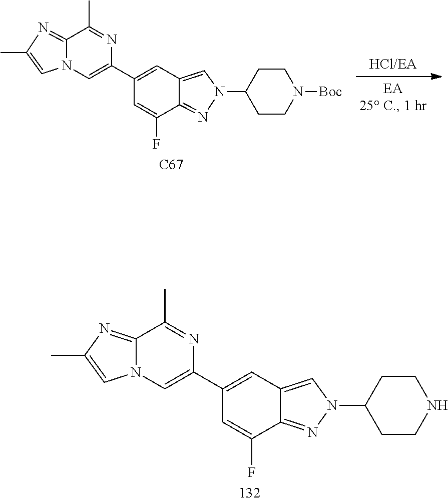

To a solution of tert-butyl 4-[5-(2,8-dimethylimidazo[1,2-a]pyrazin-6-yl)-7-fluoro-indazol-2-yl]piperidine-1-carboxylate (80.0 mg, 172 umol, 1.0 eq) in ethyl acetate (1.60 mL) was added HCl/ethyl acetate (4.0 M, 1.61 mL, 37.5 eq). The reaction mixture was stirred at 25° C. for 1 hr. The reaction mixture was filtered. The filter cake was dried in vacuum to get 6-[7-fluoro-2-(4-piperidyl)indazol-5-yl]-2,8-dimethyl-imidazo[1,2-a]pyrazine (49.6 mg, 79%) as a solid. LCMS: (ESI, m/z): 365.1 [M+H]⁺. ¹H NMR (400 MHz, D₂O) δ ppm 8.77 (s, 1H) 8.57 (d, J=2.38 Hz, 1H) 7.85-8.16 (m, 2H) 7.52 (d, J=12.63 Hz, 1H) 4.91-5.03 (m, 1H) 3.67 (br d, J=13.38 Hz, 2H) 3.30 (td, J=12.91, 2.69 Hz, 2H) 2.87 (s, 3H) 2.54 (s, 7H).

### Example 35: Synthesis of Compound 134

### Synthesis of C69

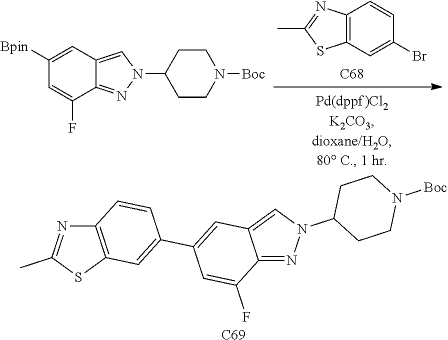

To a mixture of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1.50 eq) and 6-bromo-2-methyl-1,3-benzothiazole (68.3 mg, 299 umol, 1 eq) in dioxane (1.60 mL) was added a solution of K₂CO₃ (82.8 mg, 599 umol, 2.00 eq) in H₂O (0.400 mL). Then, [1, 1-bis(diphenylphosphino) ferrocene] dichloropalladium(II) (43.8 mg, 59.9 umol, 0.200 eq) was added to the mixture under N₂ protection. The reaction mixture was stirred at 80° C. for 1 hr. The reaction mixture was diluted with water (10.0 mL), extracted with ethyl acetate (3×20.0 mL). The organic phase was washed with brine (30.0 mL), dried over Na₂SO₄, filtered and the filtrate was concentrated under reduced pressure to give an oil. The oil was purified by chromatography on a silica gel eluted with petroleum ether/ethyl acetate (1/0 to 5/1) to give tert-butyl 4-[7-fluoro-5-(2-methyl-1,3-benzothiazol-6-yl)indazol-2-yl]piperidine-1-carboxylate (120 mg, 70.2%) as an oil. LCMS: (ESI, m/z): 467.2 [M+H]⁺. ¹H NMR (400 MHz, METHANOL-d4) δ ppm 8.47 (d, J=2.75 Hz, 1H) 8.21 (d, J=1.63 Hz, 1H) 7.95 (d, J=8.50 Hz, 1H) 7.69-7.86 (m, 2H) 7.39 (dd, J=12.88, 1.25 Hz, 1H) 4.74 (s, 1H) 4.29 (br d, J=13.76 Hz, 2H) 2.97-3.12 (m, 2H) 2.85 (s, 3H) 2.06-2.31 (m, 4H) 1.50 (s, 9H)

### Synthesis of Compound 134

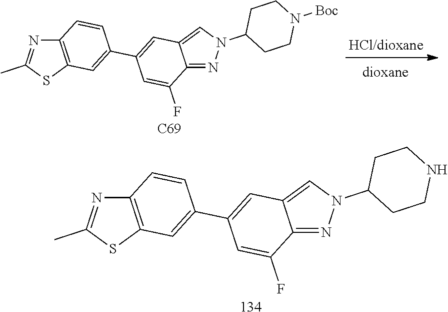 [SMILES: Cc1nc2ccc(-c3cc(F)c4nn(C5CCN(C(=O)OC(C)(C)C)CC5)cc4c3)cc2s1 ; "C69" ; Cc1nc2ccc(-c3cc(F)c4nn(C5CCNCC5)cc4c3)cc2s1 ; "134"]

To a mixture of tert-butyl 4-[7-fluoro-5-(2-methyl-1, 3-benzothiazol-6-yl) indazol-2-yl] piperidine-1-carboxylate (60.0 mg, 129 umol, 1.00 eq) in dioxane (1.20 mL) was added HCl/dioxane (4.0 M, 1.20 mL, 37.3 eq). The reaction mixture was stirred at 25° C. for 2 hrs. The reaction mixture was filtered and the filter cake was dried in vacuum to give 6-(7-fluoro-2-(piperidin-4-yl)-2H-indazol-5-yl)-2-methylbenzo[d]thiazole (30.2 mg, 57%) as a solid. LCMS: (ESI, m/z): 367.0 [M+H]⁺. ¹H NMR (400 MHz, METHANOL-d4) δ ppm 8.54 (d, J=2.69 Hz, 1H) 8.40 (d, J=1.22 Hz, 1H) 7.97-8.08 (m, 2H) 7.90 (d, J=1.22 Hz, 1H) 7.44 (dd, J=12.78, 1.28 Hz, 1H) 4.97 (tt, J=9.90, 5.14 Hz, 1H) 3.61-3.70 (m, 2H) 3.34 (br s, 1H) 3.28 (br d, J=5.14 Hz, 1H) 3.05 (s, 3H) 2.43-2.58 (m, 4H)

### Example 36: Synthesis of Compound 135

### Synthesis of Intermediate C70

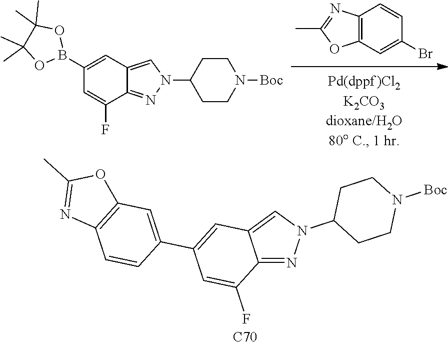 [SMILES: CC(C)(C)OC(=O)N1CCC(n2cc3cc(B4OC(C)(C)C(C)(C)O4)cc(F)c3n2)CC1 ; Cc1nc2ccc(Br)cc2o1 ; Cc1nc2ccc(-c3cc(F)c4nn(C5CCN(C(=O)OC(C)(C)C)CC5)cc4c3)cc2o1 ; "C70"]

To a mixture of tert-butyl 4-[7-fluoro-5-(4,4,5,5-tetramethyl-1,3,2-dioxaborolan-2-yl)indazol-2-yl]piperidine-1-carboxylate (135 mg, 303 umol, 1.50 eq) and 6-bromo-4-fluoro-2-methyl-1,3-benzoxazole (46.4 mg, 202 umol, 1.00 eq) in dioxane (1.08 mL) was added a solution of K₂CO₃ (55.8 mg, 404 umol, 2.00 eq) in H₂O (0.270 mL) and 1,1 [symbol] bis(diphenylphosphino)ferrocenedichloro palladium(II) (29.5 mg, 40.4 umol, 0.20 eq) successively. The reaction mixture was stirred at 80° C. for 1 hr. The reaction mixture was diluted with water (10.0 mL), extracted with ethyl acetate (3×10.0 mL), dried over Na₂SO₄, filtered and concentrated under reduced pressure to give oil. The oil was purified by column chromatography on silica gel eluted with petroleum ether/ethyl acetate (5/1 to 1/1) to give tert-butyl 4-[7-fluoro-5-(2-methyl-1,3-benzoxazol-6-yl)indazol-2-yl]piperidine-1-carboxylate (40.0 mg, 27%) as an oil. LCMS: (ESI, m/z): 451.2 [M+H]⁺. ¹H NMR (400 MHz, METHANOL-d₄) δ ppm 8.46 (d, J=2.69 Hz, 1H) 7.78-7.89 (m, 2H) 7.62-7.73 (m, 2H) 7.36 (dd, J=12.84, 1.34 Hz, 1H) 4.66-4.80 (m, 1H) 4.29 (br d, J=13.82 Hz, 2H) 3.04 (br dd, J=4.52, 2.57 Hz, 2H) 2.67 (s, 3H) 2.07-2.27 (m, 4H) 1.50 (s, 9H).

### Synthesis of Compound 135

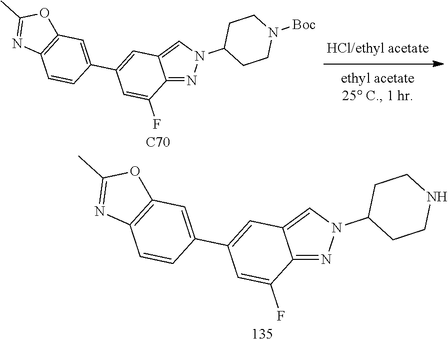

To a solution of tert-butyl 4-[7-fluoro-5-(2-methyl-1,3-benzoxazol-6-yl) indazol-2-yl]piperidine-1-carboxylate (40.0 mg, 88.7 umol, 1.00 eq) in ethyl acetate (0.800 mL) was added HCl/ethyl acetate (1.78 mmol, 0.800 mL, 20.0 eq). The reaction mixture was stirred at 25° C. for 1 hr. The reaction mixture was filtered and the filter cake was purified by prep-HPLC (column: Phenomenex C18 75*30 mm*3 um; mobile phase: [water (NH₄HCO₃)-acetonitrile]; B %: 20%-55%, 8 mins) to give 6-[7-fluoro-2-(4-piperidyl)indazol-5-yl]-2-methyl-1,3-benzoxazole (10.7 mg, 34.4%) as a solid. LCMS: (ESI, m/z): 351.2 [M+H]⁺. ¹H NMR (400 MHz, METHANOL-d₄) 6=8.45 (d, J=2.7 Hz, 1H), 7.86-7.78 (m, 2H), 7.67 (s, 2H), 7.37 (d, J=13.0 Hz, 1H), 4.72-4.63 (m, 1H), 3.27 (br d, J=13.1 Hz, 2H), 2.86 (dt, J=2.4, 12.6 Hz, 2H), 2.67 (s, 3H), 2.30-2.09 (m, 4H).

### Example 37: Synthesis of Compound 136

### Synthesis of Intermediate C72

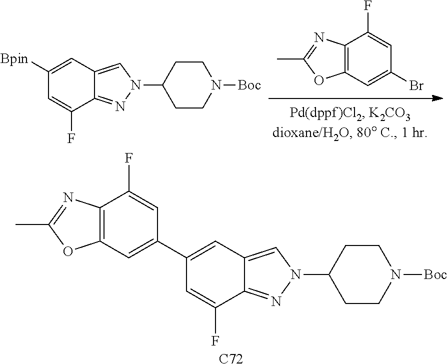

To a mixture of tert-butyl 4-[7-fluoro-5-(4,4,5,5-tetramethyl-1,3,2-dioxaborolan-2-yl)indazol-2-yl]piperidine-1-carboxylate (135 mg, 303 umol, 1.50 eq) and 6-bromo-4-fluoro-2-methyl-1,3-benzoxazole (46.5 mg, 202 umol, 1.00 eq) in dioxane (1.08 mL) was added a solution of K₂CO₃ (55.7 mg, 404 umol, 2.00 eq) in H₂O (0.270 mL). Then, 1,1-bis(diphenylphosphino)ferrocene]dichloropalladium(II) (29.6 mg, 40.4 umol, 0.200 eq) was added to the above mixture. The reaction mixture was stirred at 80° C. for 1 hr. The reaction mixture was diluted with H₂O (10 mL), extracted with ethyl acetate (3×10 mL), dried over Na₂SO₄, filtered and concentrated under reduced pressure to give an oil. The oil was purified by column chromatography on silica gel eluted with petroleum ether/ethyl acetate (1/0 to 5/1) to give tert-butyl4-[7-fluoro-5-(4-fluoro-2-methyl-1,3-benzoxazol-6-yl)indazol-2-yl]piperidine-1-carboxylate (50.0 mg, 47.0%) as a solid. LCMS: (ESI, m/z): 469.2 [M+H]⁺. ¹H NMR (400 MHz, METHANOL-d₄) δ ppm 8.48 (d, J=2.74 Hz, 1H) 7.69-7.88 (m, 2H) 7.33-7.51 (m, 2H) 4.69-4.79 (m, 1H) 4.29 (br d, J=13.47 Hz, 2H) 2.95-3.11 (m, 2H) 2.68 (s, 3H) 2.07-2.26 (m, 4H) 1.50 (s, 9H).

### Synthesis of Compound 136

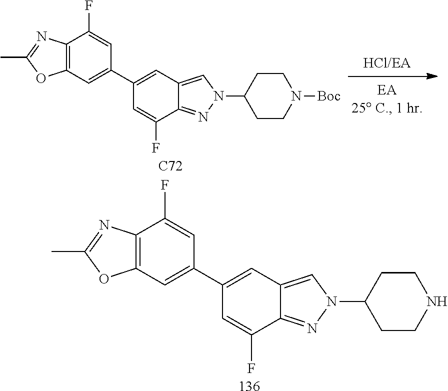

A solution of tert-butyl 4-[7-fluoro-5-(4-fluoro-2-methyl-1,3-benzoxazol-6-yl)indazol-2-yl]piperidine-1-carboxylate (45.0 mg, 96.1 umol, 1.00 eq) in ethyl acetate (0.900 mL) was added HCl/ethyl acetate (4.0 M, 912 μL, 38.0 eq). The reaction mixture was stirred at 25° C. for 1 hr. The reaction mixture was filtered and the filter cake was purified by prep-HPLC (column: Phenomenex C18 75*30 mm*3 um; mobile phase: [water (NH₄HCO₃)-acetonitrile]; B %: 15%-50%, 8 min) to afford 4-fluoro-6-[7-fluoro-2-(4-piperidyl)indazol-5-yl]-2-methyl-1,3-benzoxazole (15.4 mg, 43.5%) as a solid. LCMS: (ES, m/z): 369.2 [M+H]⁺. ¹H NMR (400 MHz, METHANOL-d₄) δ ppm 8.47 (d, J=2.57 Hz, 1H) 7.83 (s, 1H) 7.73 (s, 1H) 7.34-7.50 (m, 2H) 4.62-4.76 (m, 1H) 3.26 (br s, 2H) 2.80-2.91 (m, 2H) 2.68 (s, 3H) 2.08-2.30 (m, 4H).

### Example 38: Synthesis of Compound 145

### Synthesis of Intermediate C73

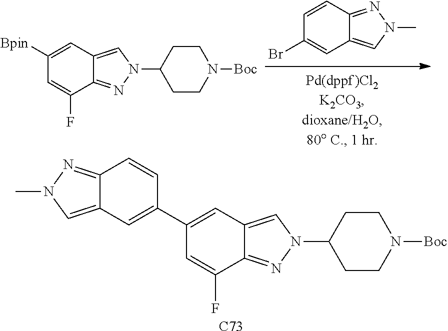

To a mixture of tert-butyl 4-[7-fluoro-5-(4,4,5,5-tetramethyl-1,3,2-dioxaborolan-2-yl)indazol-2-yl]piperidine-1-carboxylate (150 mg, 337 umol, 1.50 eq) in dioxane (2.80 mL) was added [1,1-bis(diphenylphosphino)ferrocene]dichloropalladium(II) (16.4 mg, 22.5 umol, 0.100 eq), 5-bromo-2-methyl-indazole (47.4 mg, 225 umol, 1.00 eq) and a solution of K₂CO₃ (62.1 mg, 449 umol, 2.00 eq) in H₂O (0.700 mL) under N₂ protection. The reaction mixture was stirred at 80° C. for 1 hr. The reaction mixture was diluted with water (5.00 mL), extracted with ethyl acetate (3×10.0 mL), washed with brine (15.0 mL), dried over Na₂SO₄, filtered and concentrated under reduced pressure to give residue. The residue was purified by column chromatography on a silica gel eluted with ethyl acetate/petroleum ether (0/1 to 1/1) to give tert-butyl 4-[7-fluoro-5-(2-methylindazol-5-yl)indazol-2-yl]piperidine-1-carboxy late (50.0 mg, 48%) as a solid. LCMS: (ES, m/z): 450.2 [M+H]⁺. ¹H NMR (400 MHz, CHLOROFORM-d) δ ppm 8.04 (d, J=2.63 Hz, 1H) 7.96 (s, 1H) 7.83 (d, J=0.63 Hz, 1H) 7.77 (d, J=9.01 Hz, 1H) 7.64 (d, J=1.00 Hz, 1H) 7.58 (dd, J=9.01, 1.75 Hz, 1H) 7.30 (dd, J=12.63, 1.13 Hz, 1H) 4.63 (tt, J=11.62, 4.02 Hz, 1H) 4.29-4.45 (m, 2H) 4.26 (s, 3H) 2.96 (brt, J=11.19 Hz, 2H) 2.25-2.37 (m, 2H) 2.14 (qd, J=12.26, 4.25 Hz, 2H) 1.50 (s, 9H)

### Synthesis of Compound 145

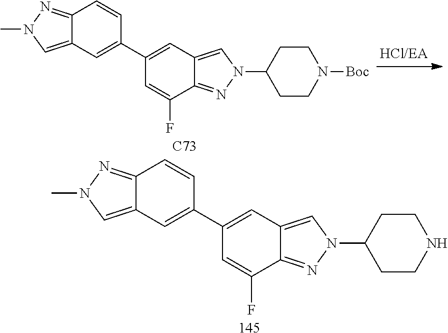

The solution of tert-butyl 4-[7-fluoro-5-(2-methylindazol-5-yl)indazol-2-yl]piperidine-1-carboxylate (50.0 mg, 111 umol, 1.00 eq) in ethyl acetate (1.00 mL) was added HCl/ethyl acetate (1.00 mL). The reaction mixture was stirred at 25° C. for 1 hr. The reaction was filtered and the filter cake was dried in vacuum to give 7-fluoro-5-(2-methylindazol-5-yl)-2-(4-piperidyl)indazole (25.0 mg, 62.4%) as a solid. LCMS: (ESI, m/z): 350.2 [M+H]⁺. ¹H NMR (400 MHz, METHANOL-d4) δ ppm 8.65 (s, 1H) 8.50 (d, J=2.62 Hz, 1H) 8.12 (s, 1H) 7.96 (br d, J=8.94 Hz, 1H) 7.84 (d, J=1.07 Hz, 1H) 7.77 (d, J=9.06 Hz, 1H) 7.41 (dd, J=12.87, 1.19 Hz, 1H) 4.95 (ddt, J=14.74, 9.79, 4.72, 4.72 Hz, 1H) 4.36 (s, 3H) 3.62-3.70 (m, 2H) 3.34 (br d, J=4.89 Hz, 1H) 3.28 (br d, J=4.65 Hz, 1H) 2.43-2.57 (m, 4H)

### Example 39: Synthesis of Compound 146

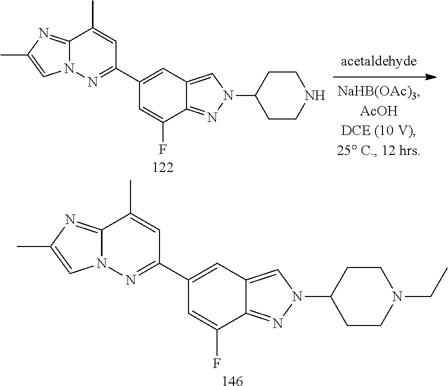

A mixture of 6-[7-fluoro-2-(4-piperidyl)indazol-5-yl]-2,8-dimethyl-imidazo[1,2-b]pyridazine (50.0 mg, 137 umol, 1.00 eq) in methanol (0.400 mL) was added triethylamine (27.8 mg, 274 umol, 2.00 eq). Then, acetaldehyde (5.0 M, 54.9 uL, 2.00 eq) and acetic acid (0.100 mL) was added to the mixture successively. After stirred for 10 mins, sodium cyanoboranuide (12.9 mg, 206 umol, 1.50 eq) was added under N₂ protection. The reaction mixture was stirred at 25° C. for 12 hrs. The mixture was concentrated under reduced pressure to give a residue. The residue was purified by prep-HPLC (Column: Phenomenex Luna 80*30 mm*3 um; mobile phase: [water (HCl)-acetonitrile]; B %: 1%-30%, 8 min) to give 6-[2-(1-ethyl-4-piperidyl)-7-fluoro-indazol-5-yl]-2,8-dimethyl-imidazo[1,2-b]pyridazine (4.38 mg, 8%) as a solid. LCMS: (ES, m/z): 393.1 [M+H]⁺. ¹H NMR (400 MHz, METHANOL-d4) δ ppm 8.68-8.75 (m, 1H) 8.44 (s, 1H) 8.33 (s, 1H) 8.30 (s, 1H) 7.84 (brd, J=12.76 Hz, 1H) 4.97-5.06 (m, 1H) 3.83 (br d, J=12.51 Hz, 2H) 3.54-3.70 (m, 1H) 3.34-3.41 (m, 1H) 3.26 (br s, 1H) 2.78 (s, 3H) 2.46-2.73 (m, 8H) 1.39-1.48 (m, 3H)

### Example 40: Synthesis of Compound 147

### Synthesis of Intermediate C75

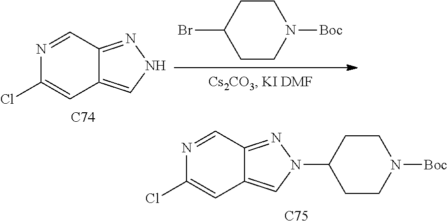 [SMILES: Clc1cc2c[nH]nc2cn1 ; CC(C)(C)OC(=O)N1CCC(Br)CC1 ; "C74" ; CC(C)(C)OC(=O)N1CCC(n2cc3cc(Cl)ncc3n2)CC1 ; "C75"]

A mixture of 5-chloro-2H-pyrazolo[3,4-c]pyridine (17-1, 1.00 g, 6.51 mmol, 1 eq), tert-butyl 4-bromopiperidine-1-carboxylate (3.44 g, 13.0 mmol, 2 eq), and Cs₂CO₃ (10.6 g, 32.5 mmol, 5 eq) and KI (1.08 g, 6.51 mmol, 1 eq) in DMF (10.0 mL) the mixture was stirred at 100° C. for 12 hrs under N₂ atmosphere. TLC (petroleum ether/ethyl acetate=1/1, Rf=0.27) showed the starting material was consumed and a new major spot was generated. After filtration and the filtrate was concentrated under reduced pressure to afford a residue, which was purified by silica gel column chromatography (petroleum ether/ethyl acetate=1:1) to give tert-butyl 4-(5-chloropyrazolo[3,4-c]pyridin-2-yl)piperidine-1-carboxylate (4.00 g, 16.4%, 90% purity) as a solid. LCMS: (ESI, m/z): 337.1 [M+H]^{+ 1}H NMR (CHLOROFORM-d, 400 MHz) δ ppm 9.05 (1H, s), 7.98 (1H, s), 7.56 (1H, d, J=1.1 Hz), 4.63 (1H, tt, J=11.5 Hz, J=4.1 Hz), 4.34 (2H, br s), 2.92-3.06 (2H, m), 2.26 (2H, br d, J=10.1 Hz), 2.06-2.18 (2H, m), 1.49 (9H, s)

### Synthesis of Intermediate C76

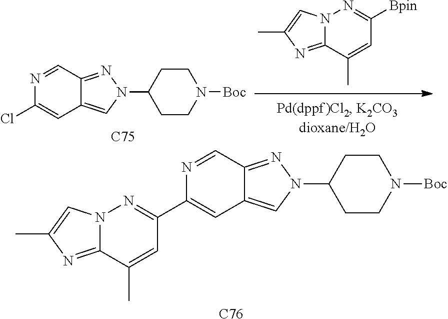

To a solution of tert-butyl 4-(5-chloropyrazolo[3,4-c]pyridin-2-yl)piperidine-1-carboxylate (17-2, 100 mg, 296 umol, 1 eq) and 2,8-dimethyl-6-(4,4,5,5-tetramethyl-1,3,2-dioxaborolan-2-yl)imidazo[1,2-b]pyridazine (97.3 mg, 356 umol, 1.2 eq) in dioxane (10.0 mL) and water (2.50 mL) was added Pd(dppf)Cl₂ (21.7 mg, 29.6 umol, 0.1 eq) and K₂CO₃ (82.0 mg, 593 umol, 2 eq) under N₂ atmosphere. The mixture was stirred at 90° C. for 12 hrs. TLC (ethyl acetate, Rf=0.44) showed the starting material was consumed and a new major spot was generated. The reaction mixture was cooled to 25° C. and quenched by addition water (10.0 mL) at 25° C., and then extracted with ethyl acetate (3×10.0 mL). The combined organic layers were dried over MgSO₄, filtered and concentrated under reduced pressure to give a residue, which was purified by silica gel column chromatography (petroleum ether/ethyl acetate=1:1) to give tert-butyl 4-[5-(2,8-dimethylimidazo[1,2-b]pyridazin-6-yl)pyrazolo[3,4-c]pyridin-2-yl]piperidine-1-carboxylate (40.0 mg, 30.1%) as a solid. LCMS: (ESI, m/z): 448.2 [M+H]^{+ 1}H NMR (CDCl₃, 400 MHz) δ ppm 9.35 (s, 1H) 8.54 (s, 1H) 8.13 (s, 1H) 7.99 (br s, 1H) 7.77 (s, 1H) 4.5-4.8 (m, 1H) 4.2-4.5 (m, 2H) 2.9-3.1 (m, 2H) 2.7-2.8 (m, 3H) 2.5-2.6 (m, 3H) 2.2-2.4 (m, 2H) 2.1-2.2 (m, 2H) 1.51 (s, 9H)

### Synthesis of Compound 147

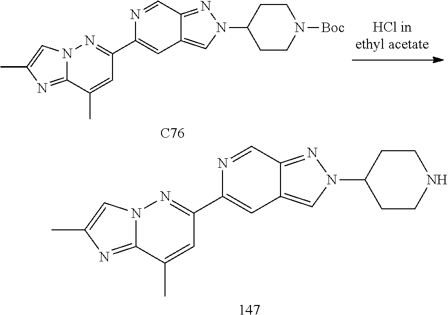 [SMILES: Cc1cn2nc(-c3cc4cn(C5CCN(C(=O)OC(C)(C)C)CC5)nc4cn3)cc(C)c2n1 ; "C76" ; Cc1cn2nc(-c3cc4cn(C5CCNCC5)nc4cn3)cc(C)c2n1 ; "147"]

To a solution of tert-butyl 4-[5-(2,8-dimethylimidazo[1,2-b]pyridazin-6-yl)pyrazolo[3,4-c]pyridin-2-yl]piperidine-1-carboxylate (40.0 mg, 89.3 umol, 1 eq) in EtOAc (1.00 mL) was added HCl/EtOAc (4 M, 2.00 mL) under N₂ atmosphere. The mixture was stirred at 20° C. for 12 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 2,8-dimethyl-6-[2-(4-piperidyl)pyrazolo[3,4-c]pyridin-5-yl]imidazo[1,2-b]pyridazine (24.9 mg, 76.4%) as a solid. LCMS: (ESI, m/z): 348.1 [M+H]^{+ 1}H NMR (MeOD-d₄, 400 MHz) δ ppm 9.67 (s, 1H) 9.12 (s, 1H) 9.04 (s, 1H) 8.64 (s, 1H) 8.41 (s, 1H) 5.2-5.3 (m, 1H) 3.6-3.7 (m, 2H) 3.3-3.4 (m, 2H) 2.85 (s, 3H) 2.69 (s, 3H) 2.5-2.6 (m, 4H)

### Example 41: Synthesis of Compound 148

### Synthesis of Intermediate C77

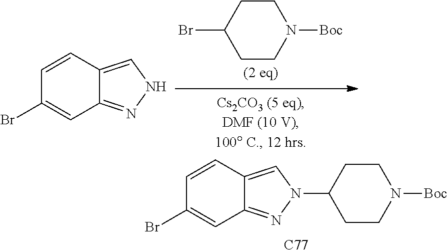 [SMILES: Brc1ccc2c[nH]nc2c1 ; CC(C)(C)OC(=O)N1CCC(Br)CC1 ; CC(C)(C)OC(=O)N1CCC(n2cc3ccc(Br)cc3n2)CC1 ; "C77"]

A mixture of 6-bromo-2H-indazole (100 mg, 507 umol, 1 eq), tert-butyl 4-iodopiperidine-1-carboxylate (315 mg, 1.02 mmol, 2 eq), Cs₂CO₃ (826 mg, 2.54 mmol, 5 eq) in DMF (1.00 mL) was degassed and purged with N₂ for 3 times, and then the mixture was stirred at 100° C. for 12 hrs under N₂ atmosphere. TLC (petroleum ether/ethyl acetate=1/1, Rf=0.27) showed the starting material was consumed and a new major spot was generated. After filtration, the filtrate was concentrated under reduced pressure evaporated to dryness to afford a residue, which was purified by silica gel column chromatography (petroleum ether/ethyl acetate=1:1) to give tert-butyl 4-(6-bromoindazol-2-yl) piperidine-1-carboxylate (100 mg, 51.8%) as a solid. LCMS: (ESI, m/z): 379.9, 381.9 [M+H]^{+ 1}H NMR (CDCl₃, 400 MHz) δ ppm 7.9-8.0 (m, 2H), 7.5-7.6 (m, 1H), 7.1-7.2 (m, 1H), 4.5-4.6 (m, 1H), 4.2-4.4 (m, 2H), 2.8-3.0 (m, 2H), 2.2-2.3 (m, 2H), 2.0-2.2 (m, 2H), 1.4-1.5 (m, 9H)

### Synthesis of Intermediate C78

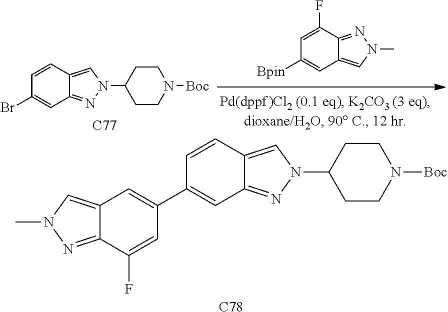

To a solution of tert-butyl 4-(6-bromoindazol-2-yl)piperidine-1-carboxylate (100 mg, 262 umol, 1 eq) and 7-fluoro-2-methyl-5-(4,4,5,5-tetramethyl-1,3,2-dioxaborolan-2-yl)indazole (72.6 mg, 262 umol, 1 eq) in dioxane (1.00 mL) was added Pd(dppf)Cl₂ (19.2 mg, 26.3 umol, 0.1 eq) and K₂CO₃ (109 mg, 262 umol, 3 eq) under N₂ atmosphere. The mixture was stirred at 90° C. for 12 hrs. TLC (ethyl acetate, Rf=0.45) showed the starting material was consumed and a new major spot was generated. The reaction mixture was cooled to 20° C. The mixture was quenched by adding water (10.0 mL) and the solution was extracted with ethyl acetate (3×20.0 mL). The combined organic layers were dried over MgSO₄, filtered and concentrated under reduced pressure to give a residue, which was purified by silica gel column chromatography (petroleum ether/ethyl acetate=1:1) to give tert-butyl 4-[6-(7-fluoro-2-methyl-indazol-5-yl)indazol-2-yl]piperidine-1-carboxylate (30.0 mg, 25%) as a solid. LCMS: (ESI, m/z): 350.1 [M+H−Boc]^{+ 1}H NMR (CDCl₃, 400 MHz) δ ppm 7.98 (s, 1H), 7.90 (s, 1H), 7.73 (d, 1H, J=8.7 Hz), 7.68 (s, 1H), 7.39 (d, 1H, J=8.7 Hz), 7.31 (d, 1H, J=12.7 Hz), 4.5-4.7 (m, 1H), 4.2-4.4 (m, 5H), 2.9-3.1 (m, 2H), 2.29 (br d, 2H, J=10.5 Hz), 2.1-2.2 (m, 2H), 1.5-1.6 (m, 9H)

### Synthesis of Compound 148

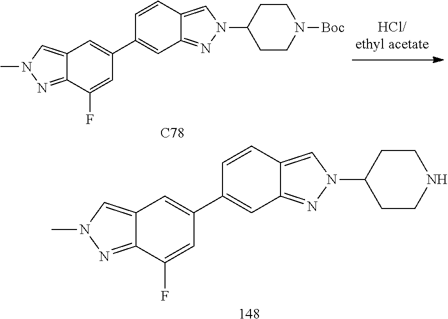 [SMILES: Cn1cc2cc(-c3ccc4cn(C5CCN(C(=O)OC(C)(C)C)CC5)nc4c3)cc(F)c2n1 ; "C78" ; Cn1cc2cc(-c3ccc4cn(C5CCNCC5)nc4c3)cc(F)c2n1 ; "148"]

To a solution of tert-butyl 4-[6-(7-fluoro-2-methyl-indazol-5-yl) indazol-2-yl]piperidine-1-carboxylate (30.0 mg, 66.7 umol, 1 eq) in EtOAc (1.00 mL) was added HCl/EtOAc (4 M, 1.00 mL) under N₂ atmosphere. The mixture was stirred at 25° C. for 6 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 7-fluoro-2-methyl-5-[2-(4-piperidyl) indazol-6-yl]indazole (25.0 mg, 92.4%, HCl) as a solid. LCMS: (ESI, m/z): 350.1 [M+H]^{+ 1}H NMR (MeOD-d₄, 400 MHz) δ ppm 8.57 (s, 1H), 8.46 (d, 1H, J=2.2 Hz), 7.8-7.9 (m, 3H), 7.57 (br d, 1H, J=8.6 Hz), 7.47 (br d, 1H, J=12.7 Hz), 5.00 (br s, 1H), 4.29 (s, 3H), 3.67 (br d, 2H, J=12.3 Hz), 3.3-3.4 (m, 2H), 2.4-2.6 (m, 4H) ¹⁹F NMR (MeOD-d₄, 376 MHz) δ ppm −131.06 (s, 1F),

### Example 42: Synthesis of Compound 154

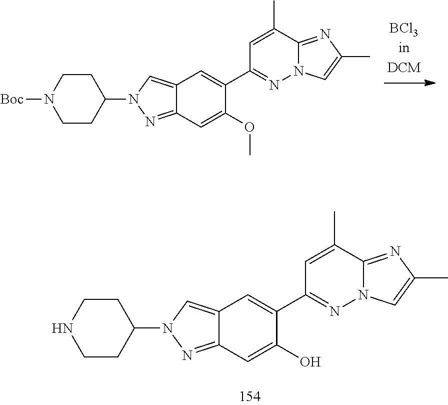 [SMILES: COc1cc2nn(C3CCN(C(=O)OC(C)(C)C)CC3)cc2cc1-c1cc(C)c2nc(C)cn2n1 ; Cc1cn2nc(-c3cc4cn(C5CCNCC5)nc4cc3O)cc(C)c2n1]

To a solution of tert-butyl 4-[5-(2,8-dimethylimidazo[1,2-b]pyridazin-6-yl)-7-methoxy-indazol-2-yl]piperidine-1-carboxylate (150 mg, 315 umol, 1 eq) in BCl₃ (1M in DCM, 2.00 mL) was stirred at 25° C. for 3 hrs. The reaction mixture was concentrated under reduced pressure to give a residue. The residue was purified by prep-HPLC (column: Phenomenex Luna 80*30 mm*3 um; mobile phase: [water(HCl)-ACN]; B %: 1%-30%, 8 min) to give 5-(2,8-dimethylimidazo[1,2-b]pyridazin-6-yl)-2-(piperidin-4-yl)-2H-indazol-6-ol (23.0 mg, 20.1%, HCl salt) as a solid. LCMS: (ESI, m/z): 363.3 [M+H]⁺. ¹HNMR (400 MHz, DMSO-d6) δ ppm 8.46 (s, 1H) 8.39 (s, 1H) 7.97 (d, J=4.77 Hz, 2H) 7.05 (s, 1H) 4.78 (dt, J=14.79, 7.52 Hz, 1H) 3.44 (br d, J=12.84 Hz, 2H) 3.06-3.18 (m, 2H) 2.66 (s, 3H) 2.54 (s, 3H) 2.23-2.37 (m, 4H)

### Example 43: Synthesis of Compound 155

### Synthesis of Intermediate C79

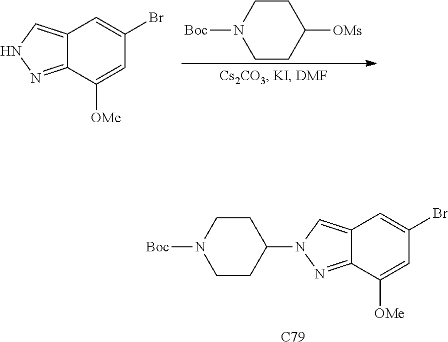 [SMILES: COc1cc(Br)cc2c[nH]nc12 ; CC(C)(C)OC(=O)N1CCC(OS(C)(=O)=O)CC1 ; COc1cc(Br)cc2cn(C3CCN(C(=O)OC(C)(C)C)CC3)nc12 ; "C79"]

To a solution of 5-bromo-7-methoxy-2H-indazole (EVAL-0123-0042-4, 4.50 g, 19.8 mmol, 1 eq) and tert-butyl 4-methylsulfonyloxypiperidine-1-carboxylate (16.6 g, 59.46 mmol, 3 eq) in DMF (90.0 mL) was added Cs₂CO₃ (32.3 g, 99.1 mmol, 5 eq) and KI (9.87 g, 59.5 mmol, 3 eq). The mixture was stirred at 110° C. for 12 hrs. TLC (petroleum ether/ethyl acetate=5/1, Rf=0.5) showed that the starting material was consumed and a new major spot was generated. The reaction mixture was cooled to 25° C. and then diluted with H₂O (100 mL) and extracted with ethyl acetate (3×100 mL). The combined organic layers were washed with brine (3×100 mL), dried over Na₂SO₄, filtered and concentrated under reduced pressure to give a residue. The residue was purified by column chromatography (SiO₂, 30% of ethyl acetate in petroleum ether) to give tert-butyl 4-(5-bromo-7-methoxy-indazol-2-yl)piperidine-1-carboxylate (2.60 g, 32.1%) as an oil. LCMS: (ESI, m/z): 409.9, 411.9 [M+H]⁺. ¹HNMR (400 MHz, CDCl₃) δ ppm 7.85 (s, 1H) 7.39 (d, J=1.43 Hz, 1H) 6.64 (d, J=1.31 Hz, 1H) 4.57 (tt, J=11.71, 4.02 Hz, 1H) 4.32 (br d, J=3.10 Hz, 2H) 4.01 (s, 3H) 2.84-3.01 (m, 2H) 2.24 (br dd, J=12.16, 2.26 Hz, 2H) 2.06-2.16 (m, 2H) 1.49 (s, 9H)

### Synthesis of Intermediate C80

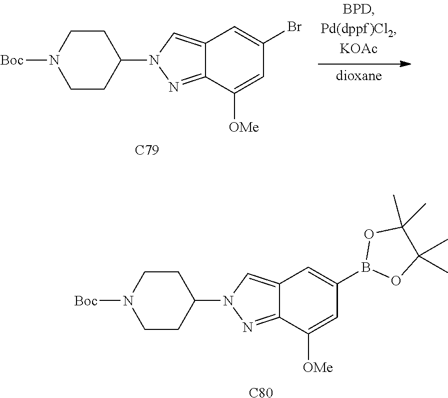 [SMILES: COc1cc(Br)cc2cn(C3CCN(C(=O)OC(C)(C)C)CC3)nc12 ; "C79" ; COc1cc(B2OC(C)(C)C(C)(C)O2)cc2cn(C3CCN(C(=O)OC(C)(C)C)CC3)nc12 ; "C80"]

To a solution of tert-butyl 4-(5-bromo-7-methoxy-indazol-2-yl)piperidine-1-carboxylate (400 mg, 975 umol, 1 eq) and 4,4,5,5-tetramethyl-2-(4,4,5,5-tetramethyl-1,3,2-dioxaborolan-2-yl)-1,3,2-dioxaborolane (371 mg, 1.46 mmol, 1.5 eq) in dioxane (8.00 mL) was added Pd(dppf)Cl₂ (35.8 mg, 48.7 umol, 0.05 eq) and KOAc (191 mg, 1.95 mol, 2 eq). The mixture was stirred at 100° C. for 12 hrs. The reaction mixture filtered and concentrated under reduced pressure to give tert-butyl 4-(7-methoxy-5-(4,4,5,5-tetramethyl-1,3,2-dioxaborolan-2-yl)-2H-indazol-2-yl)piperidine-1-carboxylate (400 mg, 90%) as an oil. The crude product was used into the next step without further purification. LCMS: (ES, m/z): 458.3 [M+H]⁺.

### Synthesis of Intermediate C81

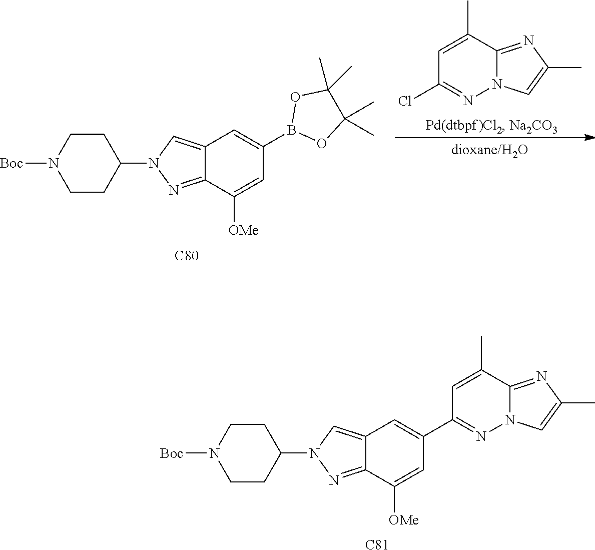

A mixture of tert-butyl 4-[7-methoxy-5-(4,4,5,5-tetramethyl-1,3,2-dioxaborolan-2-yl)indazol-2-yl]piperidine-1-carboxylate (400 mg, 874 umol, 1 eq), 6-chloro-2,8-dimethyl-imidazo[1,2-b]pyridazine (159 mg, 875 umol, 1 eq), ditert-butyl(cyclopentyl)phosphane;dichloropalladium;iron (282 mg, 437 umol, 0.5 eq) and Na₂CO₃ (185 mg, 1.75 mmol, 2 eq) in dioxane (14.0 mL) and H₂O (2.80 mL) was degassed and purged with N₂ for 3 times, and then the mixture was stirred at 80° C. for 2 hrs under N₂ atmosphere. The reaction mixture diluted with H₂O (20.0 mL) and extracted with ethyl acetate (3×20.0 mL). The combined organic layers were washed with brine (3×20.0 mL), dried over Na₂SO₄, filtered and concentrated under reduced pressure to give a residue. The residue was purified by column chromatography (SiO₂, 30% of ethyl acetate in petroleum ether) to give tert-butyl 4-(5-(2,8-dimethylimidazo[1,2-b]pyridazin-6-yl)-7-methoxy-2H-indazol-2-yl)piperidine-1-carboxylate (250 mg, 59.9%) as a solid. LCMS: (ESI, m/z): 477.0 [M+H]⁺. ¹HNMR (400 MHz, CDCl₃) δ ppm 7.96 (s, 1H) 7.69-7.75 (m, 2H) 7.22 (s, 1H) 4.57 (tt, J=11.67, 3.77 Hz, 1H) 4.21-4.39 (m, 2H) 4.10 (s, 3H) 2.80-2.97 (m, 2H) 2.67 (s, 3H) 2.50 (s, 3H) 2.19-2.30 (m, 2H) 2.09 (qd, J=12.28, 4.05 Hz, 2H) 1.45 (s, 9H)

### Synthesis of Compound 155

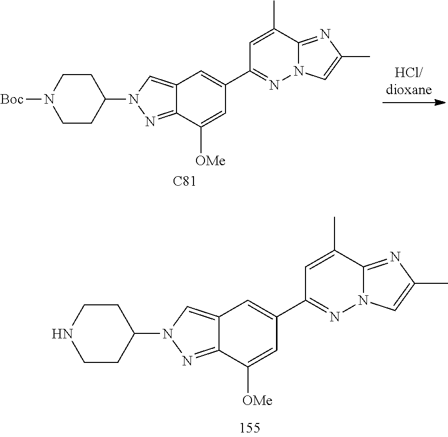 [SMILES: COc1cc(-c2cc(C)c3nc(C)cn3n2)cc2cn(C3CCN(C(=O)OC(C)(C)C)CC3)nc12 ; "C81" ; COc1cc(-c2cc(C)c3nc(C)cn3n2)cc2cn(C3CCNCC3)nc12]

To a solution of tert-butyl 4-[5-(2,8-dimethylimidazo[1,2-b]pyridazin-6-yl)-6-methoxy-indazol-2-yl]piperidine-1-carboxylate (250 mg, 524 umol, 1 eq) in HCl/dioxane (2.00 mL) was stirred at 25° C. for 1 hr. The reaction mixture was filtered and the filtrate was concentrated under reduced pressure to give 6-(7-methoxy-2-(piperidin-4-yl)-2H-indazol-5-yl)-2, 8-dimethylimidazo [1,2-b]pyridazine (24.9 mg, 12.6%) as a solid. LCMS: (ESI, m/z): 377.3 [M+H]⁺. ¹HNMR (400 MHz, DMSO-d₆) δ ppm 8.56 (s, 1H) 8.32 (d, J=0.86 Hz, 1H) 8.25 (s, 1H) 8.10 (d, J=0.86 Hz, 1H) 7.30 (d, J=0.86 Hz, 1H) 4.77-4.94 (m, 1H) 3.99 (s, 3H) 3.46 (br d, J=12.96 Hz, 2H) 3.05-3.19 (m, 2H) 2.65 (s, 3H) 2.54 (s, 3H) 2.30 (br d, J=3.18 Hz, 4H)

### Example 44: Synthesis of Compound 156

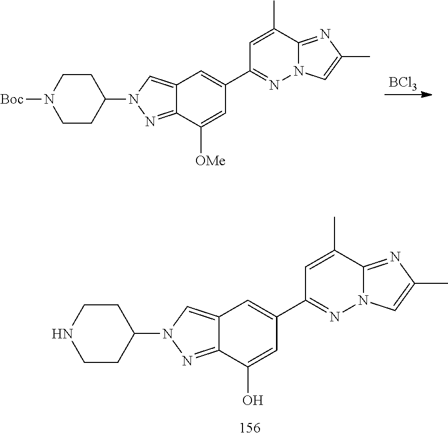

To a solution of tert-butyl 4-[5-(2,8-dimethylimidazo[1,2-b]pyridazin-6-yl)-7-methoxy-indazol-2-yl]piperidine-1-carboxylate (210 mg, 441 umol, 1 eq) in dichloromethane (1.00 mL) was added BCl₃ (1M in DCM, 5.40 mL). The mixture was stirred at 25° C. for 3 hrs. The reaction mixture was concentrated under reduced pressure to give a residue. The crude product purified by pre-HPLC (column: Phenomenex Luna 80*30 mm*3 um; mobile phase: [water(HCl)-MeOH]; B %: 15%-35%, 8 min) to give 5-(2,8-dimethylimidazo[1,2-b]pyridazin-6-yl)-2-(piperidin-4-yl)-2H-indazol-7-ol (37.5 mg, 23.4%) as a solid. LCMS: (ESI, m/z): 363.1 [M+H]⁺. ¹HNMR (400 MHz, DMSO-d₆) δ ppm 8.52 (s, 1H) 8.28 (s, 1H) 8.20 (s, 1H) 7.98 (d, J=0.73 Hz, 1H) 7.27 (d, J=0.98 Hz, 1H) 4.75-4.95 (m, 1H) 3.47 (br d, J=12.84 Hz, 2H) 3.04-3.20 (m, 2H) 2.64 (s, 3H) 2.54 (s, 3H) 2.32 (br d, J=2.81 Hz, 4H)

### Example 45: Synthesis of Compound 157

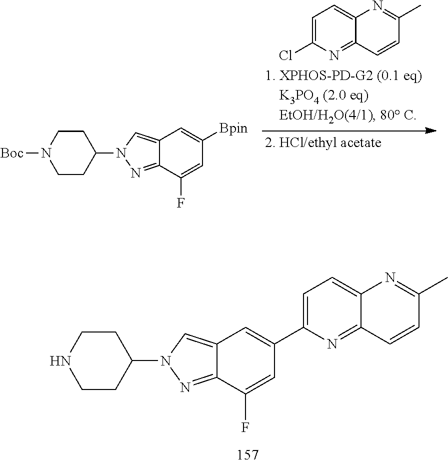

To a solution of tert-butyl 4-[7-fluoro-5-(4,4,5,5-tetramethyl-1,3,2-dioxaborolan-2-yl)indazol-2-yl]piperidine-1-carboxylate (100 mg, 224 umol, 1 eq) in EtOH (1.00 mL) and H₂O (250 uL) was added 2-chloro-6-methyl-1,5-naphthyridine (40.1 mg, 224 umol, 1 eq), K₃PO₄ (95.3 mg, 449 umol, 2 eq) and XPHOS-PD-G₂ (17.6 mg, 22.4 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water (5.00 mL) at 0° C. The reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give a crude product. The crude product was dissolved in EtOAc (2.00 mL) and then HCl/EtOAc (4 M, 1.00 mL) was added. The mixture was stirred at 25° C. for 2 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 2-[7-fluoro-2-(4-piperidyl) indazol-5-yl]-6-methyl-1,5-naphthyridine (36.1 mg, 38.5%) as a solid. LCMS: (ESI, m/z): 362.1 [M+H]^{+ 1}H NMR (DMSO-d₆, 400 MHz) δ ppm 9.18 (br d, 1H, J=8.9 Hz) 8.8-9.0 (m, 1H) 8.79 (d, 1H, J=2.7 Hz) 8.5-8.6 (m, 4H) 8.09 (dd, 1H, J=1.2, 13.5 Hz) 7.87 (d, 1H, J=8.7 Hz) 4.9-5.0 (m, 1H), 3.48 (br d, 2H, J=12.9 Hz) 3.1-3.3 (m, 2H) 2.82 (s, 3H) 2.3-2.4 (m, 4H) ¹⁹F NMR (DMSO-d₆, 376 MHz) δ ppm −128.47 (s, 1F)

### Example 46: Synthesis of Compound 158

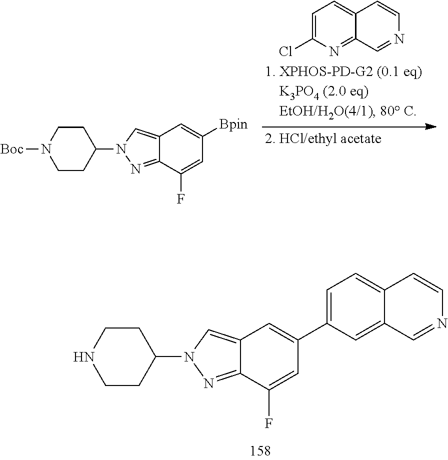

To a solution of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1 eq) in EtOH (1.00 mL) and H₂O (250 uL) was added 7-chloroisoquinoline (73.4 mg, 449 umol, 1 eq), K₃PO₄ (190 mg, 898 umol, 2 eq) and XPHOS-PD-G₂ (35.3 mg, 44.9 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water (5.00 mL) at 0° C. and the reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give a crude product. The crude product was added EtOAc (2.00 mL) and then HCl/EtOAc (4 M, 1.00 mL) was added under N₂ atmosphere. The mixture was stirred at 25° C. for 2 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 7-[7-fluoro-2-(4-piperidyl)indazol-5-yl]isoquinoline (99.6 mg, 50.7%) as a solid. LCMS: (ESI, m/z): 347.1 [M+H]^{+ 1}H NMR (DMSO-d₆, 400 MHz) δ ppm 9.80 (s, 1H) 9.3-9.4 (m, 1H) 9.0-9.2 (m, 1H) 8.85 (s, 1H) 8.75 (d, 1H, J=2.7 Hz) 8.67 (d, 1H, J=6.3 Hz) 8.58 (dd, 1H, J=1.8, 8.8 Hz) 8.44 (d, 1H, J=6.3 Hz) 8.38 (d, 1H, J=8.8 Hz) 8.15 (d, 1H, J=1.2 Hz) 7.67 (dd, 1H, J=1.3, 13.1 Hz) 4.9-5.0 (m, 1H) 3.46 (br d, 2H, J=12.9 Hz) 3.1-3.2 (m, 2H) 2.3-2.5 (m, 4H) ¹⁹F NMR (DMSO-d₆, 376 MHz) δ ppm −127.99 (s, 1F)

### Example 47: Synthesis of Compound 159

### Synthesis of Intermediate C83

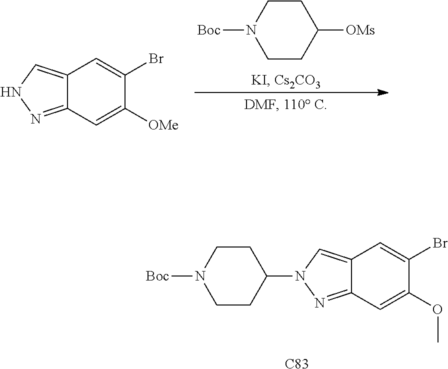

To a solution of 5-bromo-6-methoxy-2H-indazole (2.00 g, 8.81 mmol, 1 eq) and tert-butyl 4-methylsulfonyloxypiperidine-1-carboxylate (7.38 g, 26.4 mmol, 3 eq) in DMF (80.0 mL) was added Cs₂CO₃ (14.4 g, 44.0 mmol, 5 eq) and KI (4.39 g, 26.4 mmol, 3 eq). The mixture was stirred at 110° C. for 12 hrs. TLC (petroleum ether/ethyl acetate=5/1, Rf=0.5) showed that the starting material was consumed and a new major spot was generated. The reaction mixture was quenched with water (50.0 mL) at 20° C. and extracted with ethyl acetate (5×50.0 mL). The combined organic layer was dried over Na₂SO₄, filtered and the filtrate was concentrated under reduced pressure to give crude product. The crude product was purified by column chromatography on silica gel (SiO₂, 29% of ethyl acetate in petroleum ether) to give tert-butyl 4-(5-bromo-6-methoxy-2H-indazol-2-yl) piperidine-1-carboxylatenonane-9-carboxylate (1.10 g, 30.4%) as an oil. LCMS: (ES, m/z): 410.1, 412.1 [M+H]⁺. ¹HNMR (400 MHz, CDCl₃) δ ppm 7.85 (d, J=15.65 Hz, 2H) 7.03 (s, 1H) 4.49 (tt, J=11.51, 4.02 Hz, 1H) 4.32 (br s, 2H) 3.94 (s, 3H) 2.83-3.05 (m, 2H) 2.23 (br d, J=11.13 Hz, 2H) 2.07 (qd, J=12.19, 4.16 Hz, 2H) 1.49 (s, 9H)

### Synthesis of Intermediate C84

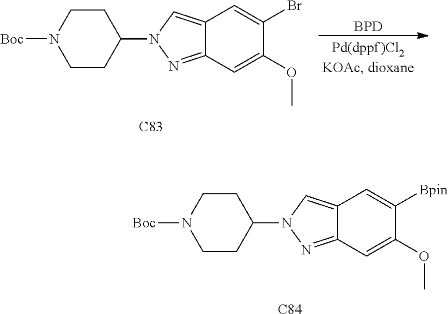

A mixture of tert-butyl 4-(5-bromo-6-methoxy-indazol-2-yl)piperidine-1-carboxylate (1.10 g, 2.68 mmol, 1 eq), 4,4,5,5-tetramethyl-2-(4,4,5,5-tetramethyl-1,3,2-dioxaborolan-2-yl)-1,3,2-dioxaborolane (1.02 g, 4.02 mmol, 1.5 eq), Pd(dppf)Cl₂ (98.1 mg, 134 umol, 0.05 eq) and KOAc (526 mg, 5.36 mmol, 2 eq) in dioxane (10.0 mL) was degassed and purged with N₂ for 3 times, and then the mixture was stirred at 100° C. for 12 hrs under N₂ atmosphere. The reaction mixture was cooled to 25° C. and quenched with water (20.0 mL), then extracted with ethyl acetate (5×20.0 mL). The combined organic layer was dried over Na₂SO₄, filtered and the filtrate was concentrated under reduced pressure. The crude product tert-butyl 4-(6-methoxy-5-(4,4,5,5-tetramethyl-1,3,2-dioxaborolan-2-yl)-2H-indazol-2-yl)piperidine-1-carboxylate (1.10 g, 90%) was obtained as a solid, which was used directly into the next step without further purification. LCMS: (ES, m/z): 458.3 [M+H]⁺.

### Synthesis of Intermediate C85

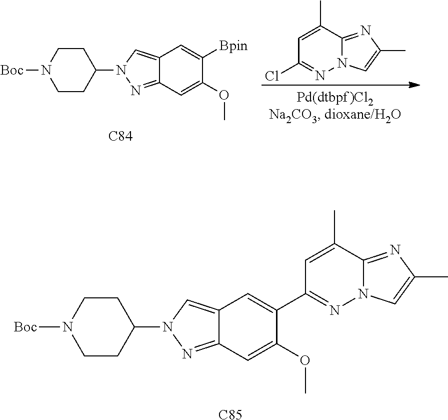

A mixture of tert-butyl 4-[6-methoxy-5-(4,4,5,5-tetramethyl-1,3,2-dioxaborolan-2-yl)indazol-2-yl]piperidine-1-carboxylate (1.10 g, 2.41 mmol, 1 eq), 6-chloro-2,8-dimethyl-imidazo[1,2-b]pyridazine (437 mg, 2.41 mmol, 1 eq), ditert-butyl(cyclopentyl)phosphane;dichloropalladium;iron (78.4 mg, 120 umol, 0.05 eq) and Na₂CO₃ (510 mg, 4.81 mmol, 2 eq) in H₂O (8.00 mL) and dioxane (40.0 mL) was degassed and purged with N₂ for 3 times, and then the mixture was stirred at 80° C. for 2 hrs under N₂ atmosphere. The reaction mixture was quenched with water (50.0 mL) and extracted with ethyl acetate (3×50.0 mL). The combined organic layer was dried over Na₂SO₄, filtered and the filtrate was concentrated under reduced pressure to give crude product which was purified by column chromatography on silica gel (SiO₂, 30% of ethyl acetate in petroleum ether) to give tert-butyl 4-(5-(2,8-dimethylimidazo[1,2-b]pyridazin-6-yl)-6-methoxy-2H-indazol-2-yl)piperidine-1-carboxylate (600 mg, 52.3%) as a solid. LCMS: (ESI, m/z): 477.2 [M+H]⁺. ¹HNMR (400 MHz, CDCl₃) δ ppm 7.95 (s, 1H) 7.83 (s, 1H) 7.74 (s, 1H) 7.17 (s, 1H) 7.12 (s, 1H) 4.53 (tt, J=11.51, 3.96 Hz, 1H) 4.25-4.44 (m, 2H) 3.90 (s, 3H) 2.96 (brt, J=11.86 Hz, 2H) 2.68 (s, 3H) 2.51-2.56 (m, 3H) 2.22-2.30 (m, 2H) 2.08-2.17 (m, 2H) 1.50 (s, 9H)

### Synthesis of Compound 159

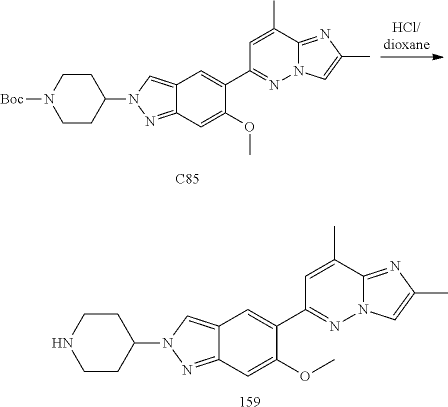

To a solution of tert-butyl 4-[5-(2,8-dimethylimidazo[1,2-b]pyridazin-6-yl)-6-methoxy-indazol-2-yl]piperidine-1-carboxylate (500 mg, 105 umol, 1 eq) in dioxane (1.00 mL) was added HCl/dioxane (1.00 mL). The mixture was stirred at 25° C. for 2 hrs The reaction mixture was concentrated under reduced pressure to give a residue. The residue was triturated with dichloromethane at 25° C. for 30 min to give 6-(6-methoxy-2-(piperidin-4-yl)-2H-indazol-5-yl)-2,8-dimethylimidazo[1,2-b]pyridazine (21.0 mg, 53.1%) as a solid. LCMS: (ESI, m/z): 377.3 [M+H]⁺. ¹HNMR (400 MHz, DMSO-d₆) δ ppm 8.47 (s, 1H) 8.35 (s, 1H) 7.94 (s, 1H) 7.83 (s, 1H) 7.16 (s, 1H) 4.74-4.87 (m, 1H) 3.84 (s, 3H) 3.45 (br d, J=12.84 Hz, 2H) 3.05-3.20 (m, 2H) 2.64 (s, 3H) 2.52 (s, 3H) 2.19-2.35 (m, 4H)

### Example 48: Synthesis of Compound 161

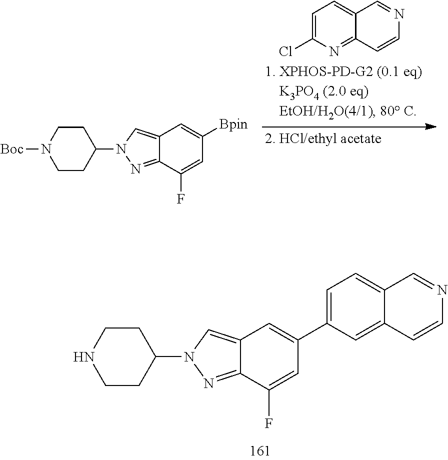

To a solution of tert-butyl 4-[7-fluoro-5-(4,4,5,5-tetramethyl-1,3,2-dioxaborolan-2-yl)indazol-2-yl]piperidine-1-carboxylate (100 mg, 224 umol, 1 eq) in EtOH (1.00 mL) and H₂O (250 uL) was added 6-chloroisoquinoline (36.7 mg, 224 umol, 1 eq), K₃PO₄ (95.3 mg, 449 umol, 2 eq) and XPHOS-PD-G₂ (17.6 mg, 22.4 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water (5.00 mL) at 0° C. and the reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give a crude product. The crude product was added EtOAc (2.00 mL) and then HCl/EtOAc (4 M, 1.00 mL) was added under N₂ atmosphere. The mixture was stirred at 25° C. for 2 hrs. TLC (ethyl acetate, Rf=0.10) showed the starting material was consumed and a new major spot was generated. The reaction mixture was concentrated under reduced pressure to remove ethyl acetate. The crude product was purified by recrystallization from MeOH (10.0 mL) at 25° C. to give 6-[7-fluoro-2-(4-piperidyl) indazol-5-yl]isoquinoline (43.6 mg, 46.3%) as a solid. LCMS: (ESI, m/z): 347.1 [M+H]^{+ 1}H NMR (DMSO-d₆, 400 MHz) δ ppm 9.77 (s, 1H) 9.14 (br d, 1H, J=10.8 Hz) 8.8-9.0 (m, 1H) 8.77 (d, 1H, J=2.7 Hz) 8.6-8.7 (m, 2H) 8.53 (d, 1H, J=8.7 Hz) 8.3-8.4 (m, 2H) 8.24 (s, 1H) 7.74 (dd, 1H, J=1.1, 13.2 Hz) 4.8-5.1 (m, 1H) 3.48 (br d, 2H, J=12.4 Hz) 3.0-3.2 (m, 2H) 2.3-2.4 (m, 4H) ¹⁹F NMR (DMSO-d₆, 376 MHz) δ ppm −128.00 (s, 1F)

### Example 49: Synthesis of Compound 162

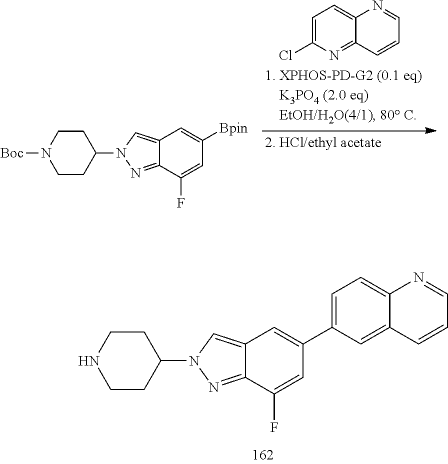

To a solution of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1 eq) in EtOH (1.00 mL) and H₂O (250 uL) was added 6-chloroquinoline (73.4 mg, 449 umol, 1 eq), K₃PO₄ (190 mg, 898 umol, 2 eq) and XPHOS-PD-G₂ (35.3 mg, 44.9 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water (5.00 mL) at 0° C. and the reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give a crude product. The crude product was added EtOAc (2.00 mL) and then HCl/EtOAc (4 M, 1.00 mL) was added under N₂ atmosphere. The mixture was stirred at 25° C. for 2 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 6-[7-fluoro-2-(4-piperidyl) indazol-5-yl]quinoline (100 mg, 53.3%) as a solid. LCMS: (ESI, m/z): 347.1 [M+H]^{+ 1}H NMR (DMSO-d₆, 400 MHz) δ ppm 9.1-9.3 (m, 2H), 8.8-9.0 (m, 2H), 8.73 (d, 1H, J=2.7 Hz), 8.60 (s, 1H), 8.4-8.5 (m, 1H), 8.3-8.4 (m, 1H), 8.12 (s, 1H), 7.9-8.0 (m, 1H), 7.68 (d, 1H, J=13.2 Hz), 4.96 (tt, 1H, J=4.8, 10.0 Hz), 3.4-3.6 (m, 2H), 3.0-3.2 (m, 2H), 2.3-2.4 (m, 4H) ¹⁹F NMR (DMSO-d₆, 376 MHz) δ ppm −128.20 (s, 1F)

### Example 50: Synthesis of Compound 163

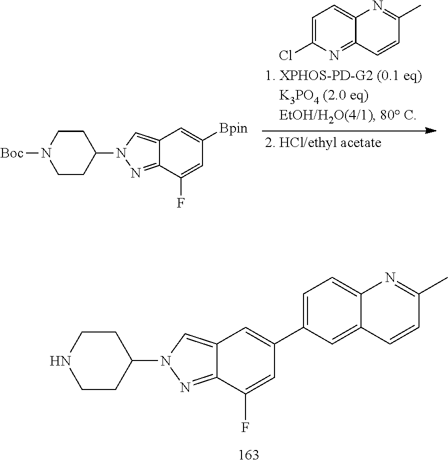

To a solution of tert-butyl 4-[7-fluoro-5-(4,4,5,5-tetramethyl-1,3,2-dioxaborolan-2-yl)indazol-2-yl]piperidine-1-carboxylate (100 mg, 224 umol, 1 eq) in EtOH (1.00 mL) and H₂O (250 uL) was added 6-chloro-2-methyl-quinoline (39.8 mg, 224 umol, 1 eq) and K₃PO₄ (95.3 mg, 449 umol, 2 eq), XPHOS-PD-G₂ (17.6 mg, 22.4 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water (5.00 mL) at 0° C. and the reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give a crude product. The crude product was added EtOAc (2.00 mL) and then HCl/EtOAc (4 M, 1.00 mL) was added under N₂ atmosphere. The mixture was stirred at 25° C. for 2 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 6-[7-fluoro-2-(4-piperidyl) indazol-5-yl]-2-methyl-quinoline (13.7 mg, 16.1%, 95.4% purity) as a solid. LCMS: (ESI, m/z): 361.1 [M+H]^{+ 1}H NMR (DMSO-d₆, 400 MHz) δ ppm 9.20 (br d, 1H, J=9.4 Hz) 8.8-9.0 (m, 2H) 8.73 (d, 1H, J=2.7 Hz) 8.61 (br s, 1H) 8.3-8.5 (m, 2H) 8.11 (d, 1H, J=1.0 Hz) 7.90 (br d, 1H, J=8.7 Hz) 7.67 (dd, 1H, J=1.2, 13.1 Hz) 4.9-5.0 (m, 1H) 3.48 (br d, 2H, J=12.6 Hz) 3.1-3.2 (m, 2H) 2.95 (s, 3H) 2.3-2.4 (m, 4H) ¹⁹F NMR (DMSO-d₆, 376 MHz) δ ppm −128.16 (s, 1F)

### Example 51: Synthesis of Compound 212

### Synthesis of Intermediate C86

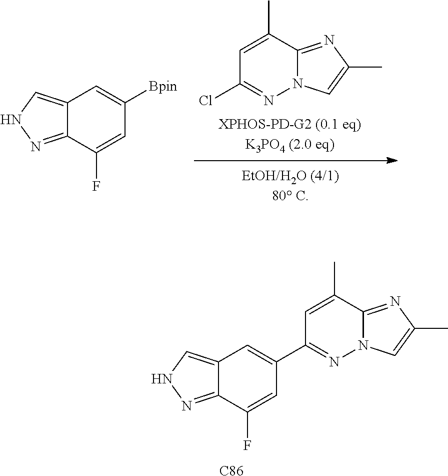

To a solution of 7-fluoro-5-(4,4,5,5-tetramethyl-1,3,2-dioxaborolan-2-yl)-2H-indazole (1.00 g, 3.82 mmol, 1 eq) in EtOH (10.0 mL) and H₂O (2.50 mL) was added 6-chloro-2,8-dimethyl-imidazo[1,2-b]pyridazine (692 mg, 3.82 mmol, 1 eq), K₃PO₄ (1.62 g, 7.63 mmol, 2 eq) and XPHOS-PD-G₂ (300 mg, 381 umol, 0.1 eq) under N₂ atmosphere. The mixture was stirred at 80° C. for 12 hrs. TLC (petroleum ether/ethyl acetate=1/1, Rf=0.55) showed the starting material was consumed and a new major spot was generated. The reaction mixture was quenched by addition water 20.0 mL at 0° C., the reaction mixture was filtered and the filter cake was washed with 10.0 mL of ethyl acetate, dried in vacuum to afford product. The crude product was triturated with ethyl acetate at 25° C. for 10 min. The reaction mixture was filtered and the filter cake was washed with 10.0 mL of ethyl acetate, dried in vacuum to give 6-(7-fluoro-2H-indazol-5-yl)-2,8-dimethyl-imidazo[1,2-b]pyridazine (2.50 g, 69.8%) as a solid. LCMS: (ESI, m/z): 282.2 [M+H]^{+ 1}H NMR (DMSO-d₆, 400 MHz) δ ppm 13.6-14.1 (m, 1H) 8.3-8.4 (m, 2H) 8.03 (d, 1H, J=0.7 Hz) 7.8-7.9 (m, 1H) 7.74 (d, 1H, J=1.0 Hz) 2.60 (d, 3H, J=0.7 Hz) 2.4-2.4 (m, 3H) ¹⁹F NMR (DMSO-d₆, 376 MHz) δ ppm −129.99 (s, 1F)

### Synthesis of Intermediate C87

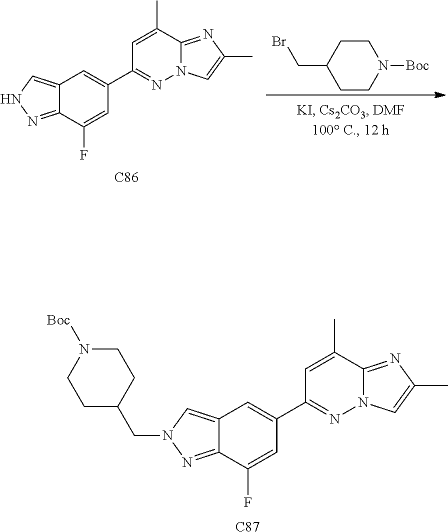

To a solution of 6-(7-fluoro-2H-indazol-5-yl)-2,8-dimethyl-imidazo[1,2-b]pyridazine (400 mg, 1.42 mmol, 1 eq) and tert-butyl 4-(bromomethyl)piperidine-1-carboxylate (1.19 g, 4.26 mmol, 3 eq) in DMF (5.00 mL) was added KI (471 mg, 2.84 mmol, 2 eq) and Cs₂CO₃ (2.31 g, 7.10 mmol, 5 eq) under N₂ atmosphere. The mixture was stirred at 100° C. for 12 hrs. TLC (ethyl acetate, Rf=0.6) showed the starting material was consumed and a new major spot was generated. The reaction mixture was filtered and the filtrate was concentrated under reduced pressure to remove N,N-dimethyl-formamide. The residue was purified by prep-HPLC (column: Phenomenex C18 75*30 mm*3 um; mobile phase: [water(NH₄HCO₃)—CH₃CN]; B %: 45%-75%, 8 min) to give tert-butyl 4-[[5-(2,8-dimethylimidazo[1,2-b]pyridazin-6-yl)-7-fluoro-indazol-2-yl]methyl]piperidine-1-carboxylate (100 mg, 13.2%) as a solid. LCMS: (ESI, m/z): 479.3 [M+H]^{+ 1}H NMR (CDCl₃, 400 MHz) δ ppm 8.05 (d, 1H, J=2.5 Hz) 8.01 (d, 1H, J=1.1 Hz) 7.78 (s, 1H) 7.69 (dd, 1H, J=1.2, 12.5 Hz) 7.31 (br s, 1H) 4.35 (d, 2H, J=7.3 Hz) 4.0-4.3 (m, 2H) 2.6-2.8 (m, 5H) 2.57 (s, 3H) 2.3-2.4 (m, 1H) 1.61 (br d, 2H, J=4.6 Hz) 1.48 (s, 9H) 1.2-1.4 (m, 2H) ¹⁹F NMR (CDCl₃, 376 MHz) δ ppm −127.49 (s, 1F)

### Synthesis of Compound 212

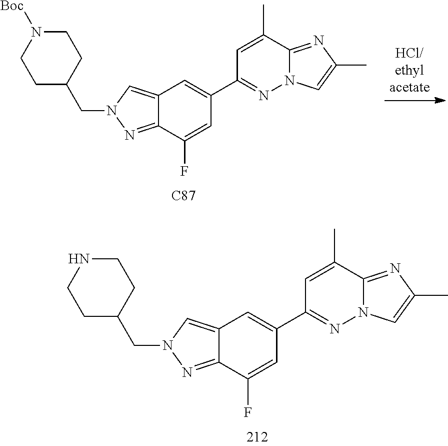 [SMILES: Cc1cn2nc(-c3cc(F)c4nn(CC5CCN(C(=O)OC(C)(C)C)CC5)cc4c3)cc(C)c2n1 ; "C87" ; Cc1cn2nc(-c3cc(F)c4nn(CC5CCNCC5)cc4c3)cc(C)c2n1]

To a solution of tert-butyl 4-[[5-(2, 8-dimethylimidazo [1,2-b]pyridazin-6-yl)-7-fluoro-indazol-2-yl]methyl]piperidine-1-carboxylate (100 mg, 208 umol, 1 eq) in EtOAc (2.00 mL) was added HCl/EtOAc (4 M, 800 uL) under N₂ atmosphere. The mixture was stirred at 25° C. for 2 hrs. TLC (petroleum ether/ethyl acetate=1/1, Rf=0.00) showed the starting material was consumed and a new major spot was generated. The reaction mixture was concentrated under reduced pressure to remove ethyl acetate. The crude product was purified by re-crystallization from MeOH (10.0 mL) at 25° C. to give 6-[7-fluoro-2-(4-piperidylmethyl) indazol-5-yl]-2,8-dimethyl-imidazo[1,2-b]pyridazine (78.1 mg, 84.1%) as a solid. LCMS: (ESI, m/z): 379.1 [M+H]^{+ 1}H NMR (MeOD-d₄, 400 MHz) δ ppm 8.63 (d, 1H, J=2.6 Hz) 8.43 (d, 1H, J=1.2 Hz) 8.30 (dd, 2H, J=1.0, 10.8 Hz) 7.85 (dd, 1H, J=1.2, 12.8 Hz) 4.52 (d, 2H, J=7.1 Hz) 3.43 (br d, 2H, J=12.8 Hz) 3.01 (dt, 2H, J=2.6, 12.9 Hz) 2.78 (d, 3H, J=0.9 Hz) 2.65 (d, 3H, J=0.9 Hz) 2.48 (qtd, 1H, J=3.6, 7.6, 11.3 Hz) 1.86 (br d, 2H, J=13.6 Hz) 1.5-1.7 (m, 2H) ¹⁹F NMR (MeOD-d₄, 376 MHz) δ ppm −129.65 (s, 1F)

### Example 52: Synthesis of Compound 165

### Synthesis of Intermediate C88

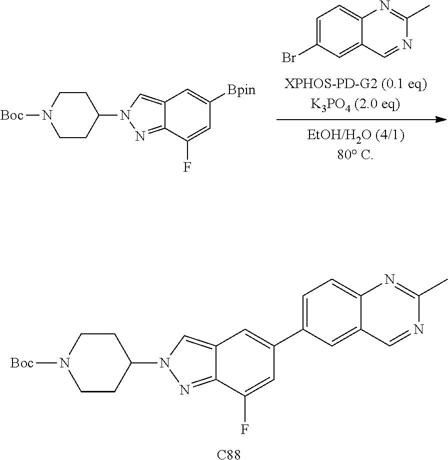

To a solution of tert-butyl 4-[7-fluoro-5-(4,4,5,5-tetramethyl-1,3,2-dioxaborolan-2-yl)indazol-2-yl]piperidine-1-carboxylate (200 mg, 449 umol, 1 eq) in EtOH (10.0 mL) and H₂O (2.50 mL) was added 6-bromo-2-methyl-quinazoline (100 mg, 449 umol, 1 eq), K₃PO₄ (190 mg, 898 umol, 2 eq) and XPHOS-PD-G₂ (35.3 mg, 44.9 umol, 0.1 eq) under N₂ atmosphere. The mixture was stirred at 80° C. for 12 hrs. TLC (petroleum ether/ethyl acetate=1/1, Rf=0.65) showed the starting material was consumed and a new major spot was generated. The reaction mixture was quenched by addition water (5.00 mL) at 0° C. and the reaction mixture was filtered and the filter cake was washed with 10.0 mL of ethyl acetate, dried in vacuum to give residue. The residue was triturated with ethyl acetate at 25° C. for 10 min. The reaction mixture was filtered and the filter cake was washed with 10.0 mL of ethyl acetate, dried in vacuum to give tert-butyl 4-[7-fluoro-5-(2-methylquinazolin-6-yl)indazol-2-yl]piperidine-1-carboxylate (200 mg, 96.4%) as a solid. LCMS: (ESI, m/z): 462.3 [M+H]^{+ 1}H NMR (CDCl₃, 400 MHz) δ ppm 9.40 (s, 1H) 8.17 (dd, 1H, J=2.1, 8.8 Hz) 8.09 (dd, 2H, J=2.2, 14.1 Hz) 8.04 (d, 1H, J=8.8 Hz) 7.75 (d, 1H, J=1.2 Hz) 7.34 (dd, 1H, J=1.3, 12.3 Hz) 4.66 (tt, 1H, J=4.0, 11.7 Hz) 4.2-4.6 (m, 2H) 2.9-3.1 (m, 5H) 2.31 (br dd, 2H, J=2.2, 12.2 Hz) 2.1-2.2 (m, 2H) 1.51 (s, 9H) ¹⁹F NMR (CDCl₃, 376 MHz) δ ppm −127.67 (s, 1F)

### Synthesis of Compound 165

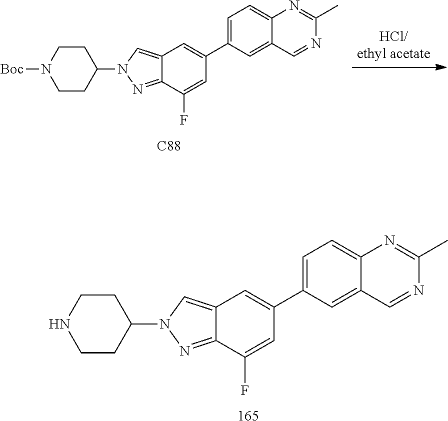

To a solution of tert-butyl 4-[7-fluoro-5-(2-methylquinazolin-6-yl) indazol-2-yl]piperidine-1-carboxylate (150 mg, 325 umol, 1 eq) in EtOAc (1.00 mL) was added HCl/EtOAc (4 M, 81.2 uL, 1 eq) under N₂ atmosphere. The mixture was stirred at 25° C. for 6 hrs. TLC (ethyl acetate, Rf=0.10) showed the starting material was consumed and a new major spot was generated. The reaction mixture was concentrated under reduced pressure to remove ethyl acetate. The crude product was purified by re-crystallization from MeOH (10.0 mL) at 25° C. to give 6-[7-fluoro-2-(4-piperidyl) indazol-5-yl]-2-methyl-quinazoline (112 mg, 86%) as a solid. LCMS: (ESI, m/z): 362.2 [M+H]^{+ 1}H NMR (MeOD-d₄, 400 MHz) δ ppm 8.52 (s, 1H), 7.8-8.0 (m, 3H), 7.3-7.4 (m, 2H), 6.21 (s, 1H), 4.97 (br dd, 1H, J=4.9, 9.9 Hz), 3.66 (br d, 2H, J=13.3 Hz), 3.35 (br s, 2H), 2.57 (s, 3H), 2.4-2.5 (m, 4H) ¹⁹F NMR (MeOD-d₄, 376 MHz) δ ppm −130.63 (s, 1F).

### Example 53: Synthesis of Compound 207

### Synthesis of Intermediate C89

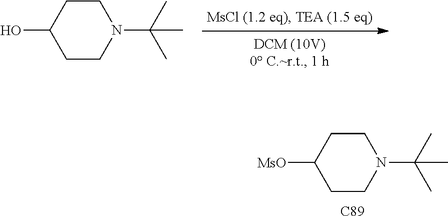

Into a 50 mL bottles were added 1-tert-butylpiperidin-4-ol (900 mg, 5.723 mmol, 1 equiv) and TEA (868.71 mg, 8.585 mmol, 1.5 equiv) in DCM (9 mL) at room temperature. To the above mixture was added MsCl (786.63 mg, 6.868 mmol, 1.2 equiv) dropwise over 1 min at 0° C. The resulting mixture was stirred for additional 1 h at room temperature. The reaction was quenched with water at room temperature. The reaction was quenched with NaHCO₃ at room temperature. The resulting mixture was extracted with DCM (3×18 mL). The combined organic layers were washed with NaCl solution (2×20 mL), dried over anhydrous Na₂SO₄. After filtration. The resulting mixture was concentrated under reduced pressure. This resulted in 1-tert-butylpiperidin-4-yl methanesulfonate (800 mg, 59.40%) as a solid. LCMS: (ESI, m/z): 235[M+H]⁺

### Synthesis of Intermediate C90

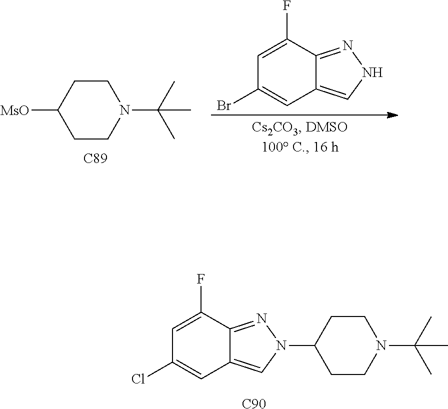

Into a 50 mL bottles were added 1-tert-butylpiperidin-4-yl methanesulfonate (840 mg, 3.569 mmol, 1.5 equiv) and 5-bromo-7-fluoro-2H-indazole (511.66 mg, 2.379 mmol, 1 equiv) and Cs₂CO₃ (2325.89 mg, 7.138 mmol, 3 equiv) in DMSO (9 mL) at room temperature. The resulting mixture was stirred for additional 16 h at 100° C. The mixture was allowed to cool down to room temperature. The reaction was quenched with H₂O at room temperature. The resulting mixture was extracted with EA (3×20 mL). The combined organic layers were washed with NaCl solution (2×20 mL), dried over anhydrous Na₂SO₄. After filtration, the filtrate was concentrated under reduced pressure. The residue was purified by silica gel column chromatography, eluted with PE/EA (1:1) to afford 5-bromo-2-(1-tert-butylpiperidin-4-yl)-7-fluoroindazole (110 mg) as a solid. LCMS: (ESI, m/z): 353[M+H]⁺

### Synthesis of Compound 207

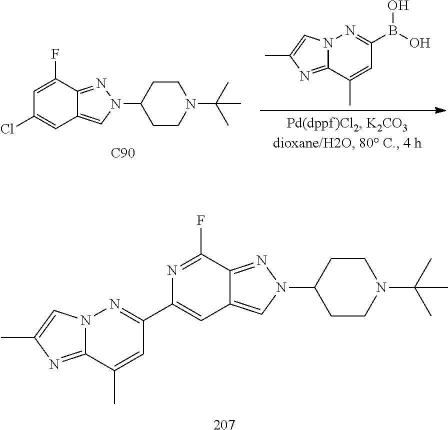 [SMILES: CC(C)(C)N1CCC(n2cc3cc(Cl)cc(F)c3n2)CC1 ; Cc1cn2nc(B(O)O)cc(C)c2n1 ; "C90" ; Cc1cn2nc(-c3cc4cn(C5CCN(C(C)(C)C)CC5)nc4c(F)n3)cc(C)c2n1 ; "207"]

A solution of 5-bromo-2-(1-tert-butylpiperidin-4-yl)-7-fluoroindazole (90 mg, 0.254 mmol, 1 equiv), Pd(dppf)Cl₂ (20.7 mg, 0.025 mmol, 0.10 equiv), 2,8-dimethylimidazo[1,2-b]pyridazin-6-ylboronic acid (72.78 mg, 0.381 mmol, 1.50 equiv) and K₃PO₄ (134.81 mg, 0.635 mmol, 2.50 equiv) in dioxane (5 mL) and H₂O (1 mL) was stirred for 4 h at 80° C. under nitrogen atmosphere. The mixture was allowed to cool down to room temperature. The resulting mixture was dried over anhydrous Na₂SO₄. After filtration, the filtrate was concentrated under reduced pressure. The residue was purified by silica gel column chromatography, eluted with CH₂Cl₂/MeOH (10:1). The crude product was purified by Chiral-Prep-HPLC with the following conditions: Column, Kinetex EVO C18 Column, 30×150, 5 um; mobile phase, water(10 mmol/L NH₄HCO₃) and ACN (20% ACN up to 55% in 8 min); Detector, UV 220 nm to afford 2-(1-tert-butylpiperidin-4-yl)-5-{2,8-dimethylimidazo[1,2-b]pyridazin-6-yl}-7-fluoroindazole (26.0 mg, 24.09%) as a solid. LCMS: (ESI, m/z): 420[M+H]^{+ 1}H NMR: (400 MHz, DMSO-d₆) δ 8.73 (d, J=2.8 Hz, 1H), 8.28 (d, J=1.3 Hz, 1H), 8.04 (d, J=1.0 Hz, 1H), 7.77-7.69 (m, 2H), 4.52 (s, 1H), 3.16 (d, J=11.0 Hz, 2H), 2.60 (d, J=1.1 Hz, 3H), 2.40 (d, J=0.8 Hz, 3H), 2.25 (t, J=11.3 Hz, 2H), 2.16-2.04 (m, 4H), 1.08 (s, 9H).

### Example 54: Synthesis of Compound 143

### Synthesis of Intermediate C92

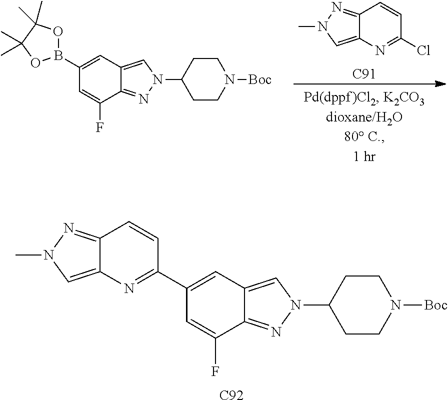 [SMILES: "C91" ; CC(C)(C)OC(=O)N1CCC(n2cc3cc(B4OC(C)(C)C(C)(C)O4)cc(F)c3n2)CC1 ; Cn1cc2nc(Cl)ccc2n1 ; Cn1cc2nc(-c3cc(F)c4nn(C5CCN(C(=O)OC(C)(C)C)CC5)cc4c3)ccc2n1 ; "C92"]

To a mixture of tert-butyl 4-[7-fluoro-5-(4,4,5,5-tetramethyl-1,3,2-dioxaborolan-2-yl)indazol-2-yl]piperidine-1-carboxylate (0.30 g, 673 umol, 1.50 eq) and 5-chloro-2-methyl-pyrazolo [4,3-b]pyridine (75.2 mg, 449 umol, 1.00 eq) in dioxane (2.40 mL) was added a solution of K₂CO₃ (124 mg, 898 umol, 2.00 eq) in H₂O (0.600 mL). Then, 1,1-bis(diphenylphosphino)ferrocene]dichloropalladium(II) (65.7 mg, 89.8 umol, 0.20 eq) was added to the above mixture under N₂ protection. The reaction mixture was stirred at 80° C. for 1 hr. The reaction mixture was diluted with H₂O (20.0 mL), extracted with ethyl acetate (3×20.0 mL), dried over Na₂SO₄, filtered and concentrated under reduced pressure to give an oil. The oil was purified by column chromatography on silica gel eluted with petroleum ether/ethyl acetate (5/1 to 0/1) to give tert-butyl 4-[7-fluoro-5-(2-methylpyrazolo[4,3-b]pyridin-5-yl)indazol-2-yl]piperidine-1-carboxylate (140 mg, 64.3%) as an oil. LCMS: (ESI, m/z): 451.2 [M+H]⁺. ¹H NMR (400 MHz, METHANOL-d₄) δ ppm 8.52 (d, J=2.75 Hz, 1H) 8.44 (s, 1H) 8.11-8.19 (m, 2H) 7.91 (d, J=9.13 Hz, 1H) 7.79 (dd, J=13.20, 1.19 Hz, 1H) 4.61-4.81 (m, 1H) 4.24-4.34 (m, 5H) 3.04 (br s, 2H) 2.07-2.30 (m, 4H) 1.51 (s, 9H)

### Synthesis of Compound 143

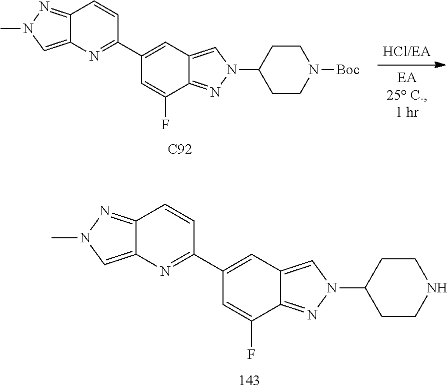

To a solution of tert-butyl 4-[7-fluoro-5-(2-methylpyrazolo[4,3-b]pyridin-5-yl)indazol-2-yl]piperidine-1-carboxylate (140 mg, 310 umol, 1.00 eq) in ethyl acetate (2.30 mL) was added HCl/ethyl acetate (4.00 M, 2.84 mL, 36.5 eq). The reaction mixture was stirred at 25° C. for 1 hr. The reaction mixture was filtered and the filter cake was concentrated in vacuum to give white solid. The white solid was triturated with methyl tert-butyl ether (3.00 mL) to give 5-[7-fluoro-2-(4-piperidyl)indazol-5-yl]-2-methyl-pyrazolo[4,3-b]pyridine (123 mg, 93.4%) as a solid. LCMS: (ESI, m/z): 351.1 [M+H]⁺. ¹H NMR (400 MHz, METHANOL-d4) δ ppm 8.89 (dd, J=9.06, 0.60 Hz, 1H) 8.77-8.82 (m, 2H) 8.44 (d, J=1.43 Hz, 1H) 8.24 (d, J=9.06 Hz, 1H) 7.72 (dd, J=12.16, 1.43 Hz, 1H) 5.06 (dt, J=10.04, 5.05 Hz, 1H) 4.44 (s, 3H) 3.61-3.73 (m, 2H) 3.32-3.39 (m, 2H) 2.44-2.62 (m, 4H)

### Example 55: Synthesis of Compound 138

### Synthesis of Intermediate C93

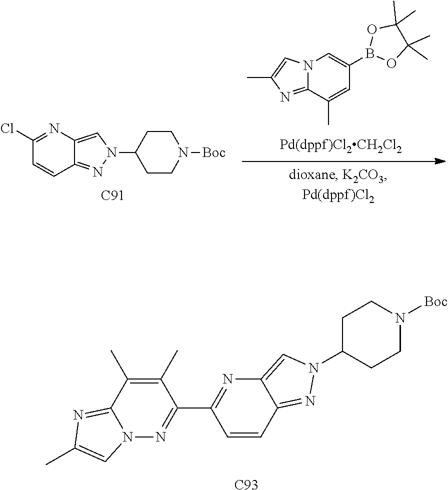

To a mixture of 2,8-dimethyl-6-(4,4,5,5-tetramethyl-1,3,2-dioxaborolan-2-yl)imidazo[1,2-b]pyridazine (48.6 mg, 178 umol, 3.00 eq) and tert-butyl 4-(5-chloropyrazolo[4,3-b]pyridin-2-yl)piperidine-1-carboxylate (20.0 mg, 59.3 umol, 1.00 eq) in dioxane (0.400 mL) was added a solution of K₂CO₃ (16.4 mg, 118 umol, 2.00 eq) in H₂O (0.100 mL). Then, [1,1-bis(diphenylphosphino)ferrocene]dichloropalladium(II) (8.69 mg, 11.8 umol, 0.200 eq) was added to the above mixture under N₂ protection. The reaction mixture was stirred at 80° C. for 1 hr. The reaction mixture was diluted with water (10.0 mL), and extracted with ethyl acetate (3×10.0 mL). The combined organic layer was washed with brine (2×20.0 mL), dried over Na₂SO₄, filtered and concentrated under reduced pressure to give solid. The solid was purified by column chromatography on silica gel eluted with petroleum ether/ethyl acetate (1/0 to 5/1) to give tert-butyl 4-[5-(2,8-dimethylimidazo[1,2-b]pyridazin-6-yl)pyrazolo[4,3-b]pyridin-2-yl]piperidine-1-carboxylate (50 mg, 37.6%) as an oil. LCMS: (ESI, m/z): 448.2 [M+H]⁺.

### Synthesis of Compound 138

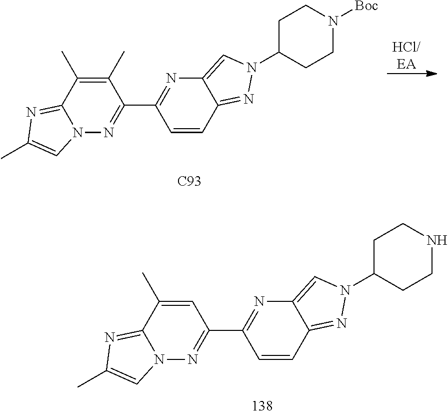 [SMILES: Cc1cn2nc(-c3ccc4nn(C5CCN(C(=O)OC(C)(C)C)CC5)cc4n3)c(C)c(C)c2n1 ; "HCl/EA" ; "C93" ; Cc1cn2nc(-c3ccc4nn(C5CCNCC5)cc4n3)cc(C)c2n1]

To a solution of tert-butyl 4-[5-(2,8-dimethylimidazo[1,2-b]pyridazin-6-yl)pyrazolo[4,3-b]pyridine-2-yl]piperidine-1-carboxylate (50.0 mg, 111 umol, 1.00 eq) in ethyl acetate (1.00 mL) was added HCl/ethyl acetate (4.00 M, 1.00 mL). The reaction was stirred at 25° C. for 1 hr. The reaction mixture was filtered and the filter cake was purified by prep-HPLC (column: Phenomenex Luna 80*30 mm*3 um; mobile phase: [water (HCl)-acetonitrile]; B %: 1%-50%, 8 min) to give 2,8-dimethyl-6-[2-(4-piperidyl) pyrazolo[4,3-b]pyridin-5-yl]imidazo[1,2-b]pyridazine (17.0 mg, 43.8%) as a solid. LCMS: (EIS, m/z): 348.1 [M+H]⁺. ¹H NMR (400 MHz, D₂O) δ ppm 8.76 (s, 1H) 8.21-8.40 (m, 3H) 8.12 (s, 1H) 5.03-5.09 (m, 1H) 3.72 (br d, J=13.45 Hz, 2H) 3.29-3.43 (m, 2H) 2.73 (s, 3H) 2.43-2.64 (m, 7H).

### Example 56: Synthesis of Compound 137

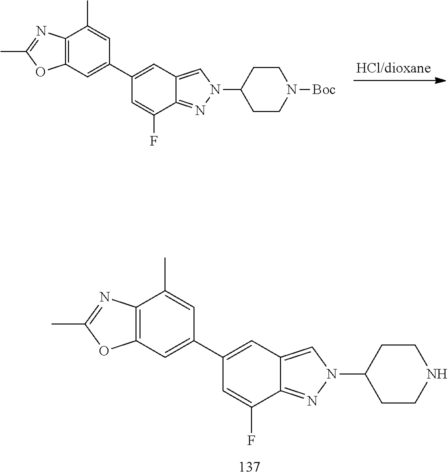

A suspension of tert-butyl 4-[5-(2,4-dimethyl-1,3-benzoxazol-6-yl)-7-fluoro-indazol-2-yl]piperidine-1-carboxylate (115 mg, 247 umol, 1.00 eq) in HCl/dioxane (4.00 M, 4.60 mL, 74.3 eq) was stirred at 25° C. for 1 hr. The mixture was concentrated under reduced pressure to give white solid. The solid was purified by prep-HPLC (column: Waters Xbridge BEH C18 100*30 mm*10 um; mobile phase: water (NH₄HCO₃)-ACN; B %: 25%-55%, 10 mins) to afford 6-[7-fluoro-2-(4-piperidyl)indazol-5-yl]-2,4-dimethyl-1,3-benzoxazole (20.0 mg, 22.1%) as a solid. LCMS: (ES, m/z): 365.1 [M+H]⁺. ¹H NMR (400 MHz, DMSO-d6) δ ppm 8.59 (d, J=2.74 Hz, 1H) 7.77-7.87 (m, 2H) 7.41-7.55 (m, 2H) 4.53-4.66 (m, 1H) 3.10 (br d, J=12.52 Hz, 2H) 2.65 (br s, 2H) 2.63 (s, 3H) 2.55 (s, 3H) 1.92-2.11 (m, 4H).

### Example 57: Synthesis of Compound 130

### Synthesis of Intermediate C95

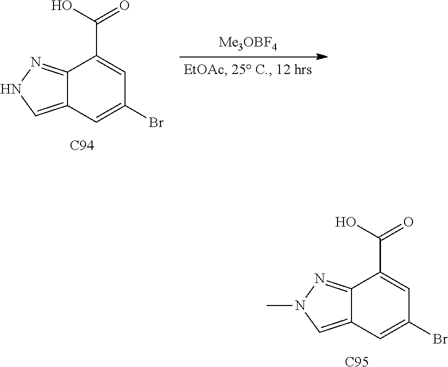

To a solution of 5-bromo-2H-indazole-7-carboxylic acid (2 g, 8.30 mmol, 1.00 eq) in ethyl acetate (40.0 mL) was added trimethyloxonium tetrafluoroborate (3.31 g, 22.4 mmol, 2.70 eq). The mixture was stirred at 25° C. for 12 hrs. The reaction mixture was diluted with water (100 mL) and extracted with ethyl acetate (3×30.0 mL), dried over Na₂SO₄, filtered and concentrated under reduced pressure to give 5-bromo-2-methyl-indazole-7-carboxylic acid (2.00 g, 94.5%) as a solid. LCMS: (ESI, m/z): 254.9 [M+H]⁺. ¹H NMR (400 MHz, DMSO-d6) δ ppm 8.50 (s, 1H) 8.26 (d, J=1.91 Hz, 1H) 7.88 (d, J=1.91 Hz, 1H) 4.22 (s, 3H).

### Synthesis of C96

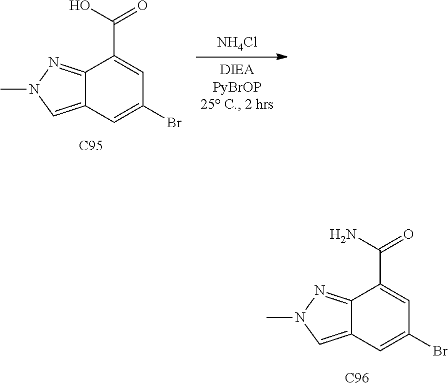

To a solution of 5-bromo-2-methyl-indazole-7-carboxylic acid (2.00 g, 7.84 mmol, 1.00 eq) and benzotriazol-1-yloxy(tripyrrolidin-1-yl)phosphonium hexafluorophosphate (4.90 g, 9.41 mmol, 1.20 eq) in dimethyl formamide (20.0 mL) was added N-ethyl-N-propan-2-ylpropan-2-amine (3.04 g, 23.5 mmol, 4.10 mL, 3.00 eq) and ammonia chloride (838 mg, 15.68 mmol, 2.00 eq). The mixture was stirred at 25° C. for 2 hrs. The reaction mixture was diluted with water (30.0 mL) and extracted with ethyl acetate (3×10.0 mL), dried over Na₂SO₄, filtered. The filtrate was concentrated under reduced pressure to give residue. The solid was triturated with methyl tert-butyl ether (20.0 mL) at 25° C. for 20 mins, then filtered. The filter cake was dried over in vacuum to give 5-bromo-2-methyl-indazole-7-carboxamide (1.40 g, 66.7%) as a solid. LCMS: (ESI, m/z): 253.9 [M+H]⁺.

### Synthesis of Intermediate C97

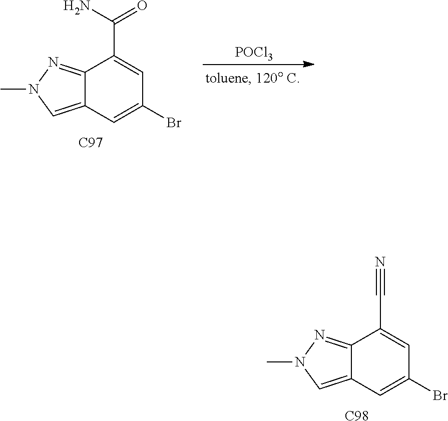

To a solution of 5-bromo-2-methyl-indazole-7-carboxamide (1.40 g, 5.51 mmol, 1.00 eq) in toluene (56.0 mL) was added phosphorus oxychloride (8.45 g, 55.1 mmol, 5.12 mL, 10.0 eq) dropwise. The mixture was stirred at 120° C. for 12 hrs. The reaction mixture was poured into water (50.0 mL), extracted with ethyl acetate (30.0 mL×3), washed with brine (30.0 mL×3), dried over Na₂SO₄, filtered and concentrated under reduced pressure to give an oil. The oil was purified by column chromatography (SiO₂, petroleum ether/ethyl acetate=10/1 to 1/1) to give 5-bromo-2-methyl-indazole-7-carbonitrile (1.00 g, 73.0%) as a solid. LCMS: (ESI, m/z): 235.9 [M+H]⁺. ¹H NMR (400 MHz, DMSO-d6) δ ppm 8.61 (s, 1H) 8.40 (d, J=1.59 Hz, 1H) 8.07 (d, J=1.59 Hz, 1H) 4.25 (s, 3H).

### Synthesis of Intermediate C99

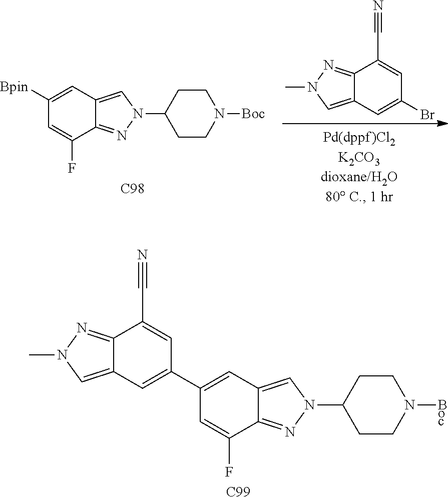

To a solution of 5-bromo-2-methyl-indazole-7-carbonitrile (70.6 mg, 299 umol, 1.00 eq) and tert-butyl 4-[7-fluoro-5-(4,4,5,5-tetramethyl-1,3,2-dioxaborolan-2-yl)indazol-2-yl]piperidine-1-carboxylate (200 mg, 449 umol, 1.50 eq) in dioxane (2.88 mL) was added a solution of potassium carbonate (82.7 mg, 598 umol, 2.00 eq) of water (0.72 mL) and cyclopentyl(diphenyl)phosphane dichloropalladiumiron (43.8 mg, 59.8 umol, 0.200 eq). The reaction was stirred at 80° C. for 1 hr. The reaction mixture was diluted with water (10 mL), extracted with ethyl acetate (3×10.0 mL), washed with brine (3×10.0 mL), dried over Na₂SO₄, filtered and concentrated under reduced pressure to give an oil. The oil was purified by column chromatography (SiO₂, petroleum ether/ethyl acetate=10/1 to 1/1) to give tert-butyl 4-[5-(7-cyano-2-methyl-indazol-5-yl)-7-fluoro-indazol-2-yl]piperidine-1-carboxylate (130 mg, 86.9%) as a solid. LCMS: (ESI, m/z): 419.1 [M+H]⁺. ¹H NMR (400 MHz, DMSO-d₆) δ=8.69-8.61 (m, 2H), 8.42 (d, J=1.6 Hz, 1H), 8.31 (d, J=1.6 Hz, 1H), 7.93 (d, J=1.1 Hz, 1H), 7.54 (dd, J=1.3, 13.4 Hz, 1H), 4.87-4.69 (m, 1H), 4.27 (s, 3H), 4.19-4.05 (m, 2H), 3.12-2.82 (m, 2H), 2.15 (br d, J=10.3 Hz, 2H), 2.05-1.99 (m, 2H), 1.44 (s, 9H).

### Synthesis of Compound 130

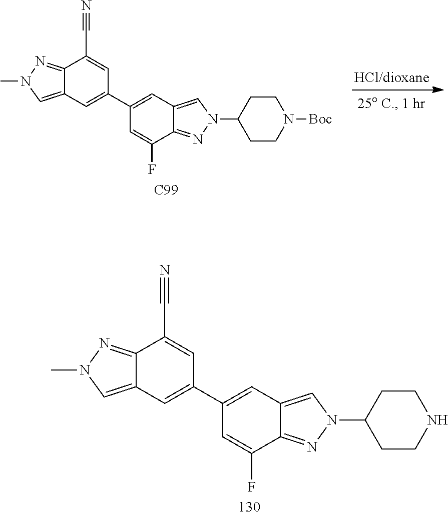

To a solution of tert-butyl 4-[5-(7-cyano-2-methyl-indazol-5-yl)-7-fluoro-indazol-2-yl]piperidine-1-carboxylate (125 mg, 263 umol, 1.00 eq) in dioxane (2.48 mL) was added HCl/dioxane (2.48 mL). The mixture was stirred at 25° C. for 1 hr. The reaction mixture was filtered to give a yellow solid, and the filter cake was dried in vacuum to give 5-[7-fluoro-2-(4-piperidyl)indazol-5-yl]-2-methyl-indazole-7-carbonitrile (60.0 mg, 52.2%) as a solid. LCMS: (ESI, m/z): 375.2 [M+H]⁺. ¹H NMR (400 MHz, deuterium oxide) δ ppm 8.00 (br d, J=1.75 Hz, 1H) 7.82 (s, 1H) 7.25 (s, 1H) 7.03 (s, 1H) 6.74 (s, 1H) 6.28 (br d, J=13.13 Hz, 1H) 4.64 (br t, J=11.26 Hz, 1H) 3.86 (s, 3H) 3.64 (br d, J=12.01 Hz, 2H) 3.25 (br t, J=12.07 Hz, 2H) 2.37 (br d, J=12.26 Hz, 2H) 2.22 (q, J=11.34 Hz, 2H).

### Example 58: Synthesis of Compound 124

### Synthesis of Intermediate C100

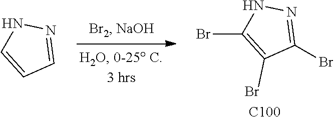 [SMILES: c1cn[nH]c1 ; Brc1n[nH]c(Br)c1Br ; "C100"]

To a solution of NaOH (58.7 g, 1.47 mol, 4.00 eq) in H₂O (1.40 L) was added 1H-pyrazole (25.0 g, 367 mmol, 1.00 eq) at 0° C. Then, Br₂ (176 g, 1.10 mol, 56.7 mL, 3.00 eq) was added to above mixture dropwise at 0° C. for 2 hrs. The reaction mixture was stirred at 25° C. for 1 hr. The reaction mixture was filtered and the filter cake was concentrated in vacuum to get solid. The solid was washed with H₂O (1000 mL) and dried in vacuum to afford a solid. The solid was triturated with acetonitrile (250 mL) and then filtered. The filter cake was concentrated in vacuum to give 3,4,5-tribromo-1H-pyrazole (50.0 g, 37.9%) as a solid. LCMS: (ESI, m/z): 306.8 [M+H]⁺.

### Synthesis of Intermediate C101

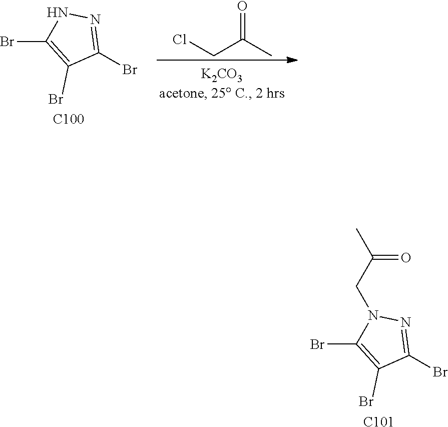

To a solution of 3,4,5-tribromo-1H-pyrazole (33.0 g, 108 mmol, 1.00 eq) and K₂CO₃ (44.9 g, 324 mmol, 3.00 eq) in acetone (428 mL) was added 1-chloropropan-2-one (10.9 g, 118 mmol, 1.10 eq). The reaction mixture was stirred at 25° C. for 2 hrs. The reaction mixture was diluted with water (200 mL), extracted with dichloromethane (3×200 mL), dried over Na₂SO₄, filtered and concentrated under reduced pressure to give an oil. The oil was triturated with methyl tertiary butyl ether (20.0 mL) at 25° C. for 40 mins and filtered. The filter cake was dried over in vacuum to give 1-(3,4,5-tribromopyrazol-1-yl)propan-2-one (22.0 g, 55.2%) as a solid. LCMS: (ESI, m/z): 362.8 [M+H]⁺. ¹H NMR (400 MHz, CHLOROFORM-d) δ ppm 4.98 (s, 2H) 2.20 (s, 3H).

### Synthesis of Intermediate C102

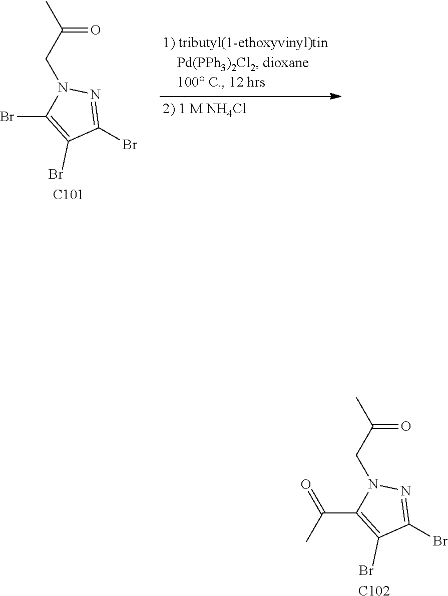

To a solution of 1-(3,4,5-tribromopyrazol-1-yl)propan-2-one (5.00 g, 13.8 mmol, 1.00 eq), tributyl(1-ethoxyvinyl)tin (5.50 g, 15.2 mmol, 5.14 mL, 1.10 eq) and bis(triphenylphosphine) palladium(ii) dichloride (486 mg, 692 umol, 0.050 eq) in dioxane (50.0 mL). The reaction mixture was stirred at 100° C. for 12 hrs. The reaction mixture was diluted with water (100 mL). The resulting mixture was extracted with ethyl acetate (2×100 mL). The combined organic layers were washed with brine (2×200 mL), dried over anhydrous Na₂SO₄. After filtration, the filtrate was concentrated under reduced pressure to give residue. The residue was diluted with tetrahydrofuran (24.0 mL) and 1 NH₄Cl (65.8 mL). The reaction mixture was stirred at 60° C. for 1 hr. The reaction mixture was diluted with water (300 mL), extracted with ethyl acetate (3×250 mL), washed with brine (4×300 mL), dried over Na₂SO₄, filtered and concentrated under reduced pressure to give oil. The oil was purified by column chromatography on silica gel eluted with petroleum ether/ethyl acetate (1/0 to 5/1) to give 1-(5-acetyl-3,4-dibromo-pyrazol-1-yl)propan-2-one (1.40 g, 16.5%) as a solid. LCMS: (ESI, m/z): 324.9 [M+H]⁺. ¹H NMR (400 MHz, CHLOROFORM-d) δ ppm 5.30 (s, 2H) 2.71 (s, 3H) 2.25 (s, 3H).

### Synthesis of C103

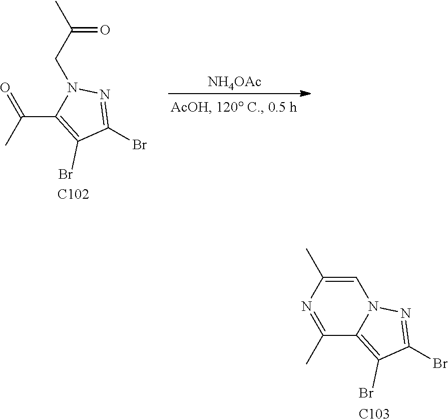

To a solution of 1-(5-acetyl-3,4-dibromo-pyrazol-1-yl)propan-2-one (0.700 g, 2.16 mmol, 1.00 eq) in acetic acid (2.90 mL) was added ammonium acetate (1.67 g, 21.6 mmol, 10.0 eq). The reaction mixture was stirred at 120° C. for 0.5 hr. The reaction mixture was diluted with water (20.0 mL), extracted with ethyl acetate (3×20.0 mL), washed with brine (4×50.0 mL), dried over Na₂SO₄, filtered and concentrated under reduced pressure to give an oil. The oil was purified by reversed-phase HPLC (column: C18 (250*50 mm*10 um), mobile phase: [water (NH₄HCO₃)-acetonitrile]; B %: 40%-80%, 10 min) to yield 2,3-dibromo-4,6-dimethyl-pyrazolo [1,5-a]pyrazine (300 mg, 16.9%) as a solid. LCMS: (ESI, m/z): 305.9 [M+H]⁺. ¹H NMR (400 MHz, DMSO-d6) δ ppm 8.53 (s, 1H) 2.85 (s, 3H) 2.39 (s, 3H).

### Synthesis of Intermediate C104

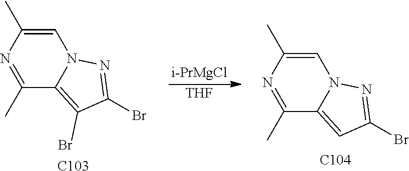

To a solution of 2,3-dibromo-4,6-dimethyl-pyrazolo[1,5-a]pyrazine (300 mg, 327 umol, 1.00 eq) in tetrahydrofuran (2.94 mL) was added isopropylmagnesium chloride (2.00 M, 655 μL, 4.00 eq) at 0° C. The reaction mixture was stirred at 0° C. for 0.5 hr. The reaction mixture was quenched by additional of methanol (20.0 mL). The reaction mixture was diluted with water (50.0 mL), extracted with ethyl acetate (3×40.0 mL), washed with brine (2×120 mL), dried over Na₂SO₄, filtered and concentrated under reduced pressure to give an oil. The oil was purified by column chromatography on silica gel eluted with petroleum ether/ethyl acetate (1/0 to 5/1) to give 2-bromo-4,6-dimethyl-pyrazolo[1,5-a]pyrazine (100 mg, 44.2%) as a solid. LCMS: (ESI, m/z): 225.8 [M+H]⁺. ¹H NMR (400 MHz, METHANOL-d₄) δ ppm 8.24 (s, 1H) 6.99 (s, 1H) 2.69 (s, 3H) 2.46 (d, J=0.63 Hz, 3H).

### Synthesis of Intermediate C105

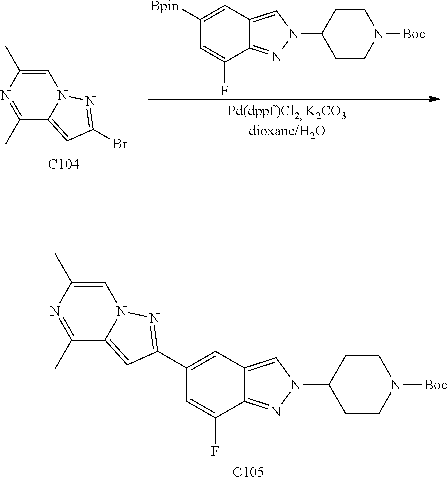

To a solution of 2-bromo-4,6-dimethyl-pyrazolo[1,5-a]pyrazine (40.0 mg, 176 umol, 1.00 eq) and tert-butyl 4-[7-fluoro-5-(4,4,5,5-tetramethyl-1,3,2-dioxaborolan-2-yl)indazol-2-yl]piperidine-1-carboxylate (118 mg, 265 umol, 1.50 eq) in dioxane (0.320 mL) was added a solution of K₂CO₃ (48.9 mg, 353 umol, 2.00 eq) in H₂O (0.080 mL) and 1,1 [symbol] bis(diphenylphosphino)ferrocenedichloro palladium(II) (12.9 mg, 17.6 umol, 0.100 eq) successively. The reaction was stirred at 80° C. for 1 hr. The reaction mixture was diluted with water (10.0 mL), extracted with ethyl acetate (3×10.0 mL), dried over Na₂SO₄, filtered and concentrated under reduced pressure to give oil. The oil was purified by column chromatography on silica gel eluted with ethyl acetate/ethanol (1/0 to 5/1) to give product tert-butyl 4-[5-(4,6-dimethylpyrazolo[1,5-a]pyrazin-2-yl)-7-fluoro-indazol-2-yl]piperidine-1-carboxylate (40.0 mg, 20.9%) as a solid. LCMS: (ESI, m/z): 465.3 [M+H]⁺. ¹H NMR (400 MHz, METHANOL-d₄) 6=8.49 (d, J=2.8 Hz, 1H), 8.28 (s, 1H), 8.16 (d, J=0.8 Hz, 1H), 7.66 (dd, J=0.9, 12.8 Hz, 1H), 7.30 (s, 1H), 4.73 (tt, J=4.1, 11.4 Hz, 1H), 4.35-4.26 (m, 2H), 3.04 (br d, J=1.3 Hz, 2H), 2.76 (s, 3H), 2.48 (s, 3H), 2.28-2.21 (m, 2H), 2.19-2.07 (m, 2H), 1.51 (s, 9H).

### Synthesis of Compound 124

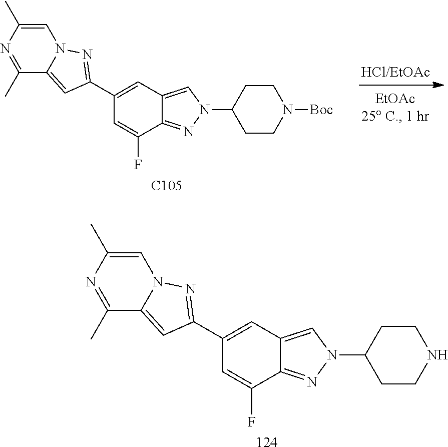

To a solution of tert-butyl 4-[5-(4,6-dimethylpyrazolo[1,5-a]pyrazin-2-yl)-7-fluoro-indazol-2-yl]piperidine-1-carboxylate (30.0 mg, 64.5 umol, 1.00 eq) in ethyl acetate (0.500 mL) was added HCl/ethyl acetate (2.00 M, 807 μL, 25.0 eq). The reaction mixture was stirred at 25° C. for 1 hr. The reaction mixture was filtered and the filter cake was purified by prep-HPLC (column: Phenomenex Luna 80*30 mm*3 um; mobile phase: [water (HCl)-acetonitrile]; B %: 1%-45%, 8 min) to give 2-[7-fluoro-2-(4-piperidyl) indazol-5-yl]-4,6-dimethyl-pyrazolo[1,5-a]pyrazine (22.7 mg, 92.7%) as a solid. LCMS: (ESI, m/z): 365.1 [M+H]⁺. ¹H NMR (400 MHz, D₂O) δ=8.39 (br s, 1H), 7.88 (br s, 1H), 7.58 (s, 1H), 7.05 (s, 1H), 6.96 (br d, J=12.4 Hz, 1H), 4.88 (br s, 1H), 3.69 (br d, J=12.6 Hz, 2H), 3.30 (br t, J=12.8 Hz, 2H), 2.56-2.45 (m, 5H), 2.44-2.31 (m, 2H), 2.09 (s, 3H).

### Example 59: Synthesis of Compound 149

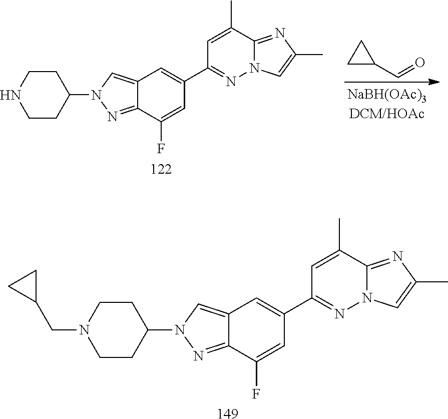

To a solution of 6-[7-fluoro-2-(4-piperidyl)indazol-5-yl]-2,8-dimethyl-imidazo[1,2-b]pyridazine (100 mg, 274 umol, 1 eq) in DCM (1.00 mL) was added AcOH (24.7 mg, 411 umol, 23.5 uL, 1.5 eq), cyclopropanecarbaldehyde (28.8 mg, 411 umol, 30.7 uL, 1.5 eq) and sodium;triacetoxyboranuide (116 mg, 548 umol, 2 eq) under N₂ atmosphere. The mixture was stirred at 25° C. for 2 hrs. TLC (ethyl acetate, Rf=0.55) showed the starting material was consumed and a new major spot was generated. The reaction mixture was quenched by addition of aq NaHCO₃ (10.0 mL) at 0° C., and then extracted with ethyl acetate (3×10.0 mL). The combined organic layers were washed with brine (10.0 mL), dried over Na₂SO₄, filtered and concentrated under reduced pressure to give a residue. The residue was purified by column chromatography (SiO₂, petroleum ether/ethyl acetate=5/1 to 10/1) to give 6-[2-[1-(cyclopropylmethyl)-4-piperidyl]-7-fluoro-indazol-5-yl]-2,8-dimethyl-imidazo[1,2-b]pyridazine (43.9 mg, 38.2%) as a solid. LCMS: (ESI, m/z): 419 [M+H]^{+ 1}H NMR (DMSO-d₆, 400 MHz) δ ppm 8.74 (d, 1H, J=2.8 Hz), 8.29 (s, 1H), 8.03 (s, 1H), 7.63 (d, 1H, J=18.8 Hz), 7.58 (s, 1H), 4.5-4.6 (m, 1H), 3.1-3.2 (m, 2H), 2.60 (s, 3H), 2.40 (s, 3H), 2.10-2.29 (m, 8H), 0.81-1.06 (m, 1H), 0.41-0.62 (m, 2H), 0.10 (q, 2H, J=4.8 Hz). ¹⁹F NMR (DMSO-d₆, 376 MHz) δ ppm −130.567 (s, 1F).

### Example 60: Synthesis of Compound 140

### Synthesis of Intermediate C106

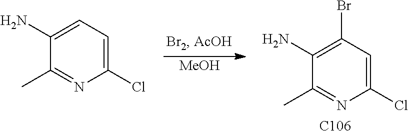

A mixture of 6-chloro-2-methyl-pyridin-3-amine (25.0 g, 175 mmol, 1.00 eq) and acetic acid (21.0 g, 349 mmol, 20.0 mL, 2.00 eq) in methanol (250 mL) was added Br₂ (50.4 g, 316 mmol, 1.80 eq) under N₂ protection at 0° C. The reaction mixture was stirred at 25° C. for 16 hrs. The reaction mixture was quenched by saturated aqueous Na₂S₂O₃ (500 mL) and saturated aqueous NaHCO₃ (500 mL), extracted with ethyl acetate (3×500 mL), washed with brine (600 mL), dried over with Na₂SO₄, filtered and concentrated under reduced pressure to give an oil. The oil was purified by chromatography on a silica gel eluted with ethyl acetate/petroleum ether=0/1 to 3/1 to give 4-bromo-6-chloro-2-methyl-pyridin-3-amine (16.0 g, 37.0%) as a solid. LCMS: (ESI, m/z): 220.9 [M+H]⁺. ¹H NMR (400 MHz, DMSO-d6) δ ppm 7.42 (s, 1H) 5.43 (br s, 2H) 2.33 (s, 3H)

### Synthesis of Intermediate C107

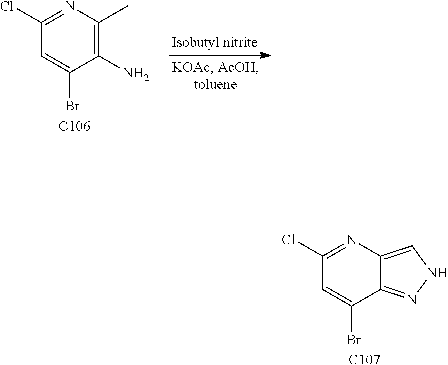

To a mixture of 4-bromo-6-chloro-2-methyl-pyridin-3-amine (14.0 g, 63.2 mmol, 1.00 eq), acetic acid (123 g, 2.05 mol, 32.5 eq) and isopentyl nitrite (11.1 g, 94.8 mmol, 12.8 mL, 1.50 eq) in toluene (350 mL) was added potassium acetate (12.4 g, 126 mmol, 2.00 eq) under N₂ protection at 0° C. The reaction mixture was stirred at 30° C. for 4 hrs. The mixture was concentrated under reduced pressure to remove solvent and saturated sodium bicarbonate solution (100 mL) was added to quench the reaction. The reaction mixture was extracted with ethyl acetate (3×200 mL), washed with brine (400 mL), dried over Na₂SO₄, filtered and the filtrate was concentrated under reduced pressure to give black solid. The black solid was purified by chromatography on a silica gel eluted with ethyl acetate/petroleum ether=0/1 to 1/2 to give 7-bromo-5-chloro-2H-pyrazolo[4,3-b]pyridine (4.50 g, 27.5%) as a solid. LCMS: (ESI, m/z): 231.9 [M+H]⁺. ¹H NMR (400 MHz, DMSO-d6) δ ppm 14.15 (br s, 1H) 8.41 (br s, 1H) 7.87 (br s, 1H)

### Synthesis of Intermediate C108

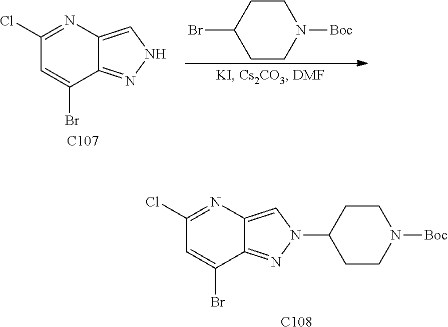 [SMILES: Clc1cc(Br)c2n[nH]cc2n1 ; CC(C)(C)OC(=O)N1CCC(Br)CC1 ; "C107" ; CC(C)(C)OC(=O)N1CCC(n2cc3nc(Cl)cc(Br)c3n2)CC1 ; "C108"]

A mixture of 7-bromo-5-chloro-2H-pyrazolo[4,3-b]pyridine (3.00 g, 12.9 mmol, 1.00 eq) in N,N-dimethylformamide (60.0 mL) was added KI (7.50 g, 45.1 mmol, 3.50 eq), tert-butyl 4-bromopiperidine-1-carboxylate (11.9 g, 45.1 mmol, 3.50 eq) and Cs₂CO₃ (21.0 g, 64.5 mmol, 5.00 eq). The reaction mixture was stirred at 100° C. for 2 hrs. The reaction mixture was diluted with water (80.0 mL), extracted with ethyl acetate (3×80.0 mL), washed with brine (4×200 mL), dried over Na₂SO₄, filtered and concentrated under reduced pressure to give oil. The oil was purified by column chromatography on silica gel eluted with petroleum ether/ethyl acetate (1/0 to 5/1) to give tert-butyl 4-(7-bromo-5-chloro-pyrazolo[4,3-b]pyridin-2-yl)piperidine-1-carboxylate (1.51 g, 22.5%) as a solid. LCMS: (ESI, m/z): 415.1 [M+H]⁺.

### Synthesis of Intermediate C109

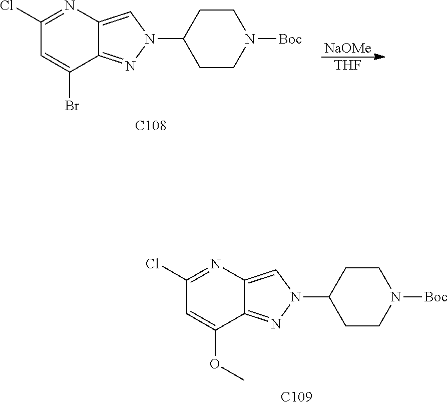 [SMILES: CC(C)(C)OC(=O)N1CCC(n2cc3nc(Cl)cc(Br)c3n2)CC1 ; "C108" ; COc1cc(Cl)nc2cn(C3CCN(C(=O)OC(C)(C)C)CC3)nc12 ; "C109" ; "NaOMe" ; "THF"]

To a solution of tert-butyl 4-(7-bromo-5-chloro-pyrazolo[4,3-b]pyridin-2-yl) piperidine-1-carboxylate (235 mg, 565 umol, 1.00 eq) in tetrahydrofuran (3.00 mL) was added sodium methanolate (91.6 mg, 1.70 mmol, 3.00 eq). The reaction mixture was stirred at 60° C. for 3.5 hrs. The reaction mixture was concentrated in vacuum to give oil. The oil was purified by column chromatography on silica gel eluted with petroleum ether/ethyl acetate (5/1 to 0/1) to give tert-butyl 4-(5-chloro-7-methoxy-pyrazolo[4,3-b]pyridin-2-yl)piperidine-1-carboxylate (150 mg, 62.9%) as a solid. LCMS: (ESI, m/z): 367.2 [M+H]⁺. ¹H NMR (400 MHz, CHLOROFORM-d) δ ppm 8.05 (s, 1H) 6.56 (s, 1H) 4.56 (tt, J=11.56, 4.17 Hz, 1H) 4.23-4.44 (m, 2H) 4.09 (s, 3H) 2.92 (br t, J=10.91 Hz, 2H) 2.20-2.27 (m, 2H) 2.06-2.18 (m, 2H) 1.49 (s, 9H).

### Synthesis of Intermediate C110

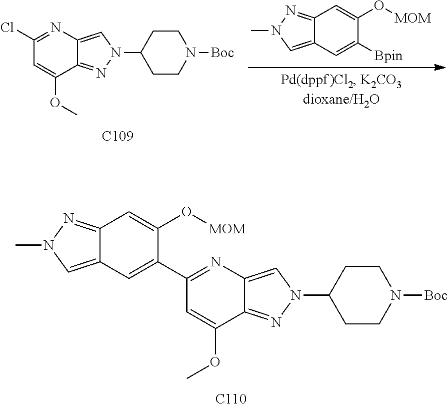

To a mixture of tert-butyl 4-(5-chloro-7-methoxy-pyrazolo[4,3-b]pyridin-2-yl) piperidine-1-carboxylate (50.0 mg, 136 umol, 1.00 eq) and 6-(methoxymethoxy)-2-methyl-5-(4,4,5,5-tetramethyl-1,3,2-dioxaborolan-2-yl)indazole (65.0 mg, 204 umol, 1.50 eq) in dioxane (0.400 mL) was added a solution of K₂CO₃ (EVAL-0114-A8, 37.6 mg, 272 umol, 2.00 eq) in H₂O (0.100 mL). Then, 1,1 [symbol] bis(diphenylphosphino) ferrocenedichloro palladium(II) (9.97 mg, 13.6 umol, 0.100 eq) was added to the above mixture under N₂ protection. The reaction mixture was stirred at 60° C. for 1 hr. The reaction mixture was quenched by additional of H₂O (10.0 mL), extracted with ethyl acetate (3×10.0 mL), dried over Na₂SO₄, filtered and concentrated under reduced pressure to give yellow oil. The oil was purified by column chromatography on silica gel eluted with petroleum ether/ethyl acetate=10/1 to 1/1 to give tert-butyl 4-[7-methoxy-5-[6-(methoxymethoxy)-2-methyl-indazol-5-yl]pyrazolo[4,3-b]pyridin-2-yl]piperidine-1-carboxylate (55.0 mg, 38.6%) as a solid. LCMS: (ESI, m/z): 523.3 [M+H]⁺.

### Synthesis of Compound 140

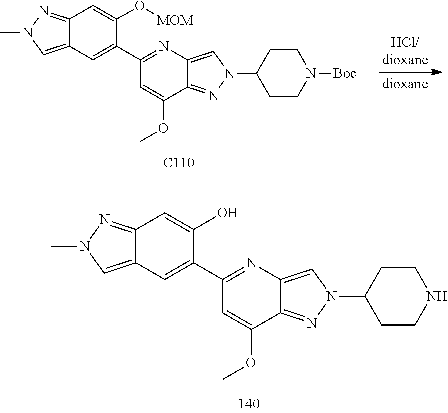

To a solution of tert-butyl 4-[7-methoxy-5-[6-(methoxymethoxy)-2-methyl-indazol-5-yl]pyrazolo[4,3-b]pyridin-2-yl]piperidine-1-carboxylate (50.0 mg, 95.6 umol, 1.00 eq) in dioxane (1.00 mL) was added HCl/dioxane (4.00 M, 956 μL, 40.0 eq). The reaction mixture was stirred at 30° C. for 1 hr. The reaction mixture was filtered and the filter cake was purified by prep-HPLC (column: Phenomenex Luna 80*30 mm*3 um; mobile phase: [water (HCl)-acetonitrile]; B %: 1%-30%, 8 min) to give 5-[7-methoxy-2-(4-piperidyl)pyrazolo[4,3-b]pyridin-5-yl]-2-methyl-indazol-6-ol (14.0 mg, 37.5%) as a solid. LCMS: (ESI, m/z): 379.1 [M+H]⁺. ¹H NMR (400 MHz, D₂O) 6=8.56 (s, 1H), 8.19 (s, 1H), 8.05 (s, 1H), 7.12 (s, 1H), 6.84 (s, 1H), 4.98 (br t, J=11.3 Hz, 1H), 4.22 (s, 3H), 4.06 (s, 3H), 3.68 (br d, J=13.2 Hz, 2H), 3.30 (br t, J=12.4 Hz, 2H), 2.58-2.48 (m, 2H), 2.46-2.34 (m, 2H).

### Example 61: Synthesis of Compound 150

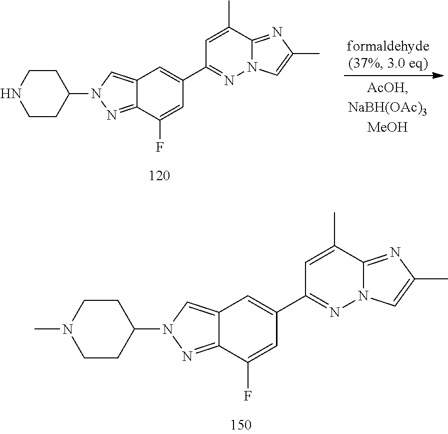

To a solution of 6-[7-fluoro-2-(4-piperidyl)indazol-5-yl]-2,8-dimethyl-imidazo[1,2-b]pyridazine (100 mg, 274 umol, 1 eq) in MeOH (1.00 mL) was added formaldehyde (66.8 mg, 823 umol, 61.2 uL, 37 wt %, 3 eq), AcOH (49.4 mg, 823 umol, 47.0 uL, 3 eq) and sodium;triacetoxyboranuide (116 mg, 548 umol, 2 eq) under N₂ atmosphere. The mixture was stirred at 25° C. for 2 hrs. TLC (ethyl acetate, Rf=0.55) showed the starting material was consumed and a new major spot was generated. The reaction mixture was quenched by addition of aq NaHCO₃ (10.0 mL) at 0° C., and then diluted with ethyl acetate (10.0 mL) and extracted with ethyl acetate (3×10.0 mL). The combined organic layers were washed with brine (10.0 mL), dried over Na₂SO₄, filtered and concentrated under reduced pressure to give a residue. The residue was purified by column chromatography (SiO₂, petroleum ether/ethyl acetate=5/1 to 10/1) to give 6-[7-fluoro-2-(1-methyl-4-piperidyl)indazol-5-yl]-2,8-dimethyl-imidazo[1,2-b]pyridazine (42.1 mg, 40.6%) as a solid. LCMS: (ESI, m/z): 379.3 [M+H]^{+ 1}H NMR (METHANOL-d₄, 400 MHz) δ ppm 8.53 (d, 1H, J=2.7 Hz), 8.20 (d, 1H, J=1.2 Hz), 7.89 (s, 1H), 7.75 (dd, 1H, J=1.2, 13.1 Hz), 7.63 (d, 1H, J=1.0 Hz), 4.4-4.7 (m, 1H), 3.0-3.1 (m, 2H), 2.66 (d, 3H, J=1.0 Hz), 2.48 (s, 3H), 2.38 (s, 3H), 2.2-2.4 (m, 6H) ¹⁹F NMR (METHANOL-d₄, 376 MHz) δ ppm −130.586 (s, 1F)

### Example 62: Synthesis of Compound 151

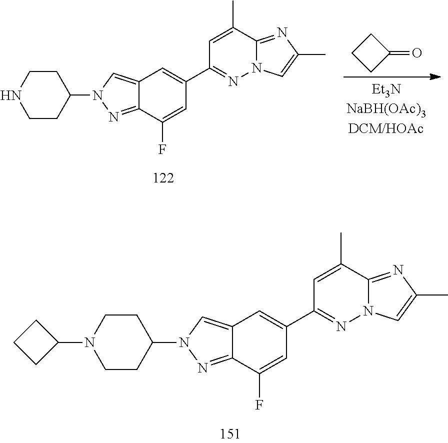

To a solution of 6-[7-fluoro-2-(4-piperidyl)indazol-5-yl]-2,8-dimethyl-imidazo[1,2-b]pyridazine (100 mg, 274 umol, 1 eq) in DCM (1.00 mL) was added N,N-diethylethanamine (27.7 mg, 274 umol, 38.1 uL, 1 eq), AcOH (32.9 mg, 548 umol, 31.3 uL, 2 eq), cyclobutanone (38.4 mg, 548 umol, 41.0 uL, 2 eq) and sodium;triacetoxyboranuide (116 mg, 548 umol, 2 eq) under N₂ atmosphere. The mixture was stirred at 25° C. for 2 hrs. TLC (ethyl acetate, Rf=0.55) showed the starting material was consumed and a new major spot was generated. The reaction mixture was quenched by addition of H₂O (10.0 mL) at 0° C., and then extracted with ethyl acetate (3×10.0 mL). The combined organic layers were washed with brine (10.0 mL), dried over Na₂SO₄, filtered and concentrated under reduced pressure to give a residue. The residue was purified by column chromatography (SiO₂, petroleum ether/ethyl acetate=5/1 to 10/1) to give 6-[2-(1-cyclobutyl-4-piperidyl)-7-fluoro-indazol-5-yl]-2, 8-dimethyl-imidazo [1,2-b] pyridazine (65.7 mg, 57.2%) as a solid. LCMS: (ESI, m/z): 419 [M+H]^{+ 1}H NMR (METHANOL-d₄, 400 MHz) δ ppm 8.51 (d, 1H, J=2.7 Hz), 8.19 (d, 1H, J=1.2 Hz), 7.89 (d, 1H, J=0.7 Hz), 7.74 (dd, 1H, J=1.3, 13.1 Hz), 7.62 (d, 1H, J=1.0 Hz), 4.51-4.64 (m, 1H), 3.09 (br d, 2H, J=12.0 Hz), 2.89 (quin, 1H, J=8.0 Hz), 2.66 (d, 3H, J=1.0 Hz), 2.48 (d, 3H, J=0.6 Hz), 2.15-2.38 (m, 8H), 1.92-2.05 (m, 2H), 1.74-1.86 (m, 2H), ¹⁹F NMR (METHANOL-d₄, 376 MHz) δ ppm −130.583 (s, 1F).

### Example 63: Synthesis of Compound 152

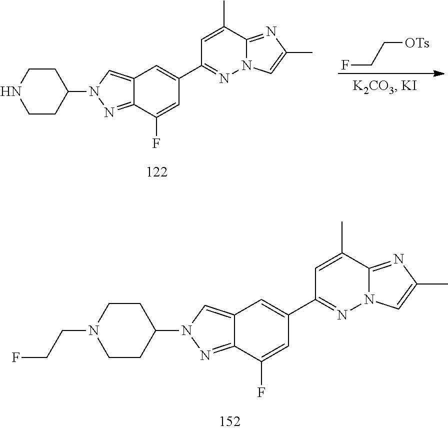

To a solution of 6-[7-fluoro-2-(4-piperidyl) indazol-5-yl]-2,8-dimethyl-imidazo[1,2-b]pyridazine (100 mg, 274 umol, 1 eq) in MeCN (1.00 mL) was added K₂CO₃ (113 mg, 823 umol, 3 eq) and 2-fluoroethyl 4-methylbenzenesulfonate (59.8 mg, 274 umol, 1 eq) under N₂ atmosphere. The mixture was stirred at 100° C. for 2 hrs. TLC (ethyl acetate, Rf=0.55) showed the starting material was consumed and a new major spot was generated. The reaction mixture was quenched by addition of H₂O (10.0 mL) at 0° C., and then extracted with ethyl acetate (3×10.0 mL). The combined organic layers were washed with brine (10.0 mL), dried over Na₂SO₄, filtered and the filtrate was concentrated under reduced pressure to give a residue. The residue was purified by column chromatography (SiO₂, petroleum ether/ethyl acetate=5/1 to 10/1) to give 6-[7-fluoro-2-[1-(2-fluoroethyl)-4-piperidyl]indazol-5-yl]-2,8-dimethyl-imidazo[1,2-b]pyridazine (25.2 mg, 22.3%) as a solid. LCMS: (ESI, m/z): 411.2 [M+H]^{+ 1}H NMR (METHANOL-d₄, 400 MHz) δ ppm 8.55 (d, 1H, J=2.7 Hz), 8.23 (d, 1H, J=1.2 Hz), 7.92 (d, 1H, J=0.7 Hz), 7.78 (dd, 1H, J=1.3, 13.1 Hz), 7.66 (d, 1H, J=1.0 Hz), 4.72-4.77 (m, 1H), 4.51-4.66 (m, 2H), 3.22 (br d, 2H, J=12.1 Hz), 2.81-2.96 (m, 1H), 2.78-2.80 (m, 1H), 2.50 (d, 3H, J=0.6 Hz), 2.40-2.52 (m, 2H), 2.31-2.45 (m, 4H) ¹⁹F NMR (METHANOL-d₄, 376 MHz) δ ppm −130.610 (s, 1F), −220.033 (s, 1F).

### Example 64: Synthesis of Compound 153

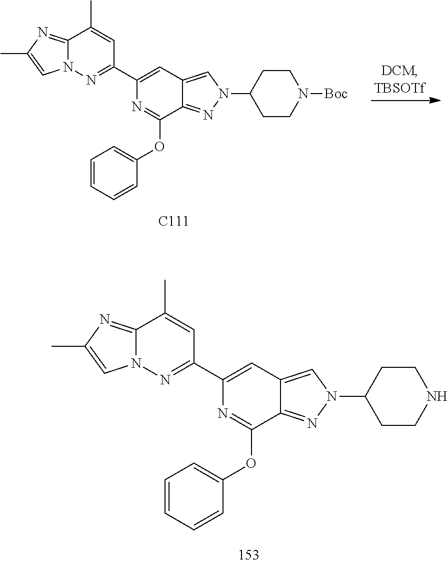 [SMILES: Cc1cn2nc(-c3cc4cn(C5CCN(C(=O)OC(C)(C)C)CC5)nc4c(Oc4ccccc4)n3)cc(C)c2n1 ; "C111" ; Cc1cn2nc(-c3cc4cn(C5CCNCC5)nc4c(Oc4ccccc4)n3)cc(C)c2n1]

To a solution of tert-butyl 4-[7-benzyloxy-5-(2,8-dimethylimidazo[1,2-b]pyridazin-6-yl)pyrazolo [3,4-c]pyridin-2-yl]piperidine-1-carboxylate (110 mg, 199 umol, 1.00 eq) in dichloromethane (2.20 mL) was added tert-butyldimethylsilyltrifluoromethanesulfonate (105 mg, 397 umol, 2.00 eq) at 0° C. The reaction mixture was stirred at 25° C. for 2 hrs. LCMS showed the reaction was completed. The mixture was quenched with saturated sodium bicarbonate solution (1.00 mL) and water (2.00 mL). Then the reaction mixture was extracted with dichloromethane (3×2.00 mL), washed with brine (3.00 mL), dried over Na₂SO₄, filtered and the filtrate was concentrated under reduced pressure to give the white solid. The white solid was purified by prep-HPLC (Column: Waters Xbridge BEH C18 100×30 mm×10 um; mobile phase: [water (NH₄HCO₃)-acetonitrile]; B %: 35%-65%, 8 min) to give 6-[7-benzyloxy-2-(4-piperidyl)pyrazolo [3,4-c]pyridin-5-yl]-2,8-dimethyl-imidazo[1,2-b]pyridazine (7.14 mg, 7.92%) as a solid. LCMS: (ESI, m/z): 454.3 [M+H]⁺. ¹H NMR (400 MHz, DMSO-d6) δ ppm 8.61 (s, 1H) 8.15 (s, 1H) 8.02 (s, 1H) 7.91 (d, J=0.98 Hz, 1H) 7.61 (d, J=7.09 Hz, 2H) 7.34-7.47 (m, 3H) 5.70 (s, 2H) 4.45-4.81 (m, 1H) 3.09 (br d, J=12.59 Hz, 2H) 2.59-2.68 (m, 6H) 2.41 (s, 3H) 1.89-2.12 (m, 4H)

### Example 65: Synthesis of Compound 160

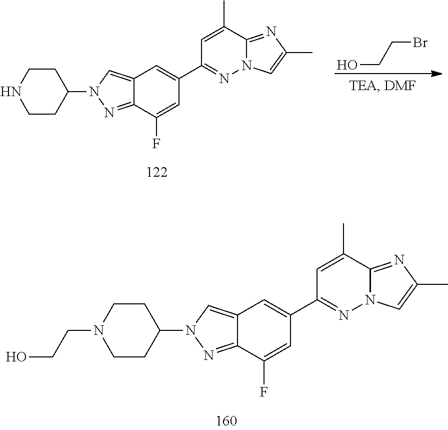

To a solution of 6-[7-fluoro-2-(4-piperidyl)indazol-5-yl]-2,8-dimethyl-imidazo[1,2-b]pyridazine (100 mg, 274 umol, 1 eq) in DMF (1.00 mL) was added 2-bromoethanol (34.2 mg, 274 umol, 19.4 uL, 1 eq) and TEA (138 mg, 1.37 mmol, 190 μL, 5 eq) under N₂ atmosphere. The mixture was stirred at 50° C. for 2 hrs. TLC (ethyl acetate, Rf=0.55) showed the starting material was consumed and a new major spot was generated. The reaction mixture was quenched by addition of water (10.0 mL) at 0° C., and then extracted with ethyl acetate (3×10.0 mL). The combined organic layers were washed with brine (10.0 mL), dried over Na₂SO₄, filtered and concentrated under reduced pressure to give a residue. The residue was purified by column chromatography (SiO₂, petroleum ether/ethyl acetate=5/1 to 10/1) to give 2-[4-[5-(2,8-dimethylimidazo[1,2-b]pyridazin-6-yl)-7-fluoro-indazol-2-yl]-1-piperidyl]ethanol (31.0 mg, 26.8%) as a solid. LCMS: (ESI, m/z): 409 [M+H]^{+ 1}H NMR (DMSO-d₆, 400 MHz) δ ppm 8.73 (d, 1H, J=2.7 Hz), 8.28 (s, 1H), 8.03 (s, 1H), 7.7-7.8 (m, 2H), 4.5-4.6 (m, 1H), 4.43 (br t, 1H, J=5.0 Hz), 3.54 (q, 2H, J=6.1 Hz), 3.04 (br d, 2H, J=9.0 Hz), 2.59 (s, 3H), 2.4-2.5 (m, 2H), 2.40 (s, 3H), 2.1-2.2 (m, 6H). ¹⁹F NMR (DMSO-d₆, 376 MHz) δ ppm −128.364 (s, 1F).

### Example 66: Synthesis of Compound 166

### Synthesis of Intermediate C113

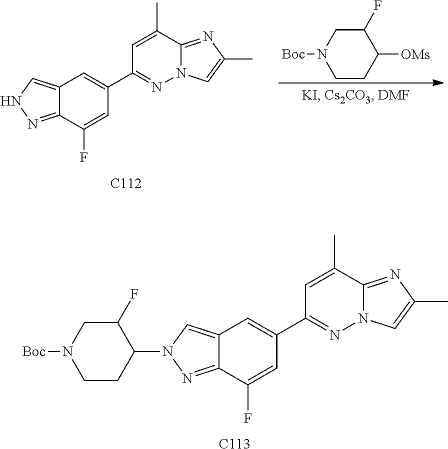

To a solution of 6-(7-fluoro-2H-indazol-5-yl)-2,8-dimethyl-imidazo[1,2-b]pyridazine (600 mg, 2.13 mmol, 1 eq) and tert-butyl 3-fluoro-4-methylsulfonyloxy-piperidine-1-carboxylate (C7, 1.90 g, 6.40 mmol, 3 eq) in DMF (1.00 mL) was added KI (1.06 g, 6.40 mmol, 3 eq) and Cs₂CO₃ (3.47 g, 10.6 mmol, 5 eq) under N₂ atmosphere. The mixture was stirred at 100° C. for 12 hrs. TLC (ethyl acetate, Rf=0.4, 0.6) showed the starting material was consumed and a new major spot was generated. The reaction mixture was cooled to 25° C., quenched by addition of water (40.0 mL) at 25° C., and then extracted with ethyl acetate (3×50.0 mL). The combined organic layers were dried over MgSO₄, filtered and concentrated under reduced pressure to give a residue, which was purified by silica gel column chromatography (petroleum ether/ethyl acetate=1:1) to give tert-butyl 4-[5-(2,8-dimethylimidazo[1,2-b]pyridazin-6-yl)-7-fluoro-indazol-2-yl]-3-fluoro-piperidine-1-carboxylate (150 mg, 14.5%) as a solid. LCMS: (ESI, m/z): 483.3 [M+H]^{+ 1}H NMR (CHLOROFORM-d, 400 MHz) δ ppm 8.18 (d, 1H, J=2.4 Hz), 8.01 (d, 1H, J=1.2 Hz), 7.77 (s, 1H), 7.70 (dd, 1H, J=1.2, 12.6 Hz), 7.3-7.3 (m, 1H), 4.9-5.2 (m, 1H), 4.60 (br s, 1H), 4.5-4.6 (m, 1H), 4.2-4.5 (m, 1H), 2.8-3.1 (m, 2H), 2.7-2.8 (m, 3H), 2.5-2.6 (m, 4H), 2.2-2.3 (m, 1H), 1.51 (s, 9H) ¹⁹F NMR (CHLOROFORM-d, 376 MHz) δ ppm −127.476 (s, 1F)

### Synthesis of Compound 166

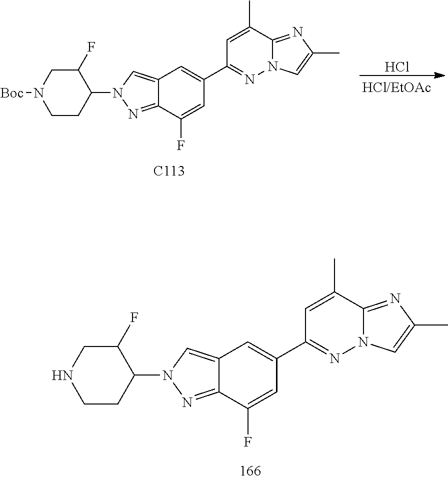 [SMILES: Cc1cn2nc(-c3cc(F)c4nn(C5CCN(C(=O)OC(C)(C)C)CC5F)cc4c3)cc(C)c2n1 ; Cl ; "C113" ; Cc1cn2nc(-c3cc(F)c4nn(C5CCNCC5F)cc4c3)cc(C)c2n1]

To a solution of tert-butyl 4-[5-(2,8-dimethylimidazo[1,2-b]pyridazin-6-yl)-7-fluoro-indazol-2-yl]-3-fluoro-piperidine-1-carboxylate (20.0 mg, 41.4 umol, 1 eq) in EtOAc (2.00 mL) was added HCl/EtOAc (4 M, 10.3 uL, 1 eq) under N₂ atmosphere. The mixture was stirred at 25° C. for 2 hrs. TLC (ethyl acetate, Rf=0.1) showed the starting material was consumed and a new major spot was generated. The reaction mixture was concentrated under reduced pressure to remove ethyl acetate. The crude product was purified by re-crystallization from MeOH (10.0 mL) at 25° C. to give 6-[7-fluoro-2-(3-fluoro-4-piperidyl) indazol-5-yl]-2, 8-dimethyl-imidazo [1, 2-b]pyridazine (4.54 mg, 27.3%) as a solid. LCMS: (ESI, m/z): 383.3 [M+H]^{+ 1}H NMR (METHANOL-d₄, 400 MHz) δ ppm 8.71 (d, 1H, J=2.6 Hz), 8.45 (d, 1H, J=1.3 Hz), 8.3-8.3 (m, 2H), 7.87 (dd, 1H, J=1.3, 12.9 Hz), 5.3-5.5 (m, 1H), 5.2-5.3 (m, 1H), 3.9-4.0 (m, 1H), 3.7-3.8 (m, 1H), 3.47 (td, 1H, J=8.3, 13.1 Hz), 3.36 (td, 1H, J=4.4, 9.1 Hz), 2.78 (d, 3H, J=1.0 Hz), 2.65 (d, 3H, J=0.8 Hz), 2.61 (br dd, 2H, J=4.2, 9.1 Hz) ¹⁹F NMR (METHANOL-d₄, 376 MHz) δ ppm −129.453 (s, 1F), −190.237 (s, 1F)

### Example 67: Synthesis of Compound 189

### Synthesis of Intermediate C114

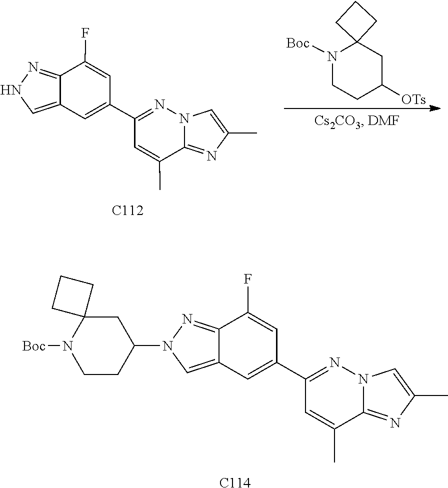 [SMILES: Cc1cn2nc(-c3cc(F)c4n[nH]cc4c3)cc(C)c2n1 ; Cc1ccc(S(=O)(=O)OC2CCN(C(=O)OC(C)(C)C)C3(CCC3)C2)cc1 ; "C112" ; Cc1cn2nc(-c3cc(F)c4nn(C5CCN(C(=O)OC(C)(C)C)C6(CCC6)C5)cc4c3)cc(C)c2n1 ; "C114"]

A mixture of 6-(7-fluoro-2H-indazol-5-yl)-2,8-dimethyl-imidazo[1,2-b]pyridazine (100 mg, 355 umol, 1.00 eq), tert-butyl 8-(p-tolylsulfonyloxy)-5-azaspiro[3.5]nonane-5-carboxylate (154 mg, 391 umol, 1.10 eq) and Cs₂CO₃ (347 mg, 1.07 mmol, 3.00 eq) in N,N-dimethylformamide (2.00 mL) was stirred at 80° C. for 12 hrs. LCMS showed the reaction was completed. The reaction mixture was diluted with ethyl acetate (3.00 mL), washed with water (3.00 mL×3). The organic layer was dried over Na₂SO₄, filtered and concentrated under reduced pressure to give crude product. The crude product was purified by column chromatography (SiO₂, petroleum ether/ethyl acetate=1/0 to 1/1) to give tert-butyl 8-[5-(2,8-dimethylimidazo[1,2-b]pyridazin-6-yl)-7-fluoro-indazol-2-yl]-5-azaspiro[3.5]nonane-5-carboxylate (35.0 mg, 6.50%) as an oil. LCMS: (ESI, m/z): 505.4 [M+H]⁺. ¹H NMR (400 MHz, CHLOROFORM-d) δ ppm 8.11 (d, J=2.51 Hz, 1H) 7.99 (d, J=1.00 Hz, 1H) 7.77 (s, 1H) 7.68 (dd, J=12.55, 1.13 Hz, 1H) 7.30 (s, 1H) 4.84-4.95 (m, 1H) 4.06-4.14 (m, 1H) 2.82-2.90 (m, 1H) 2.74-2.81 (m, 1H) 2.73 (s, 3H) 2.64 (dd, J=12.30, 3.51 Hz, 1H) 2.50-2.57 (m, 3H) 2.04-2.29 (m, 5H) 1.87-2.00 (m, 1H) 1.73-1.85 (m, 2H) 1.50 (s, 9H)

### Synthesis of Compound 189

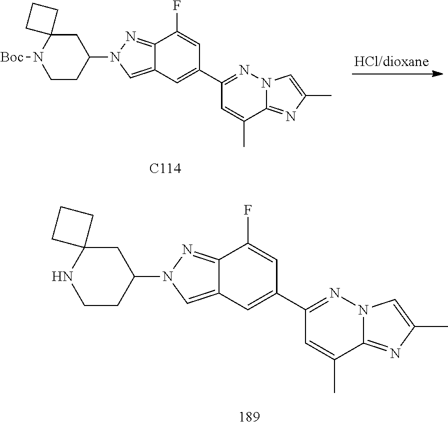 [SMILES: Cc1cn2nc(-c3cc(F)c4nn(C5CCN(C(=O)OC(C)(C)C)C6(CCC6)C5)cc4c3)cc(C)c2n1 ; "C114" ; Cc1cn2nc(-c3cc(F)c4nn(C5CCNC6(CCC6)C5)cc4c3)cc(C)c2n1]

To a solution of tert-butyl 8-[5-(2,8-dimethylimidazo[1,2-b]pyridazin-6-yl)-7-fluoro-indazol-2-yl]-5-azaspiro[3.5]nonane-5-carboxylate (30.0 mg, 59.4 umol, 1.00 eq) in 1,4-dioxane (0.500 mL) was added HCl/dioxane (4.00 M, 0.500 mL, 33.6 eq). The reaction mixture was stirred at 25° C. for 0.5 hrs. LCMS showed the reaction was completed. The reaction mixture was concentrated under reduced pressure to give a residue. The residue was purified by prep-HPLC (column: Waters Xbridge BEH C18 100*30 mm*10 um; mobile phase: [water (HCl)-acetonitrile]; B %: 1%-30%, 8 min) to give 6-[2-(5-azaspiro[3.5]nonan-8-yl)-7-fluoro-indazol-5-yl]-2,8-dimethyl-imidazo[1,2-b]pyridazine (18.9 mg, 78.9%) as a solid. LCMS: (ESI, m/z): 405.2 [M+H]⁺. ¹H NMR (400 MHz, METHANOL-d4) δ ppm 8.70 (d, J=2.63 Hz, 1H) 8.44 (d, J=1.25 Hz, 1H) 8.30 (dd, J=7.03, 1.00 Hz, 2H) 7.85 (dd, J=12.80, 1.25 Hz, 1H) 4.96-5.05 (m, 1H) 3.54 (dt, J=13.05, 3.45 Hz, 1H) 3.21-3.29 (m, 1H) 2.73-2.84 (m, 4H) 2.65 (d, J=0.88 Hz, 3H) 2.38-2.54 (m, 6H) 2.16-2.25 (m, 1H) 2.04-2.14 (m, 2H)

### Example 68: Synthesis of Compound 208

### Synthesis of Intermediate C116

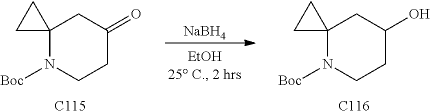 [SMILES: CC(C)(C)OC(=O)N1CCC(=O)CC12CC2 ; "NaBH4" ; CC(C)(C)OC(=O)N1CCC(O)CC12CC2 ; "C115" ; "C116"]

To a mixture of tert-butyl 7-oxo-4-azaspiro[2.5]octane-4-carboxylate (5.00 g, 22.1 mmol, 1.00 eq) in ethanol (50.0 mL) was added NaBH₄ (1.50 g, 39.7 mmol, 1.79 eq) at 0° C. under N₂ protection. The reaction mixture was stirred at 25° C. for 2 hrs. The reaction was quenched with water (150 mL) slowly and extracted with dichloromethane (3×150 mL). The combined organic phases were washed with brine (150 mL), dried over anhydrous Na₂SO₄, filtered and concentrated in vacuo to give tert-butyl 7-hydroxy-4-azaspiro[2.5]octane-4-carboxylate (5.00 g, 89.2%) as an oil. The oil was used to next step without further purification. LCMS: (ESI, m/z): 172.3 [M−55]⁺. ¹H NMR (400 MHz, DMSO-d6) δ ppm 4.66 (d, J=4.88 Hz, 1H) 3.64-3.81 (m, 2H) 2.81 (br t, J=11.82 Hz, 1H) 1.70-1.79 (m, 1H) 1.55 (br t, J=11.07 Hz, 1H) 1.39 (s, 9H) 1.15-1.26 (m, 2H) 0.99 (dt, J=10.04, 6.24 Hz, 1H) 0.74 (dt, J=9.32, 6.28 Hz, 1H) 0.36-0.51 (m, 2H)

### Synthesis of Intermediate C117

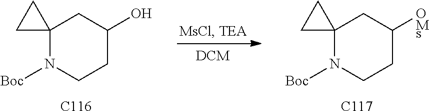 [SMILES: CC(C)(C)OC(=O)N1CCC(O)CC12CC2 ; CC(C)(C)OC(=O)N1CCC(OS(C)(=O)=O)CC12CC2 ; "C116" ; "C117"]

To a solution of tert-butyl 7-hydroxy-4-azaspiro[2.5]octane-4-carboxylate (2.50 g, 11.0 mmol, 1.00 eq) and triethylamine (2.23 g, 22.0 mmol, 3.06 mL, 2.00 eq) in dichloromethane (50.0 mL) was added methanesulfonyl chloride (2.52 g, 22.0 mmol, 2.00 eq) at 0° C. under N₂ protection. The reaction mixture was stirred at 25° C. for 3 hrs. The reaction mixture was quenched with water (100 mL), extracted with ethyl acetate (3×100 mL), dried over Na₂SO₄, filtered and the filtrate was concentrated under reduced pressure to give tert-butyl 7-methylsulfonyloxy-4-azaspiro[2.5]octane-4-carboxylate (3.00 g, 71.4%) as an oil. The oil was used to next step without further purification. LCMS: (ESI, m/z): 250.2 [M−55]⁺. ¹H NMR (400 MHz, DMSO-d6) δ ppm 4.88 (tt, J=8.90, 4.49 Hz, 1H) 3.66-3.76 (m, 1H) 3.19 (s, 3H) 3.03-3.15 (m, 1H) 1.94-2.00 (m, 1H) 1.71-1.82 (m, 1H) 1.61-1.69 (m, 1H) 1.54 (dtd, J=13.26, 9.44, 9.44, 4.13 Hz, 1H) 1.41 (s, 9H) 0.92-1.04 (m, 1H) 0.74-0.86 (m, 1H) 0.55-0.68 (m, 2H)

### Synthesis of Intermediate C119

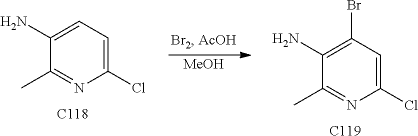

To a mixture of 6-chloro-2-methyl-pyridin-3-amine (5.00 g, 35.0 mmol, 1.00 eq), acetic acid (4.21 g, 70.1 mmol, 2.00 eq) in methanol (50.0 mL) was added Br₂ (10.0 g, 63.1 mmol, 1.80 eq) at 0° C. The reaction was stirred at 25° C. for 16 hrs. The reaction mixture was quenched with water (100 mL), extracted with ethyl acetate (3×100 mL), dried over Na₂SO₄, filtered and the filtrate was concentrated under reduced pressure to give residue. The residue was purified by column chromatography (petroleum ether/ethyl acetate=3/1) to give 4-bromo-6-chloro-2-methyl-pyridin-3-amine (6.00 g, 77.2%) as a solid. LCMS: (ESI, m/z): 223.2 [M+H]⁺. ¹H NMR (400 MHz, DMSO-d6) δ ppm 7.39 (s, 1H), 5.54-4.95 (m, 2H), 2.33 (s, 3H)

### Synthesis of C120

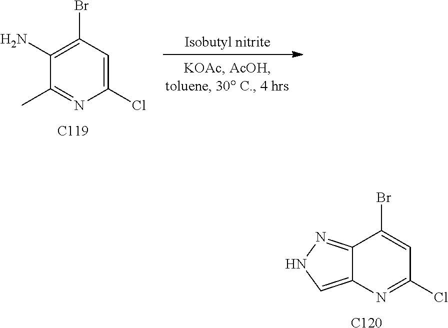

To a mixture of 4-bromo-6-chloro-2-methyl-pyridin-3-amine (3.00 g, 13.5 mmol, 1.00 eq), potassium acetate (2.66 g, 27.0 mmol, 2.00 eq), acetic acid (26.4 g, 440 mmol, 32.5 eq) in toluene (75.0 mL) was added isopentyl nitrite (2.38 g, 20.3 mmol, 1.50 eq) under nitrogen protection at 0° C. The reaction mixture was stirred at 30° C. for 4 hrs. The reaction mixture was quenched with water (100 mL), extracted with ethyl acetate (3×100 mL), dried over Na₂SO₄, filtered and the filtrate was concentrated under reduced pressure to give residue. The residue was purified by column chromatography (petroleum ether/ethyl acetate=3/1) to give 7-bromo-5-chloro-2H-pyrazolo[4,3-b]pyridine (2.00 g, 31.7%) as a solid. LCMS: (ESI, m/z): 233.9 [M+H]⁺. ¹H NMR (400 MHz, DMSO-d6) δ ppm 14.31-13.96 (m, 1H), 8.53-8.37 (m, 1H), 7.87 (s, 1H)

### Synthesis of Intermediate C121

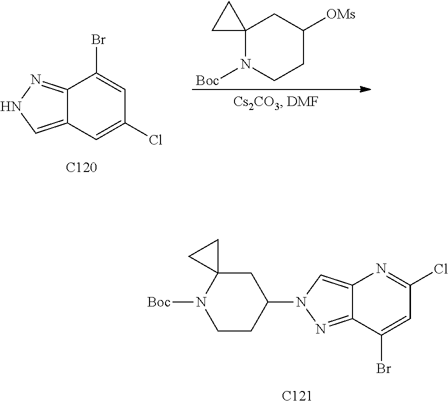 [SMILES: Clc1cc(Br)c2n[nH]cc2c1 ; CC(C)(C)OC(=O)N1CCC(OS(C)(=O)=O)CC12CC2 ; "C120" ; CC(C)(C)OC(=O)N1CCC(n2cc3nc(Cl)cc(Br)c3n2)CC12CC2 ; "C121"]

To a solution of 7-bromo-5-chloro-2H-pyrazolo[4,3-b]pyridine (500 mg, 2.15 mmol, 1.0 eq) and tert-butyl 7-methylsulfonyloxy-4-azaspiro[2.5]octane-4-carboxylate (C3, 788 mg, 2.58 mmol, 1.20 eq) in N,N-dimethylformamide (5.00 mL) was added Cs₂CO₃ (1.40 g, 4.30 mmol, 2.0 eq). The reaction mixture was stirred at 100° C. for 2 hrs. The reaction mixture was diluted with H₂O (20.0 mL), extracted with ethyl acetate (3×20.0 mL), washed with brine (3×20.0 mL), dried over Na₂SO₄, filtered and concentrated under reduced pressure to give brown oil. The brown oil was purified by column chromatography on silica gel eluted with petroleum ether/ethyl acetate (1/0 to 15/1) to give tert-butyl 7-(7-bromo-5-chloro-pyrazolo[4,3-b]pyridin-2-yl)-4-azaspiro[2.5]octane-4-carboxylate (70.0 mg, 7%) as an oil. LCMS: (ESI, m/z): 441.1 [M+H]⁺. ¹H NMR (400 MHz, METHANOL-d4) δ ppm 8.60 (s, 1H) 7.63 (s, 1H) 4.94-5.04 (m, 1H) 4.16-4.24 (m, 1H) 3.10-3.22 (m, 1H) 2.54-2.63 (m, 1H) 2.09-2.26 (m, 2H) 1.58 (ddd, J=12.69, 4.25, 1.19 Hz, 1H) 1.52 (s, 9H) 1.30-1.36 (m, 1H) 0.97 (dt, J=9.63, 6.44 Hz, 1H) 0.64-0.79 (m, 2H)

### Synthesis of Intermediate C122

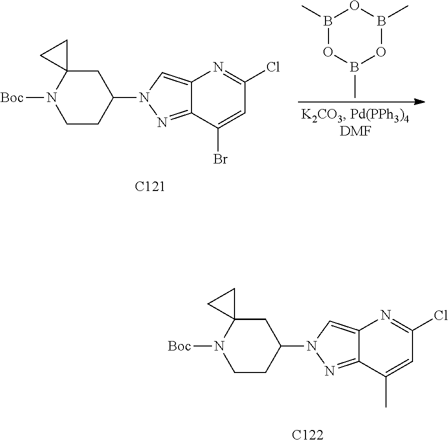

To a solution of tert-butyl 7-(7-bromo-5-chloro-pyrazolo[4,3-b]pyridin-2-yl)-4-azaspiro[2.5]octane-4-carboxylate (100 mg, 226 umol, 1.00 eq) and 2,4,6-trimethyl-1,3,5,2,4,6-trioxatriborinane (85.2 mg, 339 umol, 1.50 eq) in N,N-dimethylformamide (1.00 mL) was added K₂CO₃ (125 mg, 905 umol, 4.00 eq) and tetrakis(triphenylphosphine)palladium(0) (26.1 mg, 22.6 umol, 0.100 eq). The reaction mixture was stirred at 80° C. for 5 hrs. The reaction mixture was diluted with H₂O (5.00 mL), extracted with ethyl acetate (3×5.00 mL), washed with brine (3×5.00 mL), dried over Na₂SO₄, filtered and concentrated under reduced pressure to give an oil. The yellow oil was purified by column chromatography on silica gel eluted with petroleum ether/ethyl acetate (1/0 to 10/1) to give tert-butyl 7-(5-chloro-7-methyl-pyrazolo[4,3-b]pyridin-2-yl)-4-azaspiro[2.5]octane-4-carboxylate (60.0 mg, 56.2%) as a solid. LCMS: (ESI, m/z): 377.2 [M+H]⁺. ¹H NMR (400 MHz, METHANOL-d4) δ ppm 8.41 (s, 1H) 7.12 (d, J=1.00 Hz, 1H) 4.90-4.99 (m, 1H) 4.15-4.23 (m, 1H) 3.10-3.22 (m, 1H) 2.54-2.64 (m, 4H) 2.16-2.21 (m, 2H) 1.51 (s, 10H) 1.28-1.36 (m, 2H) 0.96 (dt, J=9.57, 6.54 Hz, 1H) 0.63-0.77 (m, 2H)

### Synthesis of Intermediate C123

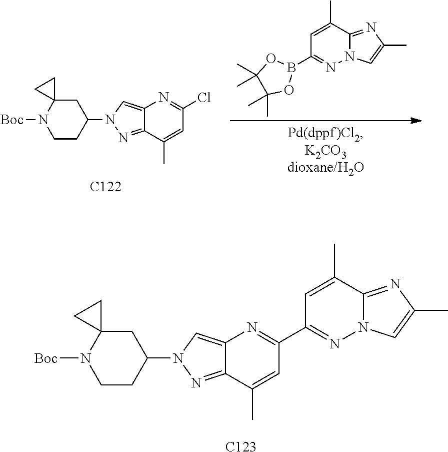

To a solution of tert-butyl 7-(5-chloro-7-methyl-pyrazolo[4,3-b]pyridin-2-yl)-4-azaspiro[2.5]octane-4-carboxylate (40.0 mg, 106 umol, 1.00 eq) and 2,8-dimethyl-6-(4,4,5,5-tetramethyl-1,3,2-dioxaborolan-2-yl)imidazo[1,2-b]pyridazine (A2, 139 mg, 509 umol, 4.80 eq) in dioxane (1.92 mL) was added a solution of K₂CO₃ (39.6 mg, 286 umol, 2.70 eq) in H₂O (0.48 mL). Then, 1,1-bis(diphenylphosphino)ferrocene]dichloropalladium(II) (13.2 mg, 18.0 umol, 0.17 eq) was added to the above mixture under N₂ protection. The reaction mixture was stirred at 80° C. for 1 hr. The reaction mixture was diluted with H₂O (10.0 mL), extracted with ethyl acetate (3×20.0 mL), dried over Na₂SO₄, filtered and concentrated under reduced pressure to give an oil. The oil was purified by column chromatography on silica gel eluted with petroleum ether/ethyl acetate (1/0 to 5/1) to give tert-butyl 7-[5-(2,8-dimethylimidazo[1,2-b]pyridazin-6-yl)-7-methyl-pyrazolo[4,3-b]pyridin-2-yl]-4-azaspiro[2.5]octane-4-carboxylate (34.0 mg, 32%) as a solid. LCMS: (ESI, m/z): 488.4 [M+H]⁺.

### Synthesis of Compound 208

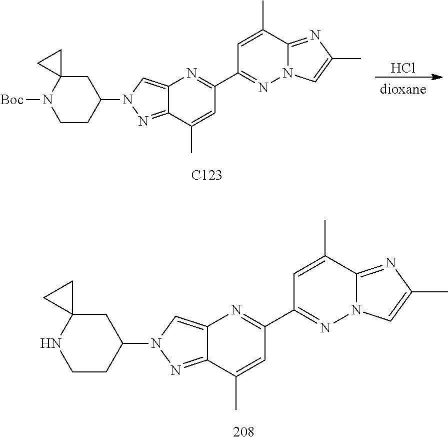

To a solution of tert-butyl 7-[5-(2,8-dimethylimidazo[1,2-b]pyridazin-6-yl)-7-methyl-pyrazolo [4,3-b]pyridin-2-yl]-4-azaspiro[2.5]octane-4-carboxylate (33.0 mg, 67.6 umol, 1.00 eq) in dioxane (1.00 mL) was added HCl/dioxane (7.0 M, 662 μL, 68.5 eq). The reaction mixture was stirred at 25° C. for 1 hr. LCMS showed the reaction mixture was completed. The reaction mixture was filtered and the filter cake was dried over in vacuum to afford a solid. The solid was purified by prep-HPLC (column: Phenomenex Luna 80*30 mm*3 um; mobile phase: [water (HCl)-acetonitrile]; B %: 1%-25%, 8 min) to afford 2-(4-azaspiro[2.5]octan-7-yl)-5-(2,8-dimethylimidazo[1,2-b]pyridazin-6-yl)-7-methyl-pyrazolo[4,3-b]pyridine (9.34 mg, 35.6%) as a solid. LCMS: (ESI, m/z): 388.3 [M+H]⁺. ¹HNMR (400 MHz, METHANOL-d4) δ ppm 8.72 (s, 2H) 8.35 (s, 1H) 8.22 (s, 1H) 5.09-5.18 (m, 1H) 3.70 (dt, J=12.91, 3.55 Hz, 1H) 3.41-3.49 (m, 1H) 2.91-3.00 (m, 1H) 2.80 (s, 3H) 2.74 (s, 3H) 2.67 (s, 3H) 2.56-2.64 (m, 2H) 2.00 (dd, J=13.88, 3.38 Hz, 1H) 1.06-1.21 (m, 3H) 0.97-1.05 (m, 1H)

### Example 69: Synthesis of Compound 211

### Synthesis of Intermediate C125

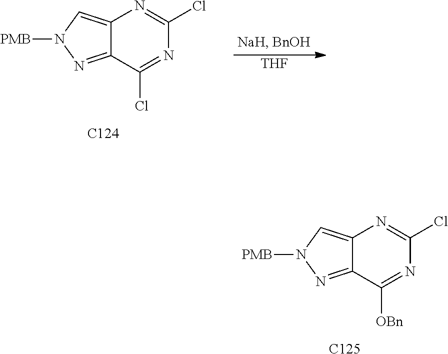

To a solution of 5,7-dichloro-2-[(4-methoxyphenyl)methyl]pyrazolo[4,3-d]pyrimidine (500 mg, 1.62 mmol, 1.00 eq) in tetrahydrofuran (5.00 mL) was added benzylalcohol (183 mg, 1.70 mmol, 176 μL, 1.05 eq) at 25° C. under N₂ atmosphere. The reaction mixture was cooled to −70° C. and then NaH (64.7 mg, 1.62 mmol, 60 wt %, 1.00 eq) was added in portions. The mixture was stirred at −70° C. for 30 min, then warmed up to −65° C. and stirred for 2 hrs. The reaction was quenched by addition of saturated NH₄Cl (20.0 mL) and extracted with ethyl acetate (20 mL×2). The combined organic layer was washed with saturated brine (20.0 mL), dried over Na₂SO₄, filtered and concentrated under reduced pressure to give 7-benzyloxy-5-chloro-2-[(4-methoxyphenyl)methyl]pyrazolo[4,3-d]pyrimidine (600 mg, 97.4%) as a solid. LCMS: (ESI, m/z): 381.1 [M+H]⁺. ¹H NMR (400 MHz, DMSO-d6) δ ppm 8.75 (s, 1H) 7.50-7.58 (m, 2H) 7.36-7.47 (m, 3H) 7.33 (d, J=8.63 Hz, 2H) 6.88-6.93 (m, 2H) 5.58 (s, 4H) 3.72 (s, 3H)

### Synthesis of Intermediate C126

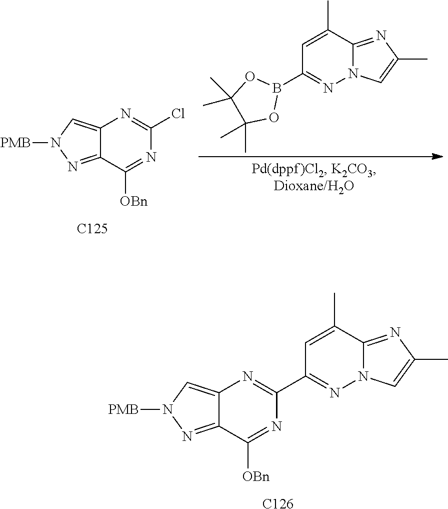

To a solution of 7-benzyloxy-5-chloro-2-[(4-methoxyphenyl)methyl]pyrazolo[4,3-d]pyrimidine (600 mg, 1.58 mmol, 1.00 eq) and 2,8-dimethyl-6-(4,4,5,5-tetramethyl-1,3,2-dioxaborolan-2-yl)imidazo[1,2-b]pyridazine (860 mg, 3.15 mmol, 2.00 eq) in H₂O (1.25 mL) and 1,4-dioxane (5.00 mL) was added 1,1 [symbol] bis(diphenylphosphino)ferrocenedichloro palladium(II) (115 mg, 157 umol, 0.10 eq) and K₂CO₃ (653 mg, 4.73 mmol, 3.00 eq) under N₂ protection. The reaction mixture was stirred at 100° C. for 3 hrs. The reaction was diluted with ethyl acetate (10.0 mL) and washed with water (5.00 mL×2), dried over Na₂SO₄, filtered and concentrated under reduced pressure to give residue. The residue was purified by column chromatography (SiO₂, ethyl acetate/methanol=1/0 to 24/1) to give 7-benzyloxy-5-(2,8-dimethylimidazo[1,2-b]pyridazin-6-yl)-2-[(4-methoxyphenyl)methyl]pyrazolo [4,3-d]pyrimidine (300 mg, 38.7%) as a solid. LCMS: (ESI, m/z): 492.2 [M+H]⁺. ¹H NMR (400 MHz, DMSO-d₆) δ ppm 8.92 (s, 1H) 8.17 (s, 1H) 8.04 (s, 1H) 7.62-7.66 (m, 2H) 7.41-7.46 (m, 2H) 7.35-7.40 (m, 3H) 6.91-6.94 (m, 2H) 5.76 (s, 2H) 5.64 (s, 2H) 3.72 (s, 3H) 2.64 (d, J=0.88 Hz, 3H) 2.44 (s, 3H)

### Synthesis of Intermediate C127

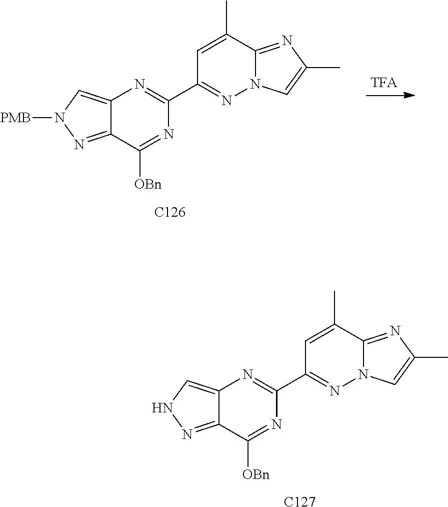

The mixture of 7-benzyloxy-5-(2,8-dimethylimidazo[1,2-b]pyridazin-6-yl)-2-[(4-methoxyphenyl) methyl]pyrazolo[4,3-d]pyrimidine (300 mg, 610 umol, 1.00 eq) in trifluoroacetic acid (3.00 mL) was stirred at 80° C. for 40 min. The reaction mixture was concentrated under reduced pressure to give an oil. The oil was diluted with ethyl acetate (5.00 mL) and washed with saturated NaHCO₃ (10.0 mL). The aqueous phase was extracted with ethyl acetate (8.00 mL×3). The combined organic layer was dried over Na₂SO₄, filtered and concentrated under reduced pressure to give crude product. The crude product was triturated with methanol (1.00 mL) at 25° C. for 15 min to give 7-benzyloxy-5-(2,8-dimethylimidazo[1,2-b]pyridazin-6-yl)-2H-pyrazolo[4,3-d]pyrimidine (35-3, 200 mg, 88.2% yield) as a solid. LCMS: (ESI, m/z): 372.2 [M+H]⁺

### Synthesis of Intermediate C128

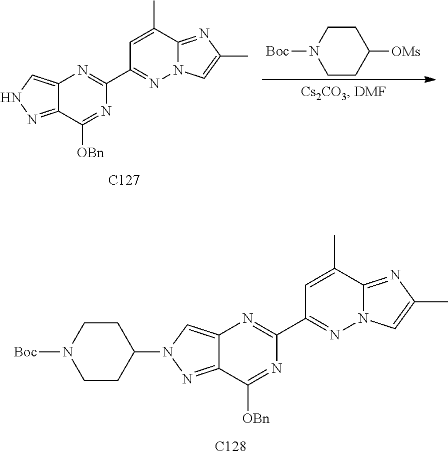

The suspension of 7-benzyloxy-5-(2,8-dimethylimidazo[1,2-b]pyridazin-6-yl)-2H-pyrazolo[4,3-d]pyrimidine (200 mg, 538 umol, 1.00 eq), tert-butyl 4-methylsulfonyloxypiperidine-1-carboxylate (300 mg, 1.08 mmol, 2.00 eq) and Cs₂CO₃ (526 mg, 1.62 mmol, 3.00 eq) in N,N-dimethylformamide (4.00 mL) was stirred at 100° C. for 8 hrs. The reaction mixture was diluted with ethyl acetate (20.0 mL) and washed with saturated brine (20.0 mL). The aqueous layer was extracted with ethyl acetate (10.0 mL×2). The organic layers were combined, dried over Na₂SO₄, filtered and concentrated under reduced pressure to give residue. The residue was purified by prep-HPLC (column: Waters Xbridge BEH C18 100×30 mm×10 um; mobile phase: [water (NH₄HCO₃)-acetonitrile]; B %: 40%-70%, 8 min) to give a mixture of tert-butyl 4-[7-benzyloxy-5-(2,8-dimethylimidazo[1,2-b]pyridazin-6-yl)pyrazolo[4,3-d]pyrimidin-2-yl]piperidine-1-carboxylate and tert-butyl 4-(7-(benzyloxy)-5-(2,8-dimethylimidazo[1,2-b]pyridazin-6-yl)-1H-pyrazolo[4,3-d]pyrimidin-1-yl)piperidine-1-carboxylate (63.0 mg, 21.0%) as a solid. N LCMS: (ESI, m/z): 555.5 [M+H]^{+ 1}H NMR (400 MHz, METHANOL-d4) δ ppm 8.28-8.67 (m, 1H) 8.06 (d, J=1.13 Hz, 1H) 7.98 (s, 1H) 7.21-7.44 (m, 3H) 7.10-7.16 (m, 2H) 5.75-5.97 (m, 3H) 3.38-3.54 (m, 4H) 2.70 (d, J=0.88 Hz, 3H) 2.49-2.54 (m, 3H) 2.02-2.19 (m, 2H) 1.78-2.01 (m, 2H) 1.48-1.51 (m, 9H)

### Synthesis of Intermediate C129

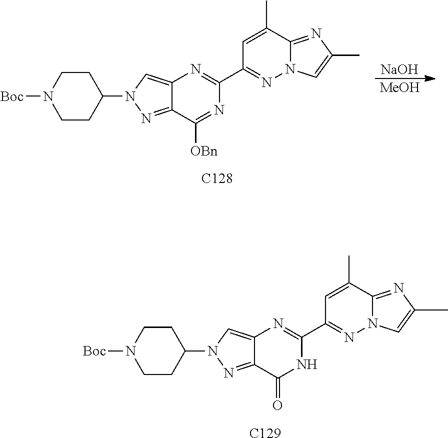

To the mixture of tert-butyl 4-[7-benzyloxy-5-(2,8-dimethylimidazo[1,2-b]pyridazin-6-yl)pyrazolo[4,3-d]pyrimidin-2-yl]piperidine-1-carboxylate and tert-butyl 4-(7-(benzyloxy)-5-(2,8-dimethylimidazo[1,2-b]pyridazin-6-yl)-1H-pyrazolo[4,3-d]pyrimidin-1-yl)piperidine-1-carboxylate (60.0 mg, 108 umol, 1.00 eq) in methanol (1.00 mL) was added NaOH (1.00 M, 1.00 mL, 9.24 eq) and the reaction mixture was stirred at 80° C. for 40 mins. The reaction mixture was poured into water (5.00 mL) and acidified to pH=7 with concentrated HCl. The resulting mixture was extracted with ethyl acetate (5.00 mL×10). The organic layer was combined, dried over Na₂SO₄, filtered and concentrated under reduced pressure to give the crude product of tert-butyl 4-[5-(2,8-dimethylimidazo[1,2-b]pyridazin-6-yl)-7-oxo-6H-pyrazolo[4,3-d]pyrimidin-2-yl]piperidine-1-carboxylate (40.0 mg, 79.6%) as a solid. The solid was used to the next step directly. LCMS: (ESI, m/z): 465.3 [M+H]⁺

### Synthesis of Compound 211

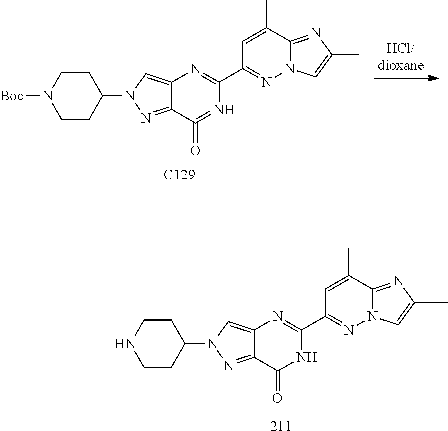

To the solution of tert-butyl 4-[5-(2,8-dimethylimidazo[1,2-b]pyridazin-6-yl)-7-oxo-6H-pyrazolo[4,3-d]pyrimidin-2-yl]piperidine-1-carboxylate (35-4, 40.0 mg, 86.1 umol, 1.00 eq) in 1,4-dioxane (0.100 mL) was added HCl/dioxane (4.00 M, 0.100 mL). The reaction was stirred at 25° C. for 2 hrs. LCMS showed the reaction was completed. The reaction mixture was concentrated under reduced pressure to give residue. The residue was purified by prep-HPLC (column: Phenomenex Luna 80×30 mm×3 um; mobile phase: [water (HCl)-acetonitrile]; B %: 1%-20%, 8 min) to give 5-(2,8-dimethylimidazo[1,2-b]pyridazin-6-yl)-2-(4-piperidyl)-6H-pyrazolo[4,3-d]pyrimidin-7-one (EVAL-0167-0035, 4.20 mg, 16.5%) as white solid. LCMS: (ESI, m/z): 365.3 [M+H]⁺. ¹H NMR (400 MHz, METHANOL-d4) δ ppm 8.48 (s, 1H) 8.43 (s, 1H) 8.32 (s, 1H) 4.92-4.95 (m, 1H) 3.64 (dt, J=13.07, 3.22 Hz, 2H) 3.22-3.30 (m, 2H) 2.78 (d, J=1.00 Hz, 3H) 2.64 (s, 3H) 2.39-2.50 (m, 4H)

### Example 70: Synthesis of Compound 213

### Synthesis of Intermediate C131

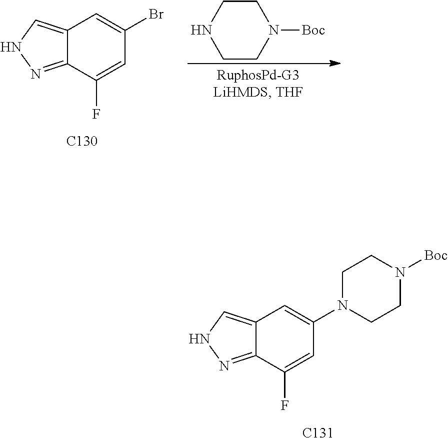 [SMILES: Fc1cc(Br)cc2c[nH]nc12 ; CC(C)(C)OC(=O)N1CCNCC1 ; "C130" ; CC(C)(C)OC(=O)N1CCN(c2cc(F)c3n[nH]cc3c2)CC1 ; "C131"]

To a solution of tert-butyl piperazine-1-carboxylate (206 mg, 1.11 mmol, 1.20 eq) and 5-bromo-7-fluoro-2H-indazole (200 mg, 0.93 mmol, 1.00 eq) in tetrahydrofuran (2.00 mL) was added (2-dicyclohexylphosphino-2′,6′-diisopropoxy-1,1′-biphenyl) [2-(2′-amino-1,1′-biphenyl)]palladium(II)methanesulfonate (77.8 mg, 0.093 mmol, 0.100 eq) and lithium bis(trimethylsilyl)amide (1.00 M, 3.72 mL, 4.00 eq). The reaction mixture was stirred at 80° C. for 12 hrs under N₂ protection. The reaction mixture was quenched with saturated ammonium chloride solution (20.0 mL), extracted with ethyl acetate (3×10.0 mL), washed with brine (10.0 mL), dried over Na₂SO₄, filtered and the filtrate was concentrated under reduced pressure to give the crude product. The crude product was purified by silica flash column chromatography (petroleum ether/ethyl acetate=1/1) to give tert-butyl 4-(7-fluoro-2H-indazol-5-yl)piperazine-1-carboxylate (150 mg, 33.5%) as a solid. LCMS: (ESI, m/z): 321.1 [M+H]⁺. ¹H NMR (400 MHz, CHLOROFORM-d) 6=8.49-7.83 (m, 1H), 7.27 (s, 1H), 7.03-6.85 (m, 2H), 3.69-3.58 (m, 4H), 3.18-3.02 (m, 4H), 1.50 (s, 9H)

### Synthesis of Intermediate C132

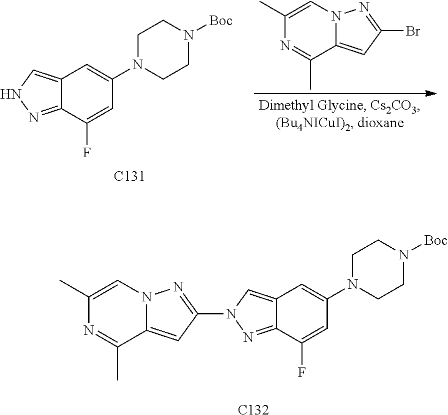 [SMILES: CC(C)(C)OC(=O)N1CCN(c2cc(F)c3n[nH]cc3c2)CC1 ; Cc1cn2nc(Br)cc2c(C)n1 ; "C131" ; Cc1cn2nc(-n3cc4cc(N5CCN(C(=O)OC(C)(C)C)CC5)cc(F)c4n3)cc2c(C)n1 ; "C132"]

To a solution of tert-butyl 4-(7-fluoro-2H-indazol-5-yl)piperazine-1-carboxylate (100 mg, 312 umol, 1.00 eq) and 2-bromo-4,6-dimethyl-pyrazolo[1,5-a]pyrazine (105 mg, 468 umol, 1.50 eq) in toluene (5.00 mL) was added Cs₂CO₃ (203 mg, 624 umol, 2.00 eq), (1R,2R)—N1,N2-dimethylcyclohexane-1,2-diamine (17.7 mg, 124 umol, 0.400 eq) and iodocopper tetrabutylammonium diiodide (69.9 mg, 62.4 umol, 0.200 eq). The reaction mixture was stirred at 120° C. for 16 hrs under N₂ protection. The reaction mixture was diluted with water (15.0 mL), extracted with ethyl acetate (3×5.00 mL), washed with brine (5.00 mL), dried over Na₂SO₄, filtered and the filtrate was concentrated under reduced pressure to give crude product. The crude product was purified by prep-HPLC (column: Waters Xbridge BEH C18 250*50 mm*10 um; mobile phase: [water(NH₄HCO₃)-acetonitrile]; B %: 45%-65%, 10 min) to give tert-butyl 4-[2-(4,6-dimethylpyrazolo[1,5-a]pyrazin-2-yl)-7-fluoro-indazol-5-yl]piperazine-1-carboxylate (EVAL-0123-0048-1, 5.00 mg, 3.44%) as a solid. LCMS: (ESI, m/z): 466.3 [M+H]⁺. ¹H NMR (400 MHz, methanol-d₄) δ ppm 8.78 (s, 1H) 8.31 (s, 1H) 7.28 (s, 1H) 7.02-7.05 (br d, J=13.88 Hz, 1H) 6.84 (s, 1H) 3.61 (br s, 4H) 3.13 (br d, J=4.63 Hz, 4H) 2.77 (s, 3H) 2.50 (s, 3H) 1.50 (s, 9H)

### Synthesis of Compound 213

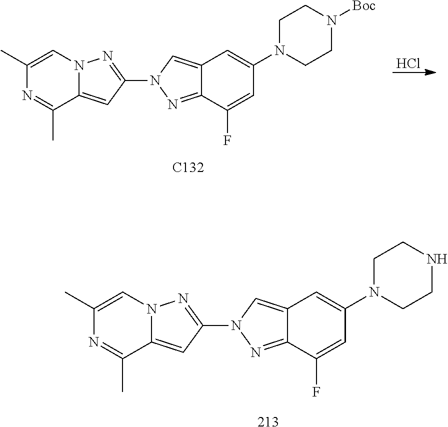 [SMILES: Cc1cn2nc(-n3cc4cc(N5CCN(C(=O)OC(C)(C)C)CC5)cc(F)c4n3)cc2c(C)n1 ; Cl ; "C132" ; Cc1cn2nc(-n3cc4cc(N5CCNCC5)cc(F)c4n3)cc2c(C)n1 ; "213"]

To a solution of tert-butyl 4-[2-(4,6-dimethylpyrazolo[1,5-a]pyrazin-2-yl)-7-fluoro-indazol-5-yl]piperazine-1-carboxylate (5.00 mg, 10.7 μmol, 1.00 eq) in dioxane (1.00 mL) was added HCl/dioxane (4.00 M, 0.500 mL) at 0° C. The reaction was stirred at 25° C. for 1 hr The reaction mixture was concentrated under reduced pressure to give the crude product. The crude product was purified by prep-HPLC (column: Phenomenex Luna 80*30 mm*3 um; mobile phase: [water(HCl)-acetonitrile]; B %: 10%-40%, 8 min) to give 2-(7-fluoro-5-piperazin-1-yl-indazol-2-yl)-4,6-dimethyl-pyrazolo[1,5-a]pyrazine (2.00 mg, 47%) as a solid. LCMS: (ESI, m/z): 366.3 [M+H]⁺. ¹H NMR (400 MHz, METHANOL-d₄) δ ppm 8.95 (br d, J=9.51 Hz, 2H) 7.97 (s, 1H) 7.12 (d, J=13.51 Hz, 1H) 3.43 (br s, 8H) 3.08 (s, 3H) 2.67 (s, 3H)

### Example 71: Synthesis of Compound 214

### Synthesis of Intermediate C134

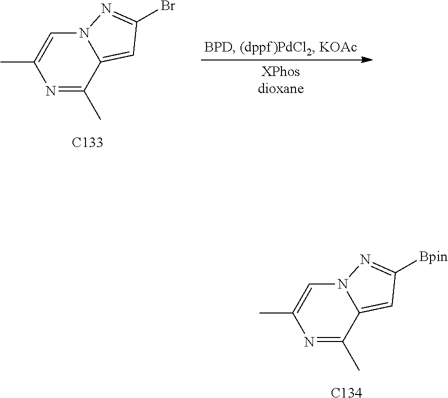

To a solution of 2-bromo-4,6-dimethyl-pyrazolo[1,5-a]pyrazine (1 g, 4.42 mmol) and 4,4,5,5-tetramethyl-2-(4,4,5,5-tetramethyl-1,3,2-dioxaborolan-2-yl)-1,3,2-dioxaborolane (2.25 g, 8.85 mmol) in dioxane (4.59 mL) was added (1,1 [symbol] bis(diphenylphosphino)ferrocenedichloro palladium(II) dichloromethane complex (485.49 mg, 663.50 μmol), 2-di-tert-butylphosphino-2,4,6-triisopropylbiphenyl (566.17 mg, 1.33 mmol) and potassium acetate (868.23 mg, 8.85 mmol) under N₂ protection. The reaction mixture was stirred at 100° C. for 2 hrs under N₂ protection. The reaction mixture was filtered via celite pad and washed with ethyl acetate (2 mL). The organic layer was concentrated under reduced pressure to give a residue. The residue was used in the next step without further purification. Crude product of 4,6-dimethyl-2-(4,4,5,5-tetramethyl-1,3,2-dioxaborolan-2-yl)pyrazolo[1,5-a]pyrazine (1 g, crude) was obtained as an oil. LCMS: (ESI, m/z): 192.4 [M+H]⁺. ¹H NMR (400 MHz, DMSO-d6) δ ppm 8.43 (s, 1H) 7.23 (d, J=0.86 Hz, 1H) 2.66 (s, 3H) 2.40 (s, 3H) 1.32 (s, 12H)

### Synthesis of Intermediate C135

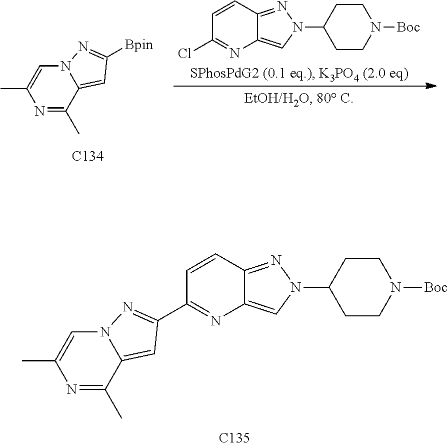

To a solution of 4,6-dimethyl-2-(4,4,5,5-tetramethyl-1,3,2-dioxaborolan-2-yl)pyrazolo[1,5-a]pyrazine (200.00 mg, 439.34 μmol) in ethanol (2 mL) and H₂O (0.2 mL) was added K₃PO₄ (186.51 mg, 878.68 μmol), [2-(2-aminophenyl)phenyl]-chloro-palladium;dicyclohexyl-[2-(2,6-dimethoxyphenyl)phenyl]phosphane (B1C2, 31.66 mg, 43.93 μmol). The reaction mixture was stirred at 80° C. for 12 hrs. TLC (ethyl acetate=1, Rf=0.31) showed that the starting material was consumed and new spot was generated. The mixture was concentrated under reduced pressure to give the crude product. The crude product was purified by prep-HPLC to give tert-butyl 4-(5-(4,6-dimethylpyrazolo[1,5-a]pyrazin-2-yl)-2H-pyrazolo[4,3-b]pyridin-2-yl)piperidine-1-carboxylate (0.15 g, yield 76.29%) as a solid. ¹H NMR (400 MHz, DMSO-d6) δ ppm 8.82 (s, 1H) 8.53 (s, 1H) 8.21 (d, J=9.16 Hz, 1H) 8.08 (d, J=9.03 Hz, 1H) 7.53 (s, 1H) 4.71-4.87 (m, 1H) 4.08-4.20 (m, 2H) 2.92-3.08 (m, 2H) 2.74 (s, 3H) 2.44 (s, 3H) 2.17 (br dd, J=12.67, 2.26 Hz, 2H) 1.98-2.06 (m, 2H) 1.45 (s, 9H)

### Synthesis of Compound 214

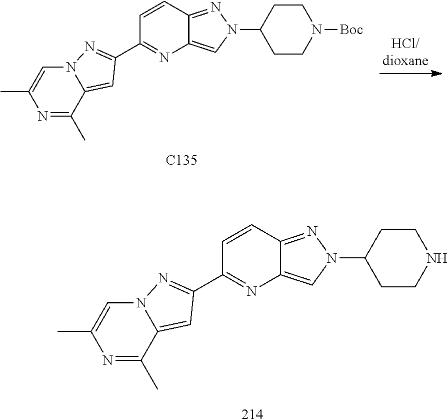 [SMILES: Cc1cn2nc(-c3ccc4nn(C5CCN(C(=O)OC(C)(C)C)CC5)cc4n3)cc2c(C)n1 ; "C135" ; Cc1cn2nc(-c3ccc4nn(C5CCNCC5)cc4n3)cc2c(C)n1 ; "214"]

A mixture of tert-butyl 4-[5-(4,6-dimethylpyrazolo[1,5-a]pyrazin-2-yl)pyrazolo[4,3-b]pyridin-2-yl]piperidine-1-carboxylate (0.15 g, 335.17 μmol) in dioxane/HCl (1 mL, 4M) was stirred for 1 hr at 25° C. under N₂. The mixture was concentrated and triturated by MTBE/DCM to give 5-(4,6-dimethylpyrazolo[1,5-a]pyrazin-2-yl)-2-(piperidin-4-yl)-2H-pyrazolo[4,3-b]pyridine (0.03 g, 23.32% yield) as a solid. LCMS: (ESI, m/z): 348.3 [M+H]⁺. ¹H NMR (400 MHz, METHANOL-d4) δ ppm 8.95 (s, 1H), 8.78 (s, 1H), 8.50 (d, J=9.1 Hz, 1H), 8.36 (d, J=9.0 Hz, 1H), 8.28 (s, 1H), 5.14-5.05 (m, 1H), 3.75-3.63 (m, 4H), 3.10 (s, 3H), 2.67 (d, J=0.8 Hz, 3H), 2.57-2.47 (m, 4H)

### Example 72: Synthesis of Compound 139

### Synthesis of Intermediate C137

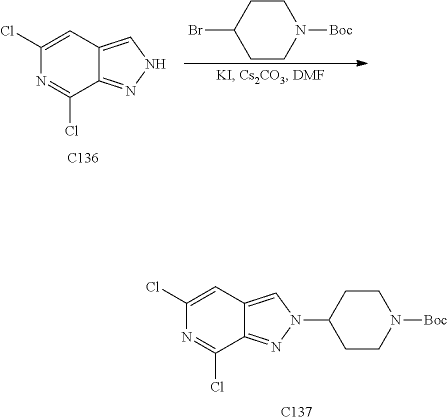 [SMILES: Clc1cc2c[nH]nc2c(Cl)n1 ; CC(C)(C)OC(=O)N1CCC(Br)CC1 ; "C136" ; CC(C)(C)OC(=O)N1CCC(n2cc3cc(Cl)nc(Cl)c3n2)CC1 ; "C137"]

To a mixture of 5,7-dichloro-2H-pyrazolo[3,4-c]pyridine (0.20 g, 1.06 mmol, 1.00 eq) in dimethyformamide (4.00 mL) was added Cs₂CO₃ (1.73 g, 5.32 mmol, 5.00 eq), KI (706 mg, 4.26 mmol, 4 eq) and tert-butyl 4-bromopiperidine-1-carboxylate (1.12 g, 4.26 mmol, 4.00 eq). The reaction mixture was stirred at 100° C. for 2 hrs. The mixture was dried under reduced pressure to give a brown solid. The brown solid was purified by prep-TLC to give tert-butyl 4-(5,7-dichloropyrazolo[3,4-c]pyridin-2-yl)piperidine-1-carboxylate (90.0 mg, 18.2%) as an oil. LCMS: (ESI, m/z): 371.1 [M+H]⁺. ¹H NMR (400 MHz, DMSO-d6) δ ppm 8.75 (s, 1H) 7.89 (s, 1H) 4.88 (tt, J=11.55, 3.96 Hz, 1H) 4.11 (br d, J=12.01 Hz, 2H) 2.95 (br d, J=7.75 Hz, 2H) 2.10-2.20 (m, 2H) 2.01 (br d, J=4.38 Hz, 1H) 1.90-1.98 (m, 1H) 1.43 (s, 9H)

### Synthesis of Intermediate C138

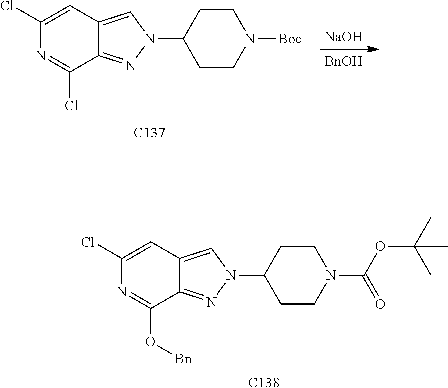

To a mixture of tert-butyl 4-(5,7-dichloropyrazolo[3,4-c]pyridin-2-yl)piperidine-1-carboxylate (50.0 mg, 134 umol, 1.00 eq) in phenyl methanol (780 mg, 7.21 mmol, 53.5 eq) was added NaOH (32.3 mg, 808 umol, 6.00 eq). The reaction mixture was stirred at 80° C. for 8 hrs. Water (10.0 mL) was added to quench the reaction and the reaction mixture was extracted with ethyl acetate (3×10.0 mL). The combined organic phase was washed with brine (10.0 mL), dried over Na₂SO₄, filtered and the filtrate was concentrated under reduced pressure to give an oil. The yellow oil was purified by chromatography on a silica gel (ethyl acetate/petroleum ether=0/1 to 1/0) to give tert-butyl 4-(7-benzyloxy-5-chloro-pyrazolo [3,4-c]pyridin-2-yl)piperidine-1-carboxylate (20.0 mg, 20.1%) as a solid. LCMS: (ESI, m/z): 443.2 [M+H]⁺. ¹H NMR (400 MHz, DMSO-d6) δ ppm 8.49 (s, 1H) 7.35-7.45 (m, 3H) 7.30-7.33 (m, 3H) 5.50 (s, 2H) 4.69-4.80 (m, 1H) 3.28-3.31 (m, 2H) 2.83-3.03 (m, 2H) 2.10 (br d, J=10.64 Hz, 2H) 1.87-1.93 (m, 2H) 1.41 (s, 9H)

### Synthesis of Intermediate C139

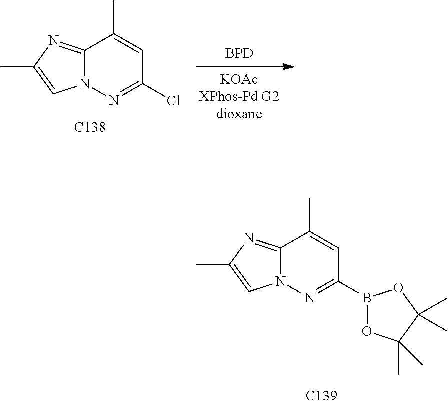

To a mixture of 6-chloro-2,8-dimethyl-imidazo[1,2-b]pyridazine (5.50 g, 30.3 mmol, 1.00 eq) in 1,4-dioxane (55.0 mL) was added bis(pinacolato)diboron (15.4 g, 60.6 mmol, 2.00 eq), potassium acetate (8.92 g, 90.9 mmol, 3.00 eq) and chloro(2-dicyclohexylphosphino-2 [symbol] [symbol] [symbol] triisopropyl-1,1 [symbol] phenyl)[2-(2 [symbol] amino-1,1 [symbol] phenyl)]palladium(ii) (2.40 g, 3.03 mmol, 0.100 eq). The reaction was stirred at 100° C. for 1 hr at argon protection. The mixture was filtered and the filtrate was concentrated under reduced pressure to give the 2,8-dimethyl-6-(4,4,5,5-tetramethyl-1,3,2-dioxaborolan-2-yl)imidazo[1,2-b]pyridazine (5.00 g, crude product) as black oil. The oil was used to next step without any purification. LCMS: (ESI, m/z): 192.2 [M−82+H]⁺.

### Synthesis of Intermediate C140

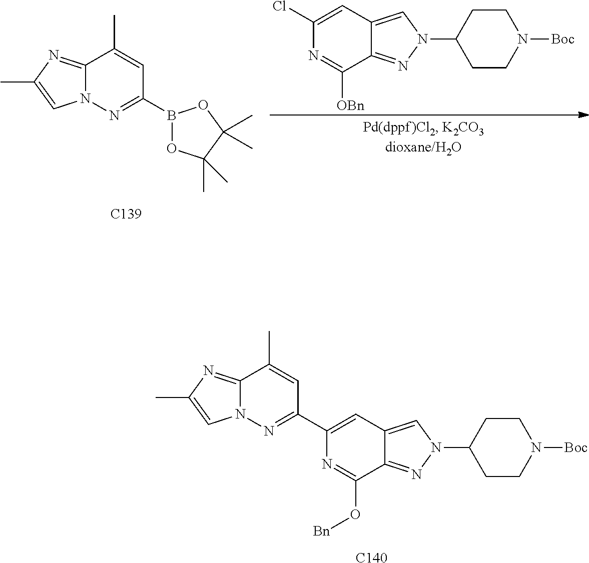

To a mixture of tert-butyl 4-(7-benzyloxy-5-chloro-pyrazolo[3,4-c]pyridin-2-yl)piperidine-1-carboxylate (200 mg, 452 umol, 1.00 eq) in 1,4-dioxane (1.60 mL) was added 2,8-dimethyl-6-(4,4,5,5-tetramethyl-1,3,2-dioxaborolan-2-yl)imidazo[1,2-b]pyridazine (185 mg, 677 umol, 1.50 eq), K₂CO₃ (187 mg, 1.35 mmol, 3.00 eq) in H₂O (0.40 mL) and [1,1-bis(diphenylphosphino)ferrocene]dichloropalladium(ii) (33.0 mg, 45.2 umol, 0.10 eq). The reaction mixture was stirred at 80° C. for 1 hr. Water (20.0 mL) was added to the reaction and the mixture was extracted with ethyl acetate (3×20.0 mL). The combined organic phase was washed with brine (30.0 mL), dried over Na₂SO₄, filtered and the filtrate was concentrated under reduced pressure to give yellow oil. The yellow oil was purified by chromatography on a silica gel (ethyl acetate/petroleum ether=0/1 to 3/1) to give tert-butyl 4-[7-benzyloxy-5-(2,8-dimethylimidazo[1,2-b]pyridazin-6-yl)pyrazolo[3,4-c]pyri-dine-2-yl]piperidine-1-carboxylate (200 mg, 80.0%) as a solid. LCMS: (ESI, m/z): 554.3 [M+H]⁺. ¹H NMR (400 MHz, DMSO-d6) δ ppm 8.65 (br s, 1H) 8.08-8.23 (m, 1H) 8.02 (br s, 1H) 7.90 (br s, 1H) 7.56-7.63 (m, 2H) 7.33-7.48 (m, 3H) 5.70 (br s, 2H) 4.64-4.86 (m, 1H) 3.93-4.21 (m, 4H) 2.81-3.08 (m, 2H) 2.62 (br s, 3H) 2.41 (br s, 3H) 2.07-2.17 (m, 2H) 1.43 (br s, 9H)

### Synthesis of Compound 139

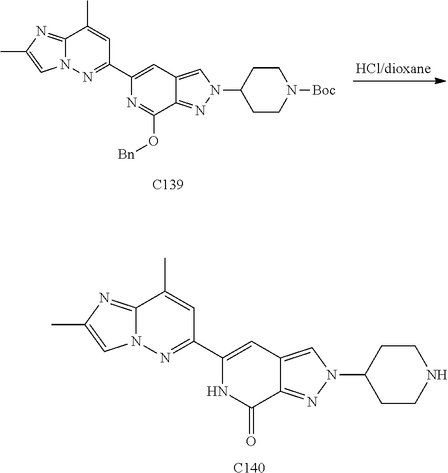 [SMILES: Cc1cn2nc(-c3cc4cn(C5CCN(C(=O)OC(C)(C)C)CC5)nc4c(OCc4ccccc4)n3)cc(C)c2n1 ; "C139" ; Cc1cn2nc(-c3cc4cn(C5CCNCC5)nc4c(=O)[nH]3)cc(C)c2n1 ; "C140"]

A suspension of tert-butyl 4-[7-benzyloxy-5-(2,8-dimethylimidazo[1,2-b]pyridazin-6-yl) pyrazolo [3,4-c]pyridin-2-yl]piperidine-1-carboxylate (50.0 mg, 90.3 umol, 1.00 eq) in HCl/dioxane (1.00 mL) was stirred at 25° C. for 2 hrs. The reaction mixture was concentrated in vacuum to give residue. The residue was purified by prep-HPLC (Column: Phenomenex Luna 80×30 mm×3 um; mobile phase: [water (HCl)-acetonitrile]; B %: 1%-35%, 8 min) to give 5-(2,8-dimethylimidazo[1,2-b]pyridazin-6-yl)-2-(4-piperidyl)-6H-pyrazolo[3,4-c]pyridin-7-one (15.0 mg, 44.3%) as a solid. LCMS (ESI, m/z): 364.0[M+H]⁺. ¹H NMR (400 MHz, METHANOL-d4) δ ppm 8.42 (s, 1H) 8.31 (dd, J=10.21, 0.79 Hz, 2H) 7.71 (s, 1H) 4.90-4.97 (m, 1H) 3.65 (dt, J=12.99, 3.35 Hz, 2H) 3.26 (br d, J=9.17 Hz, 2H) 2.78 (d, J=0.73 Hz, 3H) 2.65 (s, 3H) 2.41-2.51 (m, 4H)

### Example 73: Synthesis of Compound 142

### Synthesis of Intermediate C142

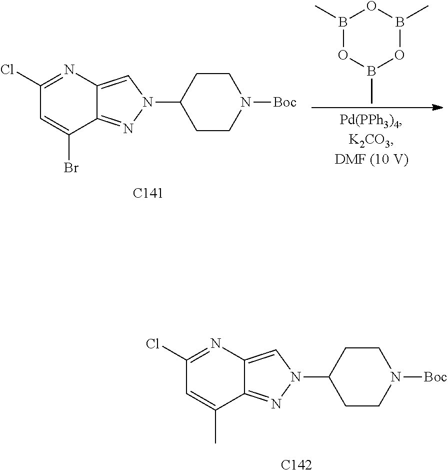

To a mixture of 2,4,6-trimethyl-1,3,5,2,4,6-trioxatriborinane (362 mg, 1.44 mmol, 3.00 eq), tert-butyl 4-(7-bromo-5-chloro-pyrazolo[4,3-b]pyridin-2-yl)piperidine-1-carboxylate (200 mg, 481 umol, 1.00 eq) and K₂CO₃ (265 mg, 1.92 mmol, 4.00 eq) in dimethyformamide (2.00 mL) was added tetrakis[triphenylphosphine]palladium(0) (55.5 mg, 48.1 umol, 0.100 eq). The reaction mixture was stirred at 100° C. for 5 hrs. The reaction mixture was diluted with water (50.0 mL), extracted with ethyl acetate (3×50.0 mL), washed with brine (100 mL), dried over Na₂SO₄, filtered and the filtrate was concentrated under reduced pressure to give an oil. The oil was purified by chromatography on a silica gel eluted with ethyl acetate/petroleum ether=0/1 to 1/1 to give tert-butyl 4-(5-chloro-7-methyl-pyrazolo[4,3-b]pyridin-2-yl)piperidine-1-carboxylate (50.0 mg, 23.7%) as a solid. LCMS: (ESI, m/z): 351.2 [M+H]⁺. ¹H NMR (400 MHz, DMSO-d6) δ ppm 8.71 (s, 1H) 7.15 (s, 1H) 4.43-4.94 (m, 1H) 4.04-4.18 (m, 2H) 2.83-3.10 (m, 2H) 2.55 (s, 3H) 2.12 (br d, J=11.49 Hz, 2H) 1.90-2.01 (m, 2H) 1.43 (s, 9H)

### Synthesis of Intermediate C143

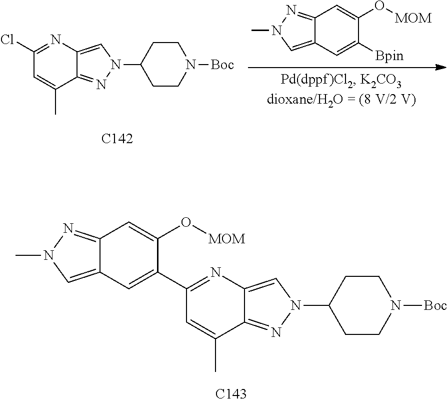

To a solution of tert-butyl 4-(5-chloro-7-methyl-pyrazolo[4,3-b]pyridin-2-yl)piperidine-1-carboxylate (80.0 mg, 228 umol, 1.00 eq) in dimethyformamide (0.100 mL) was added 6-(methoxymethoxy)-2-methyl-5-(4,4,5,5-tetramethyl-1,3,2-dioxaborolan-2-yl)indazole (108 mg, 342 umol, 1.50 eq), K₂CO₃ (126 mg, 912 umol, 4.00 eq) and bis(triphenylphosphine)palladium(II)diacetate (26.3 mg, 22.8 umol, 0.100 eq). The reaction was stirred at 100° C. for 5 hrs. The reaction mixture was purified by prep-HPLC (Column: Waters Xbridge BEH C18 100×30 mm×10 um; mobile phase: [water (NH₄HCO₃)-acetonitrile]; B %: 30%-60%, 8 min) to give tert-butyl 4-[5-[6-(methoxy methoxy)-2-methyl-indazol-5-yl]-7-methyl-pyrazolo[4,3-b]pyridin-2-yl]piperidine-1-carboxylate (17.0 mg, 14.7%) as a solid. LCMS: (ESI, m/z): 507.3 [M+H]⁺. ¹H NMR (400 MHz, DMSO-d6) δ ppm 8.69 (s, 1H) 8.31 (s, 1H) 7.82 (s, 1H) 7.36 (d, J=0.83 Hz, 1H) 7.18 (s, 1H) 5.23 (s, 2H) 4.74 (tt, J=11.40, 3.92 Hz, 1H) 4.13 (s, 5H) 3.35 (s, 3H) 2.86-3.07 (m, 2H) 2.56 (s, 3H) 2.15 (br d, J=9.78 Hz, 2H) 1.89-2.08 (m, 2H) 1.44 (s, 9H)

### Synthesis of Compound 142

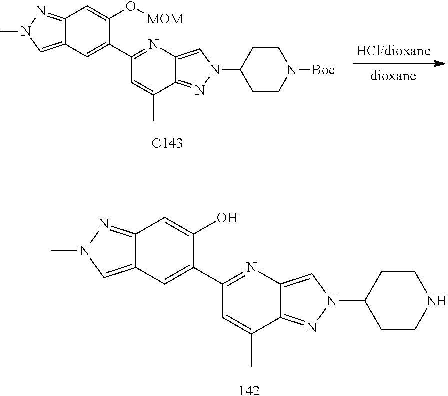

A mixture of tert-butyl 4-[5-[6-(methoxymethoxy)-2-methyl-indazol-5-yl]-7-methyl-pyrazolo [4,3-b]pyridin-2-yl]piperidine-1-carboxylate (17.0 mg, 33.5 umol, 1.00 eq) in dioxane (0.400 mL) was added HCl/dioxane (10.0 M, 0.40 mL). The reaction mixture was stirred at 25° C. for 2 hrs. The reaction mixture was filtered. The filter cake was dried over in vacuum to give 2-methyl-5-[7-methyl-2-(4-piperidyl) pyrazolo [4,3-b]pyridin-5-yl]indazol-6-ol (9.76 mg, 80.2%) as a solid. LCMS: (ESI, m/z): 363.1 [M+H]⁺. ¹H NMR (400 MHz, METHANOL-d4) δ ppm 8.85 (s, 1H) 8.54 (s, 1H) 8.38 (s, 1H) 8.04 (d, J=0.88 Hz, 1H) 7.12 (s, 1H) 5.16 (tt, J=9.88, 5.05 Hz, 1H) 4.25 (s, 3H) 3.65-3.72 (m, 2H) 3.33-3.39 (m, 2H) 2.95 (s, 3H) 2.49-2.64 (m, 4H)

### Example 74: Synthesis of Compounds 167 and 168 Synthesis of Intermediates C₁₄₅ and C₁₄₆

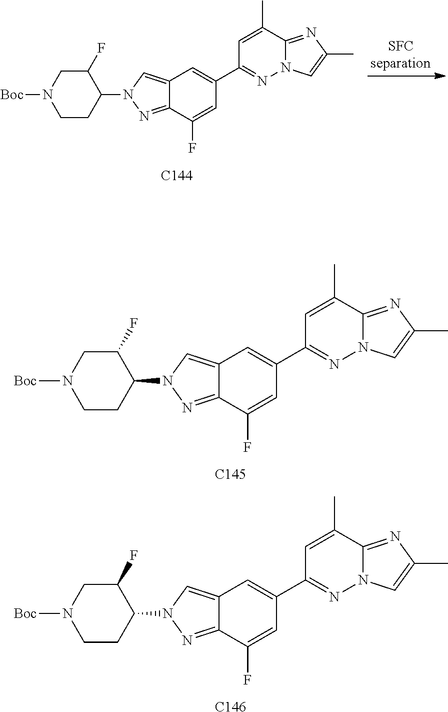 [SMILES: Cc1cn2nc(-c3cc(F)c4nn(C5CCN(C(=O)OC(C)(C)C)CC5F)cc4c3)cc(C)c2n1 ; "C144" ; Cc1cn2nc(-c3cc(F)c4nn([C@H]5CCN(C(=O)OC(C)(C)C)C[C@@H]5F)cc4c3)cc(C)c2n1 ; "C145" ; Cc1cn2nc(-c3cc(F)c4nn([C@@H]5CCN(C(=O)OC(C)(C)C)C[C@H]5F)cc4c3)cc(C)c2n1 ; "C146"]

Intermediate C144 (130 mg, 269 umol) was separated by SFC (column: Phenomenex-Cellulose-2 (250 mm×30 mm, 5 um); mobile phase: [0.1% NH₃H₂O methanol]; B %: 45%-45%, 9 min) to afford (3S,4S)-tert-butyl 4-(5-(2,8-dimethyl imidazo[1,2-b]pyridazin-6-yl)-7-fluoro-2H-indazol-2-yl)-3-fluoropiperidine-1-carboxylate (40.0 mg, 15.3%) as a solid and (3R,4R)-tert-butyl 4-(5-(2,8-dimethylimidazo[1,2-b]pyridazin-6-yl)-7-fluoro-2H-indazol-2-yl)-3-fluoropiperidine-1-carboxylate (59A, 40.0 mg, 15.3%) as a solid. Intermediate C145: LCMS: (ESI, m/z): 483.2 [M+H]⁺. ¹H NMR (400 MHz, chloroform-d) δ ppm 8.18 (d, J=2.32 Hz, 1H) 8.01 (d, J=0.86 Hz, 1H) 7.77 (s, 1H) 7.70 (dd, J=12.59, 0.98 Hz, 1H) 7.30 (s, 1H) 4.89-5.18 (m, 1H) 4.50-4.77 (m, 2H) 4.23-4.44 (m, 1H) δ ppm 2.82-3.07 (m, 2H) 2.73 (s, 3H) 2.48-2.66 (m, 4H) 2.21-2.35 (m, 1H) 1.51 (s, 9H) Intermediate C146: LCMS: (ESI, m/z): 483.2 [M+H]⁺. ¹H NMR (400 MHz, chloroform-d) δ ppm 8.18 (d, J=2.20 Hz, 1H) 8.01 (s, 1H) 7.77 (s, 1H) 7.71 (d, J=12.59 Hz, 1H) 7.31 (s, 1H) 4.92-5.19 (m, 1H) 4.48-4.82 (m, 2H) 4.18-4.45 (m, 1H) 2.83-3.08 (m, 2H) 2.73 (s, 3H) 2.48-2.64 (m, 4H) 2.21-2.34 (m, 1H) 1.51 (s, 9H)

### Synthesis of Compound 167

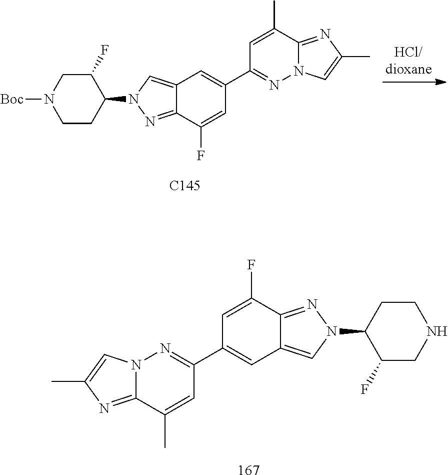

A mixture of (3 S,4S)-tert-butyl 4-(5-(2,8-dimethylimidazo[1,2-b]pyridazin-6-yl)-7-fluoro-2H-indazol-2-yl)-3-fluoropiperidine-1-carboxylate (8A, 40.0 mg, 83.0 umol, 1.00 eq) in dioxane (1.00 mL) was added HCl/dioxane (4 M, 980 μL, 38.6 eq). The reaction mixture was stirred at 25° C. for 2 hrs. The reaction was filtered and the filter cake was dried in vacuum to give 6-(7-fluoro-2-((3S,4S)-3-fluoropiperidin-4-yl)-2H-indazol-5-yl)-2,8-dimethylimidazo[1,2-b]pyridazine (35.0 mg, 77.2%) as a solid. LCMS: (ESI, m/z): 383.1 [M+H]⁺. ¹H NMR (400 MHz, deuterium oxide) δ ppm 8.67 (d, J=2.38 Hz, 1H) 8.19 (s, 1H) 7.98 (br d, J=5.50 Hz, 2H) 7.68 (d, J=12.76 Hz, 1H) 5.27-5.48 (m, 1H) 5.18 (qd, J=9.96, 5.25 Hz, 1H) 3.91-4.01 (m, 1H) 3.70 (br d, J=13.38 Hz, 1H) 3.30-3.49 (m, 2H) 2.63 (s, 5H) 2.51 (s, 3H)

### Synthesis of Compound 168

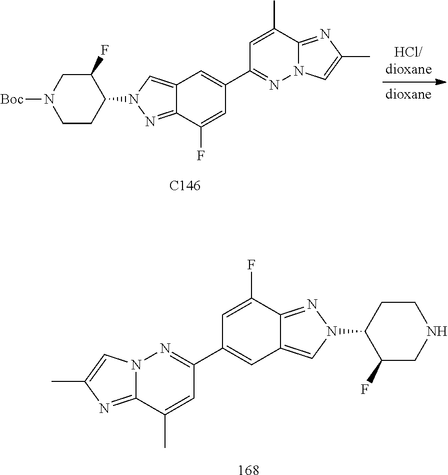

A solution of tert-butyl (3R,4R)-4-[5-(2,8-dimethylimidazo[1,2-b]pyridazin-6-yl)-7-fluoro-indazol-2-yl]-3-fluoro-piperidine-1-carboxylate (40.0 mg, 83.0 umol, 1.00 eq) in dioxane (1.00 mL) was added HCl/dioxane (4.00 M, 980 μL, 38.6 eq). The reaction mixture was stirred at 25° C. for 2 hrs. The reaction was filtered and the filter cake dried over in vacuum to give 6-(7-fluoro-2-((3R,4R)-3-fluoropiperidin-4-yl)-2H-indazol-5-yl)-2,8-dimethylimidazo [1,2-b]pyridazine (30.0 mg, 66.2%) as a solid. LCMS: (ESI, m/z): 383.1 [M+H]⁺. ¹H NMR (400 MHz, deuterium oxide) δ ppm 8.67 (d, J=2.50 Hz, 1H) 8.20 (s, 1H) 7.98 (d, J=7.00 Hz, 2H) 7.65-7.73 (m, 1H) 5.26-5.50 (m, 1H) 5.18 (qd, J=9.94, 5.32 Hz, 1H) 3.88-4.06 (m, 1H) 3.71 (br d, J=13.26 Hz, 1H) 3.27-3.51 (m, 2H) 2.55-2.68 (m, 5H) 2.51 (s, 3H)

### Example 75: Synthesis of Compound 192

### Synthesis of Intermediate C148

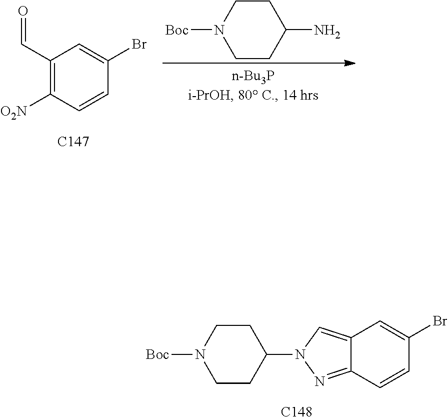 [SMILES: O=Cc1cc(Br)ccc1[N+](=O)[O-] ; CC(C)(C)OC(=O)N1CCC(N)CC1 ; "C147" ; CC(C)(C)OC(=O)N1CCC(n2cc3cc(Br)ccc3n2)CC1 ; "C148"]

To a mixture of 5-bromo-2-nitro-benzaldehyde (41-1, 1.0 g, 4.35 mmol, 1.0 eq) and tert-butyl 4-aminopiperidine-1-carboxylate (957 mg, 4.78 mmol, 1.10 eq) in propan-2-ol (15.0 mL) was stirred at 80° C. for 2 hrs. The reaction was cooled to 25° C., and added tributylphosphane (2.64 g, 13.0 mmol, 3.22 mL, 3.0 eq). The reaction mixture was then stirred at 80° C. for 12 hrs. TLC (petroleum ether/ethyl acetate=5/1, Rf=0.4) showed the reaction was completed. The reaction mixture was quenched with water (30.0 mL), extracted with ethyl acetate (3×30.0 mL), washed with brine (30.0 mL), dried over Na₂SO₄, filtered and the filtrate was concentrated under reduced pressure to give residue. The residue was purified by flash column chromatography (petroleum ether/ethyl acetate=5/1) to give tert-butyl 4-(5-bromoindazol-2-yl) piperidine-1-carboxylate (41-3, 1.20 g, 72.5% yield) as an oil. LCMS: (ESI, m/z): 381.2 [M+1]⁺. ¹H NMR (400 MHz, chloroform-d) δ ppm 7.91 (s, 1H), 7.82 (d, J=0.9 Hz, 1H), 7.59 (d, J=9.1 Hz, 1H), 7.34 (dd, J=1.6, 9.1 Hz, 1H), 4.55 (tt, J=4.0, 11.5 Hz, 1H), 2.95 (br t, J=11.6 Hz, 2H), 2.29-2.20 (m, 2H), 2.14-2.06 (m, 2H), 1.76-1.56 (m, 2H), 1.49 (s, 9H)

### Synthesis of Intermediate C149

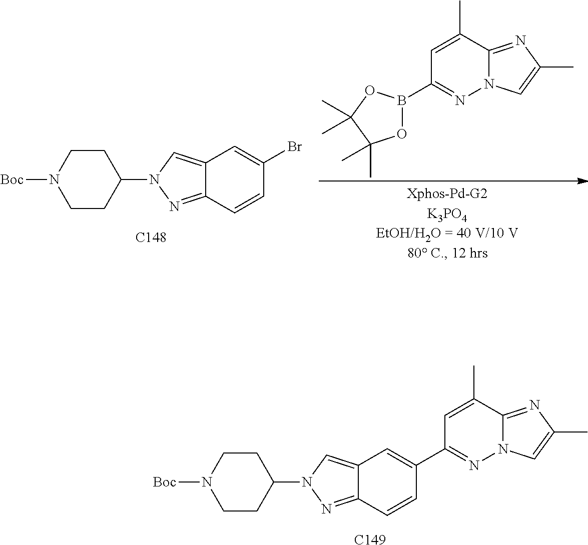

To a mixture of 2,8-dimethyl-6-(4,4,5,5-tetramethyl-1,3,2-dioxaborolan-2-yl)imidazo[1,2-b]pyridazine (143 mg, 525 umol, 1.0 eq), tert-butyl 4-(5-bromoindazol-2-yl)piperidine-1-carboxylate (200 mg, 525 umol, 1.0 eq) and potassium phosphate (446 mg, 2.10 mmol, 4.0 eq) in ethanol (20.0 mL) and H₂O (5.00 mL) was added Chloro(2-Dicyclohexylphosphino-2 [symbol] [symbol] [symbol] Triisopropyl-1,1 [symbol] Biphenyl)[2-(2 [symbol] Amino-1,1 [symbol] Biphenyl)]Palladium(Ii) (41.3 mg, 52.5 umol, 0.100 eq). The reaction mixture was then stirred at 80° C. for 12 hrs. The reaction mixture was diluted with water (50.0 mL), extracted with ethyl acetate (3×50.0 mL), washed with brine (30.0 mL), dried over Na₂SO₄, filtered and the filtrate was concentrated under reduced pressure to give residue. The residue was purified by flash column chromatography (petroleum ether/ethyl acetate=5/1) to give tert-butyl 4-[5-(2,8-dimethylimidazo[1,2-b]pyridazin-6-yl)indazol-2-yl]piperidine-1-carboxylate (200 mg, 42.5%) as a solid. LCMS: (ESI, m/z): 447.3 [M+H]⁺. ¹H NMR (400 MHz, chloroform-d) δ ppm 8.21 (s, 1H), 8.07 (s, 1H), 7.94 (dd, J=1.6, 9.1 Hz, 1H), 7.82 (d, J=9.1 Hz, 1H), 7.77 (s, 1H), 7.31 (s, 1H), 4.67-4.55 (m, 1H), 3.03-2.92 (m, 2H), 2.72 (s, 3H), 2.54 (s, 3H), 2.33-2.25 (m, 3H), 2.20-2.10 (m, 2H), 2.07 (s, 1H), 1.50 (s, 9H)

### Synthesis of Compound 192

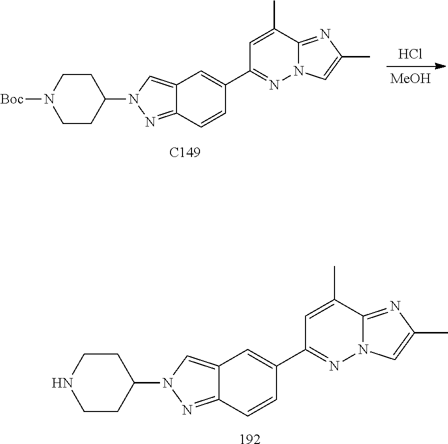

A solution of tert-butyl 4-[5-(2,8-dimethylimidazo[1,2-b]pyridazin-6-yl)indazol-2-yl]piperidine-1-carboxylate (41-4, 200 mg, 447 umol, 1.0 eq) in HCl/methanol (4.0 M, 0.100 mL) was stirred at 25° C. for 1 hr. The reaction mixture was filtered and the filter cake was dried in vacuum to give residue. The residue was triturated with methyl tert-butyl ether (5.00 mL) and filtered. The filter cake was dried in vacuum to give 2,8-dimethyl-6-[2-(4-piperidyl)indazol-5-yl]imidazo[1,2-b]pyridazine (69.9 mg, 42.8%) as a solid. LCMS: (ESI, m/z): 347.1 [M+H]⁺. ¹H NMR (400 MHz, methanol-d4) δ ppm 8.61-8.54 (m, 2H), 8.30 (dd, J=0.9, 6.1 Hz, 2H), 8.13 (dd, J=1.7, 9.2 Hz, 1H), 7.81 (d, J=9.3 Hz, 1H), 5.02-4.93 (m, 1H), 3.71-3.62 (m, 2H), 3.37-3.33 (m, 2H), 2.79 (d, J=1.0 Hz, 3H), 2.65 (d, J=0.8 Hz, 3H), 2.54-2.44 (m, 4H)

### Example 76: Synthesis of Compound 196

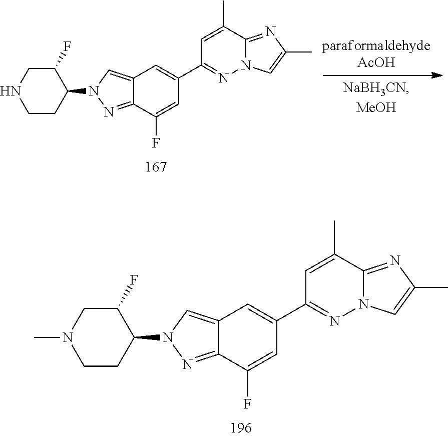

To a solution of 6-(7-fluoro-2-((3S,4S)-3-fluoropiperidin-4-yl)-2H-indazol-5-yl)-2,8-dimethylimidazo[1,2-b]pyridazine (35.0 mg, 91.6 umol, 1.00 eq) in methanol (0.300 mL) was added N,N-diethylethanamine (21.8 mg, 215 umol, 2.75 eq) and paraformaldehyde (2.36 mg, 78.5 umol, 1.00 eq). The reaction mixture was stirred at 25° C. for 10 min. Then, acetic acid (0.010 mL) and sodium cyanoboro-hydride (12.3 mg, 196 umol, 2.50 eq) was added. The reaction mixture was stirred at 25° C. for 12 hrs. The reaction mixture was filtered and the filter cake was purified by prep-HPLC (column: Waters Xbridge Prep OBD C18 150×40 mm×10 um; mobile phase: [water (NH₄HCO₃)-acetonitrile]; B %: 25%-55%, 8 min) to give 6-[7-fluoro-2-[(3S,4S)-3-fluoro-1-methyl-4-piperidyl]indazol-5-yl]-2,8-dimethyl-imidazo[1,2-b]pyridazine (4.10 mg, 13.2%) as a solid. LCMS: (ESI, m/z): 397.2 [M+H]⁺. ¹H NMR (400 MHz, DMSO-d6) δ ppm 8.80 (d, J=2.63 Hz, 1H) 8.32 (s, 1H) 8.03 (s, 1H) 7.68-7.85 (m, 2H) 4.96-5.24 (m, 1H) 4.66-4.89 (m, 1H) 3.29 (br d, J=5.38 Hz, 1H) 2.89 (br d, J=11.38 Hz, 1H) 2.60 (s, 3H) 2.40 (s, 3H) 2.32 (s, 3H) 2.08-2.29 (m, 4H)

### Example 77: Synthesis of Compound 197

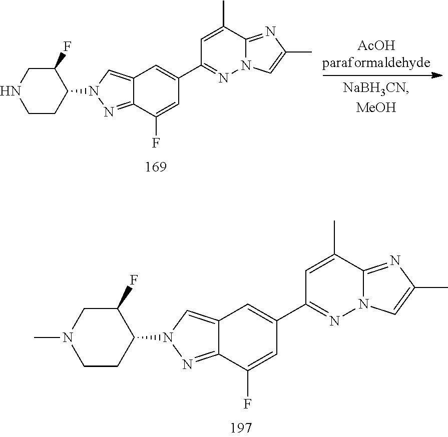

To a solution of 6-(7-fluoro-2-((3R,4R)-3-fluoropiperidin-4-yl)-2H-indazol-5-yl)-2,8-dimethylimidazo[1,2-b]pyridazine (30.0 mg, 78.5 umol, 1.00 eq) in methanol (0.300 mL) was added N,N-diethylethanamine (21.8 mg, 215 umol, 2.75 eq) and paraformaldehyde (2.36 mg, 78.5 umol, 1.00 eq). The mixture was stirred at 25° C. for 10 min. Then, acetic acid (0.01 mL) and sodium cyanoborohydride (12.3 mg, 196 umol, 2.50 eq) was added. The reaction mixture was stirred at 25° C. for 12 hrs. The mixture was quenched with water (1 mL) and purified by prep-HPLC (column: Waters Xbridge Prep OBD C18 150×40 mm×10 um; mobile phase: [water (NH₄HCO₃)-acetonitrile]; B %: 25%-55%, 8 min) to give 6-(7-fluoro-2-((3R,4R)-3-fluoro-1-methylpiperidin-4-yl)-2H-indazol-5-yl)-2,8-dimethylimidazo[1,2-b]pyridazine (8.10 mg, 26.0%) as a solid. LCMS: (ESI, m/z): 397.2 [M+H]⁺. ¹H NMR (400 MHz, DMSO-d6) δ ppm 8.80 (d, J=2.13 Hz, 1H) 8.32 (s, 1H) 8.04 (s, 1H) 7.68-7.82 (m, 2H) 4.99-5.24 (m, 1H) 4.68-4.85 (m, 1H) 3.29 (br s, 1H) 2.89 (br d, J=11.38 Hz, 1H) 2.60 (s, 3H) 2.40 (s, 3H) 2.32 (s, 3H) 2.09-2.29 (m, 4H)

### Example 78: Synthesis of Compound 198

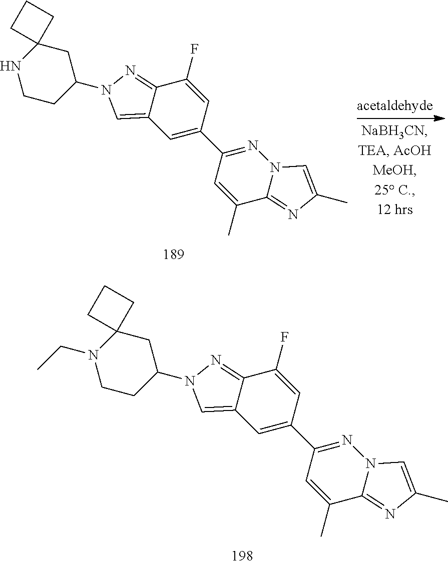

To a solution of 6-[2-(5-azaspiro[3.5]nonan-8-yl)-7-fluoro-indazol-5-yl]-2,8-dimethyl-imidazo [1,2-b]pyridazine (40.0 mg, 98.8 umol, 1.00 eq) in methanol (0.400 mL) was added triethylamine (20.0 mg, 197 umol, 2.00 eq). The reaction was stirred at 25° C. for 30 min. Then, acetaldehyde (5.00 M, 39.5 uL, 2.00 eq), acetic acid (5.94 mg, 98.8 umol, 1.00 eq) and sodium cyanoborohydride (9.32 mg, 148 umol, 1.50 eq) was added to the above mixture at 0° C. under N₂ protection. The reaction mixture was stirred at 25° C. for 12 hrs. The reaction mixture quenched by water (1.00 mL), extracted with ethyl acetate (1.00 mL×3), dried over Na₂SO₄, filtered and concentrated under reduced pressure to give residue. The residue was purified by prep-HPLC (column: Waters Xbridge BEH C18 100*30 mm*10 um; mobile phase: [water (NH₄HCO₃)-acetonitrile]; B %: 35%-65%, 8 min) to give 6-[2-(5-ethyl-5-azaspiro[3.5]nonan-8-yl)-7-fluoro-indazol-5-yl]-2,8-dimethyl-imidazo[1,2-b]pyridazine (13.0 mg, 30.3%) as a solid. LCMS: (ESI, m/z): 433.2 [M+H]⁺. ¹H NMR (400 MHz, METHANOL-d4) δ ppm 8.57 (d, J=2.63 Hz, 1H) 8.22 (s, 1H) 7.90 (s, 1H) 7.77 (d, J=13.01 Hz, 1H) 7.64 (s, 1H) 4.70-4.76 (m, 1H) 3.10-3.16 (m, 1H) 2.91-3.03 (m, 1H) 2.63-2.75 (m, 4H) 2.43-2.53 (m, 4H) 2.40 (br dd, J=12.76, 2.00 Hz, 1H) 2.23-2.36 (m, 3H) 2.12-2.23 (m, 2H) 1.95 (br d, J=12.76 Hz, 1H) 1.76-1.92 (m, 3H) 1.17 (t, J=7.19 Hz, 3H)

### Example 79: Synthesis of Compounds 204-206

### Synthesis of Intermediate C151

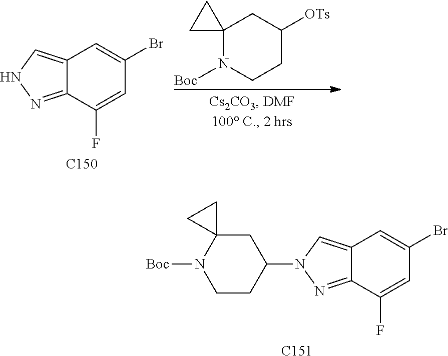

To a solution of 5-bromo-7-fluoro-2H-indazole (52-1, 1.00 g, 4.65 mmol, 1.00 eq) and tert-butyl 7-(p-tolylsulfonyloxy)-4-azaspiro[2.5]octane-4-carboxylate (1.95 g, 5.12 mmol, 1.10 eq) in N,N-dimethylformamide (25.0 mL) was added Cs₂CO₃ (4.55 g, 13.9 mmol, 3.00 eq). The reaction mixture was stirred at 100° C. for 2 hrs. The reaction mixture was diluted with H₂O (100.0 mL), extracted with ethyl acetate (3×50.0 mL). The organic layer was washed with brine (3×50.0 mL), dried over Na₂SO₄, filtered and concentrated under reduced pressure to give an oil. The oil was purified by prep-HPLC (column: Waters Xbridge Prep OBD C18 150×40 mm×10 um; mobile phase: [water (NH₄HCO₃)-acetonitrile]; B %: 60%-90%, 8 min) to give tert-butyl 7-(5-bromo-7-fluoro-indazol-2-yl)-4-azaspiro[2.5]octane-4-carboxylate (1.10 g, 27.3%). LCMS: (ESI, m/z): 368.5 [M+H]⁺. ¹H NMR (400 MHz, DMSO-d6) δ ppm 8.58 (d, J=2.75 Hz, 1H) 7.81 (d, J=1.50 Hz, 1H) 7.26 (dd, J=11.01, 1.50 Hz, 1H) 4.84-5.05 (m, 1H) 4.01-4.06 (m, 1H) 3.02 (br t, J=12.13 Hz, 1H) 2.37 (br t, J=11.88 Hz, 1H) 2.13 (br dd, J=12.51, 1.88 Hz, 1H) 1.87-1.98 (m, 1H) 1.53 (dd, J=12.44, 3.44 Hz, 1H) 1.43 (s, 9H) 1.14-1.17 (m, 1H) 0.83-0.95 (m, 1H) 0.54-0.72 (m, 2H).

### Synthesis of Intermediate C152

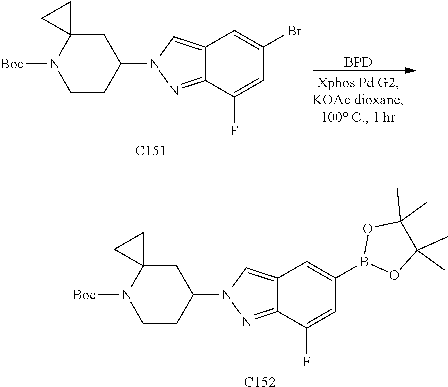

To a solution of tert-butyl 7-(5-bromo-7-fluoro-indazol-2-yl)-4-azaspiro[2.5]octane-4-carboxylate (200 mg, 471 umol, 1.00 eq) and bis(pinacolato)diboron (239 mg, 942 umol, 2.00 eq) in 1,4-dioxane (2.00 mL) was added potassium acetate (138 mg, 1.41 mmol, 3.00 eq) and chloro(2-dicyclohexylphosphino-2′,4′,6′-triisopropyl-1,1′-biphenyl)[2-(2′-amino-1,1′-biphenyl)]palladium (37.0 mg, 47.1 umol, 0.10 eq). The reaction was stirred at 100° C. for 1 hr. The reaction mixture was filtered and the filtrate was concentrated in vacuum to give tert-butyl 7-[7-fluoro-5-(4,4,5,5-tetramethyl-1,3,2-dioxaborolan-2-yl)indazol-2-yl]-4-azaspiro[2.5]octane-4-carboxylate (52-5B, 524 mg, crude) as an oil. The oil was used to the next step directly without further purification. LCMS: (ESI, m/z): 472.6 [M+H]⁺.

### Synthesis of Intermediate C153

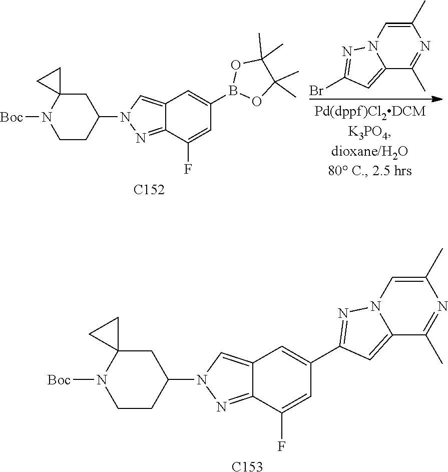

To a mixture of tert-butyl 7-[7-fluoro-5-(4,4,5,5-tetramethyl-1,3,2-dioxaborolan-2-yl)indazol-2-yl]-4-azaspiro[2.5]octane-4-carboxylate (521 mg, 1.11 mmol, 2.00 eq) and 2-bromo-4,6-dimethyl-pyrazolo[1,5-a]pyrazine (125 mg, 552 umol, 1.00 eq) in 1,4-dioxane (1.26 mL) was added a solution of K₃PO₄ (352 mg, 1.66 mmol, 3.00 eq) in H₂O (0.260 mL) and 1,1 [symbol] bis(diphenylphosphino)ferrocenedichloro palladium(II) dichloromethane complex (36.0 mg, 55.2 umol, 0.10 eq) successively under N₂ protection. The reaction was stirred at 80° C. for 2.5 hrs. The reaction mixture was diluted with H₂O (50.0 mL), extracted with ethyl acetate (3×50.0 mL). The combined organic layer was dried over Na₂SO₄, filtered and concentrated under reduced pressure to give an oil. The oil was purified by column chromatography on silica gel eluted with petroleum ether/ethyl acetate (10/1 to 0/1) to give tert-butyl 7-[5-(4,6-dimethylpyrazolo[1,5-a]pyrazin-2-yl)-7-fluoro-indazol-2-yl]-4-azaspiro[2.5]octane-4-carboxylate (52-5, 80.0 mg, 29.4%) as a solid. LCMS: (ESI, m/z): 491.6 [M+H]⁺. ¹H NMR (400 MHz, CHLOROFORM-d) δ ppm 8.09 (s, 1H) 8.03-8.06 (m, 2H) 7.58 (d, J=12.38 Hz, 1H) 6.95 (s, 1H) 4.88 (tt, J=11.74, 4.14 Hz, 1H) 4.24-4.36 (m, 1H) 3.01-3.15 (m, 1H) 2.78 (s, 3H) 2.52 (s, 4H) 2.29-2.38 (m, 1H) 2.08-2.19 (m, 1H) 1.64-1.70 (m, 1H) 1.51 (s, 9H) 0.94-1.02 (m, 1H) 0.81-0.92 (m, 1H) 0.61-0.72 (m, 2H).

### Synthesis of Compound 204

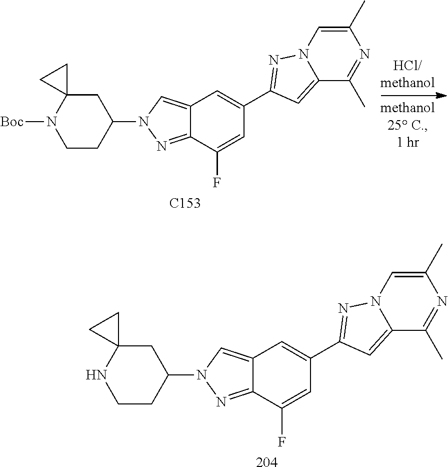

To a solution of tert-butyl 7-[5-(4,6-dimethylpyrazolo[1,5-a]pyrazin-2-yl)-7-fluoro-indazol-2-yl]-4-azaspiro[2.5]octane-4-carboxylate (52-5, 75.0 mg, 152 umol, 1.00 eq) in methanol (2.00 mL) was added HCl/methanol (4.00 M, 1.53 mL, 40.0 eq). The reaction mixture was stirred at 25° C. for 1 hr. The reaction mixture was filtered. The filter cake was dried over in vacuum to give 2-[2-(4-azaspiro[2.5]octan-7-yl)-7-fluoro-indazol-5-yl]-4,6-dimethyl-pyrazolo[1,5-a]pyrazine (4.46 mg, 7.39%) as a solid. LCMS: (ESI, m/z): 391.1 [M+H]⁺. 1H NMR (400 MHz, METHANOL-d4) δ ppm 8.91 (s, 1H) 8.59 (d, J=2.64 Hz, 1H) 8.34 (d, J=0.88 Hz, 1H) 8.06 (s, 1H) 7.76 (dd, J=12.55, 1.00 Hz, 1H) 5.04-5.17 (m, 1H) 3.68 (dt, J=12.74, 3.48 Hz, 1H) 3.38-3.50 (m, 1H) 3.08 (s, 3H) 2.88-2.97 (m, 1H) 2.66 (s, 3H) 2.52-2.59 (m, 2H) 1.96 (dd, J=13.99, 3.45 Hz, 1H) 1.12-1.21 (m, 2H) 1.05-1.11 (m, 1H) 0.96-1.02 (m, 1H).

### Synthesis of Intermediates C154 and C155

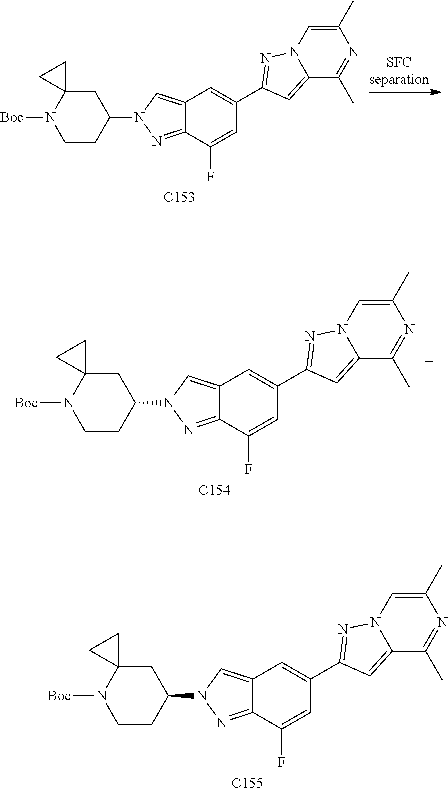 [SMILES: Cc1cn2nc(-c3cc(F)c4nn(C5CCN(C(=O)OC(C)(C)C)C6(CC6)C5)cc4c3)cc2c(C)n1 ; "C153" ; Cc1cn2nc(-c3cc(F)c4nn([C@@H]5CCN(C(=O)OC(C)(C)C)C6(CC6)C5)cc4c3)cc2c(C)n1 ; "C154" ; Cc1cn2nc(-c3cc(F)c4nn([C@H]5CCN(C(=O)OC(C)(C)C)C6(CC6)C5)cc4c3)cc2c(C)n1 ; "C155"]

Intermediate C153 (500 mg) was separated by SFC (column: (s,s) WHELK-O1 (250 mm×30 mm, 5 um); mobile phase: [0.1% NH₃H₂O ethanol]; B %: 50%-50%, 10 min) to afford tert-butyl (7R)-7-[5-(4,6-dimethylpyrazolo[1,5-a]pyrazin-2-yl)-7-fluoro-indazol-2-yl]-4-azaspiro[2.5]octane-4 carboxylate (140 mg, 28.0%) as a solid and tert-butyl (7S)-7-[5-(4,6-dimethylpyrazolo[1,5-a]pyrazin-2-yl)-7-fluoro-indazol-2-yl]-4-azaspiro[2.5]octane-4-carboxylate (150 mg, 30.0%) as a solid. Intermediate C154: LCMS: (ESI, m/z): 491.4 [M+H]⁺. ¹H NMR (400 MHz, CHLOROFORM-d) δ ppm 8.09 (s, 1H) 8.02-8.06 (m, 2H) 7.58 (dd, J=12.38, 1.00 Hz, 1H) 6.95 (s, 1H) 4.79-4.98 (m, 1H) 4.28 (br d, J=13.13 Hz, 1H) 3.04-3.16 (m, 1H) 2.78 (s, 3H) 2.49-2.62 (m, 4H) 2.33 (br d, J=11.13 Hz, 1H) 2.06-2.21 (m, 1H) 1.66-1.70 (m, 1H) 1.51 (s, 9H) 1.40 (dt, J=9.44, 6.66 Hz, 1H) 0.92-1.04 (m, 1H) 0.61-0.73 (m, 2H) Intermediate C155: LCMS: (ESI, m/z): 491.4 [M+H]⁺. ¹H NMR (400 MHz, CHLOROFORM-d) δ ppm 8.09 (s, 1H) 8.02-8.07 (m, 2H) 7.58 (dd, J=12.38, 0.88 Hz, 1H) 6.95 (s, 1H) 4.79-4.99 (m, 1H) 4.28 (br d, J=13.63 Hz, 1H) 2.98-3.19 (m, 1H) 2.78 (s, 3H) 2.48-2.64 (m, 4H) 2.22-2.39 (m, 1H) 2.06-2.19 (m, 1H) 1.66-1.70 (m, 1H) 1.48-1.54 (m, 9H) 1.40 (dt, J=9.44, 6.47 Hz, 1H) 0.93-1.03 (m, 1H) 0.62-0.72 (m, 2H)

### Synthesis of Compound 206

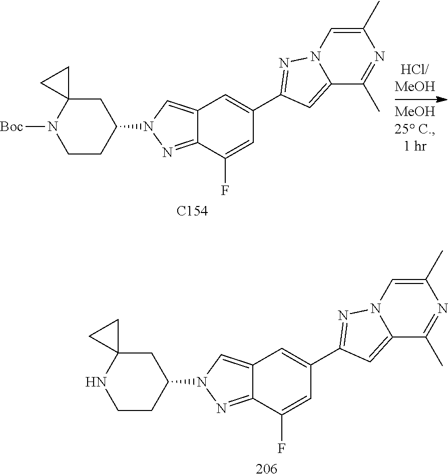

To a solution of tert-butyl (7R)-7-[5-(4,6-dimethylpyrazolo[1,5-a]pyrazin-2-yl)-7-fluoro-indazol-2-yl]-4-azaspiro[2.5]octane-4-carboxylate (120 mg, 244 umol, 1.00 eq) in methanol (2.40 mL) was added HCl/methanol (4.00 M, 2.38 mL). The reaction mixture was stirred at 25° C. for 1 hr. LCMS showed the reaction mixture was completed. The reaction mixture was filtered. The filter cake was dried in vacuum to get a solid. The solid was diluted with H₂O (5.00 mL) and lyophilized to give 2-[2-[(7R)-4-azaspiro[2.5]octan-7-yl]-7-fluoro-indazol-5-yl]-4,6-dimethyl-pyrazolo[1,5-a]pyrazine (37.0 mg, 38.8%) as a solid. LCMS: (ESI, m/z): 391.3 [M+H]⁺. ¹H NMR (400 MHz, METHANOL-d4) δ ppm 8.91 (s, 1H) 8.59 (d, J=2.51 Hz, 1H) 8.34 (d, J=0.88 Hz, 1H) 8.05 (s, 1H) 7.76 (dd, J=12.49, 0.94 Hz, 1H) 5.04-5.17 (m, 1H) 3.68 (dt, J=12.71, 3.31 Hz, 1H) 3.39-3.50 (m, 1H) 3.08 (s, 3H) 2.92 (t, J=12.49 Hz, 1H) 2.65 (s, 3H) 2.51-2.60 (m, 2H) 1.89-2.00 (m, 1H) 0.96-1.18 (m, 4H).

### Synthesis of Compound 205

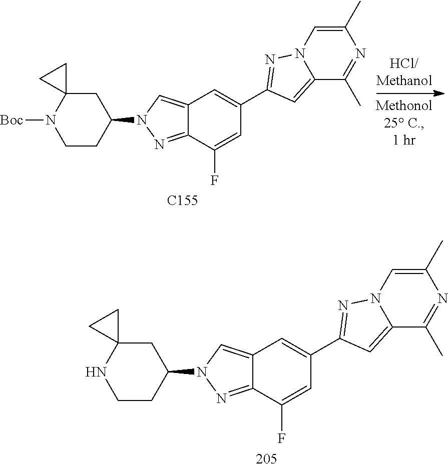

To a solution of tert-butyl (7S)-7-[5-(4,6-dimethylpyrazolo[1,5-a]pyrazin-2-yl)-7-fluoro-indazol-2-yl]-4-azaspiro[2.5]octane-4-carboxylate (52-5B, 130 mg, 265 umol, 1.00 eq) in methanol (2.60 mL) was added HCl/methanol (4.00 M, 2.58 mL). The reaction mixture was stirred at 25° C. for 1 hr. The reaction mixture was filtered. The filter cake was dried in vacuum to get yellow solid. The solid was diluted with H₂O (5.00 mL) and filtered. The filter cake was dried over in vacuum to give 2-[2-[(7S)-4-azaspiro[2.5]octan-7-yl]-7-fluoro-indazol-5-yl]-4,6-dimethyl-pyrazolo[1,5-a]pyrazine (53.5 mg, 51.7%) as a solid. LCMS: (ESI, m/z): 391.2 [M+H]⁺. ¹H NMR (400 MHz, METHANOL-d4) δ ppm 8.90 (s, 1H) 8.59 (d, J=2.51 Hz, 1H) 8.34 (d, J=1.00 Hz, 1H) 8.05 (s, 1H) 7.76 (dd, J=12.55, 1.00 Hz, 1H) 5.04-5.14 (m, 1H) 3.69 (dt, J=12.77, 3.53 Hz, 1H) 3.39-3.51 (m, 1H) 3.08 (s, 3H) 2.86-2.97 (m, 1H) 2.65 (d, J=0.75 Hz, 3H) 2.51-2.59 (m, 2H) 1.91 (br d, J=5.90 Hz, 1H) 0.96-1.20 (m, 4H).

### Example 80: Synthesis of Compound 175

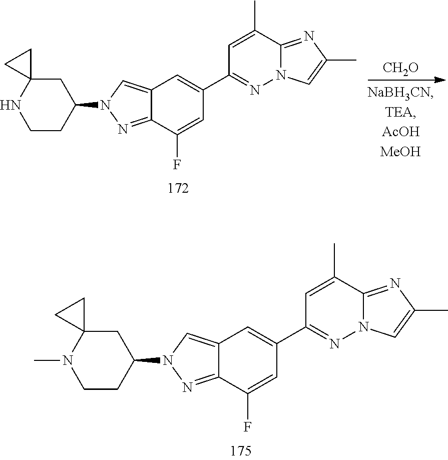

To a solution of 6-[2-[(7S)-4-azaspiro[2.5]octan-7-yl]-7-fluoro-indazol-5-yl]-2,8-dimethyl-imidazo[1,2-b]pyridazine (90.0 mg, 230 umol, 1.00 eq) in methanol (1.00 mL) was added triethylamine (46.6 mg, 461 umol, 64.1 uL, 2.00 eq) at 25° C. The reaction mixture was stirred at 25° C. for 30 min. Then formaldehyde (55.3 mg, 1.84 mmol, 50.8 uL, 8.00 eq) and acetic acid (27.6 mg, 461 umol, 26.3 uL, 2.00 eq) was added. The reaction mixture was stirred at 25° C. for 10 min and sodium cyanoborohydride (43.4 mg, 691 umol, 3.00 eq) was added to the mixture at 0° C. The reaction mixture was stirred at 25° C. for 8 hrs. The reaction mixture was poured into saturated aqueous NaHCO₃ (5.00 mL) and extracted with ethyl acetate (5.00 mL×3). The combined organic layer was dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-acetonitrile]; B %: 30%-70%, 8 min) to give 6-[7-fluoro-2-[(7S)-4-methyl-4-azaspiro[2.5]octan-7-yl]indazol-5-yl]-2,8-dimethyl-imidazo[1,2-b]pyridazine (EVAL-0167-0046, 82.0 mg, 88.0%) as white solid. LCMS: (ESI, m/z): 405.2 [M+H]⁺. ¹H NMR (400 MHz, METHANOL-d4) δ ppm 8.55 (d, J=2.62 Hz, 1H) 8.19 (s, 1H) 7.89 (s, 1H) 7.72-7.79 (m, 1H) 7.61 (s, 1H) 4.77-4.86 (m, 1H) 3.14-3.24 (m, 1H) 3.01-3.12 (m, 1H) 2.83 (br t, J=12.34 Hz, 1H) 2.63-2.69 (m, 3H) 2.51-2.61 (m, 4H) 2.45-2.51 (m, 3H) 1.97 (br dd, J=12.99, 1.55 Hz, 1H) 1.34 (br dd, J=12.93, 2.09 Hz, 1H) 0.72-0.80 (m, 2H) 0.63-0.72 (m, 1H) 0.52-0.61 (m, 1H)

### Example 81: Synthesis of Compound 176

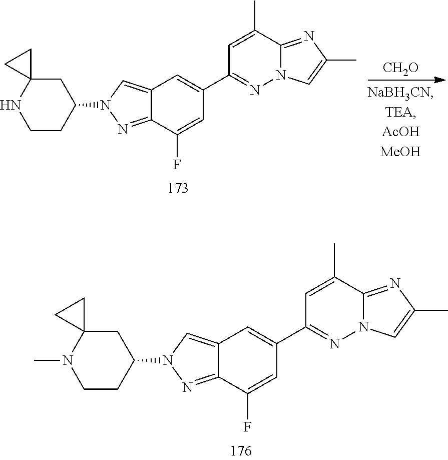

To a solution of 6-[2-[(7R)-4-azaspiro[2.5]octan-7-yl]-7-fluoro-indazol-5-yl]-2,8-dimethyl-imidazo[1,2-b]pyridazine (90.0 mg, 230 umol, 1.00 eq) in methanol (1.00 mL) was added triethylamine (46.6 mg, 461 umol, 64.1 uL, 2.00 eq) at 25° C. The reaction mixture was stirred at 25° C. for 30 min. Then formaldehyde (55.3 mg, 1.84 mmol, 50.8 uL, 8.00 eq) and acetic acid (27.6 mg, 461 umol, 26.3 uL, 2.00 eq) was added. The reaction mixture was stirred at 25° C. for 10 min and sodium cyanoborohydride (43.4 mg, 691 umol, 3.00 eq) was added to the mixture at 0° C. The reaction mixture was stirred at 25° C. for 8 hrs. The reaction mixture was poured into saturated aqueous NaHCO₃ (5.00 mL) and extracted with ethyl acetate (5.00 mL×3). The combined organic layer was dried over Na₂SO₄, filtered and concentrated under reduced pressure to give a residue. The residue was purified by prep-HPLC (column: Waters Xbridge BEH C18 100*30 mm*10 um; mobile phase: [water (NH₄HCO₃)-acetonitrile]; B %: 30%-70%, 8 min) to give 6-[7-fluoro-2-[(7R)-4-methyl-4-azaspiro[2.5]octan-7-yl]indazol-5-yl]-2,8-dimethyl-imidazo[1,2-b]pyridazine (82.7 mg, 204 umol, 88.7%) as a solid. LCMS: (ESI, m/z): 405.2 [M+H]⁺. ¹H NMR (400 MHz, METHANOL-d4) δ ppm 8.55 (d, J=2.74 Hz, 1H) 8.19 (d, J=0.95 Hz, 1H) 7.86-7.91 (m, 1H) 7.75 (dd, J=13.05, 1.13 Hz, 1H) 7.62 (d, J=0.83 Hz, 1H) 4.76-4.84 (m, 1H) 3.13-3.25 (m, 1H) 3.01-3.10 (m, 1H) 2.83 (t, J=12.40 Hz, 1H) 2.65 (d, J=0.72 Hz, 3H) 2.51-2.61 (m, 4H) 2.48 (s, 3H) 1.97 (br dd, J=12.87, 1.79 Hz, 1H) 1.30-1.40 (m, 1H) 0.70-0.80 (m, 2H) 0.62-0.70 (m, 1H) 0.52-0.61 (m, 1H)

### Example 82: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905_m1)—used for K562/suspension cell lines
GUSB: VIC-PL, ThermoFisher 4326320E (Assay: Hs99999908_m1)—used for K562/suspension cell lines
PPIA: VIC-PL, ThermoFisher 4326316E (Assay: Hs99999904_m1)—used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABKFQ/
MYB Primer 1:
CAACACCATTTCATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATC
C/3IABKFQ/

### Description

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO2 for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time |
|---|---|---|---|
| RT step | 1 | 50° C. | 5 min |
| RT inactivation/initial | 1 | 95° C. | 20 sec |
| denaturation | | | |
| Amplification | 40 | 95° C. | 3 sec |
| | | 60° C. | 30 sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 50% response in CJ decrease).

A summary of these results is illustrated in Table 2, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 2
Modulation of RNA Splicing by Exemplary Compounds

| | HTT | HTT | MYB | MYB |
|---|---|---|---|---|
| Compound | CJ | AJ | CJ | AJ |
| No. | IC₅₀ | AC₅₀ | IC₅₀ | AC₅₀ |
| 103 | B | B | C | C |
| 104 | B | B | C | C |
| 105 | B | B | B | C |
| 107 | B | B | B | B |
| 108 | B | B | B | C |
| 109 | A | B | B | B |
| 110 | B | C | B | B |
| 113 | C | C | D | D |
| 114 | D | D | D | D |
| 115 | C | C | D | C |
| 117 | D | C | D | C |
| 119 | D | D | D | D |
| 120 | C | D | D | C |
| 121 | D | D | D | D |
| 122 | A | A | A | A |
| 123 | B | B | C | C |
| 124 | A | A | B | B |
| 125 | B | B | B | B |
| 126 | C | D | D | D |
| 127 | A | B | B | B |
| 129 | A | A | B | B |
| 130 | A | A | B | B |
| 132 | B | B | C | C |
| 133 | A | A | B | B |
| 134 | C | D | C | D |
| 135 | B | B | B | B |
| 136 | A | A | B | B |
| 137 | A | A | B | B |
| 138 | A | B | B | C |
| 139 | A | A | B | B |
| 140 | C | C | C | C |
| 141 | D | D | D | D |
| 143 | B | B | B | B |
| 144 | D | D | D | D |
| 145 | B | B | C | B |
| 146 | A | A | B | B |
| 147 | B | B | B | B |
| 148 | C | D | D | D |
| 149 | A | A | B | B |
| 150 | A | A | B | B |
| 151 | A | A | A | B |
| 152 | A | B | B | B |
| 153 | D | C | C | D |
| 154 | A | B | B | B |
| 155 | A | B | B | B |
| 156 | B | B | C | C |
| 157 | D | D | C | D |
| 158 | D | D | D | D |
| 159 | D | D | D | D |
| 160 | A | A | B | B |
| 161 | B | C | C | C |
| 162 | D | D | D | D |
| 163 | D | D | D | D |
| 164 | A | A | B | B |
| 165 | D | D | D | D |
| 166 | A | A | B | B |
| 167 | B | B | C | C |
| 168 | A | B | B | B |
| 171 | A | A | B | B |
| 172 | A | A | A | A |
| 173 | A | A | B | B |
| 174 | A | A | B | B |
| 175 | A | A | B | A |
| 176 | A | A | B | C |
| 177 | A | A | B | B |
| 180 | A | A | B | B |
| 183 | A | A | B | B |
| 186 | A | A | B | B |
| 189 | A | A | B | B |
| 192 | A | B | B | B |
| 196 | C | C | D | C |
| 197 | B | B | C | C |
| 198 | A | A | B | B |
| 201 | B | C | C | C |
| 204 | B | B | C | C |
| 205 | B | B | C | B |
| 206 | A | B | B | B |
| 207 | B | B | B | B |
| 208 | D | D | D | D |
| 211 | A | B | B | B |
| 212 | A | A | B | B |

Additional studies were carried out for a larger panel of genes using the protocol provided above. The junction between flanking upstream and downstream exons was used to design canonical junction qPCR assays. At least one of the forward primer, reverse primer or the CY5-labeled 5′ nuclease probe (with 3′ quencher such as ZEN/Iowa Black FQ) was designed to overlap with the exon junction to capture the CJ mRNA transcript. BLAST was used to confirm the specificity of the probeset and parameters such as melting temperature, GC content, amplicon size, and primer dimer formation are considered during their design. Data for the decrease in CJ mRNA levels for four exemplary genes (HTT, SMN2, MYB, and Target C) analyzed in this panel are reported as IC₅₀ (compound concentration having 50% response in CJ decrease).

A summary of the results from the panel is illustrated in Table 3, wherein “A” represents an IC₅₀ of less than 100 nM; “B” represents an IC₅₀ of between 100 nM and 1 μM; and “C” represents an IC₅₀ of between 1 μM and 10 μM; and “D” represents an IC₅₀ of greater than 10 μM.

TABLE 3
Modulation of RNA Splicing by Exemplary Compounds

| | Target C | HTT | MYB | SMN2 |
|---|---|---|---|---|
| Compound | CJ | CJ | CJ | CJ |
| No. | IC₅₀ | IC₅₀ | IC₅₀ | IC₅₀ |
| 103 | C | B | C | B |
| 104 | C | B | C | A |
| 105 | B | B | B | A |
| 107 | B | B | B | A |
| 108 | C | B | B | B |
| 109 | B | A | B | A |
| 110 | C | B | B | A |
| 113 | D | C | D | C |
| 114 | D | D | D | C |
| 115 | C | C | D | B |
| 117 | D | D | D | B |
| 119 | D | D | D | C |
| 120 | D | C | D | B |
| 121 | D | D | D | C |
| 122 | A | A | A | A |
| 123 | C | B | C | B |
| 124 | B | A | B | A |
| 125 | B | B | B | A |
| 126 | C | C | D | C |
| 127 | B | A | B | A |
| 129 | B | A | B | A |
| 130 | B | A | B | A |
| 132 | C | B | C | B |
| 133 | B | A | B | A |
| 134 | D | C | C | B |
| 135 | B | B | B | A |
| 136 | B | A | B | A |
| 137 | B | A | B | A |
| 138 | B | A | B | A |
| 139 | B | A | B | A |
| 140 | C | C | C | B |
| 141 | D | D | D | C |
| 143 | B | B | B | A |
| 144 | D | D | D | C |
| 145 | B | B | C | A |
| 146 | B | A | B | A |
| 147 | B | B | B | B |
| 148 | D | C | D | C |
| 149 | A | A | B | A |
| 150 | B | A | B | A |
| 151 | A | A | A | A |
| 152 | B | A | B | A |
| 153 | D | D | C | D |
| 154 | B | A | B | A |
| 155 | B | A | B | A |
| 156 | C | B | C | B |
| 157 | D | D | C | C |
| 158 | D | D | D | C |
| 159 | D | D | D | D |
| 160 | B | A | B | A |
| 161 | C | B | C | B |
| 162 | D | D | D | C |
| 163 | D | D | D | C |
| 164 | B | A | B | A |
| 165 | D | D | D | D |
| 166 | B | A | B | A |
| 167 | B | B | C | A |
| 168 | B | A | B | A |
| 171 | A | A | B | A |
| 172 | A | A | A | A |
| 173 | B | A | B | A |
| 174 | A | A | B | A |
| 175 | A | A | B | — |
| 176 | B | A | B | — |
| 177 | A | A | B | A |
| 180 | B | A | B | A |
| 183 | A | A | B | A |
| 186 | A | A | B | A |
| 189 | B | A | B | A |
| 192 | B | A | B | A |
| 196 | D | C | D | B |
| 197 | C | B | C | B |
| 198 | A | A | B | A |
| 201 | C | B | C | B |
| 204 | B | B | C | B |
| 205 | C | B | C | B |
| 206 | B | A | B | B |
| 207 | C | B | B | A |
| 208 | D | D | D | C |
| 211 | — | A | B | A |
| 212 | B | A | B | A |

### Example 83: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials

Promega CellTiter-Glo® 2.0 Cell Viability Assay (cat #G9241)
Corning 384-well TC-treated microplates (cat #3570)

### Description

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I), (II), or (III)) were first serially diluted in DMSO then diluted 1:100 with IMDM+10% FBS. The final concentration of DMSO was 0.1% in each well. The cells were incubated for 72 hours at 37° C. and 5% CO₂ before assaying with Cell Titer Glo 2.0 reagent.

A summary of the results for viability is illustrated in Table 4, wherein A represents <100 nM; B represents 100-1000 nM; C represents 1000-9999 nM; and D represents greater than 10 μM in K562 cells.

TABLE 4
Effect of Exemplary Compounds on Cell Viability

| | K562 |
|---|---|
| Compound No. | GI₅₀ (nM) |
| 103 | C |
| 104 | C |
| 105 | B |
| 107 | B |
| 108 | B |
| 109 | B |
| 110 | C |
| 113 | C |
| 114 | D |
| 115 | C |
| 117 | C |
| 119 | D |
| 120 | C |
| 121 | C |
| 122 | A |
| 123 | C |
| 124 | B |
| 125 | B |
| 126 | D |
| 127 | B |
| 129 | B |
| 130 | B |
| 132 | C |
| 133 | B |
| 134 | C |
| 135 | B |
| 136 | B |
| 137 | B |
| 138 | C |
| 140 | D |
| 143 | B |
| 144 | C |
| 145 | C |
| 146 | B |
| 147 | B |
| 148 | D |
| 149 | A |
| 154 | B |
| 155 | B |
| 156 | C |
| 157 | D |
| 158 | C |
| 159 | D |
| 160 | B |
| 161 | C |
| 162 | C |
| 163 | C |
| 164 | B |
| 165 | D |
| 166 | C |
| 167 | C |
| 168 | B |
| 171 | B |
| 172 | B |
| 173 | B |
| 174 | B |
| 175 | B |
| 176 | B |
| 177 | B |
| 180 | B |
| 183 | B |
| 186 | B |
| 189 | B |
| 192 | B |
| 196 | D |
| 197 | C |
| 198 | B |
| 201 | C |
| 204 | C |
| 205 | C |
| 206 | B |
| 212 | B |

### Example 84: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), (II), or (III)). After treatment for 24 hours, the protein abundance of a specific target was determined by measuring luminescence.

### Materials

Promega Nano-Glo HiBiT Lytic Detection System (cat #N3030)
Corning 384-well TC-treated microplates (cat #3570)
Synthego Engineered Cells Knock-In Clones

TABLE 5
Design of genetically modified HiBiT cell lines

| | | | Guide | Guide | |
|---|---|---|---|---|---|
| Cell | | Modifi- | RNA | RNA cut | Donor |
| Line | Gene | cation | Sequence | location | Sequence |
| K562 | MYB | HIBIT | GCGCCA | chr6: | CGGTG |
| | | | TGGCCC | 135,181,526 | CGGTC |
| | | | GAAGAC | | CCCGC |
| | | | CC | | GGCTC |
| | | | | | TCGGC |
| | | | | | GGAGC |
| | | | | | CCCGC |
| | | | | | GCCCG |
| | | | | | CCGCG |
| | | | | | CCATG |
| | | | | | gtgag |
| | | | | | cggct |
| | | | | | ggcgg |
| | | | | | ctgtt |
| | | | | | caaga |
| | | | | | agatt |
| | | | | | agcGG |
| | | | | | CAGCT |
| | | | | | CCGGA |
| | | | | | GGATC |
| | | | | | TAGCG |
| | | | | | GCGCC |
| | | | | | CGAAG |
| | | | | | ACCCC |
| | | | | | GGCAC |
| | | | | | AGgta |
| | | | | | acggg |
| | | | | | gagcc |
| | | | | | ggggg |
| | | | | | gcggc |
| | | | | | cgagg |
| | | | | | g |
| K562 | HTT | HIBIT | CAGCTTT | chr4: | CGAGT |
| | | | TCCAGG | 3,074,830 | CGGCC |
| | | | GTCGCC | | CGAGG |
| | | | A | | CCTCC |
| | | | | | GGGGA |
| | | | | | CTGCC |
| | | | | | GTGCC |
| | | | | | GGGCG |
| | | | | | GGAGA |
| | | | | | CCGCC |
| | | | | | ATGgt |
| | | | | | gagcg |
| | | | | | gctgg |
| | | | | | cggct |
| | | | | | gttca |
| | | | | | agaag |
| | | | | | attag |
| | | | | | cGGCA |
| | | | | | GCTCC |
| | | | | | GGAGG |
| | | | | | ATCTA |
| | | | | | GCGGC |
| | | | | | GCGAC |
| | | | | | CCTGG |
| | | | | | AAAAG |
| | | | | | CTGAT |
| | | | | | GAAGG |
| | | | | | CCTTC |
| | | | | | GAGTC |
| | | | | | CCTCA |
| | | | | | AGTCC |
| | | | | | TTCCA |

### Description

Cells were maintained in IMDM with 10% FBS. Before the assay, cells were diluted with phenolphthalein-free growth media (IMDM+1% FBS media) and were seeded in a 384-well plate at a density of 10000 cells/well (for each cell line listed in Table 6). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 5% CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 6, the percent response for each respective cell line was calculated at each compound concentration as follows:

$$\% response = 100 * ( S - PC ) / ( NC - PC )$$

For the normalized response at each concentration, a four-parameter logistical regression was fit to the data and the response was interpolated at the 50% value to determine a concentration for protein abundance at 50% (IC₅₀) the untreated control.

A summary of the results for protein abundance is illustrated in Table 6, wherein A represents <100 nM; B represents 100-1000 nM; C represents 1000-9999 nM; and D represents greater than 10 μM.

TABLE 6
Effect of Exemplary Compounds on Protein Abundance

| Compound | | | |
|---|---|---|---|
| No. | MYB | Target C | HTT |
| 103 | C | B | B |
| 104 | B | B | B |
| 105 | B | B | B |
| 107 | B | B | B |
| 108 | B | B | B |
| 109 | B | A | A |
| 110 | B | B | B |
| 113 | C | C | C |
| 114 | D | D | D |
| 115 | C | C | C |
| 117 | C | D | C |
| 119 | C | D | D |
| 120 | C | D | C |
| 121 | D | D | D |
| 122 | A | A | A |
| 123 | C | B | B |
| 124 | B | A | A |
| 125 | B | A | A |
| 126 | D | C | C |
| 127 | B | A | A |
| 129 | B | A | A |
| 130 | B | A | A |
| 132 | C | B | B |
| 133 | B | A | A |
| 134 | C | C | C |
| 135 | B | B | A |
| 136 | B | A | A |
| 137 | B | A | A |
| 138 | B | B | B |
| 139 | B | B | B |
| 140 | C | C | C |
| 141 | D | D | D |
| 143 | B | B | B |
| 144 | C | C | C |
| 145 | B | B | B |
| 146 | B | A | A |
| 147 | B | B | B |
| 148 | D | D | C |
| 149 | B | A | A |
| 150 | B | A | A |
| 151 | A | A | A |
| 152 | B | B | A |
| 153 | C | C | C |
| 154 | B | A | A |
| 155 | B | B | A |
| 156 | C | C | B |
| 157 | C | D | C |
| 158 | C | C | C |
| 159 | D | D | D |
| 160 | B | A | A |
| 161 | C | C | B |
| 162 | C | C | C |
| 163 | C | C | C |
| 164 | B | A | A |
| 165 | D | D | D |
| 166 | B | B | A |
| 167 | C | B | B |
| 168 | B | B | A |
| 171 | B | A | A |
| 172 | A | A | A |
| 173 | B | A | A |
| 174 | B | A | A |
| 177 | B | A | A |
| 180 | B | A | A |
| 183 | B | A | A |
| 186 | B | A | A |
| 189 | B | A | A |
| 192 | B | B | A |
| 212 | B | A | A |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 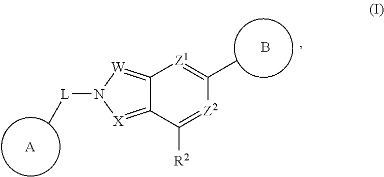
or a pharmaceutically acceptable salt, solvate, hydrate, tautomer, or stereoisomer thereof, wherein:
A and B are each independently cycloalkyl, heterocyclyl, aryl, or heteroaryl, each of which is optionally substituted with one or more R¹;
L is absent, C₁-C₆-alkylene, C₁-C₆-heteroalkylene, C(O), or —C(O)N(R^{B})—, wherein each alkylene and heteroalkylene is optionally substituted with one or more R⁸;
W, X, Z¹, and Z² are each independently C(R³) or N, wherein at least one of W and X is N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₂-C₆ alkenylene-aryl, C₁-C₆ heteroalkylene-aryl, heteroaryl, C₁-C₆ alkylene-heteroaryl, C₁-C₆ heteroalkylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁴; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁴;
R² is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵;
R³ is hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —C(O)R^{D}, or —OR^{A}; wherein each alkyl, alkenyl, alkynyl, heteroalkyl, haloalkyl, cycloalkyl, heterocyclyl, aryl, and heteroaryl is optionally substituted with one or more R⁵;
each R⁴ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkenyl, alkynyl, heteroalkyl, haloalkyl, cycloalkyl, heterocyclyl, aryl, and heteroaryl is optionally substituted with one or more R⁶;
each R⁵ is C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁶ is independently C₁-C₆-alkyl, C₁-C₆-heteroalkyl, C₁-C₆-haloalkyl, cycloalkyl, heterocyclyl, aryl, heteroaryl, halo, cyano, oxo, or —OR^{A};
each R^{A} is independently hydrogen, C₁-C₆ alkyl, C₂-C₆ alkenyl, C₂-C₆ alkynyl, C₁-C₆ haloalkyl, C₁-C₆ heteroalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D};
each of R^{B} and R^{C} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, —C(O)R^{D}, or —S(O)ₓR^{D}; or
R^{B} and R^{C} together with the atom to which they are attached form a 3-7-membered heterocyclyl ring optionally substituted with one or more R⁷;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁷ and R⁸ is independently C₁-C₆-alkyl, halo, cyano, oxo, or —OR^{A1};
each R^{A1} is hydrogen or C₁-C₆-alkyl; and
x is 0, 1, or 2.

2. The compound of claim 1, wherein each of A and B is independently heteroaryl or heterocyclyl, each of which is optionally substituted with one or more R¹.

3. The compound of any one of the preceding claims, wherein one of A and B is independently a monocyclic heteroaryl or bicyclic heteroaryl, each of which is optionally substituted with one or more R¹.

4. The compound of any one of the preceding claims, wherein one of A and B is independently a bicyclic heteroaryl optionally substituted with one or more R¹.

5. The compound of any one of the preceding claims, wherein one of A and B is independently a nitrogen-containing heteroaryl optionally substituted with one or more R¹.

6. The compound of any one of the preceding claims, wherein one of A and B is a 5-10 membered heteroaryl optionally substituted with one or more R¹.

7. The compound of any one of the preceding claims, wherein one of A and B is independently selected from 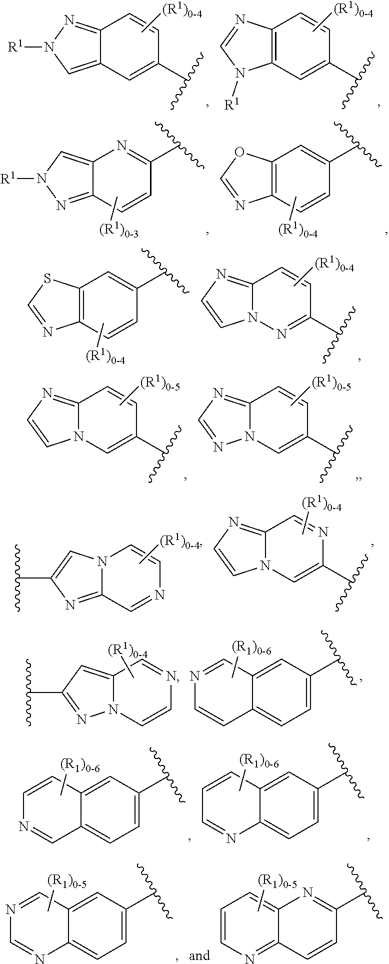
wherein R¹ is as described in claim 1.

8. The compound of any one of the preceding claims, wherein one of A and B is independently selected from 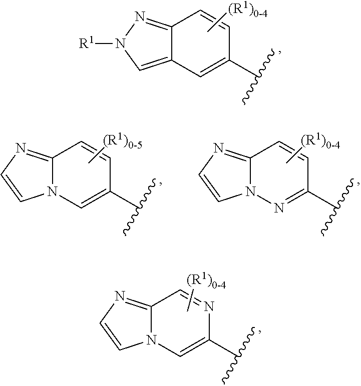
wherein R¹ is as described in claim 1.

9. The compound of any one of the preceding claims, wherein one of A and B is independently selected from 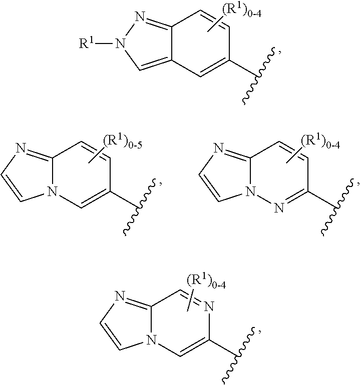
wherein each R¹ is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁴.

10. The compound of any one of the preceding claims, wherein one of A and B is independently 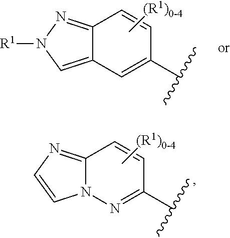
wherein each R¹ is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁴.

11. The compound of any one of the preceding claims, wherein A is selected from 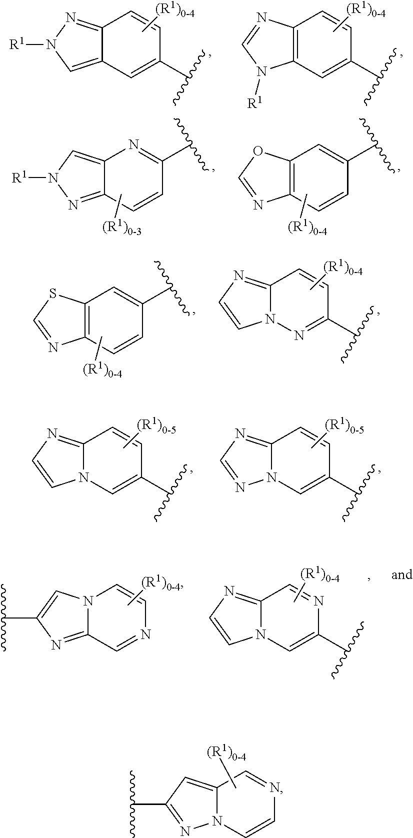
wherein R¹ is as described in claim 1.

12. The compound of any one of the preceding claims, wherein B is selected from 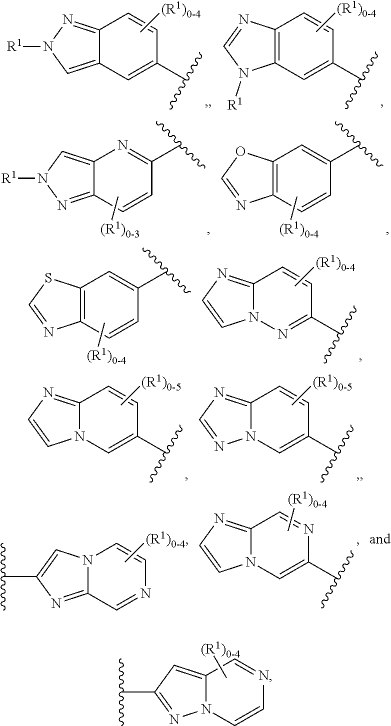
wherein R¹ is as described in claim 1.

13. The compound of any one of the preceding claims, wherein A is selected from 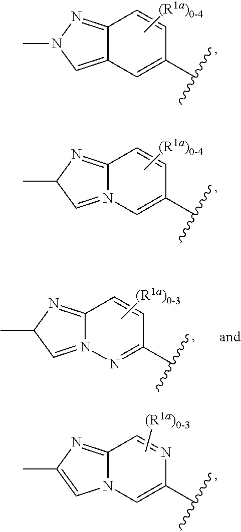
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁴.

14. The compound of any one of the preceding claims, wherein A is 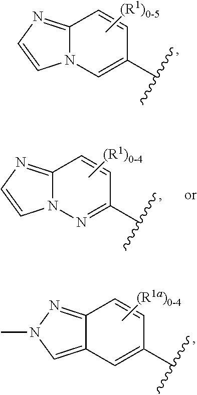
wherein R¹ is as described in claim 1.

15. The compound of any one of the preceding claims, wherein B is 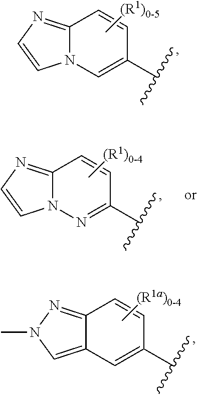
wherein R¹ is as described in claim 1.

16. The compound of any one of the preceding claims, wherein one of A and B is independently selected from

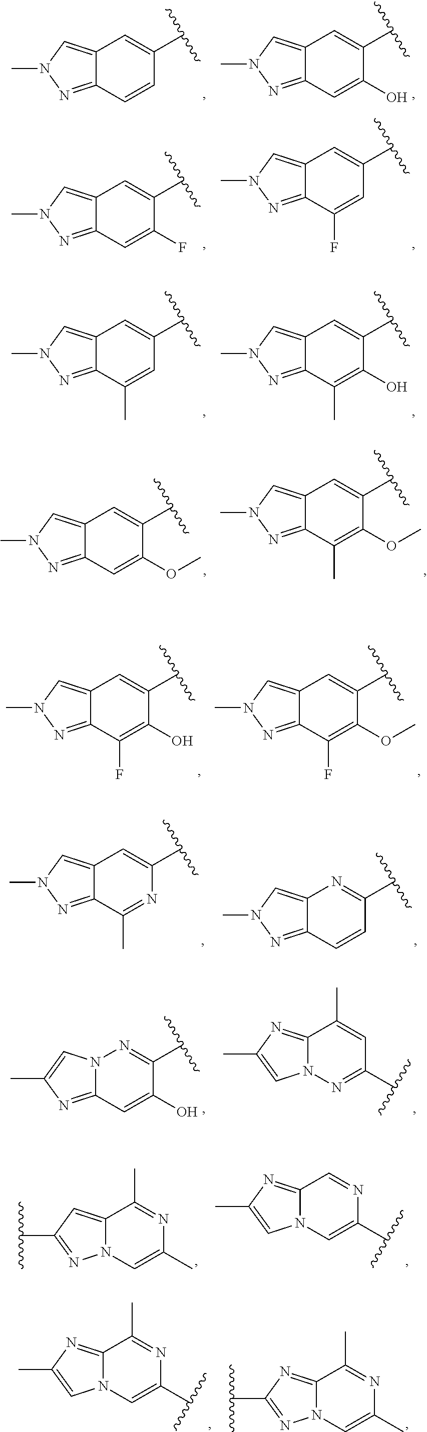

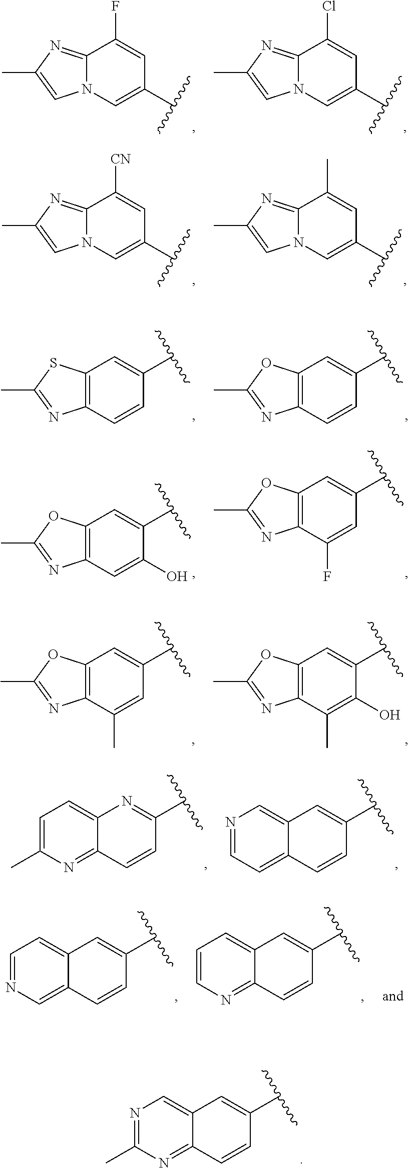

17. The compound of any one of the preceding claims, wherein one of A and B is independently selected from

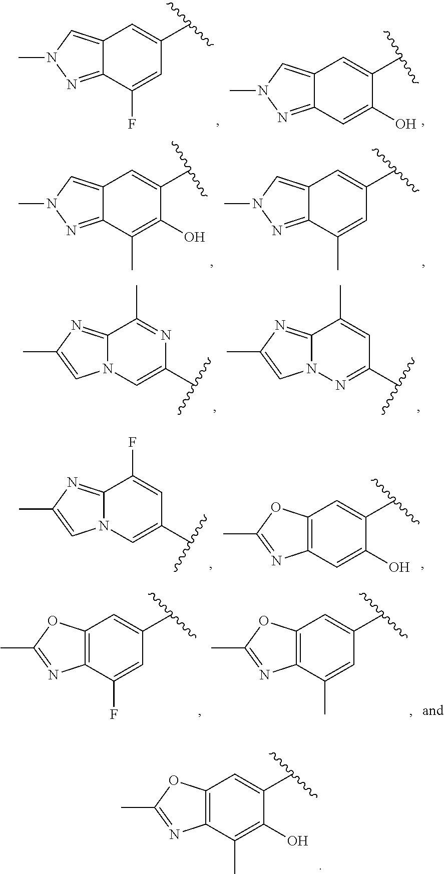

18. The compound of any one of the preceding claims, wherein one of A and B is independently selected from

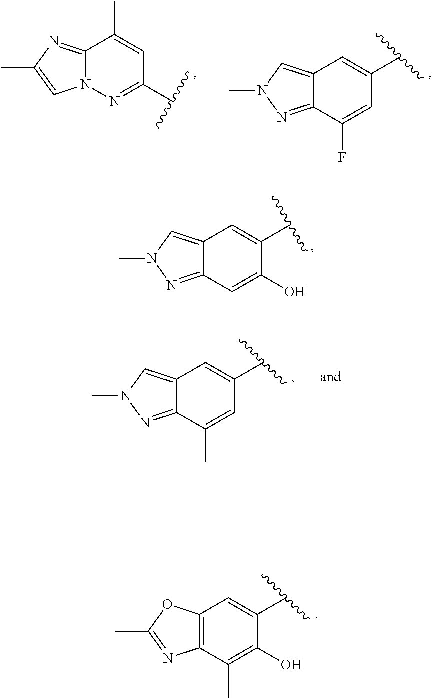 [SMILES: *c1cc(C)c2nc(C)cn2n1 ; *c1cc(F)c2nn(C)cc2c1 ; *c1cc2cn(C)nc2cc1O ; *c1cc(C)c2nn(C)cc2c1 ; *c1cc2oc(C)nc2c(C)c1O]

19. The compound of any one of the preceding claims, wherein A is selected from

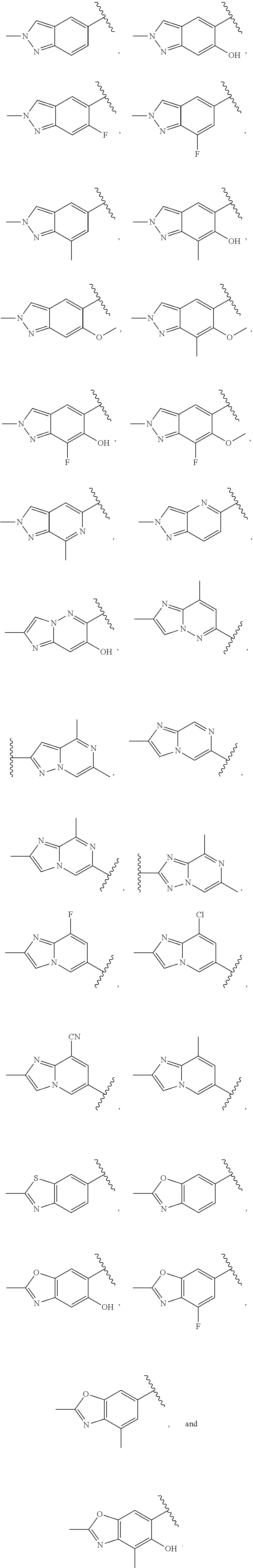

20. The compound of any one of the preceding claims, wherein B is selected from

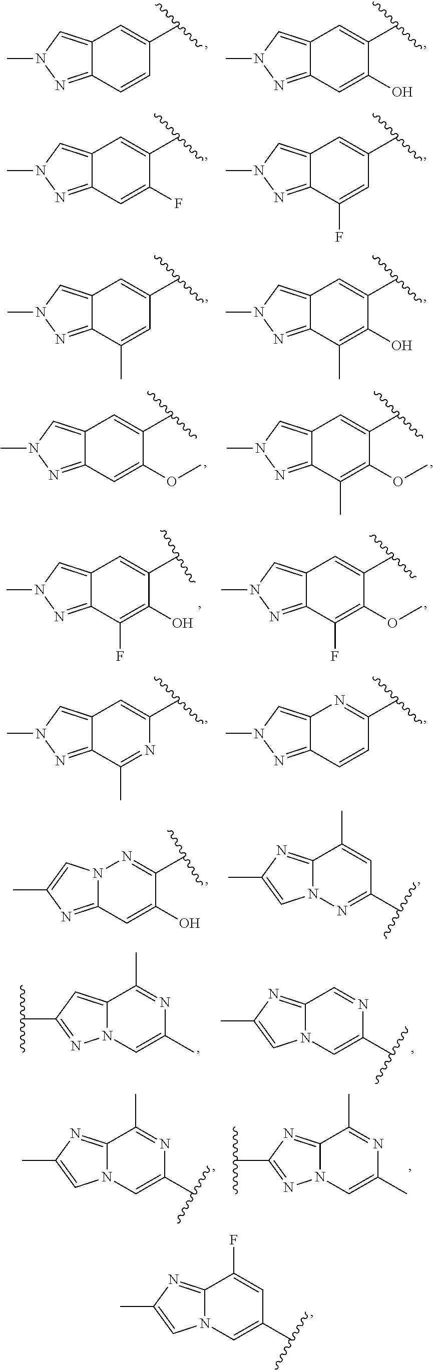

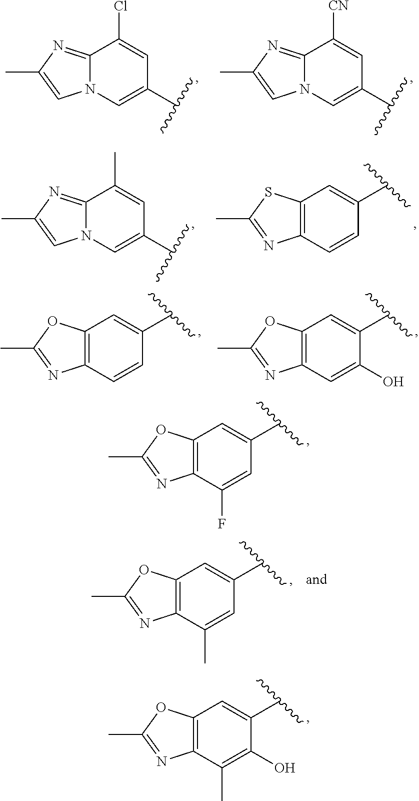

21. The compound of any one of the preceding claims, wherein A selected from

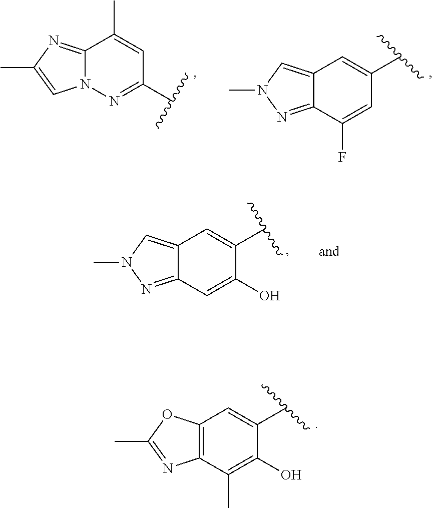 [SMILES: *c1cc(C)c2nc(C)cn2n1 ; *c1cc(F)c2nn(C)cc2c1 ; *c1cc2cn(C)nc2cc1O ; *c1cc2oc(C)nc2c(C)c1O]

22. The compound of any one of the preceding claims, wherein B selected from

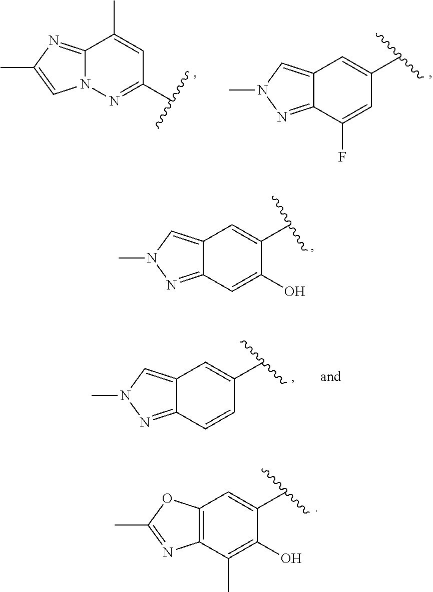 [SMILES: *c1cc(C)c2nc(C)cn2n1 ; *c1cc(F)c2nn(C)cc2c1 ; *c1cc2cn(C)nc2cc1O ; *c1ccc2nn(C)cc2c1 ; *c1cc2oc(C)nc2c(C)c1O]

23. The compound of any one of the preceding claims, wherein one of A and B is independently a monocyclic heterocyclyl or bicyclic heterocyclyl, each of which is optionally substituted with one or more R¹.

24. The compound of any one of the preceding claims, wherein one of A and B is independently a nitrogen-containing heterocyclyl optionally substituted with one or more R¹.

25. The compound of any one of the preceding claims, wherein one of A and B is independently a 4-8 membered heterocyclyl optionally substituted with one or more R¹.

26. The compound of any one of the preceding claims, wherein one of A and B is independently selected from 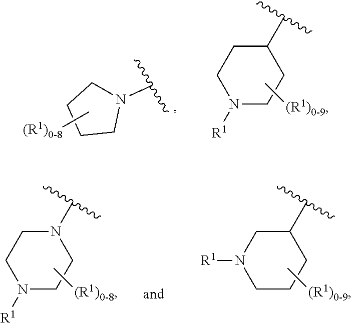
wherein R¹ is as described in claim 1.

27. The compound of any one of the preceding claims, wherein one of A and B is independently selected from 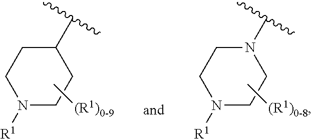
wherein R¹ is as described in claim 1.

28. The compound of any one of the preceding claims, wherein one of A and B is 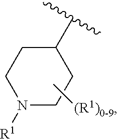
wherein R¹ is as described in claim 1.

29. The compound of any one of the preceding claims, wherein A is 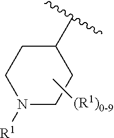
and R¹ is as described in claim 1.

30. The compound of any one of the preceding claims, wherein B is selected from 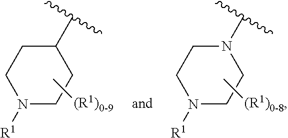
wherein R¹ is as described in claim 1.

31. The compound of any one of the preceding claims, wherein B is 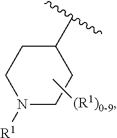
wherein R¹ is as described in claim 1.

32. The compound of any one of the preceding claims, wherein one of A and B is independently selected from

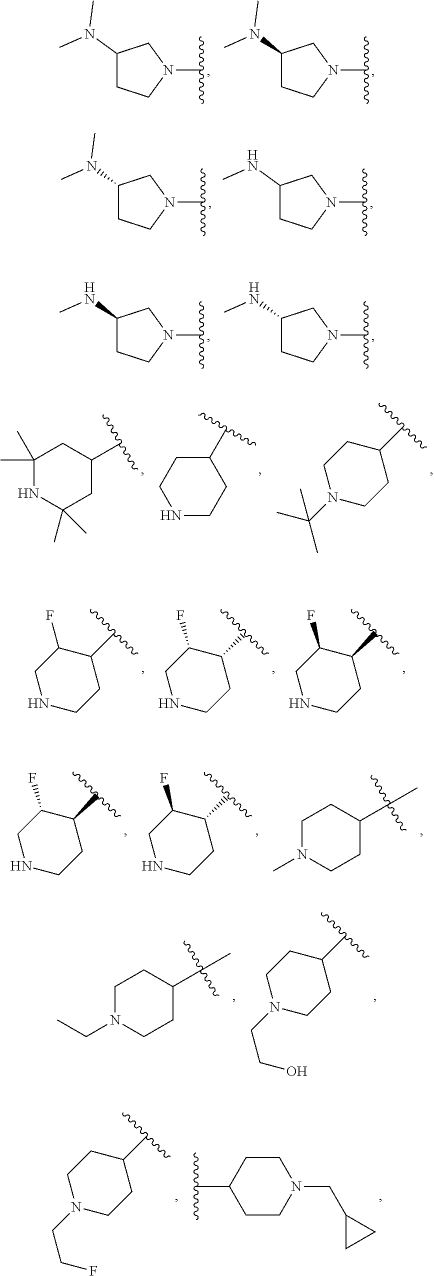

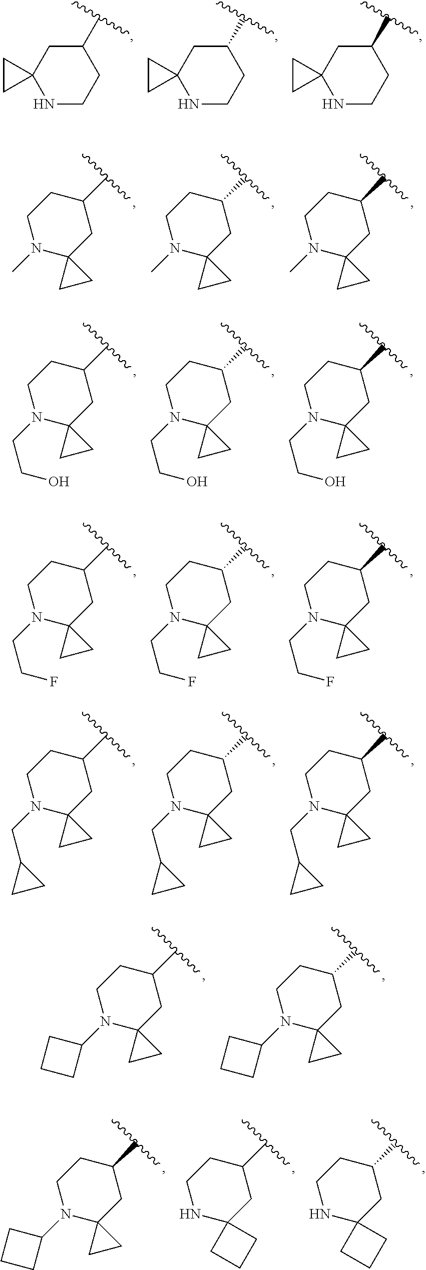

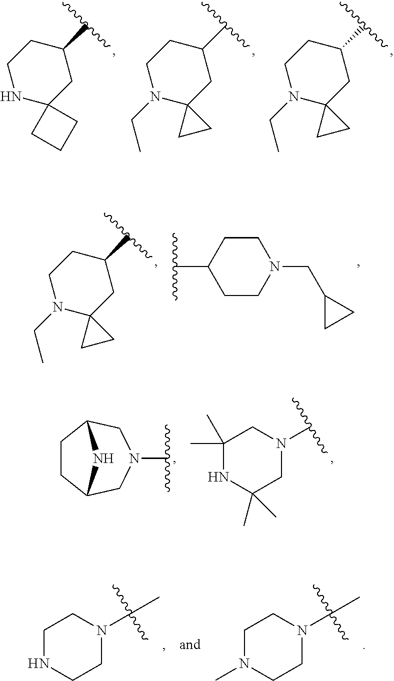

33. The compound of any one of the preceding claims, wherein one of A and B is independently selected from

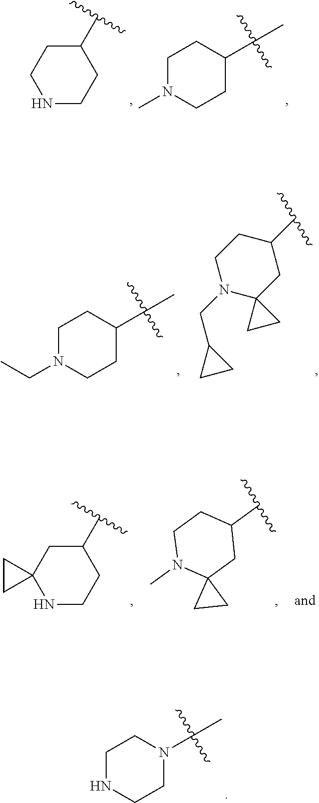

34. The compound of any one of the preceding claims, wherein one of A and B is

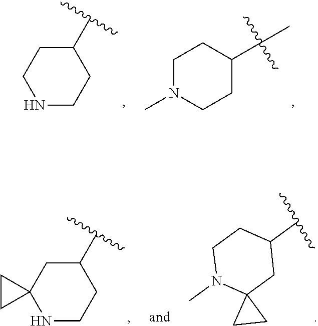

35. The compound of any one of the preceding claims, wherein A is selected from

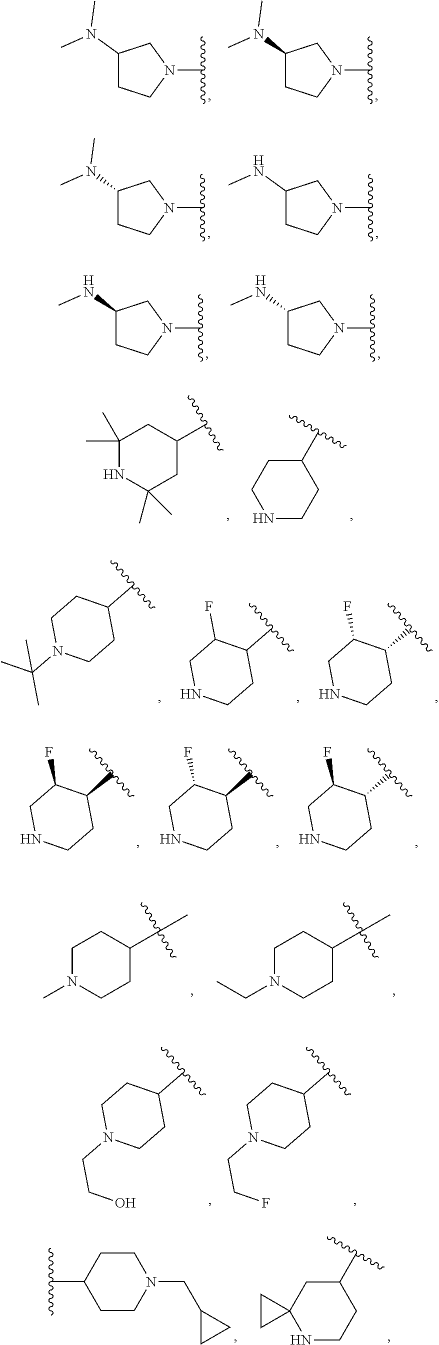

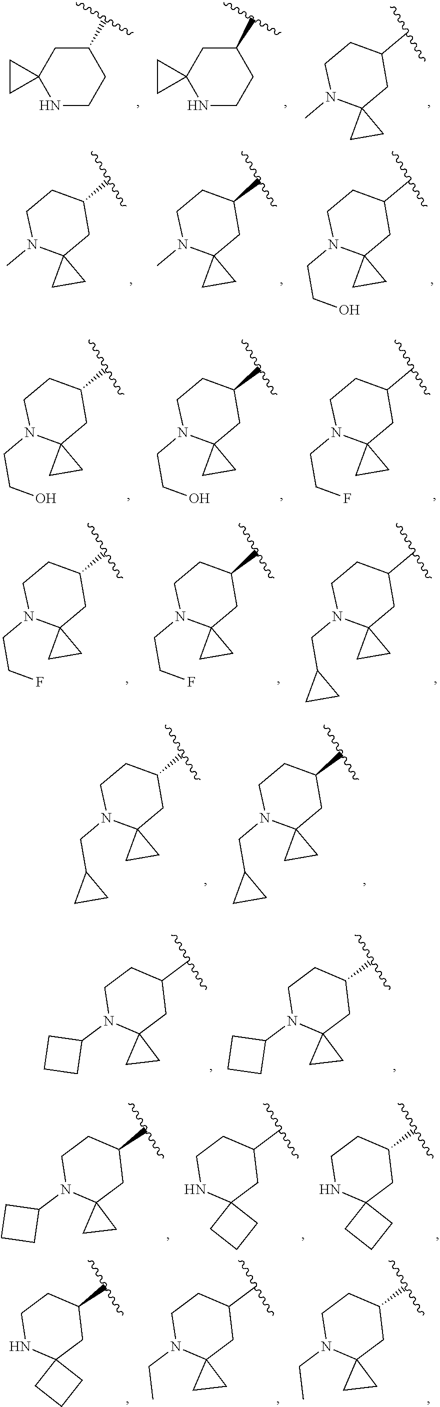

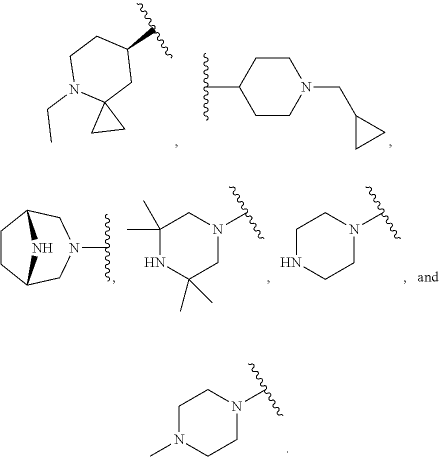

36. The compound of any one of the preceding claims, wherein B is selected from

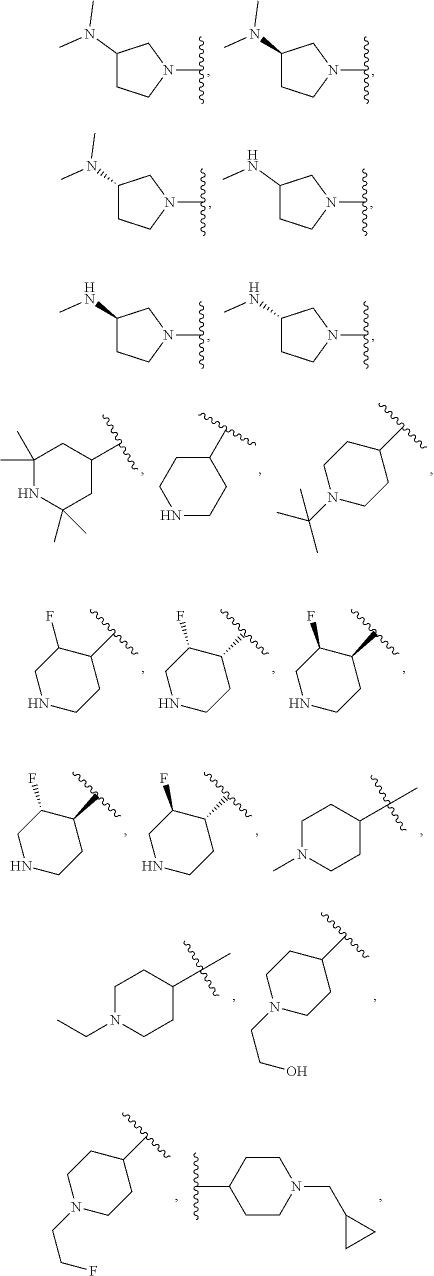

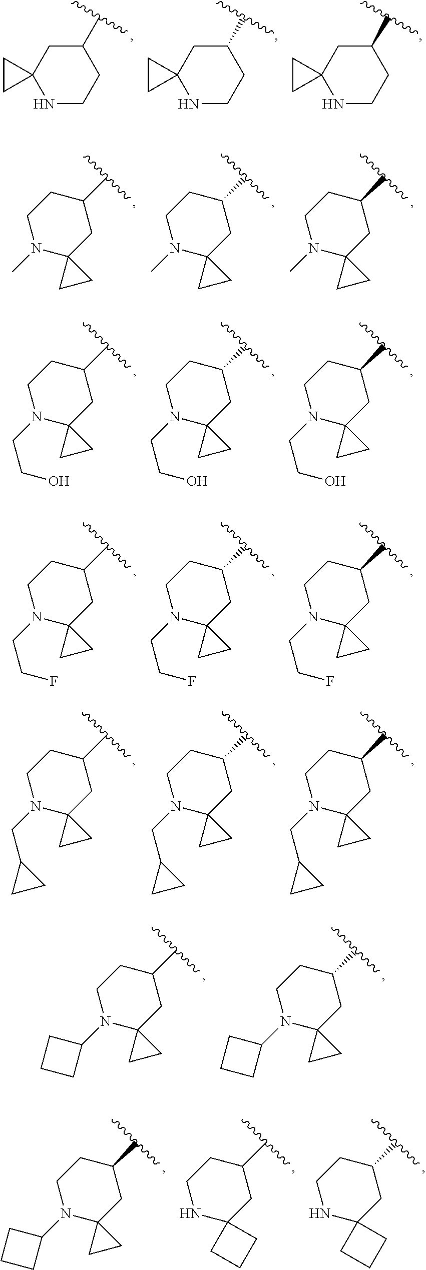

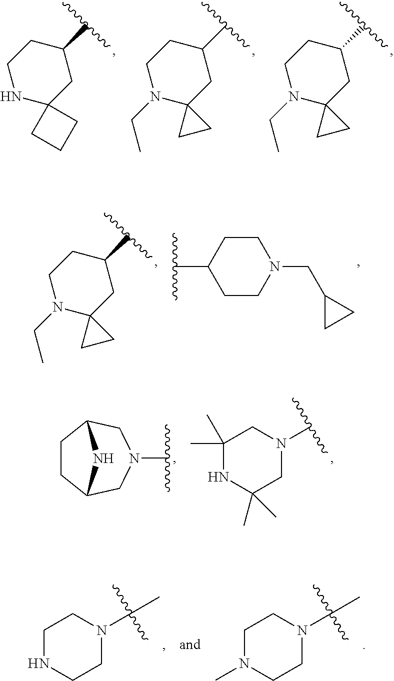

37. The compound of any one of the preceding claims, wherein A is selected from

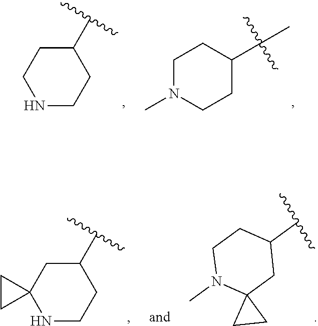

38. The compound of any one of the preceding claims, wherein B is selected from

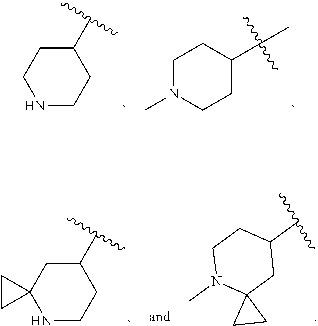

39. The compound of any one of the preceding claims, wherein X is N.

40. The compound of any one of the preceding claims, wherein W is N.

41. The compound of any one of the preceding claims, wherein both of W and X is independently N.

42. The compound of any one of the preceding claims, wherein Z¹ is C(R³) (e.g., CH).

43. The compound of any one of the preceding claims, wherein Z¹ is N.

44. The compound of any one of the preceding claims, wherein X is N and W, Z¹, and Z² are each independently C(R³).

45. The compound of any one of the preceding claims, wherein X and Z¹ are each independently N, and W and Z² are each independently C(R³).

46. The compound of any one of the preceding claims, wherein X and W are each independently N and Z¹ is C(R³).

47. The compound of any one of the preceding claims, wherein each of W, X, and Z¹ are each independently N, and Z¹ is C(R³).

48. The compound of any one of the preceding claims, wherein R² is hydrogen.

49. The compound of any one of the preceding claims, wherein R² is C₁-C₆-alkyl, halo, or —OR^{A}.

50. The compound of any one of the preceding claims, wherein R² is —CH₃, —Cl, —F, —OCH₃, or

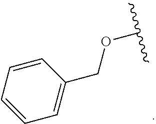 [SMILES: *OCc1ccccc1]

51. The compound of any one of the preceding claims, wherein L is absent or C₁-C₆-alkylene optionally substituted with one or more R⁸.

52. The compound of any one of the preceding claims, wherein L is absent.

53. The compound of any one of the preceding claims, wherein the compound of Formula (I) is Formula (I-a): 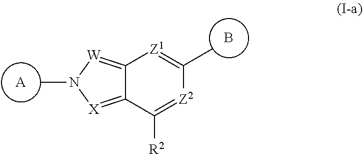
or a pharmaceutically acceptable salt, solvate, hydrate, tautomer, or stereoisomer thereof, wherein A, B, W, X, Z¹, Z², R² and subvariables thereof are as defined in claim 1.

54. The compound of any one of the preceding claims, wherein the compound of Formula (I) is Formula (I-b): 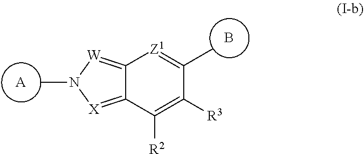
or a pharmaceutically acceptable salt, solvate, hydrate, tautomer, or stereoisomer thereof, wherein each of A, B, W, X, Z¹, R², R³, and subvariables thereof is as defined in claim 1.

55. The compound of any one of the preceding claims, wherein the compound of Formula (I) is Formula (I-c): 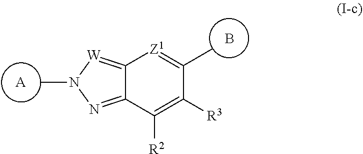
or a pharmaceutically acceptable salt, solvate, hydrate, tautomer, or stereoisomer thereof, wherein each of A, B, W, Z¹, R², R³ and subvariables thereof is as defined in claim 1.

56. The compound of any one of the preceding claims, wherein the compound of Formula (I) is Formula (I-d): 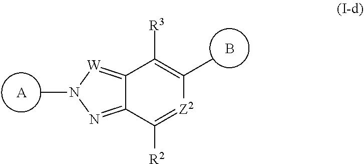
or a pharmaceutically acceptable salt, solvate, hydrate, tautomer, or stereoisomer thereof, wherein A, B, W, Z¹, R², R³ and subvariables thereof are as defined in claim 1.

57. The compound of any one of the preceding claims, wherein the compound of Formula (I) is a compound of Formula (I-e): 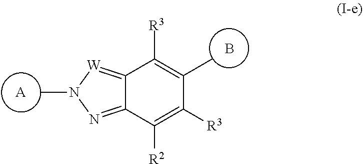
or a pharmaceutically acceptable salt, solvate, hydrate, tautomer, or stereoisomer thereof, wherein each of A, B, W, R², R³ and subvariables thereof are defined as in claim 1.

58. The compound of any one of claims 1-53, wherein the compound of Formula (I) is a compound of Formula (I-f): 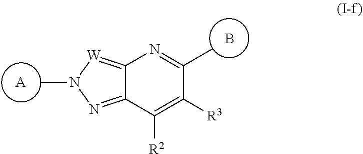
or a pharmaceutically acceptable salt, solvate, hydrate, tautomer, or stereoisomer thereof, wherein each of A, B, W, R², m, and subvariables thereof are defined as in claim 1.

59. The compound of any one of claims 1-54, wherein the compound of Formula (I) is a compound of Formula (I-g): 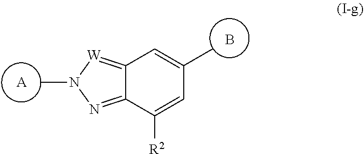
or a pharmaceutically acceptable salt, solvate, hydrate, tautomer, or stereoisomer thereof, wherein each of A, B, W, R², and subvariables thereof are defined as in claim 1.

60. The compound of any one of claims 1-51, wherein the compound of Formula (I) is a compound of Formula (I-h): 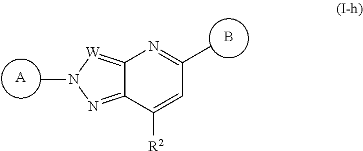
or a pharmaceutically acceptable salt, solvate, hydrate, tautomer, or stereoisomer thereof, wherein each of A, B, W, R², and subvariables thereof are defined as in claim 1.

61. The compound of any one of claims 1-50, wherein the compound of Formula (I) is a compound of Formula (I-i): 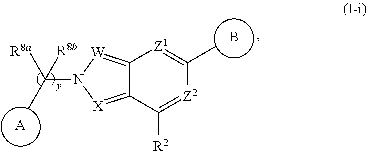
or a pharmaceutically acceptable salt, solvate, hydrate, tautomer, or stereoisomer thereof, wherein A, B, W, X, Z¹, Z², and R² are as defined in claim 1; each R^{8a} and R^{8b} is independently C₁-C₆-alkyl, halo, cyano, oxo, or —OR^{A1}; and y is 0, 1, 2, 3, or 4.

62. The compound of any one of claims 1-50, wherein the compound of Formula (I) is a compound of Formula (I-j): 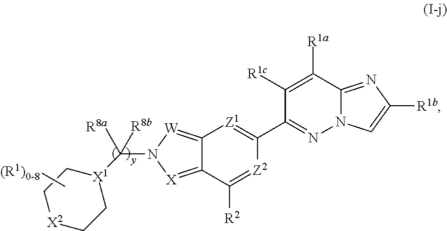
or a pharmaceutically acceptable salt, solvate, hydrate, tautomer, or stereoisomer thereof, wherein each R^{1a}, R^{1b}, R^{1c}, R^{8a} and R^{8b} is independently C₁-C₆-alkyl, halo, cyano, oxo, or —OR^{A1}; X¹ is C(R¹) or N; X² is C(R¹)₂ or N(R¹); y is 0, 1, 2, 3, or 4; and W, X, Z¹, Z², R¹ and R² are as defined in claim 1.

63. The compound of any one of claims 1-51, wherein the compound of Formula (I) is a compound of Formula (I-k): 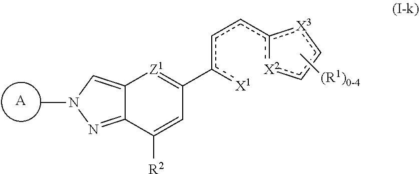
or a pharmaceutically acceptable salt, solvate, hydrate, tautomer, or stereoisomer thereof, wherein each of X¹ and X³ is independently C(R³) or N; X² is C or N; and wherein A, Z¹, R¹, R², and R³ are as defined in claim 1.

64. The compound of any one of claims 1-51, wherein the compound of Formula (I) is a compound of Formula (I-l): 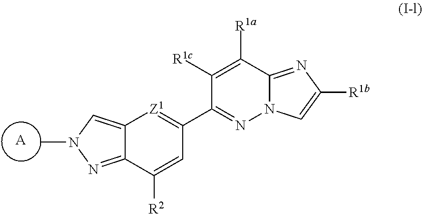
or a pharmaceutically acceptable salt, solvate, hydrate, tautomer, or stereoisomer thereof, wherein each of R^{1a}, R^{1b}, and R^{1c} is independently hydrogen, C₁-C₆-alkyl, halo, or —OR^{A}; and wherein A, R^{A}, Z¹, R¹, R², and R³ are as defined in claim 1.

65. The compound of any one of claims 1-51, wherein the compound of Formula (I) is a compound of Formula (I-m): 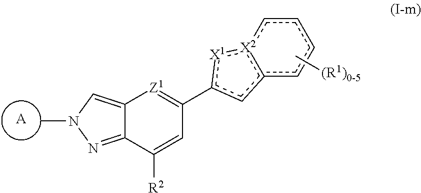
or a pharmaceutically acceptable salt, solvate, hydrate, tautomer, or stereoisomer thereof, wherein each of X¹ and X³ is independently C(R¹) or N; X² is C or N; and wherein A, Z¹, R¹ and R² are as defined in claim 1.

66. The compound of any one of claims 1-51, wherein the compound of Formula (I) is a compound of Formula (I-n): 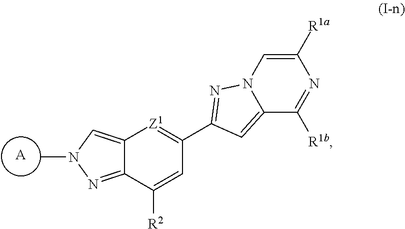
or a pharmaceutically acceptable salt, solvate, hydrate, tautomer, or stereoisomer thereof, wherein each of R^{1a} and R^{1b} is independently hydrogen, C₁-C₆-alkyl, halo, or —OR^{A}; and wherein A, R^{A}, Z¹, R¹, R², and R³ are as defined in claim 1.

67. The compound of any one of claims 1-51, wherein the compound of Formula (I) is a compound of Formula (I-0): 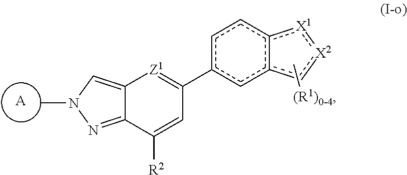
or a pharmaceutically acceptable salt, solvate, hydrate, tautomer, or stereoisomer thereof, wherein each of X¹ and X² is independently C(R¹) or N; and wherein A, Z¹, R¹ and R² are as defined in claim 1.

68. The compound of any one of claims 1-51, wherein the compound of Formula (I) is a compound of Formula (I-p): 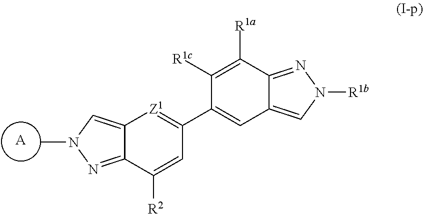
or a pharmaceutically acceptable salt, solvate, hydrate, tautomer, or stereoisomer thereof, wherein each of R^{1a}, R^{1b}, and R^{1c} is independently hydrogen, C₁-C₆-alkyl, halo, or —OR^{A}; and
wherein A, R^{A}, Z¹, R¹ and R² are as defined in claim 1.

69. The compound of any one of claims 1-51, wherein the compound of Formula (I) is a compound of Formula (I-q): 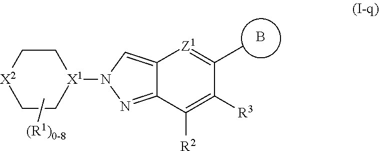
or a pharmaceutically acceptable salt, solvate, hydrate, tautomer, or stereoisomer thereof, wherein X¹ is C(R¹); X² is C(R¹)₂ or N(R¹); and wherein B, Z¹, R¹, R² and R³ are as defined in claim 1.

70. The compound of any one of claims 1-51, wherein the compound of Formula (I) is a compound of Formula (I-r): 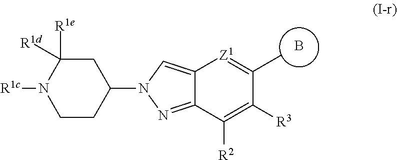
or a pharmaceutically acceptable salt, solvate, hydrate, tautomer, or stereoisomer thereof, wherein each of R^{1c}, R^{1d} and R^{1e} is independently hydrogen, alkyl, halo, or heteroalkyl; or R^{1d} and R^{1c} together with the atoms to which they are attached, form a three or four membered cycloalkyl group; and wherein each of B, Z¹, Z², R², and R³ are as defined as in claim 1.

71. The compound of any one of claims 1-51, wherein the compound of Formula (I) is a compound of Formula (I-s): 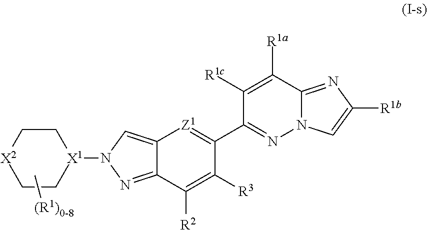
or a pharmaceutically acceptable salt, solvate, hydrate, tautomer, or stereoisomer thereof, wherein X¹ is C(R¹); X² is C(R¹)₂ or N(R¹); each of R^{1a}, R^{1b}, and R^{1c} is independently hydrogen, C₁-C₆-alkyl, halo, or —OR^{A} and wherein R^{A}, Z¹, R¹, R², and R³ are as defined in claim 1.

72. The compound of any one of claims 1-51, wherein the compound of Formula (I) is a compound of Formula (I-t): 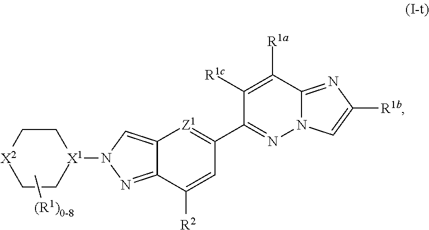
or a pharmaceutically acceptable salt, solvate, hydrate, tautomer, or stereoisomer thereof, wherein X¹ is C(R¹); X² is C(R¹)₂ or N(R¹); each of R^{1a}, R^{1b}, and R^{1c} is independently hydrogen, C₁-C₆-alkyl, halo, or —OR^{A} and wherein R^{A}, Z¹, R¹, and R² are as defined in claim 1.

73. The compound of any one of claims 1-51, wherein the compound of Formula (I) is a compound of Formula (I-u): 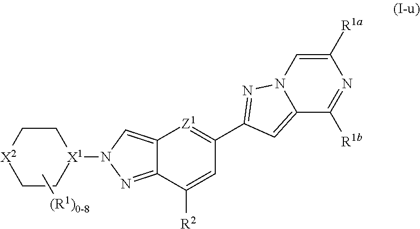
or a pharmaceutically acceptable salt, solvate, hydrate, tautomer, or stereoisomer thereof, wherein X¹ is C(R¹); X² is C(R¹)₂ or N(R¹); each of R^{1a} and R^{1b} is independently hydrogen, C₁-C₆-alkyl, halo, or —OR^{A} and wherein R^{A}, Z¹, R¹, and R² are as defined in claim 1.

74. The compound of any one of claims 1-51, wherein the compound of Formula (I) is a compound of Formula (I-v): 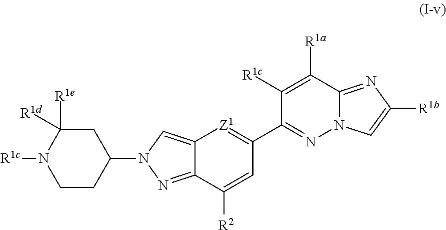
or a pharmaceutically acceptable salt, solvate, hydrate, tautomer, or stereoisomer thereof, wherein each of R^{1a}, R^{1b}, R^{1c}, R^{1d}, R^{1e}, and R^{1f} is independently hydrogen, C₁-C₆-alkyl, halo, or R^{A}; and wherein R^{A}, Z¹, R¹, and R² are as defined in claim 1.

75. The compound of the preceding claims, wherein:
A and B are each independently selected from heteroaryl (e.g., bicyclic heteroaryl) optionally substituted with R¹ and heterocyclyl (e.g., monocyclic heterocyclyl) optionally substituted with one or more R¹; and
m is 0 or 1; and
R² is C₁-C₆-alkyl, halo, or —OR^{A}.

76. The compound of any one of the preceding claims, wherein when Z¹ is N, B is bicyclic heteroaryl optionally substituted with one or more R¹, wherein at least one R¹ comprises an oxygen atom.

77. The compound of any one of the preceding claims, wherein the compound is selected from a compound listed in Table 1, or a pharmaceutically acceptable salt, solvate, hydrate, tautomer, or stereoisomer thereof.

78. A pharmaceutical composition comprising a compound of any one of the preceding claims and a pharmaceutically acceptable excipient.

79. The compound of any one of claims 1-77, or the pharmaceutical composition of claim 78, wherein the compound alters a target nucleic acid (e.g., an RNA, e.g., a pre-mRNA).

80. The compound of any one of claims 1-77, or the pharmaceutical composition of claim 78, wherein the compound binds to a target nucleic acid (e.g., an RNA, e.g., a pre-mRNA).

81. The compound of any one of claims 1-77, or the pharmaceutical composition of claim 78, wherein the compound stabilizes a target nucleic acid (e.g., an RNA, e.g., a pre-mRNA).

82. The compound of any one of claims 1-77, or the pharmaceutical composition of claim 78, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%10%, 15%, 20%, 25%, 30%, 35%, 40%, 45%, 50%, 55%, 60%, 65%, 70%, 75%, 80%, 85%, 90%, 95%, or more, e.g., as determined by qPCR.

83. The compound of any one of claims 1-77, or the pharmaceutical composition of claim 78, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

84. A method of forming a complex comprising a component of a spliceosome (e.g., a major spliceosome component or a minor spliceosome component), a nucleic acid (e.g., a DNA, RNA, e.g., a pre-mRNA), and a compound of Formula (I) or a composition thereof according to any one of claims 1-77 and the pharmaceutical composition of claim 78:
comprising contacting the nucleic acid (e.g., a DNA, RNA, e.g., a pre-mRNA) with a compound of Formula (I).

85. The method of claim 84, wherein the component of a spliceosome is recruited to the nucleic acid in the presence of the compound of Formula (I).

86. A method of altering the conformation of a nucleic acid (e.g., a DNA, RNA, e.g., a pre-mRNA) comprising contacting the nucleic acid with a compound of Formula (I) according to any one of claims 1-77, or the pharmaceutical composition of claim 78.

87. The method of claim 86, wherein the altering comprises forming a bulge in the nucleic acid.

88. The method of claim 86, wherein the altering comprises stabilizing a bulge in the nucleic acid.

89. The method of claim 86, wherein the altering comprises reducing a bulge in the nucleic acid.

90. The method of any one of any one of claims 86-89, wherein the nucleic acid comprises a splice site.

91. A method for treating a disease or disorder in a subject comprising administering to the subject a compound of Formula (I) according to any one of claims 1-77 or the pharmaceutical composition of claim 78.

92. The method of claim 91, wherein the disease or disorder comprises a proliferative disease (e.g., cancer, a benign neoplasm, or angiogenesis).

93. The method of any one of claims 91-92, wherein the proliferative disease is cancer.

94. The method of claim 91, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

95. The method of claim 94, wherein the disease or disorder comprises neurological disease or disorder.

96. The method of claim 95, wherein the disease or disorder comprises Huntington's disease.
